(19) United States
(12) Patent Application Publication
(10) Pub. No.: US 2024/0009291 A1
(43) Pub. Date: Jan. 11, 2024

(54) COMPOSITIONS AND METHODS FOR REDUCING METHANE EMISSIONS IN RUMINANT POPULATIONS

(71) Applicant: Arkea Bio Corp., Charlestown, MA (US)

(72) Inventors: Colin South, Reno, NV (US); Matthew Dunn, Louisville, CO (US); Lauren Fitch, Malden, MA (US); Joseph Koos, Somerville, MA (US); Laura Holland, Exeter, RI (US); Brigid Arciero, Boulder, CO (US); James Spoonamore, Somerville, MA (US); Caitlin Allen, Somerville, MA (US); Alexander Hsu, Belmont, MA (US); Sara Tabandeh, Cambridge, MA (US); Frederick Richard Kearney, Walpole, MA (US)

(21) Appl. No.: 18/350,526

(22) Filed: Jul. 11, 2023

Related U.S. Application Data
(60) Provisional application No. 63/524,513, filed on Jun. 30, 2023.
(60) Provisional application No. 63/359,978, filed on Jul. 11, 2022.

(51) Int. Cl. A61K 39/02; A61K 9/50; A61K 9/24; A61K 9/28; A61K 9/20; A61P 31/04

(52) U.S. Cl. CPC A61K 39/0208; A61K 9/5052; A61K 9/209; A61K 9/2866; A61K 9/205; A61K 9/2054; A61K 9/2063; A61K 9/2009; A61P 31/04; A61K 2039/552

(57) ABSTRACT

The present invention is related to compositions and methods that reduce methane production in ruminant populations.

### CROSS-REFERENCE TO RELATED APPLICATIONS

This application claims the benefit of U.S. Provisional Application No. 63/359,978, filed Jul. 11, 2022; and U.S. Provisional Application No. 63/524,513, filed Jun. 30, 2023. The entire contents of each of said applications are incorporated herein in their entirety by this reference.

### INCORPORATION BY REFERENCE OF SEQUENCE LISTING

The application contains a Sequence Listing which has been submitted electronically in the form of an XML file, created Jul. 11, 2023, and named ARD-00201_SL.xml (23953 KB), the contents of which are incorporated herein by reference in their entirety.

### BACKGROUND OF THE INVENTION

Methane (CH₄) is the world's second most abundant greenhouse gas after carbon dioxide (CO₂), accounting for 16% of total greenhouse gas emissions. The potential global warming effect of CH₄ is 28-fold higher than that of CO₂. In addition, rumen CH₄ emissions from ruminants account for 13% to 19% of the global CH₄ emissions; therefore, ruminant feeding is a major factor in exacerbating global warming. Therefore, reducing rumen CH₄ emissions could decrease the rate of global warming, which would be of great significance to efforts to reduce global greenhouse gas emissions. CH₄ emissions also represent energy losses during ruminant farming. On average, approximately 8-12% of the energy consumed in feed is wasted in the form of CH₄ emissions.

Methane is formed in the ruminant fore-stomach (rumen) by methanogens, a subgroup of the Archaea. During normal rumen function, plant material is broken down by fiber-degrading microorganisms and fermented mainly to volatile fatty acids, ammonia, hydrogen (H₂) and CO₂. Rumen methanogens principally use H₂ to reduce CO₂ to CH₄ in a series of reactions that are coupled to ATP synthesis.

Attempts have been made to inhibit the action of methanogens in the rumen using a variety of interventions but most have failed, or met with only limited success, due to low efficacy, poor selectivity, toxicity of compounds against the host, or build-up of resistance to anti-methanogen compounds. Accordingly, there is a great need in the art for effective, selective, and safe compositions and methods for inhibiting methanogens in ruminant populations.

### SUMMARY OF THE INVENTION

The present invention is based, at least in part, on the discovery that vaccines (e.g., nucleic acid vaccines) of the present disclosure against a methanogen cell surface antigen or a fragment thereof (e.g., antigenic fragment, epitope), when administered to ruminants, are surprisingly effective in inducing immune response and antibody production against the methanogen, and reducing the methane production in ruminants.

Previous attempts to vaccinate ruminants have been made using freeze-dried or formaldehyde-killed methanogens or cell wall fractions. While such vaccination induced strong antibody responses, the vaccination demonstrated only up to 8% methane reduction in vivo, largely because immunodomiant antigens present on methanogens did not represent antigens that are targets for antibody-mediated neutralization of the growth of methanogens and/or production of methane.

The vaccines of the present disclosure take a targeted approach, aiming at least one cell surface protein or a fragment thereof (e.g., antigenic fragment, epitope) of at least one methanogen that is effective in neutralizing the methanogen, thereby increasing the specificity and effectiveness of the vaccine.

### BRIEF DESCRIPTION OF FIGURES

FIG. 1A shows a schematic diagram showing an exemplary design of the nucleoside-modified mRNA-LNP vaccines. A) Design of the nucleoside-modified mRNA-LNP vaccines. The important structures of mRNA are the 5′ cap (e.g., the 7-methylguanosine cap), the 5′ and 3′ untranslated regions (UTRs), sequence encoding at least one cell surface protein or a fragment thereof of at least one methanogen, and the polyA tail. mRNA cap is incorporated either in one step during transcription in the presence of CAP analogs (e.g., Clean-Cap) or in two steps, after IVT mRNA production, by enzymatic capping reaction. Replacement of native nucleosides in in-vitro-transcribed mRNA with chemically modified versions reduces immunogenicity and increases translation efficiency. A vaccine may comprise nucleoside-modified transcripts with substitution of uridines for N1-methyl pseudouridine is (1mψ). Each of these structural elements of mRNA can be optimized and modified to modulate the stability, translation capacity, and immunestimulatory profile of mRNA. B) Schematic depiction of mRNA vaccine encapsulated into LNP formulations for improved in vivo mRNA delivery, which are typically composed of (1) an ionizable or cationic lipid (e.g., SM-102 or ALC-0315), bearing tertiary or quaternary amines to encapsulate the polyanionic mRNA; (2) a helper lipid (e.g., helper lipid 1,2-distearoyl-snglycero-3-phosphocholine (DSPC)) that resembles the lipids in the cell membrane; (3) cholesterol to stabilize the lipid bilayer of the LNP; and (4) a polyethylene glycol (PEG)-lipid [e.g., (2-[(polyethylene glycol)-2000]-N,N-ditetradecylacetamide (PEG2000-DMA), or 1,2-dimyristoyl-rac-glycero3-methoxypolyethylene glycol-2000 (PEG2000-DMG)] to lend the nanoparticle a hydrating layer, improve colloidal stability, and reduce protein absorption. Adapted from Granados-Riveron and Aquino-Jarquin (2021) Biomedicine & Pharmacotherapy 142:111953.

FIG. 1B shows a schematic diagram of an exemplary RNA vaccine.

FIG. 2A-FIG. 2B show an exemplary IVT mRNA formulated in lipid nanoparticle vaccines using a cell-free production. FIG. 2A shows in vitro transcribed (IVT) mRNA contains five structural elements: a 5′ cap containing 7-methylguanosine linked through a triphosphate bridge to a 2′-O-methylated nucleoside, flanking 5′ and 3′ untranslated regions (UTRs), an open reading frame (ORF) and a polyA tail. FIG. 2A shows mRNA that is synthetically produced and formulated into vaccines. (1) Once the sequence of at least one cell surface protein of at least one methanogen is identified, said sequence is designed and inserted into a plasmid DNA construct. (2) Plasmid DNA is transcribed into mRNA by bacteriophage polymerases in vitro and (3) mRNA transcripts are purified by high performance liquid chromatography (HPLC) to remove contaminants and reactants. (4) Purified mRNA is mixed with lipids in a microfluidic mixer to form lipid nanoparticles. Rapid mixing causes the lipids to encapsulate mRNA instantaneously and precipitate as self-assembled nanoparticles. (5) The nanoparticle solution is dialysed or filtered to remove non-aqueous solvents and any unencapsulated mRNA and (6) the filtered mRNA vaccine solution is stored in sterilized vials. Adapted from Chaudhary et al. (2021) Nature Reviews, 20:817-838.

FIG. 3A-FIG. 3B show representative mRNA delivery vehicles contain cationic or ionizable molecules. FIG. 3A shows lipid nanoparticles encapsulate mRNA in their core. They consist of four components: ionizable lipids, e.g., DLin-MC3-DMA, SM-102, ALC-0315, A18-Iso5-2DC18, A6, or 3060i10; cholesterol or its variants, e.g., β-sitosterol or 20α-hydroxycholesterol; helper lipids, e.g., DSPC and DOPE; and PEGylated lipids, e.g., ALC-0159, PEG-DMG. FIG. 3B (upper panel) shows polymers, e.g., PEI, PBAE, PEG-PAsp(DET), or CART form polymer-mRNA complexes. FIG. 3B (bottom panel) shows cationic nanoemulsions containing a squalene core surrounded by an outer shell made of cationic lipid (for example, DOTAP) and surfactants, such as Tween 80 and Span 85. The mRNA adsorbs to the surface via electrostatic binding. Adapted from Chaudhary et al. (2021) Nature Reviews, 20:817-838.

FIG. 4 shows an exemplary mechanism by which immune response is elicited by an mRNA vaccine. For example, messenger RNA vaccines elicit immunity through transfection of antigen-presenting cells. (1) Injected mRNA vaccines are endocytosed by antigen-presenting cells. (2) After escaping the endosome and entering the cytosol, mRNA is translated into protein by the ribosome. The translated antigenic protein can stimulate the immune system in several ways. (3) Intracellular antigen is broken down into smaller fragments by the proteasome complex, and the fragments are displayed on the cell surface to cytotoxic T cells by major histocompatibility complex (MHC) class I proteins. (4) Activated cytotoxic T cells kill infected cells by secreting cytolytic molecules, such as perforin and granzyme. (5) Additionally, secreted antigens can be taken up by cells, degraded inside endosomes and presented on the cell surface to helper T cells by MHC class II proteins. (6) Helper T cells facilitate the clearance of circulating pathogens by stimulating B cells to produce neutralizing antibodies, and by activating phagocytes, such as macrophages, through inflammatory cytokines. BCR, B cell receptor; ER, endoplasmic reticulum; TCR, T cell receptor. Adapted from Chaudhary et al. (2021) Nature Reviews, 20:817-838.

FIG. 5 illustrates administration of a nucleic acid vaccine (501) encoding a methanogen cell surface protein to a ruminant (502), such as a cow. Once administered, expression of the methanogen cell surface protein elicits an immune response generating salivary antibodies (503) generated in the saliva (504) that are delivered to the rumen (505). In the rumen, salivary antibodies (503) bind to an reduce the fitness of ruminal methanogens (506) thereby decreasing eructated methane (507).

FIG. 6 illustrates an exemplary testing methodology. Nucleic acids, e.g., mRNA, (601) are mixed with lipids (602) to form lipid nanoparticles (603). The lipid nanoparticles (603) are formulated into a vaccine (604) and used to vaccinate a ruminant (605), such as a cow. Blood (606) is collected and clarified into serum (607). Sera antibodies (608) are combined with methanogens (609) and the result change in fitness is measured.

FIG. 7A shows an illustrative schematic of various nucleic acid vaccine constructs: (top) comprising a 5′UTR, a sequence encoding an antigen, a 3′ UTR, and a poly A tail; (middle) a 5′UTR, a signal peptide (SP) a sequence encoding an antigen, a 3′ UTR, and a poly A tail; (bottom) a 5′UTR, a signal peptide (SP) a sequence encoding an antigen, a transmembrane domain, a 3′ UTR, and a poly A tail.

FIG. 7B shows an illustrative schematic of various protein expression and antigen presentation modalities: (701) cytosolic protein expression and antigen presentation to the immune system through major histocompatibility complex; (702) protein expression and secretion for antigen presentation to the immune system; (703) protein expression and anchoring on the extracellular surface of the cell membrane for presentation to the immune system.

FIG. 8A shows encapsulation of mRNA (801) followed by particle size and dispersity analysis (802).

FIG. 8B shows particle dispersity (y-axis) and size (x-axis) for exemplary LNP preparations.

FIG. 9 shows relative IgG (y-axis; antigen treated/empty treated) for 9 animals vaccinated with three exemplary mRNA constructs.

FIG. 10 shows average OVA-specific IgG (y-axis) as measured by ELISA for animals treated with ARK015 LNPs following intramuscular or subcutaneous vaccination.

FIG. 11 shows Methanogen abundance in ruminant microbiome samples. Methanbrevibacter ruminantium and Methanobrevibacter gottschalkii comprise ˜65% of methanogens in the ruminal microbiome.

FIG. 12 shows particle size and polydispersity of various nucleic acid LNP vaccines.

FIG. 13 shows particle size and polydispersity of various nucleic acid LNP vaccines.

FIG. 14 shows denaturing agarose gel electrophoresis analysis of various nucleic acid LNP vaccines.

FIG. 15 shows an exemplary methodology for measuring methane production from methanogen monocultures treated with sera collected from vaccinated animals. Briefly, BY medium is inoculated with a methanogen-of-interest and combined with serum. The culture tube is crimped closed and pressurized with 80% hydrogen, 20% carbon dioxide. The cultures are incubated and methane in measured intermittently using a GazoMat handheld methane monitoring device. Methane is plotted as a function of time for the various treatment groups and the control group.

FIG. 16A shows species selective reduction in methane production when treated with vaccinated sera. Briefly, animals were vaccinated with ARK007, a nucleic acid vaccine encoding a protein present in M. ruminantium but not M. gottschalkii. Methane production (y-axis) over time (x-axis) is plotted for a culture of Methanobrevibacter ruminantium combined with a serum sample collected at day 14 after vaccination with ARK007 (light gray, triangles) compared to the same animal pre-vaccination (dark gray, circles). Methane production rate is reduced by 3× in the sample treated with the day 14 serum. Methane reaches maximal production at ˜300 hrs in the sample treated with the day 14 serum as compared to ˜100 hrs when treated with pre-vaccination serum.

FIG. 16B shows methane production over time for a culture of Methanobrevibacter gottschalkii treated with the same sera samples. There is no notable reduction in methane production for M. gottschalkii when treated with either the day 14 or pre-vaccination sera. This demonstrates the ability to generate highly specific and selective antibodies by vaccination with nucleic acids encoding methanogen cell surface proteins.

FIG. 17A shows increased reduction in methane production by monocultures of Methanobrevibacter ruminantium when treated with sera collected pre-vaccination, after a first injection on day 0, and after a boost injection on day 21. Methane production rate is reduced ˜2× when treated with serum collected after a single vaccination (day 21) and ˜20× when treated with serum collected after a boost vaccination (day 35).

FIG. 17B shows reduction is methane production rate when treated with a small molecule inhibitor of methanogenesis (lauric acid) at 3 different concentrations. This demonstrates that the vaccinated sera samples are preforming with similar efficacy to known small molecule inhibitors of methanogenesis.

FIG. 18 shows a computational pipeline for selection of Methanobrevibacter ruminantium and Methanobrevibacter gottschalkii cell surface proteins for vaccine production. The pipeline includes collation of all ORFs, filtering based on cell localization, filtering based on transcriptomic analysis of genes expressed during small molecule challenge, and then filtered for function.

FIG. 19 shows a biochemical map of methanogenesis-related proteins. Several proteins identified are involved in metal uptake, specifically molybdate, cobalt, nickel, and iron, which are critical for methanogenesis.

FIG. 20 shows an exemplary methodology for characterizing nucleic acid vaccines encoding methanogen cell surface proteins.

FIG. 21A shows a computational pipeline (left) for selection of Methanogen cell surface proteins for vaccine production. The pipeline includes collation of all ORFs, filtering based on uniqueness to methanogens, cell localization, and function. A network map of key methanogen cell surface protein functions is shown to the right.

FIG. 21B shows a schematic representation of the protein fragments described in Table 21. The amino acid sequence of each folded domain is designated as a “protein fragment” in Table 21, each of which is useful as an antigen for the vaccines of the present disclosure.

FIG. 22 An exemplary vaccination protocol. In some embodiments, at least one vaccine (e.g., a nucleic acid vaccine comprising a nucleic acid sequence encoding at least one methanogen surface antigen) is prepared. The at least one vaccine is used to vaccinate a pregnant animal (e.g., ruminant). The unborn fetus is exposed to the antibodies against methanogen generated by the mother. After birth, the child ruminant receives milk laden with the antibodies against the at least one methanogen surface antigen. The child ruminant is vaccinated with at least one vaccine of the present disclosure prior to weaning. The child ruminant may be vaccinated with a vaccine encoding the same or different methanogen surface antigen.

FIG. 23 An exemplary vaccination protocol. In some embodiments, at least one vaccine (e.g., a nucleic acid vaccine comprising a nucleic acid sequence encoding at least one methanogen surface antigen) is prepared. A ruminant is treated with a combinatory therapy, which comprises any two or more selected from a vaccine, antibodies, milk, animal feed, an agent (e.g., an agent that reduces methane production in a ruminant, a probiotic bacterial strain, a small molecule inhibitor, etc.), and other composition of the present disclosure (e.g., those reducing methane production in a ruminant). Here, a reduction in methane production that is greater than the use of a single therapy alone is achieved.

FIG. 24 is a schematic of a biochemical pathway and enzymes for the production of methane from acetate (i.e., the acetoclastic pathway), hydrogen and carbon dioxide (i.e., the hydrogenotrophic pathway), and methanol and derivatives thereof (i.e., the methylotrophic pathway).

FIG. 25 is a schematic describing the reduction of methyl-CoM and coenzyme B into methane by methyl-coenzyme M reductase (MCR), a key enzyme present in methane production via the acetoclastic, hydrogenotrophic, and methylotrophic pathways.

FIG. 26 is a schematic describing the application of small molecules that affect MCR activity to modulate methane production.

FIG. 27 is a cross-sectional diagram of the application of a composition for reduction of deleterious atmospheric gases and/or precursors thereof to a flooded ecosystem.

FIG. 28 shows aerial delivery of a composition for reduction of deleterious atmospheric gases and/or precursors thereof to a flooded ecosystem.

FIG. 29 shows an illustrative example of the application of a composition for reduction of deleterious atmospheric gases and/or precursors thereof throughout the rice lifecycle.

FIG. 30 shows 3-NOP concentration (mM) v. adsorbent (˜20 mM stock solution).

FIG. 31 shows 3-NOP concentration (mM) v. adsorbent (˜8 mM stock solution).

FIG. 32 shows exemplary multilayer polyelectrolyte coatings of 15% activated carbon tablets.

FIG. 33 shows exemplary multilayer polyelectrolyte coatings of 25% activated carbon tablets.

FIG. 34 shows exemplary multilayer polyelectrolyte coatings of 15% activated carbon tablets with 5% sodium lignosulfonate.

FIG. 35 shows exemplary multilayer polyelectrolyte coatings of 15% activated carbon with 5% hydroxypropyl cellulose.

FIG. 36 is a graph showing the release profiled of a silica v. activated carbon adsorbent.

FIG. 37 is a table showing the composition of various polycaprolactone-based formulations according to some embodiments of the invention.

FIG. 38 is a bar graph showing 3-NOP release in mM for exemplary polycaprolactone-based formulations.

FIG. 39 is a graph showing normalized 3-NOP concentration v. release time in days of exemplary polybutylene succinate-based formulations.

FIG. 40 is a bar graph showing 3-NOP release (%) from exemplary PEC microcapsules. Samples are prepared with a 3-NOP concentration of 100 μM. Final pH solution ˜7. K, L, F, and E refer to lysine, leucine, phenylalanine, and glutamic acid, respectively. PSS refers to polystyrene sulfonate.

FIG. 41 is bar graph showing 3-NOP release (%) from exemplary PEC microcapsules. Samples are prepared with a 3-NOP concentration of 100 μM. Final pH solution ˜7. PLR refers to poly (L-arginine), and PLK refers to poly (L-lysine). SLS and PSS refer to sodium lignosulfonate and polystyrene sulfonate.

FIG. 42A-FIG. 42B are bar graphs showing representative ELISA data of the total IgG level (FIG. 42A) and total IgGA level (FIG. 42B) in the serum of the cows that have been vaccinated with lipid nanoparticles (LNPs) comprising one of ARK001-ARK010, ARK012, ARK013, and ARK015. Also shown are the ELISA data for cows vaccinated with empty LNPs. Three cows were vaccinated with each sample. The numbers 1-5 on the X axis correspond to Day 0, Day 14, Day 21, Day 28, and Day 35, respectively, since the time of vaccination. Each “TAMU” number refers to an individual cow.

### DETAILED DESCRIPTION OF THE INVENTION

Provided herein are vaccines (e.g., nucleic acid vaccines) against at least one cell surface antigen or a fragment thereof (e.g., antigenic fragment, epitope) of at least one methanogen, which are effective in inducing immune response and antibody production against the methanogen antigen, and reducing the methane production in ruminants.

### Definitions

The articles “a” and “an” are used herein to refer to one or to more than one (i.e. to at least one) of the grammatical object of the article. By way of example, “an element” means one element or more than one element.

As used herein, the term “about” when used before a numerical designation, e.g., temperature, time, amount, concentration, and such other, including a range, indicates approximations which may vary by (+) or (−) 10%, 9%, 8%, 7%, 6%, 5%, 4%, 3%, 2%, or 1%. The term “administering” is intended to include routes of administration which allow an agent (e.g., a vaccine composition, an agent that reduces methane production in a ruminant) to perform its intended function. Examples of routes of administration which can be used include injection (subcutaneous, intravenous, parenteral, intraperitoneal, intrathecal, intradermal, intramuscular, etc.), oral, inhalation, and transdermal routes. The injections can be bolus injections or can be continuous infusion. Depending on the route of administration, the agent (e.g., a vaccine composition, an agent that reduces methane production in a ruminant) can be coated with or disposed in a selected material to protect it from natural conditions which may detrimentally affect its ability to perform its intended function. The agent may be administered alone, or in conjunction with a pharmaceutically acceptable carrier or adjuvant. The agent (e.g., a vaccine composition, an agent that reduces methane production in a ruminant) also may be administered as a prodrug, which is converted to its active form in vivo.

The term “conjoint” or “combination” administration, as used herein, refers to the administration of two or more agents that aid in reducing methane production in a ruminant. The different agents comprising the combination may be administered concomitant with, prior to, or following the administration of one or more agents.

The term “fragment,” as used herein encompasses any and all that is less than the full length. In some embodiments, a fragment of a polypeptide of the present disclosure is an antigenic fragment of the polypeptide. Such antigenic fragment may comprise at least one epitope that binds to the antibody. In preferred embodiments, a fragment of a polypeptide of the present disclosure is a fragment of the polypeptides that is effective in eliciting immune response and/or inducing antibody production when administered to a ruminant.

The term “feed additive” refers to any agent or composition that reduces the methane production in a ruminant, which can be added to an animal feed.

The term “methanogen,” as used herein, refers to a microorganism that produces methane as a metabolic byproduct. Methanogens belong to the domain Archaea, and include, but are not limited to those of a family Methanobacteriaceae, e.g., those of genera Methanobrevibacter, Methanosphaera, Methanomicrobium, Methanobacterium, Methanocorpusulum, Methanosaeta, Methanoculleus, Methanosarcina, and Thermoplasmatales. Specific methanogens include, but are not limited to, Methanobrevibacter ruminantium (i.e., the M1 strain or strain DSM 1093 (see e.g., World Wide Web at dsmz.de/microorganisms/html/strains/strain.dsm001093. htm). Additional relevant species are further described below.

The term “nucleic acid” (also called “polynucleotide”) in its broadest sense, includes any compound and or substance that comprise a polymer of nucleotides linked via a phospohdiester bond. The nucleotides or a portion thereof may be natural or synthetic; or may be structurally or chemically modified.

The term “polydispersity index” is a ratio that describes the homogeneity of the particle size distribution of a system. A small value, e.g., less than 0.3, indicates a narrow particle size distribution.

The term “ruminant” refers to a hoofed herbivorous grazing or browsing mammal that is able to acquire nutrients from plant-based food by fermenting it in a specialized stomach prior to digestion, principally through microbial actions. The process, which takes place in the front part of the digestive system and therefore is called foregut fermentation, typically requires the fermented ingesta (known as cud) to be regurgitated and chewed again. The roughly 200 species of ruminants include both domestic and wild species. Ruminants include, but are not limited to, cattle (e.g., large domesticated ruminant animals, e.g., cows (including dairy cattle), bulls), all domesticated and wild bovines (i.e., those belonged to the family Bovidae; e.g., cows, bulls, bisons, yaks, African buffalos, water buffalos, antelopes), goats, sheep, giraffes, deer, caribou, and gazelles. In preferred embodiments, ruminants are domesticated. As used herein, the term “ruminant” includes ruminant-like animals or pseudo-ruminant animals such as macropods, llamas, camels, and alpacas. In some embodiments, a ruminant has not been administered with an agent that reduces methane. In other embodiments, a ruminant has been administered or is being administered with an agent that reduces methane.

As used herein, the term “valency” refers to the number of antigenic components in the nucleic acid vaccine or nucleic acid polynucleotide (e.g., DNA or RNA polynucleotide) or polypeptide. In some embodiments, the nucleic acid vaccines are monovalent. In some embodiments, the nucleic acid vaccines are divalent. In some embodiments the nucleic acid vaccines are trivalent. In some embodiments the nucleic acid vaccines are multi-valent. Multivalent vaccines may comprise 2, 3, 4, 5, 6, 7, 8, 9, 10, 11, 12, 13, 14, 15, 16, 17, 18, 19, 20, or more antigens or antigenic moieties (e.g., antigenic peptides, etc.). The antigenic components of the nucleic acid vaccines may be in a single nucleic acid molecule or in separate nucleic acid molecules.

### Methanogens

The diversity of the rumen methanogens is much smaller, and their diversity is much lower than that of rumen bacteria, with archaeal SSU rRNA only accounting for 6.8% of rumen total SSU rRNA. Archaea in the rumen is represented by <3.3% of the total rRNA (both 16S and 18S) therein. Representative family of methanogens includes Methanobacteriaceae.

Representative genera of methanogens include Methanobrevibacter, Methanosphaera, Methanomicrobium, Methanobacterium, Methanocorpusulum, Methanosaeta, Methanoculleus, Methanosarcina, and Thermoplasmatales.

Certain species of ruminal methanogens have been isolated into pure cultures: Methanobacterium formicicum, Methanobacterium bryantii, Methanobrevibacter ruminantium, Methanobrevibacter millerae, Methanobrevibacter olleyae, Methanomicrobium mobile, Methanoculleus olentangyi, and Methanosarcina barkeri. Additional species have been recently isolated, including Methanobrevibacter boviskoreani (isolated from the rumen of Korean native cattle), Methanobacterium beijingense (isolated from the rumen of goat), Methanoculleus marisnigri (isolated from the rumen of Indian crossbred cattle), Methanoculleus bourgensis (isolated from the rumen of Holstein cattle), and Methanosarcina mazei (isolated from the rumen of Korean Hanwoo cattle) (based on the RDP database). A Thermoplasmatales-like pyrrolysine-dependent archaeon BRNA1 was also isolated from bovine (GenBank access number: CP002916).

Collectively, 16S rRNA gene sequences from cultured methanogens only accounted for approximately 0.7% of the total archaeal sequences of rumen origin, and several taxa do not have a single cultured representative. Most of the isolates are members of the family Methanobacteriaceae. Compared to other anaerobic habitats where >100 species of methanogens of 28 genera have been isolated, the diversity and species richness of ruminal methanogens are quite low, reflecting the highly selective ruminal environment for methanogens. In addition, sequenced ruminal 16S rRNA gene clones shared >95% sequence similarity with that of Methanobrevibacter gottschalkii, Methanobrevibacter thaueri, Methanobrevibacter smithii and Methanosphaera stadtmanae, indicating that these species may be common ruminal methanogens.

Much of the ruminal methanogen diversity was characterized by 16S rRNA gene sequences. The RDP Release 11 (Update 3) contains 8623 archaeal 16S rRNA gene sequences of rumen origin. These sequences were generated using the Sanger sequencing technology, which produces higher sequence accuracy than NGS technologies, in 96 separate studies including 48 unpublished studies. About 90% of these sequences were assigned to methanogens. These sequences were classified to 10 known genera, with Methanobrevibacter being represented by 63.2% of all the sequences followed by Methanosphaera (9.8%), Methanomicrobium (7.7%), and Methanobacterium (1.2%). The order Thermoplasmatales, which was previously referred to as the rumen cluster C (RCC) group, is represented by 7.4% of the total archaeal sequences.

### Cell Surface Proteins of Methanogens

Provided herein is at least one cell surface protein or a fragment thereof (e.g., an antigenic fragment, e.g., a fragment comprising an epitope, e.g., a fragment comprising an extracellular domain or a portion thereof) of at least one methanogen and nucleic acid(s) encoding same that can be used in a vaccine composition (e.g., nucleic acid vaccine), which can elicit immune response, antibody production, and antibody-mediated neutralization of the growth of methanogens and/or production of methane.

In certain aspects, the at least one cell surface protein or a fragment thereof (e.g., an antigenic fragment, e.g., a fragment comprising an epitope, e.g., a fragment comprising an extracellular domain or a portion thereof) of at least one methanogen is of a family Methanobacteriaceae.

In some embodiments, the at least one methanogen is of a genus selected from: Methanobrevibacter, Methanosphaera, Methanomicrobium, Methanobacterium, Methanocorpusulum, Methanosaeta, Methanoculleus, Methanosarcina, and Thermoplasmatales.

In some embodiments, the at least one methanogen comprises Methanobacterium formicicum, Methanobacterium bryantii, Methanobrevibacter ruminantium, Methanobrevibacter millerae, Methanobrevibacter olleyae, Methanomicrobium mobile, Methanoculleus olentangyi, Methanosarcina barkeri, Methanobrevibacter boviskoreani, Methanobacterium beijingense, Methanoculleus marisnigri, Methanoculleus bourgensis, Methanosarcina mazei, Thermoplasmatales archaeon BRNA1, Methanobrevibacter gottschalkii, Methanobrevibacter thaueri, Methanobrevibacter smithii, Methanosphaera stadtmanae, Methanococcoides burtonii, Methanolobus psychrophilus R15, Methanobacterium paludism, Methanohalobium evestigatum, Methanomethylovorans hollandica, Methanothrix soehngenii, Methanocaldococcus vulcanius, Methanosalsum zhilinae, Methanocorpusculum labreanum, Methanoregula formicica, Methanoculleus marisnigri, Methanocella arvoryzae, Methanoculleus bourgensis, Methanolacinia petrolearia, Methanospirillum hungatei, Methanoplanus limicola, Methanohalophilus mahii, Methanococcus aeolicus, Methanosphaerula palustris, Methanocaldococcus fervens, Methanocaldococcus jannaschii, Methanocaldococcus sp. FS406-22, Methanoregula boonei, Methanobrevibacter sp. AbM4, Methanobrevibacter ruminantium, Methanosphaera, Methanobacterium formicicum, Methanocaldococcus villosus, Methanosarcina barkeri, Methanobacterium lacus, Methanotorris igneus, Methanotorris formicicus, Methanocaldococcus infernus, Methanofollis liminatans, Methanothermococcus okinawensis, Methanobrevibacter smithii, Methanobrevibacter, Methanocella conradii, Methanothermococcus thermolithotrophicus, Methanococcus maripaludis, Methanococcus maripaludis, Methanococcus vannielii, Methanothermus fervidus, Methanosarcina acetivorans, Methanosarcina mazei, Methanosaeta harundinacea 6Ac, Methanococcus maripaludis, Methanococcus voltae, Methanolinea tarda, Methanolobus psychrophilus, Methanosaeta harundinacea, or any combination thereof.

In some embodiments, the at least one methanogen comprises Methanobrevibacter ruminantium. In some embodiments, the at least one methanogen comprises Methanobrevibacter ruminantium (M1 (DSM 1093)).

In certain embodiments, a vaccine composition comprises a nucleic acid (e.g., mRNA, DNA, or a modified variant thereof) encoding at least one of cell surface protein. In some embodiments, the nucleic acid encodes at least one fragment (e.g., an antigenic fragment, an epitope) of at least one cell surface protein. In some embodiments, the nucleic acid encodes the extracellular domain, or a fragment thereof, of at least one cell surface protein. In some embodiments, the nucleic acid does not encode the transmembrane and/or intracellular domains or a fragment thereof of the at least one cell surface protein.

In some embodiments, the nucleic acid (e.g., mRNA, DNA, or a modified variant thereof) for a vaccine composition comprises at least one of the nucleic acid sequence, or a fragment thereof (e.g., those encoding at least a portion of the extracellular domain), encoding the adhesion-like proteins, adhesin-like proteins with cysteine protease domain, adhesin-like proteins with transglutaminase domain presented in Table 1. Such nucleic acids, proteins, and sequences are also provided in Leahy et al. (2010) PLoS One, 5(1):e8926 and U.S. Pat. No. 10,314,895, each of which is incorporated herein by reference. The representative nucleic acid sequences encoding the cell surface antigens of a methanogen and the representative amino acid sequences of the cell surface antigens of a methanogen are provided in Table 2A, Table 2B, Table 3, Table 19, Table 20, and Table 21.

In some embodiments, a vaccine composition comprises a polypeptide or a fragment thereof (e.g., an antigenic fragment, an epitope) of at least one cell surface protein. In some embodiments, the polypeptide comprises the extracellular domain or a fragment thereof of at least one cell surface protein. In some embodiments, the polypeptide or a fragment thereof does not comprise the transmembrane and/or intracellular domains or a fragment thereof of the at least one cell surface protein.

A person of ordinary skill in the art can determine the polypeptide sequences from the nucleic acid sequences, or determine the nucleic acid sequences from the polypeptide sequences presented herein or those known in the art.

TABLE 1
Representative cell surface antigens of a methanogen,
Methanobrevibacter ruminantium (M1 (DSM 1093))

| | | | | No. of |
|---|---|---|---|---|
| | | Nucleic Acid | Signal peptide | transmembrane |
| Locus tag | Annotation | Size (bp) | (SP) | domains |
| mru0019 | adhesin-like protein | 1220 | SP | 2 |
| mru0327 | adhesin-like protein | 2090 | SP | 2 |
| mru0687 | adhesin-like protein | 2963 | SP | 2 |
| mru1210 | adhesin-like protein | 7250 | SP | 2 |
| mru1222 | adhesin-like protein | 4055 | SP | 2 |
| mru1506 | adhesin-like protein | 857 | SP | 2 |
| mru2053 | adhesin-like protein | 3494 | SP | 2 |
| mru2134 | adhesin-like protein | 17957 | SP | 2 |
| mru2147 | adhesin-like protein | 16955 | SP | 4 |
| mru2178 | adhesin-like protein | 9239 | SP | 2 |
| mru0031 | adhesin-like protein | 4415 | Expressed | 1 |
| | | | on cell wall | |
| mru0704 | adhesin-like protein | 2858 | SP | 1 |
| mru0963 | adhesin-like protein | 8159 | Expressed | 0 |
| | | | on cell wall | |
| mru0976/0977 | adhesin-like protein | 4775 | SP | 2 |
| mru0020 | adhesin-like protein with | 6014 | SP | 1 |
| | cysteine protease domain | | | |
| mru0064 | adhesin-like protein | 3536 | SP | 1 |
| mru0072 | adhesin-like protein | 2918 | SP | 1 |
| mru0076 | adhesin-like protein | 6605 | SP | 1 |
| mru0077 | adhesin-like protein | 9161 | SP | 1 |
| mru0079 | adhesin-like protein | 3560 | SP | 1 |
| mru0083 | adhesin-like protein | 839 | SP | 1 |
| mru0084 | adhesin-like protein | 14477 | SP | 1 |
| mru0085 | adhesin-like protein | 8030 | SP | 1 |
| mru0086 | adhesin-like protein | 10175 | SP | 1 |
| mru0143 | adhesin-like protein with | 3284 | SP | 0 |
| | cysteine protease domain | | | |
| mru0160 | adhesin-like protein | 3176 | SP | 1 |
| mru0222 | adhesin-like protein with | 3302 | SP | 1 |
| | cysteine protease domain | | | |
| mru0327 | adhesin-like protein | 2060 | SP | 1 |
| mru0338 | adhesin-like protein | 6929 | SP | 1 |
| mru0417/0418 | adhesin-like protein | 1391 | SP | 1 |
| mru0419 | adhesin-like protein | 4175 | SP | 1 |
| mru0727 | adhesin-like protein with | 3788 | SP | 0 |
| | cysteine protease domain | | | |
| mru0772 | adhesin-like protein with | 3281 | SP | 1 |
| | cysteine protease domain | | | |
| mru0839 | adhesin-like protein with | 8639 | SP | 1 |
| | cysteine protease domain | | | |
| mru0842 | adhesin-like protein with | 3977 | SP | 1 |
| | cysteine protease domain | | | |
| mru0978 | adhesin-like protein | 6606 | SP | 0 |
| mru0979 | adhesin-like protein | 8753 | SP | 1 |
| mru1076 | adhesin-like protein | 2681 | SP | 1 |
| mru1077 | adhesin-like protein | 2273 | SP | 1 |
| mru1246 | adhesin-like protein | 4619 | SP | 1 |
| mru1247 | adhesin-like protein | 5060 | SP | 1 |
| mru1465 | adhesin-like protein | 2882 | SP | 1 |
| mru1513 | adhesin-like protein | 1853 | SP | 1 |
| mru1650 | adhesin-like protein | 9161 | SP | 1 |
| mru1726 | adhesin-like protein | 6767 | SP | 1 |
| mru1971 | adhesin-like protein | 1937 | SP | 1 |
| mru1996 | adhesin-like protein | 4496 | SP | 1 |
| mru2043 | adhesin-like protein | 9530 | SP | 1 |
| mru2048 | adhesin-like protein | 5417 | SP | 1 |
| mru2049 | adhesin-like protein | 10355 | SP | 1 |
| mru2052 | adhesin-like protein | 4112 | SP | 1 |
| mru2054 | adhesin-like protein | 5054 | SP | 1 |
| mru2055 | adhesin-like protein | 8906 | SP | 1 |
| mru2059 | adhesin-like protein | 4415 | SP | 0 |
| mru2090 | adhesin-like protein | 15200 | SP | 1 |
| mru0004 | adhesin-like protein | 2237 | SP | 1 |
| mru0331 | adhesin-like protein | 1622 | SP | 1 |
| mru0843 | adhesin-like protein with | 6197 | SP | 1 |
| | cysteine protease domain | | | |
| mru0015 | adhesin-like protein with | 3845 | SP | 1 |
| | cysteine protease domain | | | |
| mru0090 | adhesin-like protein | 2063 | SP | 1 |
| mru0255 | adhesin-like protein | 4187 | SP | 1 |
| mru0450 | adhesin-like protein | 803 | SP | 1 |
| mru0723 | adhesin-like protein | 7727 | SP | 1 |
| mru0962 | adhesin-like protein | 14789 | SP | 1 |
| mru0970 | adhesin-like protein | 2483 | SP | 1 |
| mru1263 | adhesin-like protein | 2585 | SP | 1 |
| mru1358 | adhesin-like protein | 2243 | SP | 1 |
| mru1386 | adhesin-like protein | 1841 | SP | 1 |
| mru1387 | adhesin-like protein with | 2957 | SP | 1 |
| | cysteine protease domain | | | |
| mru1424 | adhesin-like protein | 1445 | SP | 1 |
| mru1500 | adhesin-like protein | 3896 | SP | 1 |
| mru0493 | adhesin-like protein | 2447 | SP | 1 |
| mru0824 | adhesin-like protein with | 2027 | SP | 1 |
| | transglutaminase domain | | | |
| mru1499 | adhesin-like protein with | 3032 | SP | 0 |
| | transglutaminase domain | | | |
| mru1604 | adhesin-like protein with | 2996 | SP | 0 |
| | transglutaminase domain | | | |

| | |
|---|---|
| mru1919 | tetrahydromethanopterin S-methyltransferase subunit A MtrA1 |
| mru0441 | tetrahydromethanopterin S-methyltransferase subunit A MtrA2 |
| mru1920 | tetrahydromethanopterin S-methyltransferase subunit B MtrB |
| mru1921 | tetrahydromethanopterin S-methyltransferase subunit C MtrC |
| mru1922 | tetrahydromethanopterin S-methyltransferase subunit D MtrD |
| mru1923 | tetrahydromethanopterin S-methyltransferase subunit E MtrE |
| mru1918 | tetrahydromethanopterin S-methyltransferase subunit F MtrF |
| mru1917 | tetrahydromethanopterin S-methyltransferase subunit G MtrG |
| mru1916 | tetrahydromethanopterin S-methyltransferase subunit H MtrH |

The nucleic acid and amino acid sequence information for nucleic acid and polypeptide molecules useful in the present invention are well-known in the art and readily available on publicly available databases, such as the National Center for Biotechnology Information (NCBI).

### SEQUENCES

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA encoding a cell surface antigen nucleic acid (or any portion thereof) can be used to derive the polypeptide amino acid sequence, using the genetic code to translate the DNA or RNA into an amino acid sequence. Likewise, for polypeptide amino acid sequences, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

TABLE 2A
Representative sequences of the cell surface proteins of
Methanobrevibacter ruminantium M1 (GenBank: CP001719.1)

See SEQ ID NO. 1 to SEQ ID NO. 2217 for the native nucleic acid sequences. See SEQ
ID NO. 2218 to SEQ ID NO. 4434 for the bovine codon-optimized nucleic acid sequences. See
SEQ ID NO. 4435 to SEQ ID NO. 6651 for the aminio acid sequences.

TABLE 2B
Representative sequences of the cell surface proteins
of Methanobrevibacter gottschalkii

See SEQ ID NO. 6652 to SEQ ID NO. 8451 for the native nucleic acid sequences. See
SEQ ID NO. 8452 to SEQ ID NO. 10251 for the bovine codon-optimized nucleic acid
sequences. See SEQ ID NO. 10252 to SEQ ID NO. 12051 for the aminio acid sequences.

TABLE 3
Representative mtrE sequences of methanogens

See SEQ ID NO. 12100 to SEQ ID NO. 12147 for the nucleic acid sequences. See SEQ
ID NO. 12052 to SEQ ID NO. 12099 for the aminio acid sequences.

Included in all nucleic acid sequences disclosed herein are DNA nucleic acid molecules, RNA nucleic acid molecules (e.g., thymidine replaced with uridine), nucleic acid molecules encoding orthologs of the encoded proteins, as well as DNA or RNA nucleic acid sequences or any variant thereof (a structural variant or a chemical variant (e.g., chemically modified nucleotide)) comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO presented herein, or a portion thereof. Such nucleic acid molecules can have a function of the full-length nucleic acid (e.g., for the intended function of inducing an immune response) as described further herein.

Included in all amino acid sequences disclosed herein are amino acid sequences or any variant thereof (a structural variant or a chemical variant) comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the amino acid sequence of any SEQ ID NO listed in presented herein, or a portion thereof. Such polypeptides can have a function of the full-length polypeptide (e.g., for the intended function of inducing an immune response) as described further herein.

### Homology

Function-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A function-conservative variant also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

Homology, as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 81%, 82%, 83%, 84%, 85%, 86%, 87%, 88%, 89%, 90%, 91%, 92%, 93%, 94%, 95%, 96%, or more of the nucleotides, and more preferably at least about 97%, 98%, 99% or more of the nucleotides. Alternatively, substantial homology exists when the segments will hybridize under selective hybridization conditions, to the complement of the strand.

The percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules of the present invention. BLAST protein searches can be performed with the XBLAST program, score=50, wordlength=3 to obtain amino acid sequences homologous to the protein molecules of the present invention. To obtain gapped alignments for comparison purposes, Gapped BLAST can be utilized as described in Altschul et al., (1997) Nucleic Acids Res. 25(17):3389 3402. When utilizing BLAST and Gapped BLAST programs, the default parameters of the respective programs (e.g., XBLAST and NBLAST) can be used (available on the world wide web at the NCBI website).

### Codon Optimization

In preferred embodiments, the nucleic acid (e.g., DNA or RNA) vaccines of the present disclosure comprise those that are codon-optimized for expression in a ruminant.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database,” available World Wide Web at kazusa.or.jp/codon/, and these tables can be adapted in a number of ways. See Nakamura, Y., et al. “Codon usage tabulated from the international DNA sequence databases: status for the year 2000” Nucl. Acids Res. 28:292 (2000). As examples, a codon usage table for cow, calculated from GenBank Release 128.0, is reproduced below as Table A. Table A uses mRNA nomenclature, and so instead of thymine (T) which is found in DNA, the tables use uracil (U) which is found in RNA. The table has been adapted so that frequencies are calculated for each amino acid, rather than for all 64 codons.

TABLE A
Codon Usage Table for Cow Genes (Bos taurus)

| Amino Acid | Codon | Number | Frequency of usage |
|---|---|---|---|
| Phe | UUU | 13002 | 0.4112 |
| Phe | UUC | 18614 | 0.5888 |
| Total | | 31616 | |
| Leu | UUA | 4467 | 0.059 |
| Leu | UUG | 9024 | 0.1192 |
| Leu | CUU | 9069 | 0.1198 |
| Leu | CUC | 16003 | 0.2114 |
| Leu | CUA | 4608 | 0.0609 |
| Leu | CUG | 32536 | 0.4298 |
| Total | | 75707 | |
| Ile | AUU | 12474 | 0.3313 |
| Ile | AUC | 19800 | 0.5258 |
| Ile | AUA | 5381 | 0.1429 |
| Total | | 37655 | |
| Met | AUG | 17770 | 1 |
| Total | | 17770 | |
| Val | GUU | 8212 | 0.1635 |
| Val | GUC | 12846 | 0.2558 |
| Val | GUA | 4932 | 0.0982 |
| Val | GUG | 24222 | 0.4824 |
| Total | | 50212 | |
| Ser | UCU | 10287 | 0.1804 |
| Ser | UCC | 13258 | 0.2325 |
| Ser | UCA | 7678 | 0.1347 |
| Ser | UCG | 3470 | 0.0609 |
| Ser | AGU | 8040 | 0.141 |
| Ser | AGC | 14279 | 0.2505 |
| Total | | 57012 | |
| Pro | CCU | 11695 | 0.2684 |
| Pro | CCC | 15221 | 0.3493 |
| Pro | CCA | 11039 | 0.2533 |
| Pro | CCG | 5621 | 0.129 |
| Total | | 43576 | |
| Thr | ACU | 9372 | 0.2203 |
| Thr | ACC | 16574 | 0.3895 |
| Thr | ACA | 10892 | 0.256 |
| Thr | ACG | 5712 | 0.1342 |
| Total | | 42550 | |
| Ala | GCU | 13923 | 0.2592 |
| Ala | GCC | 23073 | 0.4295 |
| Ala | GCA | 10704 | 0.1992 |
| Ala | GCG | 6025 | 0.1121 |
| Total | | 53725 | |
| Tyr | UAU | 9441 | 0.3882 |
| Tyr | UAC | 14882 | 0.6118 |
| Total | | 24323 | |
| His | CAU | 6528 | 0.3649 |
| His | CAC | 11363 | 0.6351 |
| Total | | 17891 | |
| Gln | CAA | 8060 | 0.243 |
| Gln | CAG | 25108 | 0.757 |
| Total | | 33168 | |
| Asn | AAU | 12491 | 0.4088 |
| Asn | AAC | 18063 | 0.5912 |
| Total | | 30554 | |
| Lys | AAA | 17244 | 0.3897 |
| Lys | AAG | 27000 | 0.6103 |
| Total | | 44244 | |
| Asp | GAU | 16615 | 0.4239 |
| Asp | GAC | 22580 | 0.5761 |
| Total | | 39195 | |
| Glu | GAA | 21102 | 0.4007 |
| Glu | GAG | 31555 | 0.5993 |
| Total | | 52657 | |
| Cys | UGU | 7556 | 0.42 |
| Cys | UGC | 10436 | 0.58 |
| Total | | 17992 | |
| Trp | UGG | 10706 | 1 |
| Total | | 10706 | |
| Arg | CGU | 3391 | 0.0824 |
| Arg | CGC | 7998 | 0.1943 |
| Arg | CGA | 4558 | 0.1108 |
| Arg | CGG | 8300 | 0.2017 |
| Arg | AGA | 8237 | 0.2001 |
| Arg | AGG | 8671 | 0.2107 |
| Total | | 41155 | |
| Gly | GGU | 8508 | 0.1616 |
| Gly | GGC | 18517 | 0.3518 |
| Gly | GGA | 12838 | 0.2439 |
| Gly | GGG | 12772 | 0.2427 |
| Total | | 52635 | |
| Stop | UAA | 555 | |
| Stop | UAG | 394 | |
| Stop | UGA | 392 | |

By utilizing Table A or the like available at e.g., the website provided above, one of ordinary skill in the art can apply the frequencies to any given polypeptide sequence, and produce a nucleic acid fragment of a codon-optimized coding region which encodes the polypeptide, but which uses codons more optimal for a given species.

Accordingly, the term “codon-optimized” encompasses any modification of the nucleic acid sequence to comprise at least one codon that is more frequently used in a given ruminant. The term “codon-optimized” is not intended to mean that all codons in the nucleic acid are optimized for expression in a given ruminant.

### Protein Vaccines

In certain aspects, provided herein are vaccines that utilize peptides and/or polypeptides that comprise the sequence of at least one cell surface protein of at least one methanogen. Such peptides and/or polypeptides can be chemically synthesized, or produced using an expression vector (e.g., bacteria, yeast, insect cells, mammalian cells) or in vitro translated, e.g., via methods described herein and/or those known in the art.

The protein vaccines of the present disclosure may comprise a single peptide or a single polypeptide. Alternatively, the protein vaccines of the present disclosure may comprise a mixture of various peptides and/or polypeptides that target at least 1, 2, 3, 4, 5, 6, 7, 8, 9, 10, 11, 12, 13, 14, 15, 16, 17, 18, 19, or 20 cell surface protein of at least 1, 2, 3, 4, 5, 6, 7, 8, 9, 10, 11, 12, 13, 14, 15, 16, 17, 18, 19, or 20 methanogens. The protein vaccines of the present disclosure may be administered in a pharmaceutical composition described herein. The protein vaccines of the present disclosure may be administered with an adjuvant and/or other agents that enhance immune response. Multiple dosings of the protein vaccine is contemplated herein as described.

### Nucleic Acid Vaccines

Provided herein are nucleic acid vaccines that induce an immune response against at least one cell surface protein or a fragment thereof of at least one methanogen. The nucleic acid may be DNA or RNA; in both cases it provides the instructions for making a specific protein (e.g., an antigenic fragment of at least one cell surface protein of at least one methanogen), which the immune system will recognize as foreign. Once inserted into host cells, this nucleic acid is read by the cell's own protein-making machinery and used to manufacture the protein, which then trigger an immune response.

As used herein, the term “nucleic acid” (also called “polynucleotide”) in its broadest sense, includes any compound and or substance that comprise a polymer of nucleotides linked via a phospohdiester bond. The nucleotide or a portion thereof may be natural or synthetic. In some embodiments, the nucleotide or a portion thereof may be structurally or chemically modified.

Exemplary nucleic acids include ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA), or hybrids or combinations thereof. They may comprise RNAi-inducing agents, RNAi agents, siRNAs, shRNAs, miRNAs, antisense RNAs, ribozymes, catalytic DNA, tRNA, RNAs that induce triple helix formation, aptamers, vectors, etc.

In some embodiments, the nucleic acid comprises coding sequences and non-coding sequences.

In some embodiments, the shortest length of a nucleic acid of the present disclosure can be the length of the nucleic acid sequence that may be sufficient to encode for a dipeptide. In some embodiments, the length of the nucleic acid sequence may be sufficient to encode for a tripeptide. In some embodiments, the length of the nucleic acid sequence may be sufficient to encode for a tetrapeptide. In some embodiments, the length of the nucleic acid sequence may be sufficient to encode for a pentapeptide. In some embodiments, the length of the nucleic acid sequence may be sufficient to encode for a hexapeptide. In some embodiments, the length of the nucleic acid sequence may be sufficient to encode for a heptapeptide. In some embodiments, the length of the nucleic acid sequence may be sufficient to encode for an octapeptide. In some embodiments, the length of the nucleic acid sequence may be sufficient to encode for a nonapeptide. In some embodiments, the length of the nucleic acid sequence may be sufficient to encode for a decapeptide. In some embodiments, the length of the nucleic acid (e.g., mRNA) sequence may be sufficient to encode an antigen that triggers the immune response in a ruminant.

Generally, the length of a nucleic acid of the present disclosure may be greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the nucleic acid of the present invention includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In some embodiments, the nucleic acid of the present disclosure may comprise sequences that encode at least one peptide or polypeptide of interest. In some embodiments, the nucleic acid of the present disclosure may comprise sequences that are non-coding.

In some embodiments, the length of a portion/region of the nucleic acid encoding at least one peptide polypeptide of interest is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

### DNA Vaccine

The first proof of concept of a DNA vaccine was made in 1990 and involved the injection of DNA molecules, expressing chloramphenicol acetyltransferase, luciferase, and beta-galactosidase into mouse skeletal muscle, and the expression of reporter genes in vivo, which can be detected for up to two months after infection. In brief, DNA vaccine consists of delivering genes or fragments of it, encoding immunogenic antigens to the host's cells by using DNA plasmids as a vector. This approach induces both humoral and cell-mediated immune responses efficiently. The vaccine formulation is made such that the genetic material is translocated to the host's cell nucleus. Once it reaches there, the mammalian promoter present in the vector structure is activated, triggering the transcription of the gene used for the vaccine through the host's cellular machinery. The antigen-presenting cells (APCs) are the major target cells to receive the genetic material. In addition, myocytes have been reported to play a crucial role. After the translation of the translocated gene into a protein or protein fragment, it is further processed into peptides that bind to major histocompatibility complex (MHC) class I or II. Cells other than APC, such as the myocytes, use MHCI for the antigen presentation, and APC, such as dendritic cells (DCs), can use MHC-II, resulting in cross-priming and presentation of antigens to both CD4+ and CD8+ T cells. In addition to this cellular immune response, humoral responses can be triggered if the produced immunogen is released from the cells and recognized by B cell receptors.

In addition, intrinsic elements of plasmid DNA, such as CpG unmethylated sequences, can activate innate immune responses, thereby enhancing adaptive immune responses against the expressed antigens. Clinical trials using DNA vaccines in humans induced both cellular and humoral responses. DNA vaccines have been licensed for use in veterinary medicine (see e.g., Silveira et al. (2017) Vaccine, 35:5559-5567; Ingolotti et al. (2014) Expert Review of Vaccines, 747-763), making it an appropriate way of vaccinating a ruminant.

Compared with traditional live or attenuated vaccines, DNA vaccines have several advantages, such as induction of broad immune responses without any risk being associated with replicating microorganisms; stimulation of both cellular and humoral immunity; construction of a vector encoding different antigens in a single vaccine; efficient largescale, low-cost, production; and high storage stability. In the vaccinology field, storage is a crucial factor, as preserving the high quality of the vaccine contents and, thus, the protective potential is necessary. Hence, cold storage is essential to ensure the survival of live vaccines and preserve their content. On the other hand, DNA vaccines are highly stable and have less need for refrigeration, which may be highly practical for use in endemic areas.

### Chemical Modification(s) of Nucleic Acid (e.g., DNA)

In some embodiments, the nucleic acid may be modified (e.g., with chemical modification). In some embodiments, such chemical modification(s) protect the nucleic acid (e.g., DNA) from being degraded by DNA exonucleases. Exemplary modifications are further described below.

### 1. Phosphorothioate Linkages

A phosphorothioate (pt) bond is a phosphodiester linkage where one of the two non-bridging oxygens has been replaced by a sulfur. This modification has been used for decades to inhibit nuclease phosphodiesterase and phosphoryl transferase activities. Chemically, the substitution of oxygen with sulfur does not dramatically change the reactivity of the bond, and pt-containing polynucleotides can still function in many enzymatic reactions. In a typical phosphodiester bond, the two non-bridging oxygens are chemically equivalent. When one of these oxygens is replaced by sulfur, however, the phosphorus is now connected to four distinct groups, rendering it a chiral center with two possible configurations referred to as “S_{P}” and “R_{P}.” It is this key feature that confers resistance for the majority of nuclease enzymes; one configuration will react at rates similar to a phosphodiester, while the other is significantly inhibitory or completely unreactive. Isomer reactivity varies from enzyme to enzyme, and different pt isomers can inhibit enzymes that catalyze the same reaction (e.g., phosphoryl transfer). For example, DNA Polymerase I can incorporate deoxynucleotide triphosphates with a pt ester at the a phosphate (dNTPαS), allowing formation of pt-bonded polynucleotides. However, it can only react with S_{P} configured dNTPαS molecules, and does so with inversion of the stereocenter to form exclusively R_{P}-configured pt bonds in the product. Conversely, the 3′-5′exo activity of this polymerase cleaves R_{P} but not S_{P} configured bonds. Alternatively, the 3′-5′ exo activity of E. coli Exonuclease III cleaves S_{P} but not R_{P} configured pt bonds (24). Therefore, DNA created from the incorporation of dNTPαS by DNA Pol I is highly resistant to exonuclease cleavage by Exo III.

Phosphorothioates can block many, but not all, exonucleases. To block exonuclease cleavage, the pt bonds must be placed at the end(s) where the enzyme initiates, e.g., the 5′ end for Lambda Exo and the 3′ end for Exo III. It is important to note that a single pt bond is insufficient to fully protect an oligonucleotide from exonuclease digestion. When the pt bond is installed via an oxidation step during phosphoramidite synthesis, a nearly equal amount of each isomer (S_{P} and R_{P}) is formed at each pt linkage. Since most enzymes can cleave one of these isomers, a single chemically installed pt will protect only half the molecules from digestion by a given exonuclease. Thus, it is typically recommended that 3-6 pt bonds be used to block exonuclease digestion, to prevent this read-through. One might expect that because each bond is a 50:50 mixture of isomers, when presented with 5 consecutive isomers, a given enzyme could cleave the first bond on half the molecules, then half of the molecules that had the first bond hydrolyzed would have the second hydrolyzed, and so on, such that there would be a range of partially degraded products. In practice, it has been reported that five consecutive pt bonds completely block all exonuclease activity at all pt bond positions. The exact reasons for this are not currently known, but it is likely that exonucleases engage multiple bases at once, and the net effect of the isomeric mixture somehow prevents the active site from properly organizing around bonds that are the normally cleavable pt isomer.

There are several commonly used exonucleases that are not blocked even by 5 consecutive pt bonds; for example, Exo V, Exo VII and T5 Exonuclease all can cleave, leaving short oligos instead of cutting at every bond in a series, and thus can digest DNA by skipping over termini blocked by multiple pt bonds and cleaving at the first phosphodiester. Importantly, any enzyme with endonuclease activity, like DNase I, will simply ignore the ends and degrade the polynucleotides from the inside out (unless every phosphodiester bond is replaced by a phosphorothioate). Keeping these important exceptions in mind, phosphorothioate bonds remain the most generally applicable (and relatively inexpensive) way to protect oligonucleotides from digestion by exonucleases.

### 2. 2′-Modified Nucleosides

Certain 2′-O-modified riboses are both stable to spontaneous hydrolysis and offer strong resistance to exonuclease activity. 2′-O-methyl, 2′-fluoro, or 2′-O-methoxyethyl (MOE) nucleosides, which contain bulky substituents off the sugar ring, have been shown to grant strong resistance to nucleases and additionally increase the strength of annealing to complementary DNA and RNA.

These sugar modifications also work in vitro to block exonuclease activity quite strongly. While a single terminal MOE nucleoside only weakly inhibits exonuclease activity, three successive MOE modifications provide enhanced resistance to many exonucleases, including Exo I, Exo III, Lambda Exo, RecJ_{F} and polymerase exonucleases activities, such as that of DNA Polymerase I, Large (Klenow) Fragment. Similar to pt bonds, several exonucleases can digest through these regions, notably T5 Exo, T7 Exo, Exo V, Exo VII and Exo VIII. Overall, exonuclease inhibition by MOE is quite strong, but pt bonds are cheaper to install for most manufacturers. However, if for some reason the pt chemistry is not desired, 2′-O-modified ribose moieties are a viable alternative.

### 3. Other 5′/3′ End Modifications

Several other modifications, such as the inverted deoxythymidine bases and dideoxynucleotides have been reported to suppress serum nuclease activity when appended to the end of synthetic oligonucleotides. Many other modifications may be attached through “linkers” at either the 5′ or 3′ end, including fluorescent tags, biotin or other affinity labels, or reactive groups for attachment to beads or surfaces. These linkers are typically connected to the 5′ or 3′ end via a phosphodiester. Such modifications can be used alone or in combination with other modifications, e.g., pt bonds and/or 2′-modified nucleosides.

In sum, various useful terminal modifications (e.g., 5′ terminus and/or 3′ terminus) protect DNA from degradation. Exemplary modifications include but are not limited to, biotin, phosphorothioate, triethylene glycol (TEG), Locked Nucleic Acid (LNA, a 2′-oxygen-4′-carbon methylene linkage), hexaethylene glycol (Sp18), 1,3-propanediol (SpC3), 2′-O-methoxyethyl (MOE) ribonucleotides, 2′-O-methyl ribonucleotides (2′-OMe), 2′-fluoro (2′-F) nucleotides, or any combination thereof. These modifications are further described in WO2021/081358, which is incorporated herein by reference.

### Types of DNA

The DNA in DNA vaccines can be liner or circular. In some embodiments, the DNA is double-stranded or single stranded.

In some embodiments, the DNA is single-stranded and comprises at least one hairpin. In some such embodiments, the hairpin comprises a portion of a protelomerase target sequence (see e.g., U.S. Pat. No. 11,149,302, the methods and compositions in which are incorporated herein by reference).

In some embodiments, the DNA is double-stranded and is a closed linear DNA. In some such embodiments, the DNA may be a doggybone DNA (dbDNA™; Touchlight, Hampton, UK), which is a linear, double-stranded DNA that is covalently closed through the action of the protelomerase enzyme TelN. The dbDNA™ comprises ˜28 bps of telomeric sequence at both ends, and can encode long, complex, or unstable DNA sequences, eliminates bacterial sequences and has a strong expression profile. The dbDNA™ is often synthesized via in vitro amplification process, which uses rolling-circle amplication by phi29 polymerase to produce long concatameric repeats of the template DNA. These concatamers are subsequently reduced to individual closed linear DNA called dbDNA™ through the action of the protelomerase enzyme TelN. In addition to a fast in vitro production, the dbDNA™ may be more safer in a clinical setting, as it does not evoke recognition by TLR9 such that it minimizes the immune response. The methods of producing the dbDNA™ is described in Karda et al. (2019) Gene Therapy 26:86-92, which is incorporated herein by reference.

In some embodiments, the DNA vaccines may be synthesized according to the compositions and methods as described in U.S. Pat. No. 11,149,302 B2, US 2021/0269793 A1, U.S. Pat. No. 9,499,847 B2, U.S. Pat. No. 9,109,250 B2, U.S. Pat. No. 9,029,134 B2, U.S. Pat. No. 9,765,343 B2; each of which is incorporated herein by reference.

### Certain Exemplary Features

Accordingly, in some embodiments, the vaccine comprises DNA (e.g., encoding at least one cell surface protein or a fragment thereof of at least one methanogen). Such DNA may comprise a coding sequence and/or a non-coding sequence.

In some embodiments, the DNA is operably linked to a promoter. In some embodiments, the coding sequence is operably linked to a promoter.

In some embodiments, the promoter is selected form CMV promoter, CAG promoter, SCP promoter, CMVe-SCP, CMVmax, JET, PGK, EF-1a, AHSP promoter, MND promoter, Wiskott-Aldrich promoter, and PKLR promoter.

In some embodiments, the DNA comprises: (a) a transcription regulatory element (e.g., an enhancer, a transcription termination sequence, a proximal promoter element, a locus control region); and/or (b) a translation regulatory element (e.g., Kozak sequence, an untranslated region (5′ UTR or 3′ UTR), a polyadenylation signal sequence).

In some embodiments, the DNA further comprises a sequence operatively coding for the secretion of the at least one cell surface protein or a fragment thereof.

In some embodiments, the DNA is linear or circular.

In some embodiments, the DNA comprises a telomeric sequence.

In some embodiments, the DNA is double-stranded or single-stranded.

In some embodiments, the DNA is single-stranded and comprises at least one hairpin.

In some embodiments, the DNA is double-stranded and comprises a telomeric sequence (e.g., a closed linear DNA, e.g., dbDNA™) In some embodiments, the the DNA comprises at least one chemical modification. In some embodiments, the at least one chemical modification is a terminal modification, which is present at 5′ end and/or 3′ end. In some embodiments, the at least one chemical modification comprises phosphorothioate, triethylene glycol (TEG), Locked Nucleic Acid (LNA, a 2′-oxygen-4′-carbon methylene linkage), hexaethylene glycol (Sp18), 1,3-propanediol (SpC3), 2′-O-methoxyethyl (MOE) ribonucleotides, 2′-O-methyl ribonucleotides (2′-OMe), 2′-fluoro (2′-F) nucleotides, or any combination thereof. In some embodiments, the at least one chemical modification comprises at least five consecutive phosphorothioate bonds. In some embodiments, the at least one chemical modification comprises at least three consecutive 2′-O-methyl nucleosides and/or 2′-O-methoxyethyl nucleosides.

In some embodiments, the DNA is in a vector. In some embodiments, the vector is a plasmid. In some embodiments, the DNA is packaged in a virus, e.g., AAV, e.g., bovine AAV (e.g., for transduction to a subject).

In some embodiments, the DNA vaccine is in a composition (e.g., pharmaceutical composition) further comprising an adjuvant.

In some embodiments, the adjuvant comprises: (a) (±)-N-(3-aminopropyl)-N,N-dimethyl-2,3-bis(syn-9-tetradeceneyloxy)-1-propanaminium bromide (GAP-DMORIE) and a neutral lipid; (b) a cytokine; (c) mono-phosphoryl lipid A and trehalosedicorynomycolateAF (MPL+TDM); (d) a solubilized mono-phosphoryl lipid A formulation; and/or (e) CRL1005/BAK.

In some embodiments, the neutral lipid in (a) comprises (a) 1,2-dioleoyl-sn-glycero-3-phosphoethanolamine (DOPE); (b) 1,2-diphytanoyl-sn-glycero-3-phosphoethanolamine (DPyPE); and/or (c) 1,2-dimyristoyl-glycer-3-phosphoethanolamine (DMPE).

In some embodiments, the DNA vaccine is in a composition (e.g., pharmaceutical composition) further comprising a transfection facilitating compound.

In some embodiments, the transfection facilitating compound comprises (±)-N-(2-hydroxyethyl)-N,N-dimethyl-2,3-bis(tetradecyloxy)-1-propanaminium bromide) (DMRIE).

### RNA Vaccine

The RNA vaccine (e.g., comprising mRNA) does not need to reach the cell nucleus like the DNA ones, which is one of the potential practical advantages. Thus, the mode of application and effectiveness of RNA vaccines may be increased. In preferred embodiments, the RNA vaccine comprises mRNA that encodes a protein antigen. Although RNA is known to be a relatively unstable molecule, it has been proven that the RNA vaccine designs improve its stability and protein translation efficiency, which enhanced immune response.

Accordingly, in some embodiments, the nucleic acid is messenger RNA (mRNA). The mRNA of the present disclosure may encode one or more peptides or polypeptides.

In some embodiments, the nucleic acids of the present disclosure are linear. In preferred embodiments, the linear nucleic acids are produced by in vitro transcription. The linear nucleic acids encoding one or more antigens of the nucleic acid vaccines of the present disclosure which are made using in vitro transcription (IVT) enzymatic synthesis methods are referred to as “IVT polynucleotides” or “IVT mRNA.” In some embodiments, the nucleic acids of the present disclosure are circular or cyclic.

The circular nucleic acid is a single stranded nucleic acid whose termini are joined in some fashion, whether by ligation, covalent bond, common association with the same protein or other molecule or complex or by hybridization. As used herein, the circular nucleic acid acts substantially like, and has the properties of, an RNA. The circular nucleic acid or circular polynucleotides are described in WO2015/034925 and WO2016/011222, each of which is incorporated herein by reference.

Mode of Action of mRNA Vaccines

According to mechanistic studies on the fate of mRNA LNPs in rhesus monkeys, primarily monocytes and dendritic cell (DC) subsets translate the mRNA, likely involving ApoE dependent endocytosis. These locally transfected antigen-presenting cells (APCs) subsequently migrate to the draining lymph nodes (LN) where they present the mRNAencoded antigens to B cells and T cells. Moreover, owing to their relatively small size (˜100 nm), neutral surface charge and diffusible PEG lipid coating, mRNA LNPs might also enter the lymphatics to directly target LN-resident APCs and B cells. Finally, although these cells are often overlooked in flow cytometry studies, it is very likely that cell types such as myocytes, epithelial cells and fibroblasts also contribute to local mRNA expression. At the same time, mRNA vaccines also need to engage the innate immune system to improve their ability to induce, and tailor, antigenspecific immune responses. Upon sensing inflammatory stimuli, lymphatic migration of innate immune cells is promoted, while APCs become activated (i.e. maturation), in their turn providing costimulatory signals and cytokine responses. mRNA can mediate type I IFN responses upon cellular uptake, which can vary greatly depending on their structural design. Among other effects on antiviral immunity, type I IFNs restrict viral replication in infected host cells and induce stimulatory-genes involved in the maturation process of DCs. Furthermore, IFN-α directly acts as a third cytokine signal during T cell priming. The type I IFN response can act as a driving force for mRNA vaccines to elicit cytotoxic T cell responses. Several studies have indicated that an unmodified mRNA platform induces a more pronounced type-I IFN-polarized innate immune response.

Both the mRNA and the LNP vehicle can have intrinsic adjuvant properties. Cationic lipids have been associated with the activation of several cellular pathways like proapoptotic and pro-inflammatory cascades. For Moderna's LNP formulation, Hassett et al. reported that the SM-102 lipid was selected based on its improved tolerability profile in non-human primates, as evidenced by a reduced local reactogenicity (e.g., edema and erythema) and lowest induction of systemic cytokine responses (e.g., IL-6), without affecting the ability to induce antibody production.

### Exemplary Design to Reduce Innate Immune Response

Several modifications to the mRNA structure can drastically improve the final outcome. The design of (non-coding) structural elements of the mRNA such as the CAP structure, polyA tail and untranslated regions (UTRs) all have a major impact on the mRNA stability and translation capacity (see e.g., Orlandini von Niessen et al. (2019) Mol Therapy 27(4):824-836; Holtkamp et al. (2006) Blood 108(13): 4009-4017). For example, mRNA comprising a 5′-cap that is the diastereomer D 1 of beta-S-ARCA has been shown to improve mRNA stability and translation capacity (WO2011015347A1). Codon optimization in the mRNA sequence to e.g., match host transfer (t)RNA abundances, or as a determinant of introducing secondary structures, can drastically impact the protein synthesis rate and ribosome dwell time (i.e. mRNA functional half-life). In this context, N1-methylpseudouridine (1mψ) nucleotide-modifications for uridine were shown to provide additional base pair stability, giving rise to a high degree of secondary structure which significantly improves the mRNA translation (Mauger et al. (2019) Proc Natl Acad Sci U.S.A., 116:24075). Furthermore, the secondary structure design of mRNA can be optimized in order to improve mRNA stability against cleavage by endonucleases and chemical degradation processes, including hydrolysis (Wayment-Steele et al. (2021) Nucleic Acids Res, 49(18):10604-10617). BNT162b2 and mRNA-1273 implement a combination of modified nucleotide 1mΨ replacement and removal of dsRNA fragments in the mRNA production process, which strongly reduces the innate immune signaling in response to mRNA through decreased activation of TLR signaling and cytosolic RNA sensors. Moderna demonstrated that with such an approach, both local and systemic innate immune effects upon mRNA (vaccine) administration can be limited to a bare minimum in mice, which not only allows improved mRNA expression but also repeat dosing of the mRNA vaccine (Nelson et al. (2020) Sci Adv 6(26): 1-13). In contrast to BNT162b2 and mRNA-1273, the CVnCoV vaccine candidate contains an “unmodified” mRNA, which employs sequence engineering (e.g., reduction in uridine content), selected UTRs, and a stringent purification protocol to remove dsRNA fragments, demonstrating diverse approach to mRNA vaccines (Lutz et al. (2017) npj Vaccines 2(29): 1-9).

In some embodiments, nucleic acid vaccines of the present disclosure do not substantially induce an innate immune response of a cell into which the nucleic acid (e.g., mRNA) is introduced. Features of an induced innate immune response include 1) increased expression of pro-inflammatory cytokines, 2) activation of intracellular pattern recognition receptors (PRRs) (e.g., RIG-I, MDA5, etc.), and/or 3) termination or reduction in protein translation.

### RNA Design and Modifications

In some embodiments, the nucleic acid of the present disclosure is modified. In some embodiments, the nucleic acid may be structurally modified or chemically modified. As used herein, a “structural” modification is one in which two or more linked nucleosides are inserted, deleted, duplicated, inverted or randomized in a polynucleotide without significant chemical modification to the nucleotides themselves. Accordingly, the structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, the nucleic acid of the present disclosure may comprise one or more naturally occurring components, including any of the canonical nucleotides A (adenosine), G (guanosine), C (cytosine), U (uridine), or T (thymidine). In some embodiments, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

In some embodiments, the nucleic acid may be naturally or non-naturally occurring. Nucleic acids may include one or more modified (e.g., altered or alternative) nucleobases, nucleosides, nucleotides, or combinations thereof.

Nucleic acids may or may not be uniformly altered along the entire length of the molecule. In some embodiments, one or more or all types of nucleotide (e.g., purine or pyrimidine, or any one or more or all of A, G, U, C) may or may not be uniformly altered in a nucleic acid, or in a given predetermined sequence region thereof.

Different sugar alterations and/or internucleoside linkages (e.g., backbone structures) may exist at various positions in a nucleic acid. One of ordinary skill in the art will appreciate that the nucleotide analogs or other alteration(s) may be located at any position(s) of a nucleic acid such that the function of the nucleic acid is not substantially decreased. An alteration may also be a 5′- or 3′-terminal alteration. In some embodiments, the polynucleotide includes an alteration at the 3′-terminus. The nucleic acid may contain from about 1% to about 100% alternative nucleotides (either in relation to overall nucleotide content, or in relation to one or more types of nucleotide, i.e., any one or more of A, G, U, or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100% from 90% to 95%, from 90% to 100%, and from 95% to 100%). It will be understood that any remaining percentage is accounted for by the presence of a canonical nucleotide (e.g., A, G, U, or C).

Nucleic acids may contain at a minimum zero and at a maximum 100% alternative nucleotides, or any intervening percentages, such as at least 5% alternative nucleotides, at least 10% alternative nucleotides, at least 25% alternative nucleotides, at least 50% alternative nucleotides, at least 80% alternative nucleotides, or at least 90% alternative nucleotides. In some embodiments, nucleic acids may comprise an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure, or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide may be replaced with an alternative cytosine (e.g., a 5-substituted cytosine). The alternative cytosine can be replaced by a compound having a single unique structure, or can be replaced by a plurality of compounds having different structures (e.g., 2, 3, 4 or more unique structures).

In some embodiments, a nucleic acid molecule, formula, composition or method associated therewith comprises one or more polynucleotides comprising features as described in WO2002/098443, WO2003/051401, WO2008/052770, WO2009/127230, WO2006/122828, WO2008/083949, WO2010/088927, WO2010/037539, WO2004/004743, WO2005/016376, WO2006/024518, WO2007/095976, WO2008/014979, WO2008/077592, WO2009/030481, WO2009/095226, WO2011/069586, WO2011/026641, WO2011/144358, WO2012/019780, WO2012/013326, WO2012/089338, WO2012/113513, WO2012/116811, WO2012/116810, WO2013/113502, WO2013/113501, WO2013/113736, WO2013/143698, WO2013/143699, WO2013/143700, WO2013/120626, WO2013/120627, WO2013/120628, WO2013/120629, WO2013/174409, WO2014/127917, WO2015/024669, WO2015/024668, WO2015/024667, WO2015/024665, WO2015/024666, WO2015/024664, WO2015/058069, WO2015101415, WO2015101414, WO2015024667, WO2015062738, WO2015101416, WO2015/164674, and 2020/160397, each of which is incorporated by reference herein.

Nucleic acids useful in the disclosure typically include a first region of linked nucleosides encoding a polypeptide of interest (e.g., a coding region), a first flanking region located at the 5′ terminus of the first region (e.g., a 5′ UTR), a second flanking region located at the 3′ terminus of the first region (e.g., a 3′ UTR) at least one 5′cap region, and a 3′ stabilizing region. In some embodiments, a nucleic acid further comprises a polyA region (e.g., in the 3′ UTR) or a Kozak sequence (e.g., in the 5′ UTR).

In some embodiments, a polynucleotide or nucleic acid (e.g., an mRNA) may include a 5′ cap structure, a chain terminating nucleotide, a stem loop, a polyA sequence, and/or a polyadenylation signal. Any one of the regions of a nucleic acid may include one or more alternative components (e.g., an alternative nucleoside). In some embodiments, the 3′-stabilizing region may contain an alternative nucleoside such as an L-nucleoside, an inverted thymidine, or a 2′-O-methyl nucleoside and/or the coding region, 5;-UTR, 3′-UTR, or cap region may include an alternative nucleoside such as a 5-substituted uridine (e.g., 5-methoxy uridine), a 1-substituted pseudouridine (e.g., -methyl-pseudouridine), and/or a 5-substituted cytidine (e.g., 5-methyl-cytidine).

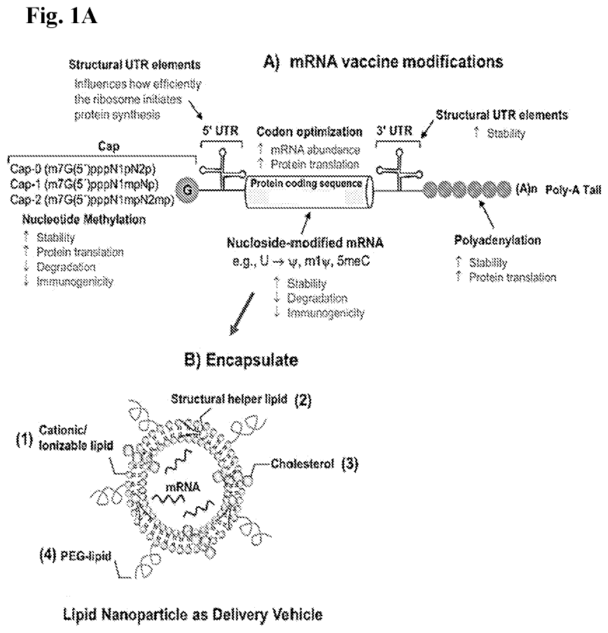

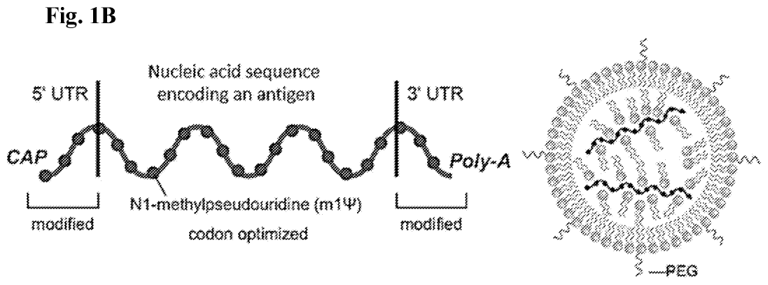

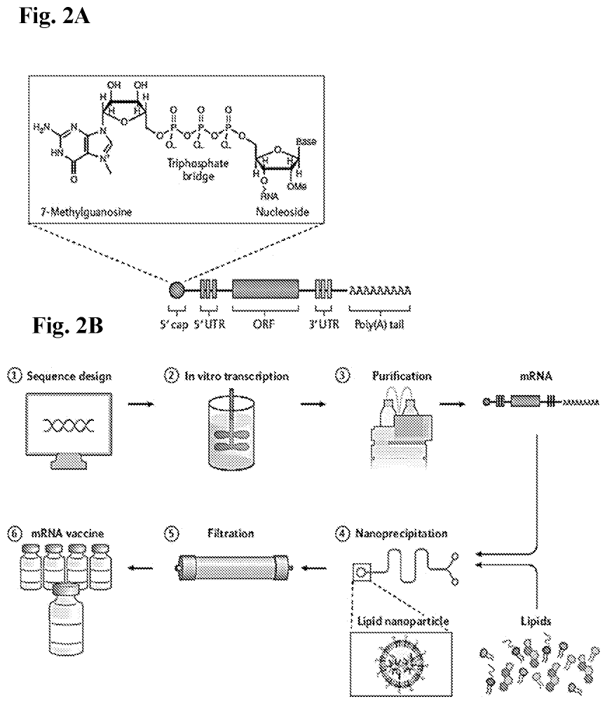

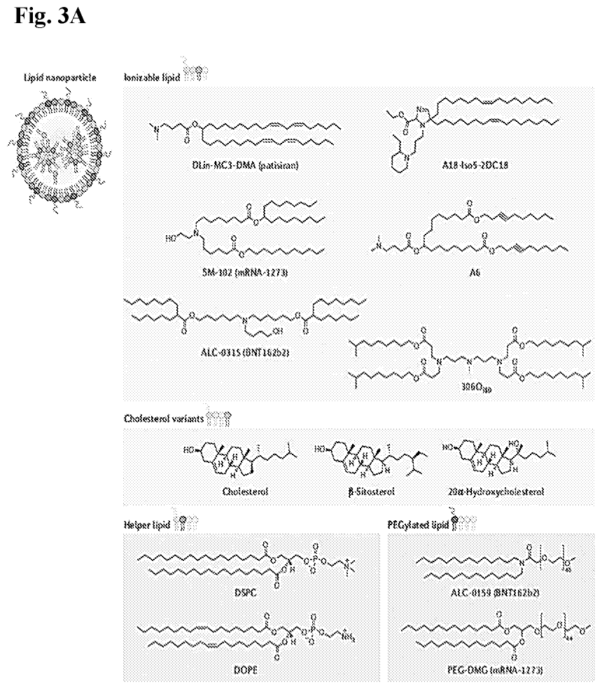

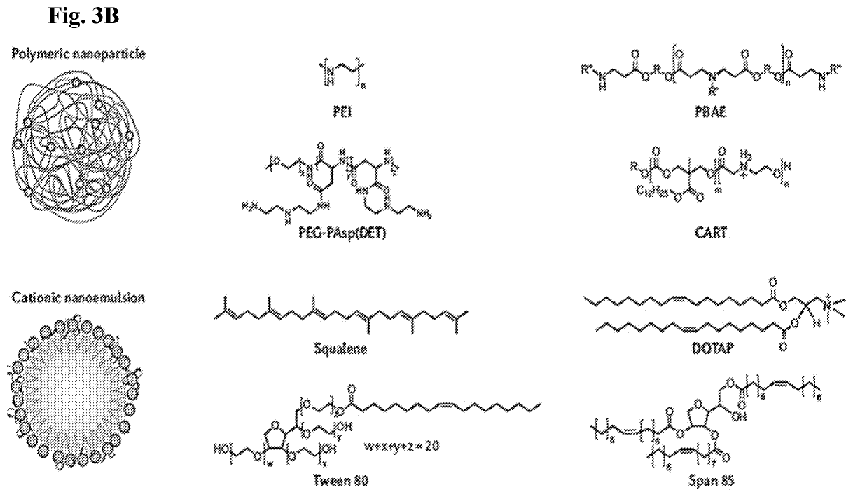

Accordingly, mRNA vaccines may comprise synthetic mRNA molecules that direct the production of the antigen that will generate an immune response. In vitro-transcribed (IVT) mRNA mimics the structure of endogenous mRNA, with five sections, from 5′ to 3′: 5′ cap, 5′ untranslated region (UTR), an open reading frame that encodes the antigen, 3′ UTR and a polyA tail (see FIG. 1). A variation on IVT mRNA, self-amplifying mRNA, also contains replicase genes that encode RNA-dependent RNA polymerase. The virus-derived polymerase amplifies mRNA transcripts intracellularly, enabling the expression of large amounts of antigen with reduced mRNA doses.

### 5′ Cap Structure

The 5′ cap structure, like that of natural eukaryotic mRNAs, contains a 7-methylguanosine nucleoside linked through a triphosphate bridge to the 5′ end of mRNA. As in mammals, the first or second nucleotide from the 5′ end is methylated on the 2′ hydroxyl of the ribose (2′-O-methylation), which prevents recognition by cytosolic sensors of viral RNA, and hence prevents unintended immune responses. Further, the 5′ cap protects the mRNA sterically from degradation by exonucleases, and it works synergistically with the polyA tail at the 3′ end, polyA binding proteins and translation initiation factor proteins to circularize mRNA and recruit ribosomes for initiating translation. The length of the polyA tail indirectly regulates both mRNA translation and half-life. A sufficiently long tail (100-150 bp) is necessary to interact with polyA binding proteins that form complexes necessary for initiating translation and protecting the cap from degradation by decapping enzymes.

A nucleic acid molecule (e.g., an mRNA) may include a 5′ cap structure. The 5′ cap structure of a nucleic acid is involved in nuclear export and increasing nucleic acid stability and binds the mRNA Cap Binding Protein (CBP), which is responsible for nucleic acid stability in the cell and translation competency through the association of CBP with polyA binding protein to form the mature cyclic mRNA species. The cap further assists the removal of 5′ proximal introns during mRNA splicing.

Endogenous nucleic acid molecules may be 5′-end capped generating a 5′-ppp-5′-triphosphate linkage between a terminal guanosine cap residue and the 5′-terminal transcribed sense nucleotide of the nucleic acid molecule. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5′-decapping through hydrolysis and cleavage of the guanylate cap structure may target a nucleic acid molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. In some embodiments, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap. Additional alternative guanosine nucleotides may be used such as α-methyl-phosphonate and seleno-phosphate nucleotides.

Additional alterations include, but are not limited to, 2′-O-methylation of the ribose sugars of 5′-terminal and/or 5′-anteterminal nucleotides of the nucleic acid molecules on the 2′-hydroxy group of the sugar. Multiple distinct 5′ cap structures can be used to generate the 5′ cap of a polynucleotide, such as an mRNA molecule.

5′ cap structures include those described in International Patent Publication Nos. WO2008/127688, WO 2008/016473, and WO 2011/015347, the cap structures of each of which are incorporated herein by reference.

Cap analogs, which herein are also referred to as synthetic cap analogs, chemical caps, chemical cap analogs, or structural or functional cap analogs, differ from natural (e.g., endogenous, wild-type, or physiological) 5′ caps in their chemical structure, while retaining cap function. Cap analogs may be chemically (i.e., non-enzymatically) or enzymatically synthesized and/linked to a polynucleotide.

For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7,3′-O-dimethyl-guanosine-5′-triphosphate-5′-guanosine, m⁷G-3′mppp-G, which may equivalently be designated 3′ O-Me-m7G(5′)ppp(5′)G). The 3′-O atom of the other, unaltered, guanosine becomes linked to the 5′-terminal nucleotide of the capped polynucleotide (e.g., an mRNA). The N7- and 3′-O-methylated guanosine provides the terminal moiety of the capped polynucleotide (e.g., mRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxyethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G(5′)ppp(5′)G and a N7-(4-chlorophenoxyethyl)-m3′-OG(5)ppp(5′)G cap analog (see, e.g., the various cap analogs and the methods of synthesizing cap analogs described in Kore et al. (2013) Bioorganic & Medicinal Chemistry 21:4570-4574; the cap structures of which are herein incorporated by reference). In other instances, a cap analog useful in the nucleic acids of the present disclosure is a 4-chloro/bromophenoxy ethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a“more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). In some embodiments, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Cap 1 structure. This cap results in a higher translational-competency, cellular stability, and a reduced activation of cellular pro-inflammatory cytokines, as compared, e.g., to other 5′ cap analog structures known in the art. Other exemplary cap structures include 7mG(5′)ppp(5′)N1pN2p (Cap 0), 7mG(5′)ppp(5′)N1mpNp (Cap 1), 7mG(5′)-ppp(5′)N1mpN2mp (Cap 2), and m(7)Gpppm(3)(6,6,2′)Apm(2′)Apm(2′)Cpm(2)(3,2′)Up (Cap 4).

Because the alternative nucleic acid molecule may be capped post-transcriptionally, and because this process is more efficient, nearly 100% of the alternative polynucleotides may be capped. This is in contrast to ˜80% when a cap analog is linked to a polynucleotide in the course of an in vitro transcription reaction.

5′-terminal caps may include endogenous caps or cap analogs. A 5′-terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine.

In some cases, a nucleic acid molecule contains a modified 5′-cap. A modification on the 5′-cap may increase the stability of polynucleotide, increase the half-life of the polynucleotide, and could increase the polynucleotide translational efficiency. The modified 5′-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

### 5′ UTR, 3′ UTR, and Translation Enhancer Elements (TEEs)

The 5′ and 3′ UTRs flanking the coding region regulate mRNA translation, half-life and subcellular localization. Naturally occurring UTRs from highly expressed genes, such as the α- and β-globin genes, are preferred for synthetic mRNA. However, because UTR performance can vary by cell type, alternative UTR sequences may be used that have been optimized for the desired application and intended cell target. These engineered UTR sequences minimize mRNA degradation by excluding miRNA-binding sites and AU rich regions in the 3′ UTR. Furthermore, they minimize regions that prevent ribosomes from scanning the mRNA transcript, such as sequences with secondary and tertiary structure (for example, hairpins) in the 5′ UTR. The open reading frame of the mRNA vaccine is the most crucial component because it contains the coding sequence that is translated into protein. Although the open reading frame is not as malleable as the non-coding regions, it can be optimized to increase translation without altering the protein sequence by replacing rarely used codons with more frequently occurring codons that encode the same amino acid residue. Although replacement of rare codons is an attractive optimization strategy, it must be used judiciously. This is because, in the case of some proteins, the slower translation rate of rare codons is necessary for proper protein folding.

Natural 5′UTRs bear features which play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of the polynucleotides of the invention. For example, use of 5′ UTR from other tissue-specific mRNA to improve expression in that tissue is possible for muscle (MyoD, Myosin, Myoglobin, Myogenin, Herculin), for endothelial cells (Tie-1, CD36), and for lung epithelial cells (SP-A/B/C/D). Such 5′ UTRs are especially useful for intramuscular, intradermal, and nasal administration of the vaccines of the present disclosure.

A 5′-UTR may be provided as a flanking region to polynucleotides (e.g, mRNAs). A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

Shown in Table 21 in U.S. Provisional Application No. 61/775,509, and in Table 21 and in Table 22 in U.S. Provisional Application No. 61/829,372, of which are incorporated herein by reference, is a listing of the start and stop site of alternative polynucleotides (e.g., mRNA). In Table 21, each 5′-UTR (5′-UTR-005 to 5′-UTR 68511) is identified by its start and stop site relative to its native or wild type (homologous) transcript (ENST; the identifier used in the ENSEMBL database).

To alter one or more properties of a polynucleotide (e.g, mRNA), 5′-UTRs which are heterologous to the coding region of an alternative polynucleotide (e.g, mRNA) may be engineered. The polynucleotides (e.g, mRNA) may then be administered to cells, tissue or organisms and outcomes such as protein level, localization, and/or half-life may be measured to evaluate the beneficial effects the heterologous 5′-UTR may have on the alternative polynucleotides (mRNA). Variants of the 5′-UTRs may be utilized wherein one or more nucleotides are added or removed to the termini, including A, T, C or G. 5′-UTRs may also be codon-optimized, or altered in any manner described herein.

The 5′-UTR of a polynucleotides (e.g, mRNA) may include at least one translation enhancer element. The term“translational enhancer element” refers to sequences that increase the amount of polypeptide or protein produced from a polynucleotide. As a non-limiting example, the TEE may be located between the transcription promoter and the start codon. The polynucleotides (e.g, mRNA) with at least one TEE in the 5′-UTR may include a cap at the 5-UTR. Further, at least one TEE may be located in the 5-UTR of polynucleotides (e.g, mRNA) undergoing cap-dependent or cap-independent translation.

In some aspects, TEEs are conserved elements in the UTR which can promote translational activity of a polynucleotide such as, but not limited to, cap-dependent or cap-independent translation. The conservation of these sequences has been previously shown by Panek et al. (Nucleic Acids Research, 2013, 1-10) across 14 species.

In some embodiments, the TEEs known may be in the 5-leader of the Gtx homeodomain protein (Chappell et al, Proc. Natl. Acad. Sci. USA 101:9590-9594, 2004, the TEEs of which are incorporated herein by reference).

In other embodiments, TEEs are disclosed in US Patent Publication Nos. 2009/0226470 and 2013/0177581, International Patent Publication Nos. WO2009/075886, WO2012/009644, and WO1999/024595, U.S. Pat. Nos. 6,310,197, and 6,849,405, the TEE sequences of each of which are incorporated herein by reference.

In yet other embodiments, the TEE may be an internal ribosome entry site (IRES), HCV-IRES or an IRES element such as, but not limited to, those described in U.S. Pat. No. 7,468,275, US Patent Publication Nos. 2007/0048776 and 2011/0124100 and International Patent Publication Nos. WO2007/025008 and WO2001/055369, the IRES sequences of each of which are incorporated herein by reference. The IRES elements may include, but are not limited to, the Gtx sequences (e.g., Gtx9-nt, Gtx8-nt, Gtx7-nt) described by Chappell et al. (Proc. Natl. Acad. Sci. USA 101:9590-9594, 2004) and Zhou et al. (PNAS 102:6273-6278, 2005) and in US Patent Publication Nos. 2007/0048776 and 2011/0124100 and International Patent Publication No. WO2007/025008, the IRES sequences of each of which are incorporated herein by reference.

“Translational enhancer polynucleotides” are polynucleotides which include one or more of the specific TEE exemplified herein and/or disclosed in the art (see e.g., U.S. Pat. Nos. 6,310,197, 6,849,405, 7,456,273, 7,183,395, U.S. Patent Publication Nos. 2009/226470, 2007/0048776, 2011/0124100, 2009/0093049, 2013/0177581, International Patent Publication Nos. WO2009/075886, WO2007/025008, WO2012/009644, WO2001/055371, WO 1999/024595, and European Patent Nos. 2610341 and 2610340; the TEE sequences of each of which are incorporated herein by reference) or their variants, homologs or functional derivatives. One or multiple copies of a specific TEE can be present in a polynucleotide (e.g., mRNA). The TEEs in the translational enhancer polynucleotides can be organized in one or more sequence segments. A sequence segment can harbor one or more of the specific TEEs exemplified herein, with each TEE being present in one or more copies. When multiple sequence segments are present in a translational enhancer polynucleotide, they can be homogenous or heterogeneous. Thus, the multiple sequence segments in a translational enhancer polynucleotide can harbor identical or different types of the specific TEEs exemplified herein, identical or different number of copies of each of the specific TEEs, and/or identical or different organization of the TEEs within each sequence segment.

A polynucleotide (e.g., mRNA) may include at least one TEE that is described in International Patent Publication Nos. WO1999/024595, WO2012/009644, WO2009/075886, WO2007/025008, WO1999/024595, European Patent Publication Nos. 2610341 and 2610340, U.S. Pat. Nos. 6,310,197, 6,849,405, 7,456,273, 7,183,395, and US Patent Publication Nos. 2009/0226470, 2011/0124100, 2007/0048776, 2009/0093049, and 2013/0177581 the TEE sequences of each of which are incorporated herein by reference.

The TEE may be located in the 5′-UTR of the polynucleotides (e.g., mRNA).

A polynucleotide (e.g., mRNA) may include at least one TEE that has at least 50%, at least 55%, at least 60%, at least 65%, at least 70%, at least 75%, at least 80%, at least 85%, at least 90%, at least 95% or at least 99% identity with the TEEs described in US Patent Publication Nos. 2009/0226470, 2007/0048776, 2013/0177581 and 2011/0124100, International Patent Publication Nos. WO1999/024595, WO2012/009644, WO2009/075886 and WO2007/025008, European Patent Publication Nos. 2610341 and 2610340, U.S. Pat. Nos. 6,310,197, 6,849,405, 7,456,273, 7,183,395, the TEE sequences of each of which are incorporated herein by reference.

The 5′-UTR of a polynucleotide (e.g, mRNA) may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 5′-UTR of a polynucleotide (e.g, mRNA) may be the same or different TEE sequences. The TEE sequences may be in a pattern such as ABABAB, AABBAABBAABB, or ABCABCABC, or variants thereof, repeated once, twice, or more than three times. In these patterns, each letter, A, B, or C represent a different TEE sequence at the nucleotide level.

In some cases, the 5-UTR may comprise a spacer to separate two TEE sequences. As a non-limiting example, the spacer may be a 15 nucleotide spacer and/or other spacers known in the art. As another non-limiting example, the 5′-UTR may include a TEE sequence-spacer module repeated at least once, at least twice, at least 3 times, at least 4 times, at least 5 times, at least 6 times, at least 7 times, at least 8 times, at least 9 times, or more than 9 times in the 5′-UTR.

In other instances, the spacer separating two TEE sequences may include other sequences known in the art which may regulate the translation of the polynucleotides (e.g., mRNA) of the present disclosure such as, but not limited to, miR sequences (e.g., miR binding sites and miR seeds). As a non-limiting example, each spacer used to separate two TEE sequences may comprise a different miR sequence or component of a miR sequence (e.g., miR seed sequence).

In some instances, the TEE in the 5′-UTR of a polynucleotide (e.g., mRNA) may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in US Patent Publication Nos. 2009/0226470, 2007/0048776, 2013/0177581 and 2011/0124100, International Patent Publication Nos. WO1999/024595, WO2012/009644, WO2009/075886 and WO2007/025008, European Patent Publication Nos. 2610341 and 2610340, and U.S. Pat. Nos. 6,310,197, 6,849,405, 7,456,273, and 7,183,395, the TEE sequences of each of which are incorporated herein by reference. In other embodiments, the TEE in the 5′-UTR of the polynucleotides (e.g., mRNA) of the present disclosure may include a 5-30 nucleotide fragment, a 5-25 nucleotide fragment, a 5-20 nucleotide fragment, a 5-15 nucleotide fragment, a 5-10 nucleotide fragment of the TEE sequences disclosed in US Patent Publication Nos. 2009/0226470, 2007/0048776, 2013/0177581 and 2011/0124100, International Patent Publication Nos. WO 1999/024595, WO2012/009644, WO2009/075886, and WO2007/025008, European Patent Publication Nos. 2610341 and 2610340, and U.S. Pat. Nos. 6,310,197, 6,849,405, 7,456,273, and 7,183,395; the TEE sequences of each of which are incorporated herein by reference.

In certain cases, the TEE in the 5′-UTR of the polynucleotides (e.g., mRNA) of the present disclosure may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in Chappell et al. (Proc. Natl. Acad. Sci. USA 101:9590-9594, 2004) and Zhou et al. (PNAS 102:6273-6278, 2005), in Supplemental Table 1 and in Supplemental Table 2 disclosed by Wellensiek et al (Nature Methods, 2013; DOI: 10.1038/NMETH.2522); the TEE sequences of each of which are herein incorporated by reference.

In other embodiments, the TEE in the 5′-UTR of the polynucleotides (e.g., mRNA) of the present disclosure may include a 5-30 nucleotide fragment, a 5-25 nucleotide fragment, a 5-20 nucleotide fragment, a 5-15 nucleotide fragment, a 5-10 nucleotide fragment of the TEE sequences disclosed in Chappell et al. (Proc. Natl. Acad. Sci. USA 101:9590-9594, 2004) and Zhou et al. (PNAS 102:6273-6278, 2005), in Supplemental Table 1 and in Supplemental Table 2 disclosed by Wellensiek et al (Nature Methods, 2013; DOI: 10.1038/NMETH.2522); the TEE sequences of each of which is incorporated herein by reference.

In some cases, the TEE used in the 5′-UTR of a polynucleotide (e.g., mRNA) is an IRES sequence such as, but not limited to, those described in U.S. Pat. No. 7,468,275 and International Patent Publication No. WO2001/055369, the TEE sequences of each of which are incorporated herein by reference.

In some instances, the TEEs used in the 5′-UTR of a polynucleotide (e.g., mRNA) may be identified by the methods described in US Patent Publication Nos. 2007/0048776 and 2011/0124100 and International Patent Publication Nos. WO2007/025008 and

WO2012/009644, the methods of each of which are incorporated herein by reference.

In some cases, the TEEs used in the 5-UTR of a polynucleotide (e.g., mRNA) of the present disclosure may be a transcription regulatory element described in U.S. Pat. Nos. 7,456,273 and 7,183,395, US Patent Publication No. 2009/0093049, and International Publication No. WO2001/055371, the TEE sequences of each of which is incorporated herein by reference. The transcription regulatory elements may be identified by methods known in the art, such as, but not limited to, the methods described in U.S. Pat. Nos. 7,456,273 and 7,183,395, US Patent Publication No. 2009/0093049, and International Publication No. WO2001/055371, the methods of each of which is incorporated herein by reference.

In yet other instances, the TEE used in the 5-UTR of a polynucleotide (e.g., mRNA) is a polynucleotide or portion thereof as described in U.S. Pat. Nos. 7,456,273 and 7,183,395, US Patent Publication No. 2009/0093049, and International Publication No. WO2001/055371, the TEE sequences of each of which are incorporated herein by reference.

The 5′-UTR including at least one TEE described herein may be incorporated in a monocistronic sequence such as, but not limited to, a vector system or a polynucleotide vector. As a non-limiting example, the vector systems and polynucleotide vectors may include those described in U.S. Pat. Nos. 7,456,273 and 7,183,395, US Patent Publication Nos. 2007/0048776, 2009/0093049 and 2011/0124100, and International Patent Publication Nos. WO2007/025008 and WO2001/055371, the TEE sequences of each of which are incorporated herein by reference.

The TEEs described herein may be located in the 5′-UTR and/or the 3′-UTR of the polynucleotides (e.g., mRNA). The TEEs located in the 3′-UTR may be the same and/or different than the TEEs located in and/or described for incorporation in the 5′-UTR.

In some cases, the 3′-UTR of a polynucleotide (e.g., mRNA) may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 3′-UTR of the polynucleotides (e.g., mRNA) of the present disclosure may be the same or different TEE sequences. The TEE sequences may be in a pattern such as ABABAB, AABBAABBAABB, or ABCABCABC, or variants thereof, repeated once, twice, or more than three times. In these patterns, each letter, A, B, or C represent a different TEE sequence at the nucleotide level.

In one instance, the 3′-UTR may include a spacer to separate two TEE sequences. As a non-limiting example, the spacer may be a 15 nucleotide spacer and/or other spacers known in the art. As another non-limiting example, the 3′-UTR may include a TEE sequence-spacer module repeated at least once, at least twice, at least 3 times, at least 4 times, at least 5 times, at least 6 times, at least 7 times, at least 8 times, at least 9 times, or more than 9 times in the 3′-UTR.

In other cases, the spacer separating two TEE sequences may include other sequences known in the art which may regulate the translation of the polynucleotides (e.g., mRNA) of the present disclosure such as, but not limited to, miR sequences described herein (e.g., miR binding sites and miR seeds). As a non-limiting example, each spacer used to separate two TEE sequences may include a different miR sequence or component of a miR sequence (e.g., miR seed sequence).

In some embodiments, a polyribonucleotide of the disclosure comprises a miR and/or TEE sequence. In some embodiments, the incorporation of a miR sequence and/or a TEE sequence into a polyribonucleotide of the disclosure can change the shape of the stem loop region, which can increase and/or decrease translation. See e.g., Kedde et al, Nature Cell Biology 2010 12(10): 1014-20, herein incorporated by reference in its entirety.

TABLE 4A
Exemplary 5′ untranslated regions (UTRs)

See SEQ ID NO: 12148 to SEQ ID NO: 12164 for the representative 5′ UTR sequences.
SEQ ID NO: 16953 is a 5′ UTR sequence used in the BNT162b2 vaccine
(BioNTech/Pfizer) and has the following nucleic acid sequence:
GAGAATAAACTAGTATTCTTCTGGTCCCCACAGACTCAGAGAGAACCCGCCACC
(*The above sequence includes RNA sequence in which T is replaced with U or any variant thereof)

TABLE 4B
Exemplary 3′ untranslated regions (UTRs)

See SEQ ID NO: 12165 to SEQ ID NO: 12181 for the representative 3′ UTR sequences.
SEQ ID NO: 16954 is a 3′ UTR sequence used in the BNT162b2 vaccine
(BioNTech/Pfizer) and has the following nucleic acid sequence:
CTCGAGCTGGTACTGCATGCACGCAATGCTAGCTGCCCCTTTCCCGTCCTGGGTACC
CCGAGTCTCCCCCGACCTCGGGTCCCAGGTATGCTCCCACCTCCACCTGCCCCACTC
ACCACCTCTGCTAGTTCCAGACACCTCCCAAGCACGCAGCAATGCAGCTCAAAACG
CTTAGCCTAGCCACACCCCCACGGGAAACAGCAGTGATTAACCTTTAGCAATAAAC
GAAAGTTTAACTAAGCTATACTAACCCCAGGGTTGGTCAATTTCGTGCCAGCCACAC
CCTGGAGCTAGC
(*The above sequence includes RNA sequence in which T is replaced with U or any variant thereof)

* Included in Tables 4 and 5 are RNA nucleic acid molecules (e.g., thymidine replaced with uridine), as well as DNA or RNA nucleic acid sequences or any variant thereof (a structural variant or a chemical variant (e.g., chemically modified nucleotide)) comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Tables 4 or 5, or a portion thereof. Such nucleic acid molecules can have a function of the full-length nucleic acid (e.g., for the intended function of inducing an immune response) as described further herein. Further included are orthologous nucleic acid sequences. A person of ordinary skill in the art understands how to determine the orthologous nucleic acid sequences (e.g., of ruminants) described herein based on the sequence similarity (e.g., by BLAST search).

### Internal Ribosome Entry Sites

Polynucleotides may contain an internal ribosome entry site (IRES). An IRES may act as the sole ribosome binding site, or may serve as one of multiple ribosome binding sites of an mRNA. A polynucleotide containing more than one functional ribosome binding site may encode several peptides or polypeptides that are translated independently by the ribosomes (e.g., multicistronic mRNA). When polynucleotides are provided with an IRES, further optionally provided is a second translatable region. Examples of IRES sequences that can be used according to the present disclosure include without limitation, those from picomaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Nucleic Acid Sequence Encoding the Antigen

In some embodiments, the nucleic acid sequences that encode at least one cell surface protein or a fragment thereof of at least one methanogen are present on multiple (e.g., at least 2, 3, 4, 5, 6, 7, 8, 9, or 10) nucleic acid (e.g., mRNA) molecules.

In other embodiments, the nucleic acid sequences that encode at least one cell surface protein or a fragment thereof of at least one methanogen is present on one nucleic acid (e.g., mRNA) molecule.

In certain embodiments, nucleic acid sequences encoding multiple (e.g., at least 2, 3, 4, 5, 6, 7, 8, 9, or 10) polypeptides are in a nucleic acid (e.g., mRNA) molecule such that translation of the nucleic acid produces a concatemeric polypeptide, which comprises two or more heterologous polypeptides that are translated as a single fusion polypeptide. In some embodiments, such polypeptides are fragments of one cell surface protein. In other embodiments, such polypeptides are fragments of multiple cell surface proteins.

Any variation of the above is contemplated. In preferred embodiments, nucleic acid comprises sequences that encode multiple cell surface proteins or fragments thereof (e.g., antigenic fragment) to maximize the immune response against methanogens.

### Nucleoside Modification

To maximize translation, the mRNA sequence typically incorporates modified nucleosides, such as pseudouridine, N1-methylpseudouridine or other nucleoside analogues. Because all native mRNAs include modified nucleosides, the immune system has evolved to recognize unmodified single-stranded RNA, which is a hallmark of viral infection. Specifically, unmodified mRNA is recognized by pattern recognition receptors (PRRs), such as Toll-like receptor 3 (TLR3), TLR7 and TLR8, and the retinoic acid-inducible gene I (RIGI) receptor. TLR7 and TLR8 receptors bind to guanosine- or uridine-rich regions in mRNA and trigger the production of type I interferons, such as IFNα, that can block mRNA translation.

The use of modified nucleosides, particularly modified uridine, prevents recognition by pattern recognition receptors, enabling sufficient levels of translation to produce prophylactic amounts of protein. Another strategy to avoid detection by pattern recognition receptors, pioneered by CureVac, uses sequence engineering and codon optimization to deplete uridines by boosting the GC content of the vaccine mRNA. In addition to improvements to the mRNA sequence, significant advances have also been made to streamline mRNA production. Clinically used synthetic mRNA is transcribed in vitro from a DNA plasmid by using the bacteriophage RNA polymerase T7 (T3 and SP6 polymerases can also be used). It is co-transcriptionally capped (e.g., CleanCap, developed by TriLink BioTechnologies) with a 2′-O-methylated cap and purified to remove double-stranded RNA (dsRNA) contaminants, reactants and incomplete transcripts. Other methods add the cap with a post-transcriptional reaction using capping and 2′-O-methyltransferase enzymes derived from the vaccinia virus. The polyA tail is encoded in the DNA template, which eliminates reaction steps and reduces overall production time and material loss.

### 1. Nucleobase Alternatives

The alternative nucleosides and nucleotides can comprise an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may comprise, for example, one or more substitutions or modifications including, but are not limited to, alkyl, aryl, halo, oxo, hydroxyl, alkyloxy, and/or thio substitutions; one or more fused or open rings; oxidation; and/or reduction.

Alternative nucleotide base pairing encompasses not only the standard adenine-thymine, adenine-uracil, or guanine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

In some embodiments, the nucleobase is an alternative uracil. Exemplary nucleobases and nucleosides having an alternative uracil include, but are not limited to, pseudouridine (Ψ), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (memo⁵U), 5-carboxy methyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethy 1-uracil (mnm⁵U), 5-methylaminomethyl-2-thio-uracil (mnm⁵s²U), 5-methylaminomethyl-2-seleno-uracil (mnm⁵se²U), 5-carbamoylmethy 1-uracil (ncm⁵U), 5-carboxymethylaminomethyl-uracil (cmnm⁵U), 5-carboxymethylaminomethyl-2-thio-uracil (cmnm⁵s²U), 5-propyny 1-uracil, 1-propynyl-pseudouracil, 5-taurinomethy 1-uracil (xm⁵U), 1-taurinomethyl-pseudouridine, 5-taurinomethyl-2-thio-uracil(xm⁵s²U), 1-taurinomethyl-4-thio-pseudouridine, 5-methyl-uracil (m⁵U, i.e., having the nucleobase deoxythymine), 1-methyl-pseudouridine (m¹T), 5-methyl-2-thio-uracil (m⁵s²U), 1-methyl-4-thio-pseudouridine (m¹s⁴Ψ). 4-thio-1-methyl-pseudouridine, 3-methyl-pseudouridine (m³Ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, Ni-methyl-pseudouridine, 3-(3-amino-3-carboxypropyl)uracil (acp³U), 1-methyl-3-(3-amino-3-carboxypropyl)pseudouridine (acp³′P), 5-(isopentenylaminomethyl)uracil (inm⁵U), 5-(isopentenylaminomethyl)-2-thio-uracil (inm⁵s²U), 5, 2′-O-dimethy 1-uridine (m⁵Um), 2-thio-2′-O_methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3, 2′-O-dimethy 1-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethy 1-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the nucleobase is an alternative cytosine. Exemplary nucleobases and nucleosides having an alternative cytosine include, but are not limited to, 5-aza-cytosine, 6-aza-cytosine, pseudoisocytidine, 3-methyl-cytosine (m3C), N4-acetyl-cytosine (ac4C), 5-formyl-cytosine (f5C), N4-methy 1-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocyti dine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include, but are not limited to, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methy 1-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2 g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acety 1-adenine (ac6A), 7-methy 1-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (ml Am), 2-amino-N6-methy 1-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include, but are not limited to, inosine (I), 1-methyl-inosine (mlI), wyosine (imG), methylwyosine (mimG), 4-demethyl-wyosine (imG-14), isowyosine (imG2), wybutosine (yW), peroxywybutosine (o2yW), hydroxy wybutosine (OHyW), undermodified hydroxy wybutosine (OHyW*), 7-deaza-guanine, queuosine (Q), epoxy queuosine (oQ), galactosyl-queuosine (galQ), mannosyl-queuosine (manQ), 7-cyano-7-deaza-guanine (preQO), 7-aminomethyl-7-deaza-guanine (preQl), archaeosine (G+), 7-deaza-8-aza-guanine, 6-thio-guanine, 6-thio-7-deaza-guanine, 6-thio-7-deaza-8-aza-guanine, 7-methy 1-guanine (m7G), 6-thio-7-methy 1-guanine, 7-methyl-inosine, 6-methoxy-guanine, 1-methyl-guanine (mlG), N2-methyl-guanine (m2G), N2,N2-dimethyl-guanine (m22G), N2, 7-dimethyl-guanine (m2,7G), N2, N2,7-dimethy 1-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (mlGm), N2,7-dimethyl-2′-O-methyl-guanosine (m2,7Gm), 2′-O-methyl-inosine (Im), 1,2′-O-dimethyl-inosine (mllm), 1-thio-guanine, and 0-6-methyl-guanine.

The alternative nucleobase of a nucleotide can be independently a purine, a pyrimidine, a purine or pyrimidine analog. In some embodiments, the nucleobase can be an alternative to adenine, cytosine, guanine, uracil, or hypoxanthine. In another embodiment, the nucleobase can also include, for example, naturally-occurring and synthetic derivatives of a base, including, but not limited to, pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d] pyrimidine, imidazo[1,5-a] 1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine.

When the nucleotides are depicted using the shorthand A, G, C, T or U, each letter refers to the representative base and/or derivatives thereof, e.g., A includes adenine or adenine analogs, e.g., 7-deaza adenine).

Additional modifications of the nucleotides are taught by e.g., Tables 22, 23, and 25 of WO2015/164674, which is incorporated herein by reference.

### 2. Alteration on the Sugar

Nucleosides include a sugar molecule (e.g., a 5-carbon or 6-carbon sugar, such as pentose, ribose, arabinose, xylose, glucose, galactose, or a deoxy derivative thereof) in combination with a nucleobase, while nucleotides are nucleosides containing a nucleoside and a phosphate group or alternative group (e.g., boranophosphate, thiophosphate, selenophosphate, phosphonate, alkyl group, amidate, and glycerol). A nucleoside or nucleotide may be a canonical species, e.g., a nucleoside or nucleotide including a canonical nucleobase, sugar, and, in the case of nucleotides, a phosphate group, or may be an alternative nucleoside or nucleotide including one or more alternative components. In some embodiments, alternative nucleosides and nucleotides can be altered on the sugar of the nucleoside or nucleotide. In some embodiments, the alternative nucleosides or nucleotides include the structures described in WO2020/160397, which is incorporated herein by reference.

In some embodiments, the 2′-hydroxy group (OH) can be modified or replaced with a number of different substituents. Exemplary substitutions at the 2′-position include, but are not limited to, H, azido, halo (e.g., fluoro), optionally substituted Ci-6 alkyl (e.g., methyl); optionally substituted Ci-6 alkoxy (e.g., methoxy or ethoxy); optionally substituted C6-io aryloxy; optionally substituted C3-8 cycloalkyl; optionally substituted G,-in aryl-Ci-6 alkoxy, optionally substituted Ci-12 (heterocyclyl)oxy; a sugar (e.g., ribose, pentose, or any described herein); a polyethyleneglycol (PEG), —O(CH2CH2O)nCH2CH2OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxy is connected by a Ci-6 alkylene or Ci-6 heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein.

Generally, RNA includes the sugar group ribose, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′ 2′)), and peptide nucleic acid (PNA, where 2-amino-ethyl-glycine linkages replace the ribose and phosphodiester backbone).

In some embodiments, the sugar group contains one or more carbons that possess the opposite stereochemical configuration of the corresponding carbon in ribose. Thus, a polynucleotide molecule can include nucleotides containing, e.g., arabinose or L-ribose, as the sugar.

In some embodiments, the polynucleotide includes at least one nucleoside wherein the sugar is L-ribose, 2′-O-methy 1-ribose, 2′-fluoro-ribose, arabinose, hexitol, an LNA, or a PNA.

### 3. Alterations on the Internucleoside Linkage

Alternative nucleotides can be altered on the intemucleoside linkage (e.g., phosphate backbone). Herein, in the context of the polynucleotide backbone, the phrases “phosphate” and“phosphodiester” are used interchangeably. Backbone phosphate groups can be altered by replacing one or more of the oxygen atoms with a different substituent.

The alternative nucleotides can include the wholesale replacement of an unaltered phosphate moiety with another intemucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BFE), sulfur (thio), methyl, ethyl, and/or methoxy. As a non-limiting example, two non-bridging oxygens at the same position (e.g., the alpha (a), beta (b) or gamma (g) position) can be replaced with a sulfur (thio) and a methoxy.

The replacement of one or more of the oxygen atoms at the a position of the phosphate moiety (e.g., a-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Other intemucleoside linkages that may be employed according to the present disclosure, including intemucleoside linkages which do not contain a phosphorous atom, are described herein.

### Stemp Loops

Polynucleotides (e.g., mRNAs) may include a stem loop such as, but not limited to, a histone stem loop. The stem loop may be a nucleotide sequence that is about 25 or about 26 nucleotides in length such as, but not limited to, those as described in International Patent Publication No. WO2013/103659, which is incorporated herein by reference. The histone stem loop may be located 3′-relative to the coding region (e.g., at the 3′-terminus of the coding region). As a non-limiting example, the stem loop may be located at the 3′-end of a polynucleotide described herein. In some cases, a polynucleotide (e.g., an mRNA) includes more than one stem loop (e.g., two stem loops). Examples of stem loop sequences are described in International Patent Publication Nos. WO2012/019780 and WO2015/02667, the stem loop sequences of which are herein incorporated by reference. In some instances, a polynucleotide comprises the stem loop sequence CAAAGGCTCTTTTCAGAGCCACCA (SEQ ID NO: 16955). In others, a polynucleotide includes the stem loop sequence

(SEQ ID NO: 16956)
CAAAGGCUCUUUUCAGAGCCACCA.

A stem loop may be located in a second terminal region o a polynucleotide. As a non-limiting example, the stem loop may be located within an untranslated region (e.g., 3′-UTR) in a second terminal region.

In some cases, a polynucleotide such as, but not limited to mRNA, which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide.

In other cases, a polynucleotide such as, but not limited to mRNA, which includes the histone stem loop may be stabilized by an alteration to the 3′-region of the polynucleotide that can prevent and/or inhibit the addition of oligio (U) (see e.g., International Patent Publication No. WO2013/103659).

In yet other cases, a polynucleotide such as, but not limited to mRNA, which includes the histone stem loop may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

In some instances, the polynucleotides of the present disclosure may include a histone stem loop, a polyA region, and/or a 5′-cap structure. The histone stem loop may be before and/or after the polyA region. The polynucleotides including the histone stem loop and a polyA region sequence may include a chain terminating nucleoside described herein.

In other instances, the polynucleotides of the present disclosure may include a histone stem loop and a 5′-cap structure. The 5′-cap structure may include, but is not limited to, those described herein and/or known in the art.

In certain instances, the conserved stem loop region may comprise a miR sequence described herein and may also comprise a TEE sequence.

In some cases, the incorporation of a miR sequence and/or a TEE sequence changes the shape of the stem loop region which may increase and/or decrease translation. (See, e.g., Kedde et al. (2010) Nature Cell Biology, herein incorporated by reference in its entirety).

Polynucleotides may comprise at least one histone stem-loop and a polyA region or polyadenylation signal. Non-limiting examples of polynucleotide sequences encoding for at least one histone stem loop and a polyA region or a polyadenylation signal are described in International Patent Publication No. WO2013/120497, WO2013/120629, WO2013/120500, WO2013/120627, WO2013/120498, WO2013/120626, WO2013/120499 and WO2013/120628, the sequences of each of which are incorporated herein by reference.

### PolyA Tail

Incorporating the polyA tail in the DNA plasmid also overcomes the tail length variability that arises from enzymatic polyadenylation using polyA polymerase. PolyA tails of >100 bp are optimal for therapeutic mRNAs; however, the DNA sequences that encode these long polyA stretches can destabilize the DNA plasmids used for transcription. A solution to overcome this stability issue is to include a short UGC linker in the polyA tail (US 2017/0166905, which is incorporated herein by reference). The Pfizer-BioNTech vaccine BNT162b2 against SARS-CoV-2 uses this strategy and contains a 10 bp UGC linker to produce the sequence A30(10 bp UGC linker)A70. Together, these innovations have overcome significant manufacturing bottlenecks and facilitated the development of a simple, cost-effective and scalable one-step mRNA synthesis process.

A polynucleotide or nucleic acid (e.g., an mRNA) may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid.

During RNA processing, a long chain of adenosine nucleotides (polyA region) is normally added to messenger RNA (mRNA) molecules to increase the stability of the molecule. Immediately after transcription, the 3′-end of the transcript is cleaved to free a 3′-hydroxy. Then polyA polymerase adds a chain of adenosine nucleotides to the RNA. The process, called polyadenylation, adds a polyA region that is between 100 and 250 residues long (SEQ ID NO: 16957).

Unique polyA region lengths may provide certain advantages to the alternative polynucleotides of the present disclosure.

Generally, the length of a polyA region of the present disclosure is at least 30 nucleotides in length. In another embodiment, the polyA region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides.

In some instances, the polyA region may be 80 nucleotides (SEQ ID NO: 16993), 120 nucleotides (SEQ ID NO: 16988), 160 nucleotides (SEQ ID NO: 16994) in length on an alternative polynucleotide molecule described herein.

In other instances, the polyA region may be 20 (SEQ ID NO: 16995), 40 (SEQ ID NO: 16996), 80 (SEQ ID NO: 16993), 100 (SEQ ID NO: 16997), 120 (SEQ ID NO: 16988), 140 (SEQ ID NO: 16998) or 160 nucleotides (SEQ ID NO: 16994) in length on an alternative polynucleotide molecule described herein.

In some cases, the polyA region is designed relative to the length of the overall alternative polynucleotide. This design may be based on the length of the coding region of the alternative polynucleotide, the length of a particular feature or region of the alternative polynucleotide (such as mRNA), or based on the length of the ultimate product expressed from the alternative polynucleotide. When relative to any feature of the alternative polynucleotide (e.g., other than the mRNA portion which includes the poly-A region) the polyA region may be 10, 20, 30, 40, 50, 60, 70, 80, 90 or 100% greater in length than the additional feature. The polyA region may also be designed as a fraction of the alternative polynucleotide to which it belongs. In this context, the polyA region may be 10, 20, 30, 40, 50, 60, 70, 80, or 90% or more of the total length of the construct or the total length of the construct minus the polyA region.

In certain cases, engineered binding sites and/or the conjugation of polynucleotides (e.g., mRNA) for polyA binding protein may be used to enhance expression. The engineered binding sites may be sensor sequences which can operate as binding sites for ligands of the local microenvironment of the polynucleotides (e.g., mRNA). As a non-limiting example, the polynucleotides (e.g., mRNA) may include at least one engineered binding site to alter the binding affinity of polyA binding protein (PABP) and analogs thereof. The incorporation of at least one engineered binding site may increase the binding affinity of the PABP and analogs thereof.

Additionally, multiple distinct polynucleotides (e.g., mRNA) may be linked together to the PABP (polyA binding protein) through the 3′-end using alternative nucleotides at the 3′-terminus of the polyA region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site.

In certain cases, a polyA region may be used to modulate translation initiation. While not wishing to be bound by theory, the polyA region recruits PABP which in turn can interact with translation initiation complex and thus may be essential for protein synthesis.

In some cases, a polyA region may also be used in the present disclosure to protect against 3′-5′-exonuclease digestion.

In some instances, a polynucleotide (e.g., mRNA) may include a polyA-G Quartet. The G-quartet is a cyclic hydrogen bonded array of four guanosine nucleotides that can be formed by G-rich sequences in both DNA and RNA. In such embodiments, the G-quartet is incorporated at the end of the polyA region. The resultant polynucleotides (e.g., mRNA) may be assayed for stability, protein production and other parameters including half-life at various time points. It has been discovered that the polyA-G quartet results in protein production equivalent to at least 75% of that seen using a polyA region of 120 nucleotides alone (SEQ ID NO: 16988).

In some cases, a polynucleotide (e.g., mRNA) may include a polyA region and may be stabilized by the addition of a 3′-stabilizing region. The polynucleotides (e.g., mRNA) with a poly-A region may further include a 5′-cap structure.

In other cases, a polynucleotide (e.g., mRNA) may include a poly-A-G Quartet. The polynucleotides (e.g., mRNA) with a poly-A-G Quartet may further include a 5′-cap structure.

In some cases, the 3′-stabilizing region which may be used to stabilize a polynucleotide (e.g., mRNA) including a polyA region or poly-A-G Quartet may be, but is not limited to, those described in International Patent Publication No. WO2013/103659, the polyA regions and poly-A-G Quartets of which are incorporated herein by reference. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxythymine, 2′,3′-dideoxynucleosides, such as 2′, 3′-dideoxyadenosine, 2′,3′-dideoxyuridine, 2′, 3′-dideoxycytosine, 2′, 3′-dideoxyguanosine, 2′,3′-dideoxythymine, a 2′-deoxynucleoside, or an O-methylnucleoside.

In other cases, a polynucleotide such as, but not limited to mRNA, which includes a polyA region or a poly-A-G Quartet may be stabilized by an alteration to the 3′-region of the polynucleotide that can prevent and/or inhibit the addition of oligio (U) (see e.g., International Patent Publication No. WO2013/103659).

In yet other instances, a polynucleotide such as, but not limited to mRNA, which includes a polyA region or a poly-A-G Quartet may be stabilized by the addition of an oligonucleotide that terminates in a 3′-deoxynucleoside, 2′,3′-dideoxynucleoside 3-O-methylnucleosides, 3′-O-ethylnucleosides, 3′-arabinosides, and other alternative nucleosides known in the art and/or described herein.

### Chain Terminating Nucleosides

A nucleic acid may include a chain terminating nucleoside. In some embodiments, a chain terminating nucleoside may include those nucleosides deoxy genated at the 2′ and/or 3′ positions of their sugar group. Such species may include 3′-deoxyadenosine (cordycepin), 3′-deoxy uridine, 3′-deoxy cytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxy cytosine, 2′,3′-dideoxyguanosine, and 2′,3′-dideoxythymine.

An exemplary utility of a chain terminating nucleoside is described in e.g., US20140342402, which is incorporated herein by reference. For example, a chain terminating nucleoside incorporated at the 3′ end of an mRNA comprising a histone loop stabilizes said mRNA. Specifically, the chain-terminating nucleoside blocks the addition of a 3′-terminal oligo(U) sequence to the mRNA containing the histone stem-loop. When the 3′-terminal oligo(U) sequence cannot be added, degradation of the mRNA is retarded. The mRNA then remains available to the translational machinery for a longer time, resulting in higher levels of protein synthesis.

### Nucleic Acid Vaccine Expression

In certain aspects, a nucleic acid vaccine of the present disclosure (e.g., a vaccine comprising DNA, RNA, or any variant thereof) may present at least one antigen (e.g., methanogen antigen, cell surface protein of a methanogen or a fragment thereof) as a free antigen, as a secreted antigen, or as a membrane-tethered antigen.

In some embodiments, a nucleic acid vaccine presents at least one antigen as a free antigen. In some such embodiments, the nucleic acid vaccine comprises a sequence encoding at least one antigen, but does not comprise a signal peptide sequence or a transmembrane domain sequence. In some embodiments, a polypeptide encoded by such a nucleic acid vaccine may reside in the cell cytoplasm.

In some embodiments, a nucleic acid vaccine presents at least one antigen as a secreted antigen. In some such embodiments, the nucleic acid vaccine comprises a sequence encoding at least one antigen and a signal peptide sequence, but does not comprise a transmembrane domain sequence. In some embodiments, a polypeptide encoded by such a nucleic acid vaccine is secreted from a cell.

In some embodiments, a nucleic acid vaccine presents at least one antigen as a membrane-tethered antigen. In some such embodiments, the nucleic acid vaccine comprises a sequence encoding at least one antigen, a signal peptide sequence, and a transmembrane domain sequence. In some embodiments, a polypeptide encoded by such a nucleic acid vaccine is tethered to a cell membrane. In preferred embodiments, a polypeptide encoded by such a nucleic acid vaccine is tethered to a cell cytoplasmic membrane.

In certain aspects, any signal peptide sequence can be used in a nucleic acid vaccine of the present disclosure.

In some embodiments, a signal peptide sequence is of Bos taurus (cow). Signal peptide sequences are known in the art. For example, signal peptide sequences of various mammals (e.g., ruminants) are available at World Wide Web at signalpeptide.de and uniprot.org. Representative sequences are shown in Table 5A.

In some embodiments, a signal peptide sequence is from any one of the mRNA vaccines that are known in the art. For example, in some embodiments, a signal peptide sequence is from a COVID-19 mRNA vaccine. In some embodiments, a signal peptide sequence is from the BNT162b2 vaccine (BioNTech/Pfizer). In some embodiments, a signal peptide sequence is from the mRNA-1273 vaccine (Moderna).

In some embodiments, a signal peptide sequence of the BNT162b2 SARS CoV2 spike-encoding mRNA vaccine (BioNTech/Pfizer) is used for the nucleic acid vaccine of the present disclosure. An exemplary sequence is shown in Table 5C. Said sequence is also described at World Wide Web at github.com/NAalytics/Assemblies-of-putative-SARS-CoV2-spike-encoding-mRNA-sequences-for-vaccines-BNT-162b2-and-mRNA-1273/.

In certain aspects, any transmembrane domain sequence can be used in a nucleic acid vaccine of the present disclosure.

In some embodiments, a transmembrane domain sequence is from any one of the mRNA vaccines that are known in the art. For example, in some embodiments, a transmembrane domain sequence is from a COVID-19 mRNA vaccine. In some embodiments, a transmembrane domain sequence is from the BNT162b2 vaccine (BioNTech/Pfizer). In some embodiments, a transmembrane domain sequence is from the mRNA-1273 vaccine (Moderna).

In some embodiments, a transmembrane domain sequence is of Bos taurus (cow). Comprehensive signal peptide sequences are known in the art. For example, transmembrane domain sequences of various mammals (e.g., ruminants) are available at World Wide Web at membranome.org and also described in Lomize et al., (2017) Nucleic Acids Research 45:250-255, which is incorporated herein by reference. Representative sequences are shown in Table 5B.

In some embodiments, a transmembrane domain sequence of the BNT162b2 SARS CoV2 spike-encoding mRNA vaccine (BioNTech/Pfizer) is used for the nucleic acid vaccine of the present disclosure. An exemplary sequence is shown in Table 5C. In some embodiments, a cytoplasmic domain sequence of the BNT162b2 SARS CoV2 spike-encoding mRNA vaccine (BioNTech/Pfizer) is used for the nucleic acid vaccine of the present disclosure. In some embodiments, both the transmembrane domain and cytoplasmic domain sequences of the BNT162b2 SARS CoV2 spike-encoding mRNA vaccine (BioNTech/Pfizer) are used for the nucleic acid vaccine of the present disclosure. An exemplary sequence is shown in Table 5C. Such transmembrane domain and cytoplasmic domain sequences are available at World Wide Web at github.com/NAalytics/Assemblies-of-putative-SARS-CoV2-spike-encoding-mRNA-sequences-for-vaccines-BNT-162b2-and-mRNA-1273/.

TABLE 5A
Representative signal peptide sequences of Bos Taurus (cow)

See SEQ ID NO: 12182 to SEQ ID NO: 12361 for the representative signal peptide
sequences of Bos Taurus (cow).

TABLE 5B
Representative transmembrane (TM) sequences of Bos Taurus (cow) genome

See SEQ ID NO: 12362 to SEQ ID NO: 16672 for the representative transmembrane
sequences of Bos Taurus (cow).

TABLE 5C
Exemplary signal peptide sequence, transmembrane (TM) domain sequence,
and cytoplasmic domain sequence of the BNT162b2 SARS CoV2 spike-encoding
mRNA vaccine

| | Amino acid | Native nucleic | Bovine codon-optimized |
|---|---|---|---|
| Domain | sequence | acid sequence | nucleic acid |
| Signal | MFVFLVLLPLVS | ATGTTCGTGTTCCTGGTG | ATGTTCGTGTTCCTC |
| peptide | S | CTGCTGCCTCTGGTGTCC | GTGCTCCTGCCCCTC |
| | (SEQ ID NO: | AGCCAGTGTGTG | GTCTCCAGCCAGTGC |
| | 16958) | (SEQ ID NO: 16961) | GTG |
| | | | (SEQ ID NO: 16964) |
| TM domain | PWYIWLGFIAGL | CCCTGGTACATCTGGCTG | CCCTGGTACATCTGG |
| | IAIVMVTIMLCC | GGCTTTATCGCCGGACTG | CTGGGCTTCATCGCA |
| | M | ATTGCCATCGTGATGGTC | GGACTGATCGCCATC |
| | (SEQ ID NO: | ACAATCATGCTGTGTTGC | GTCATGGTCACCATA |
| | 16959) | ATG (SEQ ID NO: 16962) | ATGCTCTGTTGCATG |
| | | | (SEQ ID NO: 16991) |
| Cytoplasmic | MTSCCSCLKGCC | ATGACCAGCTGCTGTAG | ACCTCCTGCTGTAGC |
| domain | SCGSCCKFDEDD | CTGCCTGAAGGGCTGTTG | TGCCTGAAAGGCTGT |
| | SEPVLKGVKLHY | TAGCTGTGGCAGCTGCTG | TGCAGCTGCGGGAG |
| | T | CAAGTTCGACGAGGACG | CTGCTGCAAATTCGA |
| | (SEQ ID NO: | ATTCTGAGCCCGTGCTGA | CGAAGACGATAGCG |
| | 16960) | AGGGCGTGAAACTGCAC | AACCTGTGCTCAAAG |
| | | TACACA | GGGTGAAGTTGCATT |
| | | (SEQ ID NO: 16963) | ACACA |
| | | | (SEQ ID NO: 16992) |

### Pharmaceutical Composition

Vaccines, antibodies, milk, animal feed, agents (e.g., an agent that reduces methane production in a ruminant, a probiotic bacterial strain, etc.), or other compositions of the present disclosure may be in a pharmaceutical composition, and thus further comprise at least one excipient and/or carrier described herein or those known in the art.

The vaccine of the present disclosure (e.g., protein vaccine, nucleic acid vaccine (e.g., DNA vaccine, RNA vaccine, etc.) may comprise at least one excipient that (1) increases stability; (2) increases cell transfection; (3) permits the sustained or delayed release (e.g., from a depot formulation); (4) alters the biodistribution (e.g., target to specific tissues or cell types); (5) increases the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure include, without limitation, lipidoids, liposomes, lipid nanoparticles, polymers, lipoplexes, core-shell nanoparticles, peptides, proteins, hyaluronidase, nanoparticle mimics, and combinations thereof.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient (e.g., the vaccine). The amount of the active ingredient may be generally equal to the dosage of the active ingredient which would be administered to a ruminant and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the ruminant being treated and further depending upon the route by which the composition is to be administered. For example, the composition may comprise between 0.01% and 99% (w/w) of the active ingredient. By way of example, the composition may comprise between 0.01% and 100%, e.g., between 0.05 and 50%, between 0.1-30%, between 5-80%, at least 80% (w/w) active ingredient.

In some embodiments, the active ingredient is a nucleic acid. Accordingly, in some embodiments, the composition may comprise between 0.01% and 99% (w/w) of the nucleic acid (DNA or RNA). By way of example, the composition may comprise between 0.01% and 100%, e.g., between 0.05 and 50%, between 0.1-30%, between 5-80%, at least 80% (w/w) nucleic acid (DNA or RNA).

In some embodiments, the formulations described herein may comprise at least one nucleic acid, e.g., an antigen-encoding polynucleotide. As a non-limiting example, the formulations may comprise at least 1, 2, 3, 4, or 5 nucleic acids. In some embodiments, the formulation comprises at least 1, 2, 3, 4, or 5 nucleic acids encoding at least one cell surface protein or a fragment thereof of at least one methanogen.

In some embodiments, the formulations described herein may comprise more than one type of polynucleotide, e.g., antigen-encoding polynucleotide. In some embodiments, the formulation may comprise a polynucleotide in linear and circular form. For example, in some embodiments, the formulation may comprise a circular polynucleotide and an IVT polynucleotide.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, the particle size of the lipid nanoparticle may be increased and/or decreased. The change in particle size may be able to help counter biological reaction such as, but not limited to, inflammation or may increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

### Delivery System—Overview

How much protein will be produced from the mRNA template will initially be determined by the amount of intact mRNA that reaches the cytosolic compartment.

In certain embodiments, lipid nanoparticle (“LNP”) delivery technology is used. Upon administration, proteins and other biological components present in the extracellular space can bind at the surface of the nucleic acid LNPs. The polyethylene glycol (PEG)-lipid stabilizing the nucleic acid LNP system against aggregation during manufacturing and storage contains short acyl chains. This design facilitates that the PEG-lipid (also referred to as PEGylated lipid) rapidly dissociates from the LNP following injection, as an essential first step to allow cellular interactions (Harvie et al. (2000) J Pharm Sci, 89:652-663; Chen et al. (2016) J Control Release, 235:236-244). Upon intravenous administration, it was found that the surface of neutrally-charged LNPs is strongly enriched with apolipoprotein E (ApoE), which leads to enhanced uptake by hepatocytes through low density lipoprotein (LDL) receptor-mediated endocytosis (Akinc et al. (2010) Mol Therapy, 18(7): 1357-1364). ApoE binding also plays a critical role in the uptake of the nucleic acid LNP vaccines after intramuscular injection. Vaccine targeted cell types, such as dendritic cells (DCs) and monocytes, highly express LDL receptors and other scavenger receptors (Liang et al. (2017) Mol Therapy, 25(12):2635-2647). Moreover, the transfection of human DCs with nucleic acid LNPs in an in vitro setting was also reported to be promoted in the presence of ApoE. Upon internalization, nucleic acid LNPs are routed through the endo-lysosomal compartment, where most of the nucleic acid LNPs remain entrapped in endosomes and degrade over time. While the intracellular trafficking and underlying mechanisms on how LNPs enable the escape of nucleic acid from the endosomes to reach the cytoplasm are still not fully understood, it may be facilited by the changes in the LNPs. Specifically, the ionizable lipid components of the LNPs (pKa <7) become protonated due to the acidic pHs in the endosomes, and leads to lipid exchange with anionic phospholipids of the endosomal membrane. This lipid mixing also induces a non-bilayer structure conversion in the LNPs (i.e. lamellar-to-inverted hexagonal phase), which facilitates the release of nucleic acid (e.g., mRNA) from the LNPs (Sayers et al. (2019) Mol Therapy, 27(11): 1950-1962). These effects of membrane fusion and structural changes in the LNPs are suggested to drive the destabilization of the endosomal membrane and eventually the endosomal escape of the nucleic acid (e.g., mRNA). It was recently demonstrated that ApoE binding also affects the internal structure of LNPs, which might contribute to the endosomal escape and successful cytosolic delivery of mRNA (Sebastiani et al. (2021) ACS Nano, 15(4):6709-6722). Thus, depending on the fusogenic properties of the LNPs a fraction of the mRNA can be released into the cytosol, where the mRNA molecules need to be recruited in ribosomes in order to be translated into proteins.

The vaccines of the present disclosure contemplates the use of the classical LNPs as well as the “next-generation” LNP delivery systems, which exemplary COVID-19 mRNA vaccines of BioNTech and Moderna employ. Such LNP delivery systems are composed of biodegradable ionizable lipids that introduces ester-linkages in the lipid tails. As an example, the usage of the SM-102 lipid in Moderna's mRNA-1273 vaccine was found to outperform Onpattro's MC3 LNPs for the intramuscular (i.m.) delivery of mRNA in rodents and non-human primates, given its improved tolerability and higher endosomal escape efficiency (Sabnis et al. (2018) Mol Therapy, 26(6):1509-1519; Hassett et al. (2019) Mol Therapy Nucl Acids, 15:1-11).

A strong chemical similarity can be found between the ionizable lipid ALC-0315 (Acuitas' proprietary lipid; see e.g., U.S. Pat. No. 10,166,298 and WO 2017/075531) and SM-102 (Moderna) used in the LNP formulations of BNT162b2 and mRNA-1273, respectively. The LNPs may also contain similar helper lipids; 1,2-distearoyl-snglycero-3-phosphocholine (DSPC), cholesterol and a diffusible PEG-lipid (2-[(polyethylene glycol)-2000]-N,N-ditetradecylacetamide, PEG2000-DMA in BNT162b2 or 1,2-dimyristoyl-rac-glycero3-methoxypolyethylene glycol-2000, PEG2000-DMG in mRNA-1273). All three mRNA LNPs contain a lipid formulation of ionizable lipid: DSPC: cholesterol: PEG-lipid at molar ratios of 50:10:38.5:1.5 mol %, and an mRNA-to-lipid ratio of 0.05 (wt/wt).

In preferred embodiments, the vaccines of the present disclosure have similar or identical LNP formulations as those of the vaccines that have been validated in a mammal (e.g., human) (e.g., Moderna's SPIKEVAX, each 0.5 mL dose of which contains a total lipid content of 1.93 mg (SM-102, polyethylene glycol [PEG] 2000 dimyristoyl glycerol [DMG], cholesterol, and 1,2-distearoyl-sn-glycero-3-phosphocholine [DSPC]), 0.31 mg tromethamine, 1.18 mg tromethamine hydrochloride, 0.043 mg acetic acid, 0.20 mg sodium acetate trihydrate, and 43.5 mg sucrose) (e.g., Pfizer/BioNTech's COMIRNATY purple caps, each 0.3 mL dose of which contains lipids (0.43 mg ((4-hydroxybutyl)azanediyl)bis(hexane-6,1-diyl)bis(2-hexyldecanoate), 0.05 mg 2-(polyethylene glycol 2000)-N,N-ditetradecylacetamide, 0.09 mg 1,2-distearoyl-sn-glycero-3-phosphocholine, and 0.2 mg cholesterol), 0.01 mg potassium chloride, 0.01 mg monobasic potassium phosphate, 0.36 mg sodium chloride, 0.07 mg dibasic sodium phosphate dihydrate, and 6 mg sucrose. The diluent (sterile 0.9% Sodium Chloride Injection, USP) contributes an additional 2.16 mg sodium chloride per dose) (e.g., Pfizer/BioNTech's COMIRNATY gray caps, each 0.3 mL dose of which contains lipids (0.43 mg ((4-hydroxybutyl)azanediyl)bis(hexane-6,1-diyl)bis(2-hexyldecanoate), 0.05 mg 2-(polyethylene glycol 2000)-N,N-ditetradecylacetamide, 0.09 mg 1,2-distearoyl-sn-glycero-3-phosphocholine, and 0.19 mg cholesterol), 0.06 mg tromethamine, 0.4 mg tromethamine hydrochloride, and 31 mg sucrose).

### Lipidoids

The synthesis of lipidoids (e.g., any material having characteristics of a lipid) has been extensively described and formulations comprising these compounds are particularly suited for delivery of nucleic acids (see Mahon et al., Bioconjug Chem. 2010 21: 1448-1454; Schroeder et al., J Intern Med. 2010 267:9-21; Akinc et al., Nat Biotechnol. 2008 26:561-569; Love et al., Proc Natl Acad Sci USA. 2010 107: 1864-1869; Siegwart et al., Proc Natl Acad Sci USA. 2011 108: 12996-3001; all of which are incorporated herein in their entireties). Accordingly, lipoids are suitable means to deliver the nucleic acid vaccine of the present disclosure (e.g., especially those comprising DNA or RNA).

While these lipidoids have been used to effectively deliver double-stranded small interfering RNA molecules in rodents and non-human primates (see Akinc et al., Nat Biotechnol. 2008 26:561-569; Frank-Kamenetsky et al., Proc Natl Acad Sci USA. 2008 105: 11915-11920; Akinc et al., Mol Ther. 2009 17:872-879; Love et al., Proc Natl Acad Sci USA. 2010 107: 1864-1869; Leuschner et al., Nat Biotechnol. 2011 29: 1005-1010; all of which are incorporated herein in their entirety), the present disclosure encompasses their formulation and use in delivering nucleic acid vaccines or polynucleotides contained therein.

Complexes, micelles, liposomes or particles can be prepared comprising these lipidoids and therefore, can result in an effective delivery of the nucleic acid, as judged by the production of an encoded protein, following the administration of a lipidoid formulation to a ruminant.

In vivo delivery of nucleic acids may be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010); herein incorporated by reference in its entirety), C12-200 (including derivatives and variants), and MD1, can be tested for in vivo activity.

The lipidoid referred to herein as “98N12-5” is disclosed by Akinc et al., Mol Ther. 2009 17:872-879 and is incorporated by reference in its entirety. The lipidoid referred to herein as “C12-200” is disclosed by Love et al., Proc Natl Acad Sci USA. 2010 107: 1864-1869 and Liu and Huang, Molecular Therapy. 2010 669-670; both of which are herein incorporated by reference in their entirety. The lipidoid formulations can comprise particles comprising either 3 or 4 or more components in addition to the nucleic acid. As an example, formulations with certain lipidoids, include, but are not limited to, 98N12-5 and may comprise 42% lipidoid, 48% cholesterol and 10% PEG (C14 alkyl chain length). As another example, formulations with certain lipidoids, include, but are not limited to, C12-200 and may comprise 50% lipidoid, 10% disteroylphosphatidyl choline, 38.5% cholesterol, and 1.5% PEG-DMG.

In some embodiments, a polynucleotide formulated with a lipidoid for systemic intravenous administration can target the liver. For example, a final optimized intravenous formulation using polynucleotides, and comprising a lipid molar composition of 42% 98N12-5, 48% cholesterol, and 10% PEG-lipid with a final weight ratio of about 7.5 to 1 total lipid to polynucleotides, and a C14 alkyl chain length on the PEG lipid, with a mean particle size of roughly 50-60 nm, can result in the distribution of the formulation to be greater than 90% to the liver.(see Akinc et al., Mol Ther. 2009 17:872-879; herein incorporated by reference in its entirety). In another example, an intravenous formulation using a C12-200 (see U.S. provisional application 61/175,770 and published international application WO2010129709, each of which is herein incorporated by reference in their entirety) lipidoid may have a molar ratio of 50/10/38.5/1.5 of C12-200/disteroylphosphatidyl choline/cholesterol/PEG-DMG, with a weight ratio of 7 to 1 total lipid to polynucleotides, and a mean particle size of 80 nm may be effective to deliver polynucleotides to hepatocytes (see, Love et al., Proc Natl Acad Sci USA. 2010 107: 1864-1869 herein incorporated by reference in its entirety).

The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879 herein incorporated by reference in its entirety), use of a lipidoid-formulated nucleic acid vaccines to deliver the formulation to other cells types including, but not limited to, endothelial cells, myeloid cells, and muscle cells may not be similarly size-limited.

Use of lipidoid formulations to deliver siRNA in vivo to other non-hepatocyte cells such as myeloid cells and endothelium has been reported (see Akinc et al., Nat Biotechnol. 2008 26:561-569; Leuschner et al., Nat Biotechnol. 2011 29: 1005-1010; Cho et al. Adv. Funct. Mater. 2009 19:3112-3118; 8^{th} International Judah Folkman Conference, Cambridge, MA Oct. 8-9, 2010; each of which is herein incorporated by reference in its entirety). Effective delivery to myeloid cells, such as monocytes, lipidoid formulations may have a similar component molar ratio. Different ratios of lipidoids and other components including, but not limited to, disteroylphosphatidyl choline, cholesterol, and PEG-DMG, may be used to optimize the formulation of the nucleic acid vaccines for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and % 1.5 PEG-DMG (see Leuschner et al., Nat Biotechnol 2011 29: 1005-1010; herein incorporated by reference in its entirety). In certain cases, the use of lipidoid formulations for the localized delivery of nucleic acids to cells (such as, but not limited to, adipose cells and muscle cells) via either subcutaneous or intramuscular delivery, may not require all of the formulation components desired for systemic delivery, and as such may comprise only the lipidoid and the nucleic acid vaccine.

Combinations of different lipidoids may be used to improve the efficacy of the polynucleotide-directed protein production as the lipidoids may be able to increase cell transfection by the nucleic acid vaccine; and/or increase the translation of encoded protein (see Whitehead et al., Mol. Ther. 2011, 19: 1688-1694, herein incorporated by reference in its entirety).

In some embodiments, the vaccines of the present disclosure are formulated in lipid or saline. In preferred embodiments, the nucleic acid vaccines are formulated in lipid.

### Liposomes, Lipoplexes, and Lipid Nanoparticles

The nucleic acid vaccines of the invention can be formulated using one or more liposomes, lipoplexes, or lipid nanoparticles.

### Liposomes

In some embodiments, pharmaceutical compositions of nucleic acid vaccines comprise liposomes. Liposomes are artificially-prepared vesicles which may primarily be composed of a lipid bilayer and may be used as a delivery vehicle for the administration of pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity, and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, PA). In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6: 1438-1447; Jeffs et al. Pharm Res. 2005 22:362-372; Morrissey et al., Nat Biotechnol. 2005 2: 1002-1007; Zimmermann et al., Nature. 2006 441: 111-114; Heyes et al. J Contr Rel. 2005 107:276-287; Semple et al. Nature Biotech. 2010 28: 172-176; Judge et al. J Clin Invest. 2009 119:661-673; deFougerolles Hum Gene Ther. 2008 19: 125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations may be composed of 3 to 4 lipid components in addition to the polynucleotide. As an example a liposome can comprise, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may comprise, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In preferred embodiments, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In some embodiments, pharmaceutical compositions may include liposomes which may be formed to deliver polynucleotides which may encode at least one antigen or any other polypeptide of interest. The nucleic acid vaccine may be encapsulated by the liposome and/or it may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In other embodiments, liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are herein incorporated by reference in its entirety.

In other embodiments, the polynucleotide which may encode an antigen may be formulated in a cationic oil-in-water emulsion where the emulsion particle comprises an oil core and a cationic lipid which can interact with the polynucleotide anchoring the molecule to the emulsion particle (see International Pub. No. WO2012006380; herein incorporated by reference in its entirety).

In some embodiments, the nucleic acid vaccines may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in its entirety.

In other embodiments, the lipid formulation may include at least cationic lipid, a lipid which may enhance transfection and a least one lipid which contains a hydrophilic head group linked to a lipid moiety (International Pub. No. WO2011076807 and U.S. Pub. No. 20110200582; the contents of each of which is herein incorporated by reference in their entirety).

In other embodiments, the polynucleotides encoding an immunogen may be formulated in a lipid vesicle which may have crosslinks between functionalized lipid bilayers (see U.S. Pub. No. 20120177724, the contents of which is herein incorporated by reference in its entirety).

In some embodiments, the polynucleotides may be formulated in a lipsome as described in International Patent Publication No. WO2013086526, the contents of which is herein incorporated by reference in its entirety. The nucleic acid vaccines may be encapsulated in a liposome using reverse pH gradients and/or optimized internal buffer compositions as described in International Patent Publication No. WO2013086526, the contents of which is herein incorporated by reference in its entirety.

In some embodiments, the nucleic acid vaccine pharmaceutical compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In some embodiments, the cationic lipid may be a low molecular weight cationic lipid such as those described in US Patent Application No. 20130090372, the contents of which are herein incorporated by reference in its entirety.

In some embodiments, the nucleic acid vaccines may be formulated in a lipid vesicle which may have crosslinks between functionalized lipid bilayers.

In some embodiments, the nucleic acid vaccines may be formulated in a liposome comprising a cationic lipid. The liposome may have a molar ratio of nitrogen atoms in the cationic lipid to the phophates in the RNA (N:P ratio) of between 1:1 and 20:1 as described in International Publication No. WO2013006825, herein incorporated by reference in its entirety. In another embodiment, the liposome may have a N:P ratio of greater than 20:1 or less than 1:1.

In some embodiments, the nucleic acid vaccines may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In other embodiments, the nucleic acid vaccines may be formulated in a lipid-polycation complex which may further include a non-cationic lipid such as, but not limited to, cholesterol or dioleoyl phosphatidylethanolamine (DOPE).

In some embodiments, the nucleic acid vaccines may be formulated in an aminoalcohol lipidoid. Aminoalcohol lipidoids which may be used in the present invention may be prepared by the methods described in U.S. Pat. No. 8,450,298, herein incorporated by reference in its entirety.

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28: 172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver nucleic acid to various antigen presenting cells (Basha et al. Mol Ther. 2011 19:2186-2200; herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about about 5: 1 to about 20: 1, from about 10: 1 to about 25: 1, from about 15: 1 to about 30: 1 and/or at least 30: 1.

### Lipid Nanoparticles

In certain aspects, the vaccines (e.g., nucleic acid vaccines) of the present disclosure may be formulated in a lipid nanoparticle (LNP) described herein or of those known in the art.

In some embodiments, the lipid nanoparticle comprises an ionizable lipid, a helper lipid, a PEGylated lipid, a structural lipid (e.g., sterol), or any combination thereof.

In some embodiments, the lipid nanoparticle comprises an ionizable lipid, a helper lipid, a PEGylated lipid, and a structural lipid (e.g., sterol).

In some embodiments, the ionizable lipid is an ionizable cationic lipid. In some embodiments, the helper lipid is a neutral lipid or a non-cationic lipid.

In some embodiments, the lipid nanoparticle formulations described herein may comprise an ionizable lipid (e.g., a cationic lipid), a PEGylated lipid (also referred to as PEG lipid), and a structural lipid; and optionally comprise a helper lipid (e.g., a non-cationic lipid, phospholipid). In some embodiments, the lipid nanoparticle may comprise a molar ratio of about 20-60% ionizable lipid: about 5-25% helper lipid: about 25-55% structural lipid; and about 0.5-15% PEGylated lipid. In some embodiments, the lipid nanoparticle may comprise a molar ratio of about 40-60% of ionizable lipid (e.g., cationic lipid), about 5-15% of a helper lipid, about 1-2% of a PEG lipid and about 30-50% of a structural lipid. In some embodiments, the lipid nanoparticle may comprise a molar ratio of about 50% ionizable lipid (e.g., cationic lipid), about 10% helper lipid, about 1.5% PEG lipid and about 38.5% structural lipid. In other embodiments, the lipid nanoparticle may comprise a molar ratio of about 55% ionizable lipid (e.g., cationic lipid), about 10% helper lipid, about 2.5% PEG lipid and about 32.5% structural lipid.

In some embodiments, the lipid nanoparticle formulations described herein may be 4 component lipid nanoparticles. The lipid nanoparticle may comprise a ionizable lipid (e.g., cationic lipid), a helper lipid, a PEG lipid, and a structural lipid. In some embodiments, the lipid nanoparticle may comprise a molar ratio of about 20-60% ionizable lipid: about 5-25% helper lipid: about 25-55% structural lipid; and about 0.5-15% PEGylated lipid. In some embodiments, the lipid nanoparticle may comprise a molar ratio of about 40-60% of ionizable lipid (e.g., cationic lipid), about 5-15% of a helper lipid, about 1-2% of a PEG lipid and about 30-50% of a structural lipid. In some embodiments, the lipid nanoparticle may comprise a molar ratio of about 50% ionizable lipid (e.g., cationic lipid), about 10% helper lipid, about 1.5% PEG lipid and about 38.5% structural lipid. In other embodiments, the lipid nanoparticle may comprise a molar ratio of about 55% ionizable lipid (e.g., cationic lipid), about 10% helper lipid, about 2.5% PEG lipid and about 32.5% structural lipid.

In some embodiments, the lipid nanoparticle formulations described herein may comprise an ionizable lipid (e.g., cationic lipid), a helper lipid, a PEG lipid, and a structural lipid. In some embodiments, the lipid nanoparticle may comprise a molar ratio of about 20-60% ionizable lipid: about 5-25% helper lipid: about 25-55% structural lipid; and about 0.5-15% PEGylated lipid. In some embodiments, the lipid nanoparticle may comprise a molar ratio of about 40-60% of ionizable lipid (e.g., cationic lipid), about 5-15% of a helper lipid, about 1-2% of a PEG lipid and about 30-50% of a structural lipid. In some embodiments, the lipid nanoparticle may comprise a molar ratio of about 50% ionizable lipid (e.g., cationic lipid), about 10% helper lipid, about 1.5% PEG lipid and about 38.5% structural lipid. In other embodiments, the lipid nanoparticle may comprise a molar ratio of about 55% ionizable lipid (e.g., cationic lipid), about 10% helper lipid, about 2.5% PEG lipid and about 32.5% structural lipid.

Exemplary lipid nanoparticle compositions and methods of making same are described, for example, in Semple et al. (2010) Nat. Biotechnol. 28: 172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In certain aspects, the nucleic acid (e.g., DNA or RNA) to total lipid ratio is between 0.01 and 10 (wt/wt). In some embodiments, the nucleic acid (e.g., DNA or RNA) to total lipid ratio is at least about 0.005, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 (wt/wt). In some embodiments, the nucleic acid (e.g., DNA or RNA) to total lipid ratio is at least about 0.03, 0.04, 0.05, 0.06, 0.07, or 0.08 (wt/wt). In some embodiments, the nucleic acid (e.g., DNA or RNA) to total lipid ratio is at least about 0.05 (wt/wt).

In some embodiments, a lipid nanoparticle may be relatively homogenous. A polydispersity index (PDI) may be used to indicate the homogeneity of a lipid nanoparticle, e.g., the particle size distribution of the lipid nanoparticles. A small (e.g., less than 0.3) polydispersity index generally indicates a narrow particle size distribution.

In some embodiments, a lipid nanoparticle may have a polydispersity index from about 0 to about 0.3. For example, a lipid nano particle may have a PDI of at least about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.3. In some embodiments, the polydispersity index of a lipid nanoparticle may be from about 0.10 to about 0.20.

In other embodiments, a lipid nanoparticle may have a polydispersity index that is less than about 0.6. For example, a lipid nanoparticle may have a PDI that is less than about 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, or 0.6. In some embodiments, the polydispersity index of a lipid nanoparticle may be less than 0.4.

In some embodiments, the nucleic acid vaccine formulation comprises the polynucleotide is a nanoparticle which may comprise at least one lipid. In some embodiments, the lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9, 12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z, 12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9, 12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

In some embodiments, the lipid nanoparticle comprises SM-102, polyethylene glycol 2000 dimyristoyl glycerol (PEG2000-DMG), 1,2-distearoyl-sn-glycero-3-phosphocholine (DSPC), and cholesterol.

In some embodiments, the lipid nanoparticle comprises SM-102, polyethylene glycol 2000 dimyristoyl glycerol (PEG2000-DMG), 1,2-distearoyl-sn-glycero-3-phosphocholine (DSPC), and cholesterol, wherein (a) the nucleic acid to total lipid ratio is at least about 0.05 (wt/wt), and/or (b) the nanoparticle has a molar ratio of about 50% ionizable lipid: about 10% helper lipid: about 38.5% structural lipid; and about 1.5% PEGylated lipid.

In some embodiments, the lipid nanoparticle comprises ((4-hydroxybutyl)azanediyl)bis(hexane-6,1-diyl)bis(2-hexyldecanoate, 2-(polyethylene glycol 2000)-N,N-ditetradecylacetamide (PEG2000-DMA), 1,2-distearoyl-sn-glycero-3-phosphocholine (DSPC), and cholesterol.

In some embodiments, the lipid nanoparticle comprises ((4-hydroxybutyl)azanediyl)bis(hexane-6,1-diyl)bis(2-hexyldecanoate, 2-(polyethylene glycol 2000)-N,N-ditetradecylacetamide (PEG2000-DMA), 1,2-distearoyl-sn-glycero-3-phosphocholine (DSPC), and cholesterol, wherein (a) the nucleic acid to total lipid ratio is at least about 0.05 (wt/wt), and/or (b) the nanoparticle has a molar ratio of about 50% ionizable lipid: about 10% helper lipid: about 38.5% structural lipid; and about 1.5% PEGylated lipid.

In some embodiments, the lipid nanoparticle further comprises potassium chloride, monobasic potassium phosphate, sodium chloride, dibasic sodium phosphate dihydrate, sucrose, or any combination thereof.

In some embodiments, the lipid nanoparticle further comprises tromethamine, tromethamine hydrochloride, sucrose, or any combination thereof.

In some embodiments, a unit comprises a total lipid content of 1.93 mg (SM-102, polyethylene glycol [PEG] 2000 dimyristoyl glycerol [DMG], cholesterol, and 1,2-distearoyl-sn-glycero-3-phosphocholine [DSPC]), 0.31 mg tromethamine, 1.18 mg tromethamine hydrochloride, 0.043 mg acetic acid, 0.20 mg sodium acetate trihydrate, and 43.5 mg sucrose. In some embodiments, a single unit is administered to a ruminant as a single dose. In other embodiments, a plurality of such a unit is administered to a ruminant as a single dose.

In some embodiments, each unit comprises lipids (0.43 mg ((4-hydroxybutyl)azanediyl)bis(hexane-6,1-diyl)bis(2-hexyldecanoate), 0.05 mg 2-(polyethylene glycol 2000)-N,N-ditetradecylacetamide, 0.09 mg 1,2-distearoyl-sn-glycero-3-phosphocholine, and 0.2 mg cholesterol), 0.01 mg potassium chloride, 0.01 mg monobasic potassium phosphate, 0.36 mg sodium chloride, 0.07 mg dibasic sodium phosphate dihydrate, and 6 mg sucrose. In some embodiments, a single unit is administered to a ruminant as a single dose. In other embodiments, a plurality of such a unit is administered to a ruminant as a single dose.

In some embodiments, each unit comprises lipids (0.43 mg ((4-hydroxybutyl)azanediyl)bis(hexane-6,1-diyl)bis(2-hexyldecanoate), 0.05 mg 2-(polyethylene glycol 2000)-N,N-ditetradecylacetamide, 0.09 mg 1,2-distearoyl-sn-glycero-3-phosphocholine, and 0.19 mg cholesterol), 0.06 mg tromethamine, 0.4 mg tromethamine hydrochloride, and 31 mg sucrose. In some embodiments, a single unit is administered to a ruminant as a single dose. In other embodiments, a plurality of such a unit is administered to a ruminant as a single dose.

In some embodiments, the lipid nanoparticle has a net neutral charge at a neutral pH.

In some embodiments, the lipid nanoparticle has a mean diameter of about 50 nm to about 200 nm. In some embodiments, the lipid nanoparticle has a mean diameter of about 80 nm to about 100 nm.

In some embodiments, the lipid nanoparticle has a polydispersity index (PDI) of less than 0.4.

### 1. Ionizable Lipid

In some embodiments, the ionizable lipid is selected from 3-(didodecylamino)-N 1,N 1,4-tridodecyl-1-piperazineethanamine (KL 10), N 1-[2-(didodecylamino)ethyl]-N1,N4,N4-tridodecyl-1,4-piperazinediethanamine (KL22), 14,25-ditridecy 1-15,18,21,24-tetraaza-octatriacontane (KL25), di((Z)-non-2-en-1-yl) 9-((4-(dimethylamino)butanoyl)oxy)heptadecanedioate (L319), 1,2-dilinoley loxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-((8-|(3P)-cholest-5-en-3-yloxy|octyl}oxy)-N.N-dimethyl-3-[(9Z,12Z)-octadeca-9,12-dien-1-yloxy]propan-1-amine (Octyl-CLinDMA (2R)), (2S)-2-({8-[(3)-cholest-5-en-3-yloxy]octyl}oxy)-N,N-dimethyl-3-[(9Z,12Z)-octadeca-9,12-dien-1-yloxy] propan-1-amine (Octyl-CLinDMA (2S)), ALC-0315, and SM-102.

In some embodiments, the the ionizable lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), 4-hydroxybutyl)azanediyl)bis(hexane-6,1-diyl)bis(2-hexyldecanoate (ALC-0315), SM-102, A18-Iso5-2DC18, A6, 3060i10, or any combination thereof.

In some embodiments, the ionizable lipid (e.g., cationic lipid) may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836; the contents of each of which are herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365, WO2012044638 and WO2013116126 or US Patent Publication No. US20130178541 and US20130225836; the contents of each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115, formula I of US Patent Publication No US20130123338; each of which is herein incorporated by reference in their entirety. As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-1 6, 19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z, 18Z)-N,N-dimethyltetracosa-15,18-dien-5-amine, (14Z, 17Z)-N,N-dimethyltricosa-14,17-dien-4-amine, (19Z,22Z)-N,N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl] pyrrolidine, (20Z)-N,N-dimethylheptacos-20-en-1 0-amine, (15Z)-N,N-dimethyl eptacos-15-en-1 O-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-1 O-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-i-amine, (13Z,16Z)-N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan- 10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl] methyl} cyclopropyl] nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl} dodecan-i-amine, i-[(1R,2S)-2-heptylcyclopropyl]-N,N-dimethyloctadecan-9-amine, 1-[(1 S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z, 12Z)-octadeca-9, 12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, i-{2-[(9Z,12Z)-octadeca-9,12-dien-i-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethylIazetidine, (2S)-1-(hexyloxy)-N,N-dimethyl-3-[(9Z,12Z)-octadeca-9, 12-dien-i-yloxy]propan-2-amine, (2S)-1-(heptyloxy)-N,N-dimethyl-3-[(9Z, 12Z)-octadeca-9, 12-dien-1-yloxy]propan-2-amine, N,N-dimethyl-1-(nonyloxy)-3-[(9Z,12Z)-octadeca-9, 12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(1 1Z,14Z)-icosa-1 1,14-dien-1-yloxy]-N,N-dimethylpropan-2-amine, 1-[(11Z,14Z)-icosa-1 1,14-dien-1-yloxy]-N,N-dimethy 1-3-(octyloxy)propan-2-amine, 1-[(13Z, 16Z)-docosa-13, 16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9, 12-dien-1-yloxy]propan-2-amine, (2R)-1-[(3,7-dimethyloctyl)oxy]-N,N-dimethyl-3-[(9Z, 12Z)-octadeca-9, 12-dien-1-yloxy]propan-2-amine, N,N-dimethyl-1-(octyloxy)-3-({8-[(1S,2S)-2-{[(1R,2R)-2-pentylcyclopropyl] methyl} cyclopropyl] octyl} oxy)propan-2-amine, N,N-dimethyl-1-{[8-(2-oclylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In some embodiments, the lipid may be a cleavable lipid such as those described in International Publication No. WO2012170889, herein incorporated by reference in its entirety.

In some embodiments, the lipid may be a cationic lipid such as, but not limited to, Formula (I) of U.S. Patent Application No. US20130064894, the contents of which are herein incorporated by reference in its entirety.

In some embodiments, the the ionizable lipid (e.g., cationic lipid) may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724, WO201021865, WO2013086373 and WO2013086354; the contents of each of which are herein incorporated by reference in their entirety. In other embodiments, the cationic lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

In some embodiments, the ionizable lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

### 2. PEGylated Lipid

The lipid component of a lipid nanoparticle or lipid nanoparticle formulation may include one or more molecules comprising polyethylene glycol, such as PEGylated, PEG, or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid.

In some embodiments, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In some embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In some embodiments, the PEG-OH lipid includes one or more hydroxyl groups on the PEG chain. In some embodiments, a PEG-OH or hydroxy-PEGylated lipid comprises an —OH group at the terminus of the PEG chain. Each possibility represents a separate embodiment of the present invention.

In some embodiments, the formulation includes from about 0.5% to about 20% on a molar basis of the PEGylated or PEG-modified lipid (e.g., about 0.5 to about 10%, about 0.5 to about 5%, about 1.5%, about 0.5%, about 1.5%, about 3.5%, or about 5% on a molar basis. Exemplary PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in its entirety).

In some embodiments, the lipid moiety of the PEG lipids includes those having lengths of from about C14 to about C22, e.g., from about C14 to about C16. In some embodiments, a PEG moiety, for example an mPEG-NEb, has a size of about 1000, 2000, 5000, 10,000, 15,000 or 20,000 daltons. In some embodiments, the PEG lipid is PEG2000-DMG.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the LNP formulations.

Non-limiting examples of PEG lipids include PEG-modified phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, (2-[(polyethylene glycol)-2000]-N,N-ditetradecylacetamide (PEG2000-DMA), and 1,2-dimyristoyl-rac-glycero3-methoxypolyethylene glycol-2000 (PEG2000-DMG). Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid.

In some embodiments, the PEG lipid includes, but are not limited to, 1,2-dimyristoyl-sn-glycerol methoxypoly ethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1, 2-dimyristyloxlpropy 1-3-amine (PEG-c-DMA).

In some embodiments, the lipid nanoparticles described herein can comprise a PEG lipid which is a non-diffusible PEG. Non-limiting examples of non-diffusible PEGs include PEG-DSG and PEG-DSPE.

PEG lipids are known in the art, such as those described in U.S. Pat. Nos. 8,158,601, 8,492,359, PCT/US2016/000129, WO2012/099755, and WO 2015/130584 A2, which are incorporated herein by reference in their entirety.

In preferred embodiments, the Pegylated lipid comprises (2-[(polyethylene glycol)-2000]-N,N-ditetradecylacetamide (PEG2000-DMA (also called ALC-0159)); and/or polyethylene glycol 2000 dimyristoyl glycerol (PEG2000-DMG).

### 3. Structural Lipid

As used herein, the term“structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a mixture of two or more components each independently selected from cholesterol, fecosterol, sitosterol, ergosterol, campesterol, stigmasterol, brassicasterol, tomatidine, tomatine, ursolic acid, alpha-tocopherol, hopanoids, phytosterols, and steroids. In some embodiments, the structural lipid is a sterol. In some embodiments, the structural lipid is a mixture of two or more sterols.

As used herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the structural lipid comprises: cholesterol, β-Sitosterol, 20α-Hydroxycholesterol, sterol, or any combination thereof.

In some embodiments, the structural lipids may be one or more structural lipids described in U.S. Application No. 62/520,530.

### 4. Helper Lipid

Helper lipids contribute to their stability and delivery efficiency. Helper lipids with cone-shape geometry favoring the formation hexagonal II phase, such as dioleoylphosphatidylethanolamine (DOPE), can promote endosomal release of the nucleic acid. Meanwhile, cylindrical-shaped lipid phosphatidylcholine can provide greater bilayer stability, which is important for in vivo application of LNPs.

Helper lipids include phospholipids. Phospholipids may assemble into one or more lipid bilayers. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin (SM).

In some embodiments, a phospholipid useful or potentially useful in the present invention is an analog or variant of DSPC.

In some embodiments, the helper lipid comprises the phospholipid selected from one or more of the phospholipids described in U.S. Application No. 62/520,530 and WO 2020/160397, each of which is incorporated herein by reference.

In some embodiments, the helper lipid comprises the phospholipid selected from the non-limiting group of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1.2-di-O-octadecenyl-s7i-glycero-3-phosphocholine (18:0 Diether PC), 1-oleoyl-2-cholesterylhemisuccinoyl-s7i-glycero-3-phosphocholine (OChemsPC), 1-hexadecyl-sn-glycero-3-phosphocholine (Cl 6 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-gly cero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some embodiments, a LNP includes DSPC. In some embodiments, a LNP includes DOPE.

In some embodiments, the helper lipid comprises 1,2-distearoyl-sn-glycero-3-phosphocholine (DSPC) and/or 1,2-dioleoyl-sn-glycero-3-phosphoethanolamine (DOPE).

### Lipoplexes

In some embodiments, the nucleic acid vaccine is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13: 1222-1234; Santel et al., Gene Ther 2006 13: 1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31: 180-188; Pascolo Expert Opin. Biol. Ther. 4: 1285-1294; Fotin-Mleczek et al., 2011 J. Immunother. 34: 1-15; Song et al., Nature Biotechnol. 2005, 23:709-717; Peer et al., Proc Natl Acad Sci USA. 2007 6; 104:4095-4100; deFougerolles Hum Gene Ther. 2008 19: 125-132; all of which are incorporated herein by reference in its entirety).

In some embodiments, such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18: 1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13: 1222-1234; Santel et al., Gene Ther 2006 13: 1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18: 1127-1133; all of which are incorporated herein by reference in its entirety). Examples of passive targeting of formulations to liver cells include the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18: 1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8: 197-206; Musacchio and Torchilin, Front Biosci. 2011 16: 1388-1412; Yu et al., Mol Membr Biol. 2010 27:286-298; Patil et al., Crit Rev Ther Drug Carrier Syst. 2008 25: 1-61; Benoit et al., Biomacromolecules. 2011 12:2708-2714; Zhao et al., Expert Opin Drug Deliv. 2008 5:309-319; Akinc et al., Mol Ther. 2010 18: 1357-1364; Srinivasan et al., Methods Mol Biol. 2012 820: 105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18: 1127-1133; all of which are incorporated herein by reference in its entirety).

Liposomes, lipoplexes, or lipid nanoparticles may be used to improve the efficacy of polynucleotides directed protein production as these formulations may be able to increase cell transfection by the nucleic acid vaccine; and/or increase the translation of encoded protein. The liposomes, lipoplexes, or lipid nanoparticles may also be used to increase the stability of the polynucleotide.

Additional lipids useful in preparing the vaccines of the present disclosure are known in the art. Representative references are listed in Table 6A below, each of which is incorporated herein by reference.

TABLE 6A

| | | | | |
|---|---|---|---|---|
| US7758869B2 | US11357856B2 | EP3808177A1 | US20230090515A1 | US20220409536A1 |
| EP1420818B1 | US11453639B2 | EP3988104A1 | US8703906B2 | US20220411361A1 |
| EP4169579A1 | US11524932B2 | EP4074344A1 | US9314535B2 | US20230000773A1 |
| EP4169580A1 | US11542225B2 | US10077232B2 | US9421255B2 | US9533047B2 |
| EP4186528A1 | US11634379B2 | US10117941B2 | US9907862B2 | US9867888B2 |
| EP3532094A1 | US11639329B2 | US10456473B2 | EP3350157B1 | US9868692B2 |
| US20200163878A1 | US11648324B2 | US10821186B2 | EP3386484B1 | US9868693B2 |
| US20210251898A1 | US20190022247A1 | US10835612B2 | EP3394030B1 | US9950068B2 |
| EP3289083A4 | US20200121809A1 | US10953095B2 | EP3458590B9 | EP1747262B1 |
| EP3532097A1 | US20200172472A1 | US20090247608A1 | EP3596041B1 | EP2678038B1 |
| US20190274968A1 | US20210395188A1 | US20210162053A1 | EP3638678A1 | EP2955230A1 |
| EP3551169A4 | US20220081392A1 | US20220023427A1 | EP3736261A1 | EP4076647A1 |
| EP3456317A1 | US20220106257A1 | US20220040308A1 | EP3852732A1 | US10369216B2 |
| US9701623B2 | US20220204439A1 | US20220072154A1 | EP3853202A1 | US10568958B2 |
| EP2512449B1 | US20230097090A1 | US20220074925A1 | EP3853305A1 | US10751424B2 |
| US10653780B2 | US20230123534A1 | US20230109183A1 | EP3964200A1 | US11110166B2 |
| EP2224912B1 | US9737619B2 | US20230174459A1 | EP4009955A1 | US20200338215A1 |
| EP3100718B1 | US9738593B2 | US8034376B2 | EP4031521A1 | US20210361764A1 |
| EP4169534A1 | EP2788006A1 | US8158601B2 | EP4031524A1 | EP3097908A1 |
| EP3737386A1 | EP2788316B1 | US8642076B2 | EP4031527A1 | EP3156077B1 |
| US20220001025A1 | EP3860561B1 | US8722082B2 | EP4036079A3 | EP3192788A1 |
| US20210023100A1 | EP3988537A1 | US8802644B2 | EP4186888A1 | EP3207944B1 |
| EP2830593B1 | EP4183400A1 | US8883202B2 | US10195156B2 | EP3238738B1 |
| EP3427723B1 | US10369226B2 | US8999351B2 | US10207010B2 | EP3284482B1 |
| EP3484445B1 | US11071784B2 | US9139554B2 | US10266485B2 | EP3431076B1 |
| EP3881827A1 | US11110174B2 | US9186325B2 | US10392341B2 | EP3699172A3 |
| US11318195B2 | US11246933B1 | US9220683B2 | US10442756B2 | US8569256B2 |
| US11559587B2 | US11400158B2 | US9352042B2 | US10485885B2 | US9868691B2 |
| US20220313811A1 | US11679158B2 | US9394234B2 | US10494636B2 | EP4148083A1 |
| EP3595727A1 | US20220008541A1 | US9663449B2 | US10519455B2 | EP4182297A1 |
| EP2760477B1 | US20220370356A1 | US9682139B2 | US10556018B2 | US10106490B2 |
| EP4169578A1 | US8450467B2 | US9687550B2 | US10799463B2 | US10166298B2 |
| EP2350043B9 | US8828956B2 | US9694077B2 | US10857105B2 | US10221127B2 |
| EP2743265B1 | US8987377B2 | US9764036B2 | US10898574B2 | US10723692B2 |
| EP1999137B1 | US9061063B2 | US9814777B2 | US11220476B2 | US11040112B2 |
| EP2109459B1 | US9463247B2 | EP2903652B1 | US11285222B2 | US11168051B2 |
| US8758763B2 | EP2068886B1 | EP3145550B1 | US11597698B2 | EP2823810B9 |
| EP3160938B1 | EP2231195B1 | EP3197433A1 | US11649461B2 | US9018187B2 |
| EP3313829A1 | EP2279254B1 | EP4085930A1 | US20190382774A1 | US20220296517A1 |
| EP3368507B1 | EP2355658B1 | US10039714B2 | US20210154148A1 | US20220380299A1 |
| EP3397613A1 | EP2355851B1 | US10729785B2 | US20210161829A1 | US9677073B2 |
| EP3615510A1 | EP2391343B1 | US11045418B2 | US20210198200A1 | EP2331138B1 |
| EP3668833A1 | EP2440183B1 | US11173120B2 | US20220031631A1 | EP2817287B1 |
| EP3668834A1 | EP2509636B1 | US11541010B2 | US20220047518A1 | EP3033325B1 |
| EP3766916B1 | EP2695608B1 | US20220023212A1 | US20220265857A1 | EP3908568A1 |
| EP3867225A1 | | | | |

### Controlled/Sustained Release

In some embodiments, the nucleic acid vaccines of the present invention can be formulated for controlled release and/or targeted delivery. As used herein, “controlled release” refers to a pharmaceutical composition or compound release profile that conforms to a particular pattern of release to effect a specific outcome.

In some embodiments, the nucleic acid vaccines may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulation” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012131104 and WO2012131106; the contents of each of which is herein incorporated by reference in its entirety).

In other embodiments, the nucleic acid vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc Deerfield, IL), PEG-based sealants, and COSEAL® (Baxter International, Inc Deerfield, IL).

In other embodiments, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the the nucleic acid vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In some embodiments, the nucleic acid vaccine controlled release and/or targeted delivery formulation may comprise at least one degradable polyester which may comprise polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In some embodiments, the nucleic acid vaccine controlled release and/or targeted delivery formulation comprising at least one polynucleotide may comprise at least one PEG and/or PEG related polymer derivatives as described in U.S. Pat. No. 8,404,222, herein incorporated by reference in its entirety.

In other embodiments, the nucleic acid vaccine controlled release delivery formulation comprising at least one polynucleotide may be the controlled release polymer system described in US20130130348, herein incorporated by reference in its entirety.

In some embodiments, the the nucleic acid vaccines of the present invention may be encapsulated in a nanoparticle formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In other embodiments, polymer nanoparticles may be identified by the methods described in US Pub No. US20120140790, the contents of which is herein incorporated by reference in its entirety.

In some embodiments, the nanoparticle nucleic acid vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the the polynucleotides of the present invention (see International Pub No. 2010075072 and US Pub No. US20100216804, US20110217377 and US20120201859, each of which is herein incorporated by reference in their entirety). In another non-limiting example, the sustained release formulation may comprise agents which permit persistent bioavailability such as, but not limited to, crystals, macromolecular gels and/or particulate suspensions (see US Patent Publication No US20130150295, the contents of which is herein incorporated by reference in its entirety).

### Nanocarriers

In some embodiments, the nucleic acid vaccines may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422, each of which is herein incorporated by reference in their entirety.

In some embodiments, the synthetic nanocarrier formulations may be lyophilized by methods described in International Pub. No. WO2011072218 and U.S. Pat. No. 8,211,473; the content of each of which is herein incorporated by reference in their entirety.

In some embodiments, formulations of the present disclosure, including, but not limited to, synthetic nanocarriers, may be lyophilized or reconstituted by the methods described in US Patent Publication No. US20130230568, the contents of which are herein incorporated by reference in its entirety.

In some embodiments, the synthetic nanocarriers may comprise reactive groups to release the polynucleotides described herein (see International Pub. No. WO20120952552 and US Pub No. US20120171229, each of which is herein incorporated by reference in their entirety). In some embodiments, the synthetic nanocarriers may comprise an immuno stimulatory agent to enhance the immune response from delivery of the synthetic nanocarrier. As a non-limiting example, the synthetic nanocarrier may comprise a Th1 immuno stimulatory agent which may enhance a Th1-based response of the immune system (see International Pub No. WO2010123569 and US Pub. No. US20110223201, each of which is herein incorporated by reference in its entirety).

In some embodiments, the synthetic nanocarriers may be formulated for targeted release. In one embodiment, the synthetic nanocarrier is formulated to release the polynucleotides at a specified pH and/or after a desired time interval. As a non-limiting example, the synthetic nanoparticle may be formulated to release the nucleic acid vaccines after 24 hours and/or at a pH of 4.5 (see International Pub. Nos. WO2010138193 and WO2010138194 and US Pub Nos. US20110020388 and US20110027217, each of which is herein incorporated by reference in their entireties).

In some embodiments, the synthetic nanocarriers may be formulated for controlled and/or sustained release of the polynucleotides described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In some embodiments, the nucleic acid vaccine may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In some embodiments, the synthetic nanocarrier may be formulated for use as a vaccine. In some embodiments, the synthetic nanocarrier may encapsulate at least one polynucleotide which encode at least one antigen. As a non-limiting example, the synthetic nanocarrier may include at least one antigen and an excipient for a vaccine dosage form (see International Pub No. WO2011150264 and US Pub No. US20110293723, each of which is herein incorporated by reference in their entirety). As another non-limiting example, a vaccine dosage form may include at least two synthetic nanocarriers with the same or different antigens and an excipient (see International Pub No. WO2011150249 and US Pub No. US20110293701, each of which is herein incorporated by reference in their entirety). The vaccine dosage form may be selected by methods described herein, known in the art and/or described in International Pub No. WO2011150258 and US Pub No. US20120027806, each of which is herein incorporated by reference in their entirety).

In some embodiments, the synthetic nanocarrier may comprise at least one polynucleotide which encodes at least one adjuvant. As non-limiting example, the adjuvant may comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a mycobacterium (See e.g, U.S. Pat. No. 8,241,610; herein incorporated by reference in its entirety).

In some embodiments, the synthetic nanocarrier may comprise at least one polynucleotide and an adjuvant. As a non-limiting example, the synthetic nanocarrier comprising and adjuvant may be formulated by the methods described in International Pub No. WO2011150240 and US Pub No. US20110293700, each of which is herein incorporated by reference in its entirety.

In some embodiments, the synthetic nanocarrier may encapsulate at least one polynucleotide which encodes a peptide, fragment or region from a virus. As a non-limiting example, the synthetic nanocarrier may include, but is not limited to, the nanocarriers described in International Pub No. WO2012024621, WO201202629, WO2012024632 and US Pub No. US20120064110, US20120058153 and US20120058154, each of which is herein incorporated by reference in their entirety.

In some embodiments, the synthetic nanocarrier may be coupled to a polynucleotide which may be able to trigger a humoral and/or cytotoxic T lymphocyte (CTL) response (See e.g., International Publication No. WO2013019669, herein incorporated by reference in its entirety).

In some embodiments, the nucleic acid vaccine may be encapsulated in, linked to and/or associated with zwitterionic lipids. Non-limiting examples of zwitterionic lipids and methods of using zwitterionic lipids are described in US Patent Publication No. US20130216607, the contents of which are herein incorporated by reference in its entirety.

In certain aspects, the zwitterionic lipids may be used in the liposomes and lipid nanoparticles described herein.

In some embodiments, the nucleic acid vaccine may be formulated in colloid nanocarriers as described in US Patent Publication No. US20130197100, the contents of which are herein incorporated by reference in its entirety.

In some embodiments, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety.

In some embodiments, LNPs comprise the lipid KL52 (an amino-lipid disclosed in U.S. Application Publication No. 2012/0295832 incorporated herein by reference in its entirety). Activity and/or safety (as measured by examining one or more of ALT/AST, white blood cell count and cytokine induction) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

### Additional Encapsulation Agents

Exemplary encapsulation agents include, but are not limited to, ethyl lauroyl arginate, ethyl myristoyl arginate, ethyl palmitoyl arginate, ethyl cholesterol-arginate, ethyl oleic arginate, ethyl capric arginate, and ethyl carprylic arginate.

In certain embodiments, the encapsulation agent is ethyl lauroyl arginate,

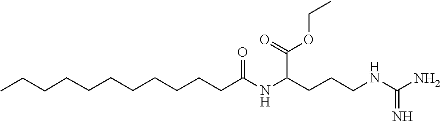 [SMILES: CCCCCCCCCCCC(=O)NC(CCCNC(=N)N)C(=O)OCC]

(ELA-1) or a salt or isomer thereof.

### Lipid Nanoparticle Size/Synthesis

The lipid nanoparticles of the present disclosure may comprise various diameters.

In some embodiments, particles may comprise a diameter less than 1000 nm. In some embodiments, the LNP particle may comprise a diameter less than 900 nm, 890 nm, 880 nm, 870 nm, 860 nm, 850 nm, 840 nm, 830 nm, 820 nm, 810 nm, 800 nm, 790 nm, 780 nm, 770 nm, 760 nm, 750 nm, 740 nm, 730 nm, 720 nm, 710 nm, 700 nm, 690 nm, 680 nm, 670 nm, 660 nm, 650 nm, 640 nm, 630 nm, 620 nm, 610 nm, 600 nm, 590 nm, 580 nm, 570 nm, 560 nm, 550 nm, 540 nm, 530 nm, 520 nm, 510 nm, 500 nm, 490 nm, 480 nm, 470 nm, 460 nm, 450 nm, 440 nm, 430 nm, 420 nm, 410 nm, 400 nm, 390 nm, 380 nm, 370 nm, 360 nm, 350 nm, 340 nm, 330 nm, 320 nm, 310 nm, 300 nm, 290 nm, 280 nm, 270 nm, 260 nm, 250 nm, 240 nm, 230 nm, 220 nm, or 210 nm.

In some embodiments, the LNP particle may comprise a diameter that is at least about 1 nm, 2 nm, 4 nm, 6 nm, 8 nm, 10 nm, 12 nm, 14 nm, 16 nm, 18 nm, 20 nm, 22 nm, 24 nm, 26 nm, 28 nm, 30 nm, 32 nm, 34 nm, 36 nm, 38 nm, 40 nm, 42 nm, 44 nm, 46 nm, 48 nm, 50 nm, 52 nm, 54 nm, 56 nm, 58 nm, 60 nm, 62 nm, 64 nm, 66 nm, 68 nm, 70 nm, 72 nm, 74 nm, 76 nm, 78 nm, 80 nm, 82 nm, 84 nm, 86 nm, 88 nm, 90 nm, 92 nm, 94 nm, 96 nm, 98 nm, 100 nm, 102 nm, 104 nm, 106 nm, 108 nm, 110 nm, 112 nm, 114 nm, 116 nm, 118 nm, 120 nm, 122 nm, 124 nm, 126 nm, 128 nm, 130 nm, 132 nm, 134 nm, 136 nm, 138 nm, 140 nm, 142 nm, 144 nm, 146 nm, 148 nm, 150 nm, 152 nm, 154 nm, 156 nm, 158 nm, 160 nm, 162 nm, 164 nm, 166 nm, 168 nm, 170 nm, 172 nm, 174 nm, 176 nm, 178 nm, 180 nm, 182 nm, 184 nm, 186 nm, 188 nm, 190 nm, 192 nm, 194 nm, 196 nm, 198 nm, or 200 nm.

In some embodiments, the LNP particle may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm, or from about 70 to about 90 nm.

In some embodiments, the nucleic acid vaccines of the invention may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm. In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the LNP particle may comprise a diameter from about 50 nm to about 200 nm. In some embodiments, the LNP particle may comprise a diameter from about 80 nm to about 100 nm.

### Peptides and Proteins

The nucleic acid vaccines of the invention can be formulated with peptides and/or proteins in order to increase transfection of cells by the polynucleotide.

In some embodiments, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention includes a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton FL, 2002); El-Andaloussi et al., Curr. Pharm. Des. 11(28):3597-611 (2003); and Deshayes et al., Cell. Mol. Life Sci. 62(16): 1839-49 (2005), all of which are incorporated herein by reference in their entirety). The compositions can also be formulated to include a cell penetrating agent, e.g., liposomes, which enhance delivery of the compositions to the intracellular space. nucleic acid vaccines of the invention may be complexed to peptides and/or proteins such as, but not limited to, peptides and/or proteins from Aileron Therapeutics (Cambridge, MA) and Permeon Biologies (Cambridge, MA) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in its entirety).

Formulations of the including peptides or proteins may be used to increase cell transfection by the nucleic acid vaccine, alter the biodistribution of the polynucleotide (e.g., by targeting specific tissues or cell types), and/or increase the translation of encoded protein. (See e.g., International Pub. No. WO2012110636 and WO2013123298; the contents of which are herein incorporated by reference in its entirety).

### Hyaluronidase

The intramuscular or subcutaneous localized injection of nucleic acid vaccines of the invention can include hyaluronidase, which catalyzes the hydrolysis of hyaluronan. By catalyzing the hydrolysis of hyaluronan, a constituent of the interstitial barrier, hyaluronidase lowers the viscosity of hyaluronan, thereby increasing tissue permeability (Frost, Expert Opin. Drug Deliv. (2007) 4:427-440; herein incorporated by reference in its entirety). It is useful to speed their dispersion and systemic distribution of encoded proteins produced by transfected cells. Alternatively, the hyaluronidase can be used to increase the number of cells exposed to a polynucleotide of the invention administered intramuscularly or subcutaneously.

### Nanotubes

The nucleic acid vaccines of the present disclosure can be attached or otherwise bound to at least one nanotube such as, but not limited to, rosette nanotubes, rosette nanotubes having twin bases with a linker, carbon nanotubes and/or single-walled carbon nanotubes, The nucleic acid vaccines may be bound to the nanotubes through forces such as, but not limited to, steric, ionic, covalent and/or other forces.

In some embodiments, the nanotube can release one or more nucleic acid vaccines into cells. The size and/or the surface structure of at least one nanotube may be altered so as to govern the interaction of the nanotubes within the body and/or to attach or bind to the nucleic acid vaccines disclosed herein. In one embodiment, the building block and/or the functional groups attached to the building block of the at least one nanotube may be altered to adjust the dimensions and/or properties of the nanotube. As a non-limiting example, the length of the nanotubes may be altered to hinder the nanotubes from passing through the holes in the walls of normal blood vessels but still small enough to pass through the larger holes in the blood vessels of tumor tissue.

In some embodiments, at least one nanotube may also be coated with delivery enhancing compounds including polymers, such as, but not limited to, polyethylene glycol. In another embodiment, at least one nanotube and/or the nucleic acid vaccines may be mixed with pharmaceutically acceptable excipients and/or delivery vehicles.

In some embodiments, the nucleic acid vaccines are attached and/or otherwise bound to at least one rosette nanotube. The rosette nanotubes may be formed by a process known in the art and/or by the process described in International Publication No. WO2012094304, herein incorporated by reference in its entirety. At least one nucleic acid vaccine may be attached and/or otherwise bound to at least one rosette nanotube by a process as described in International Publication No. WO2012094304, herein incorporated by reference in its entirety, where rosette nanotubes or modules forming rosette nanotubes are mixed in aqueous media with at least one nucleic acid vaccine under conditions which may cause at least one nucleic acid vaccines to attach or otherwise bind to the rosette nanotubes.

In some embodiments, the nucleic acid vaccines may be attached to and/or otherwise bound to at least one carbon nanotube. As a non-limiting example, the nucleic acid vaccines may be bound to a linking agent and the linked agent may be bound to the carbon nanotube (See e.g., U.S. Pat. No. 8,246,995; herein incorporated by reference in its entirety). The carbon nanotube may be a single-walled nanotube (See e.g., U.S. Pat. No. 8,246,995; herein incorporated by reference in its entirety).

### Conjugates

The nucleic acid vaccines of the invention include conjugates, such as a polynucleotide covalently linked to a carrier or targeting group, or including two encoding regions that together produce a fusion protein (e.g., bearing a targeting group and therapeutic protein or peptide). The conjugates of the invention include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Representative U.S. patents that teach the preparation of polynucleotide conjugates, particularly to RNA, include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference in their entireties.

In some embodiments, the conjugate of the present invention may function as a carrier for the nucleic acid vaccines of the present invention. The conjugate may comprise a cationic polymer such as, but not limited to, polyamine, polylysine, polyalkylenimine, and polyethylenimine which may be grafted to with poly(ethylene glycol). As a non-limiting example, the conjugate may be similar to the polymeric conjugate and the method of synthesizing the polymeric conjugate described in U.S. Pat. No. 6,586,524 herein incorporated by reference in its entirety.

A non-limiting example of a method for conjugation to a substrate is described in US Patent Publication No. US20130211249, the contents of which are herein incorporated by reference in its entirety. The method may be used to make a conjugated polymeric particle comprising a nucleic acid vaccine.

### Excipients

Vaccines, antibodies, milk, animal feed, agents (e.g., an agent that reduces methane production in a ruminant, a probiotic bacterial strain, etc.), or other compositions of the present disclosure may comprise at least one excipient and/or carrier described herein or those known in the art (e.g., a pharmaceutically acceptable excipient and/or carrier).

A pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents, pH adjusting agents and the like, as suited to the particular dosage form desired.

In some embodiments, one or more excipients or accessory ingredients may make up greater than 50% of the total mass or volume of a pharmaceutical composition. In some embodiments, the one or more excipients or accessory ingredients may make up at least about 50%, 60%, 70%, 80%, 90%, or more of a pharmaceutical convention.

Various excipients for formulating pharmaceutical compositions and techniques for preparing the composition are known in the art (see Remington: The Science and Practice of Pharmacy, 21^{st} Edition, A. R. Gennaro (Lippincott, Williams & Wilkins, Baltimore, M D, 2006; incorporated herein by reference in its entirety).

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ©45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRU®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); amino acids (e.g., glycine); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and/or combinations thereof.

Exemplary preservatives may include, but are not limited to, antioxidants, chelating agents, antimicrobial preservatives, antifungal preservatives, alcohol preservatives, acidic preservatives, and/or other preservatives. Oxidation is a potential degradation pathway for mRNA, especially for liquid mRNA formulations. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®!!, NEOLONE™, KATHON™, and/or EUXYL®.

In some embodiments, the pH of the nucleic acid vaccine solutions are maintained between pH 5 and pH 8 to improve stability. Exemplary buffers to control pH may include, but are not limited to sodium phosphate, sodium citrate, sodium succinate, histidine (or histidine-HCl), sodium carbonate, and/or sodium malate. In another embodiment, the exemplary buffers listed above may be used with additional monovalent counterions (including, but not limited to potassium). Divalent cations may also be used as buffer counterions; however, these are not preferred due to complex formation and/or mRNA degradation.

Exemplary buffering agents may also include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and/or combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

### Cryoprotectants

In some embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about ˜20° C., −30° C., −40° C., −50° C., −60° C., −70° C., or −80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150C).

In some embodiments, nucleic acid vaccine formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with nucleic acid vaccines in order to stabilize them during freezing. Frozen storage of nucleic acid vaccines between −20° C. and −80° C. may be advantageous for long term (e.g. 36 months) stability of polynucleotide. In some embodiments, cryoprotectants are included in nucleic acid vaccine formulations to stabilize polynucleotide through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

### Bulking Agents

In some embodiments, nucleic acid vaccine formulations may comprise bulking agents. As used herein, the term “bulking agent” refers to one or more agents included in formulations to impart a desired consistency to the formulation and/or stabilization of formulation components.

In some embodiments, bulking agents are included in lyophilized nucleic acid vaccine formulations to yield a “pharmaceutically elegant” cake, stabilizing the lyophilized nucleic acid vaccines during long term (e.g. 36 month) storage. Bulking agents of the present invention may include, but are not limited to sucrose, trehalose, mannitol, glycine, lactose and/or raffinose. In some embodiments, combinations of cryoprotectants and bulking agents (for example, sucrose/glycine or trehalose/mannitol) may be included to both stabilize nucleic acid vaccines during freezing and provide a bulking agent for lyophilization.

Non-limiting examples of formulations and methods for formulating the nucleic acid vaccines of the present invention are also provided in International Publication No WO2013090648 filed Dec. 14, 2012, the contents of which are incorporated herein by reference in their entirety.

### Inactive Ingredients

In some embodiments, nucleic acid vaccine formulations may comprise at least one excipient which is an inactive ingredient. As used herein, the term “inactive ingredient” refers to one or more inactive agents included in formulations. Exemplary non-exhaustive lists of inactive ingredients and the routes of administration the inactive ingredients may be formulated in are described in Tables 6B and 7.

TABLE 6B
Exemplary Inactive Ingredients

| Inactive Ingredient | Route of Administration |
|---|---|
| Alpha-Terpineol | Topical |
| Alpha-Tocopherol | Intravenous; Topical |
| Alpha-Tocopherol Acetate, Dl- | Topical |
| Alpha-Tocopherol, Dl- | Intravenous; Topical |
| 1,2,6-Hexanetriol | Topical |
| 1,2-Dimyristoyl-Sn-Glycero-3-(Phospho-S- | Intravenous; Infusion (IV) |
| (1-Glycerol)) | |
| 1,2-Dimyristoyl-Sn-Glycero-3- | Intravenous; Infusion (IV) |
| Phosphocholine | |
| 1,2-Dioleoyl-Sn-Glycero-3-Phosphocholine | Epidural |
| 1,2-Dipalmitoyl-Sn-Glycero-3-(Phospho- | Epidural |
| Rac-(1-Glycerol)) | |
| 1,2-Distearoyl-Sn-Glycero-3-(Phospho-Rac- | Intravenous |
| (1-Glycerol)) | |
| 1,2-Distearoyl-Sn-Glycero-3-Phosphocholine | Intravenous |
| 1-O-Tolylbiguanide | Topical |

TABLE 7
Exemplary Inactive Ingredients

| Route of Administration | Inactive Ingredient |
|---|---|
| Intrathecal (AN, CNBLK) | Acetone Sodium Bisulfite; Citric Acid; Hydrochloric Acid; Sodium |
| | Chloride; Sodium Hydroxide; Sodium Metabisulfite |
| Infiltration (AN) | Acetic Acid; Acetone Sodium Bisulfite; Ascorbic Acid; Benzyl |
| | Alcohol; Calcium Chloride; Carbon Dioxide; Chlorobutanol; Citric |
| | Acid; Citric Acid Monohydrate; Edetate Calcium Disodium; Edetate |
| | Disodium; Hydrochloric Acid; Hydrochloric Acid, Diluted; Lactic Acid; |
| | Methylparaben; Monothioglycerol; Nitrogen; Potassium Chloride; |
| | Potassium Metabisulfite; Potassium Phosphate, Monobasic; |
| | Propylparaben; Sodium Bisulfite; Sodium Carbonate; Sodium Chlorate; |
| | Sodium Chloride; Sodium Citrate; Sodium Hydroxide; Sodium Lactate; |
| | Sodium Metabisulfite; Sodium Phosphate, Dibasic, Heptahydrate |
| Sympathetic NBLK (AN) | Hydrochloric Acid; Sodium Chloride; Sodium Hydroxide |
| Auricular (Otic) | Acetic Acid; Aluminum Acetate; Aluminum Sulfate Anhydrous; |
| | Benzalkonium Chloride; Benzethonium Chloride; Benzyl Alcohol; |
| | Boric Acid; Calcium Carbonate; Cetyl Alcohol; Chlorobutanol; |
| | Chloroxylenol; Citric Acid; Creatinine; Cupric Sulfate; Cupric Sulfate |
| | Anhydrous; Edetate Disodium; Edetic Acid; Glycerin; Glyceryl |
| | Stearate; Hydrochloric Acid; Hydrocortisone; Hydroxyethyl Cellulose; |
| | Isopropyl Myristate; Lactic Acid; Lecithin, Hydrogenated; |
| | Methylparaben; Mineral Oil; Petrolatum; Petrolatum, White; |
| | Phenylethyl Alcohol; Polyoxyl 40 Stearate; Polyoxyl Stearate; |
| | Polysorbate 20; Polysorbate 80; Polyvinyl Alcohol; Potassium |
| | Metabisulfite; Potassium Phosphate, Monobasic; Povidone K90f; |
| | Povidones; Propylene Glycol; Propylene Glycol Diacetate; |
| | Propylparaben; Sodium Acetate; Sodium Bisulfite; Sodium Borate; |
| | Sodium Chloride; Sodium Citrate; Sodium Hydroxide; Sodium |
| | Phosphate, Dibasic, Anhydrous; Sodium Phosphate, Dibasic, |
| | Heptahydrate; Sodium Phosphate, Monobasic, Anhydrous; Sodium |

In Table 7, “AN” means anesthetic, “CNBLK” means cervical nerve block, “NBLK” means nerve block, and “IV” means intravenous.

### Naked Delivery

The nucleic acid vaccines of the present invention may be delivered to a cell naked or in saline. As used herein in, “naked” refers to delivering nucleic acid vaccines free from agents which promote transfection. The naked nucleic acid vaccines may be delivered to the cell using routes of administration known in the art and described herein.

### Formulated Delivery

The nucleic acid vaccines of the present invention may be formulated, using the methods described herein. The formulations may comprise polynucleotides which may be modified and/or unmodified. The formulations may further comprise, but are not limited to, cell penetration agents, a pharmaceutically acceptable carrier, a delivery agent, a bioerodible or biocompatible polymer, a solvent, and a sustained-release delivery depot. The formulated nucleic acid vaccines may be delivered to the cell using routes of administration known in the art and described herein. The compositions may also be formulated for direct delivery to an organ or tissue in any of several ways in the art including, but not limited to, direct soaking, via a catheter, by gels, powder, ointments, creams, gels, lotions, and/or drops, by using substrates such as fabric or biodegradable materials coated or impregnated with the compositions, and the like.

### Administration

Vaccines, antibodies, milk, animal feed, agents (e.g., an agent that reduces methane production in a ruminant, a probiotic bacterial strain, a small molecule inhibitor, etc.), or other compositions of the present disclosure (e.g., those reducing methane production in a ruminant) may be administered to a ruminant by any route which results in a therapeutically effective outcome. These include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intrapro static (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the auras media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique, ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

Non-limiting routes of administration for the nucleic acid vaccines of the present disclosure are described below.

### Parenteral and Injectable Administration

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

A pharmaceutical composition for parenteral administration may comprise at least one inactive ingredient. A non-exhaustive list of inactive ingredients for use in pharmaceutical compositions for parenteral administration includes hydrochloric acid, mannitol, nitrogen, sodium acetate, sodium chloride and sodium hydroxide. Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S. P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. The sterile formulation may also comprise adjuvants such as local anesthetics, preservatives and buffering agents.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Injectable formulations may be for direct injection into a region of a tissue, organ and/or subject. As a non-limiting example, a tissue, organ and/or subject may be directly injected a formulation by intramyocardial injection into the ischemic region. (See e.g., Zangi et al. Nature Biotechnology 2013; the contents of which are herein incorporated by reference in its entirety).

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal (e.g., transvaginal) administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

As a non-limiting example, the formulations for rectal and/or vaginal administration may be prepared by mixing the drug with a suitable non-irritating excipient that is solid at ordinary temperatures but liquid at the rectal temperature and will therefore melt in the rectum and/or vagina to release the drug. Such materials include cocoa butter and polyethylene glycols. A pharmaceutical composition for rectal administration may comprise at least one inactive ingredient. A non-exhaustive list of inactive ingredients for use in pharmaceutical compositions for rectal administration includes alcohol, alcohol, dehydrated, aluminum subacetate, anhydrous citric acid, aniseed oil, ascorbic acid, ascorbyl palmitate, balsam peru, benzoic acid, benzyl alcohol, bismuth subgallate, butylated hydroxyanisole, butylated hydroxytoluene, butylparaben, caramel, carbomer 934, carbomer 934p, carboxypolymethylene, cerasynt-se, cetyl alcohol, cocoa butter, coconut oil, hydrogenated, coconut oil/palm kernel oil glycerides, hydrogenated, cola nitida seed extract, d&c yellow no. 10, dichlorodifluoromethane, dichlorotetrafluoroethane, dimethyldioctadecylammonium bentonite, edetate calcium disodium, edetate disodium, edetic acid, epilactose, ethylenediamine, fat, edible, fat, hard, fd&c blue no. 1, fd&c green no. 3, fd&c yellow no. 6, flavor FIG. 827118, flavor raspberry pfc-8407, fructose, galactose, glycerin, glyceryl palmitate, glyceryl stearate, glyceryl stearate/peg stearate, glyceryl stearate/peg-40 stearate, glycine, hydrocarbon, hydrochloric acid, hydrogenated palm oil, hypromelloses, lactose, lanolin, lecithin, light mineral oil, magnesium aluminum silicate, magnesium aluminum silicate hydrate, methylparaben, nitrogen, palm kernel oil, paraffin, petrolatum, white, polyethylene glycol 1000, polyethylene glycol 1540, polyethylene glycol 3350, polyethylene glycol 400, polyethylene glycol 4000, polyethylene glycol 6000, polyethylene glycol 8000, polysorbate 60, polysorbate 80, potassium acetate, potassium metabisulfite, propylene glycol, propylparaben, saccharin sodium, saccharin sodium anhydrous, silicon dioxide, colloidal, simethicone, sodium benzoate, sodium carbonate, sodium chloride, sodium citrate, sodium hydroxide, sodium metabisulfite, sorbitan monooleate, sorbitan sesquioleate, sorbitol, sorbitol solution, starch, steareth-10, steareth-40, sucrose, tagatose, d-, tartaric acid, dl-, trolamine, tromethamine, vegetable oil glyceride, hydrogenated, vegetable oil, hydrogenated, wax, emulsifying, white wax, xanthan gum and zinc oxide.

A pharmaceutical composition for vaginal administration may comprise at least one inactive ingredient. Any or none of the inactive ingredients used may have been approved by the US Food and Drug Administration (FDA). A non-exhaustive list of inactive ingredients for use in pharmaceutical compositions for vaginal administration includes adipic acid, alcohol, denatured, allantoin, anhydrous lactose, apricot kernel oil peg-6 esters, barium sulfate, beeswax, bentonite, benzoic acid, benzyl alcohol, butylated hydroxyanisole, butylated hydroxytoluene, calcium lactate, carbomer 934, carbomer 934p, cellulose, microcrystalline, ceteth-20, cetostearyl alcohol, cetyl alcohol, cetyl esters wax, cetyl palmitate, cholesterol, choleth, citric acid, citric acid monohydrate, coconut oil/palm kernel oil glycerides, hydrogenated, crospovidone, edetate disodium, ethylcelluloses, ethylene-vinyl acetate copolymer (28% vinyl acetate), ethylene-vinyl acetate copolymer (9% vinylacetate), fatty alcohols, fd&c yellow no. 5, gelatin, glutamic acid, dl-, glycerin, glyceryl isostearate, glyceryl monostearate, glyceryl stearate, guar gum, high density polyethylene, hydrogel polymer, hydrogenated palm oil, hypromellose 2208 (15000 mpa·s), hypromelloses, isopropyl myristate, lactic acid, lactic acid, dl-, lactose, lactose monohydrate, lactose, hydrous, lanolin, lanolin anhydrous, lecithin, lecithin, soybean, light mineral oil, magnesium aluminum silicate, magnesium aluminum silicate hydrate, magnesium stearate, methyl stearate, methylparaben, microcrystalline wax, mineral oil, nitric acid, octyldodecanol, peanut oil, peg 6-32 stearate/glycol stearate, peg-100 stearate, peg-120 glyceryl stearate, peg-2 stearate, peg-5 oleate, pegoxol 7 stearate, petrolatum, white, phenylmercuric acetate, phospholipon 90 g, phosphoric acid, piperazine hexahydrate, poly(dimethylsiloxane/methylvinylsiloxane/methylhydrogensiloxane) dimethylvinyl or dimethylhydroxy or trimethyl endblocked, polycarbophil, polyester, polyethylene glycol 1000, polyethylene glycol 3350, polyethylene glycol 400, polyethylene glycol 4000, polyethylene glycol 6000, polyethylene glycol 8000, polyglyceryl-3 oleate, polyglyceryl-4 oleate, polyoxyl palmitate, polysorbate 20, polysorbate 60, polysorbate 80, polyurethane, potassium alum, potassium hydroxide, povidone k29/32, povidones, promulgen d, propylene glycol, propylene glycol monopalmitostearate, propylparaben, quaternium-15 cis-form, silicon dioxide, silicon dioxide, colloidal, silicone, sodium bicarbonate, sodium citrate, sodium hydroxide, sodium lauryl sulfate, sodium metabisulfite, sodium phosphate, dibasic, anhydrous, sodium phosphate, monobasic, anhydrous, sorbic acid, sorbitan monostearate, sorbitol, sorbitol solution, spermaceti, stannous 2-ethylhexanoate, starch, starch 1500, pregelatinized, starch, corn, stearamidoethyl diethylamine, stearic acid, stearyl alcohol, tartaric acid, dl-, tert-butylhydroquinone, tetrapropyl orthosilicate, trolamine, urea, vegetable oil, hydrogenated, wecobee fs, white ceresin wax and white wax.

### Oral Administration

Liquid dosage forms for oral administration include, but are not limited to, pharmaceutically acceptable emulsions, microemulsions, solutions, suspensions, syrups, and/or elixirs. In addition to active ingredients, liquid dosage forms may comprise inert diluents and/or excipients commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

Suspensions for oral dosage may contain the active materials in a mixture with excipients suitable for the manufacture of aqueous suspensions. Such excipients may be suspending agents, as a non-limiting example the suspending agents may be sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate; or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions for oral dosage can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

The oral dosage may also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents. The solid dosage forms may also dissolve once they come in contact with liquid such as, but not limited to, salvia and bile.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations.

Solid dosage forms may be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Dosage forms for oral delivery may also be chewable. The chewable dosages forms may be sustained release formulations such as, but not limited to, the sustained release compositions described in International Publication No WO2013082470 and US Publication No US20130142876, each of which is herein incorporated by reference in its entirety. The chewable dosage forms may comprise amphipathic lipids such as, but not limited to, those described in International Publication No WO2013082470 and US Publication No US20130142876, each of which is herein incorporated by reference in its entirety.

### Topical or Transdermal Administration

As described herein, compositions of the present disclosure may be formulated for administration transdermally. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of nucleic acid delivery. Two routes are commonly considered to deliver nucleic acid vaccines to the skin: (ii) intradermal injection; and (iii) systemic delivery (e.g. for treatment of dermatologic diseases that affect both cutaneous and extracutaneous regions). Nucleic acid vaccines can be delivered to the skin by several different approaches known in the art. After delivery of the nucleic acid, gene products have been detected in a number of different skin cell types, including, but not limited to, basal keratinocytes, sebaceous gland cells, dermal fibroblasts and dermal macrophages.

In some embodiments, the invention provides for the nucleic acid vaccine compositions to be delivered in more than one injection.

In some embodiments, before transdermal administration at least one area of tissue, such as skin, may be subjected to a device and/or solution which may increase permeability. In one embodiment, the tissue may be subjected to an abrasion device to increase the permeability of the skin (see U.S. Patent Publication No. 20080275468, herein incorporated by reference in its entirety). In another embodiment, the tissue may be subjected to an ultrasound enhancement device. An ultrasound enhancement device may include, but is not limited to, the devices described in U.S. Publication No. 20040236268 and U.S. Pat. Nos. 6,491,657 and 6,234,990; each of which are herein incorporated by reference in their entireties. Methods of enhancing the permeability of tissue are described in U.S. Publication Nos. 20040171980 and 20040236268 and U.S. Pat. No. 6,190,315; each of which are herein incorporated by reference in their entireties.

In some embodiments, a device may be used to increase permeability of tissue before delivering formulations of modified mRNA described herein. The permeability of skin may be measured by methods known in the art and/or described in U.S. Pat. No. 6,190,315, herein incorporated by reference in its entirety. As a non-limiting example, a modified mRNA formulation may be delivered by the drug delivery methods described in U.S. Pat. No. 6,190,315, herein incorporated by reference in its entirety.

In another non-limiting example tissue may be treated with a eutectic mixture of local anesthetics (EMLA) cream before, during and/or after the tissue may be subjected to a device which may increase permeability. Katz et al. (Anesth Analg (2004); 98:371-76; herein incorporated by reference in its entirety) showed that using the EMLA cream in combination with a low energy, an onset of superficial cutaneous analgesia was seen as fast as 5 minutes after a pretreatment with a low energy ultrasound.

In some embodiments, enhancers may be applied to the tissue before, during, and/or after the tissue has been treated to increase permeability. Enhancers include, but are not limited to, transport enhancers, physical enhancers, and cavitation enhancers. Non-limiting examples of enhancers are described in U.S. Pat. No. 6,190,315, herein incorporated by reference in its entirety.

In some embodiments, a device may be used to increase permeability of tissue before delivering formulations of nucleic acid vaccines described herein, which may further contain a substance that invokes an immune response. In another non-limiting example, a formulation containing a substance to invoke an immune response may be delivered by the methods described in U.S. Publication Nos. 20040171980 and 20040236268; each of which are herein incorporated by reference in their entireties.

Dosage forms for transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required.

Additionally, the compositions of the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

A pharmaceutical nucleic acid vaccine composition for transdermal administration may comprise at least one inactive ingredient. A non-exhaustive list of inactive ingredients for use in pharmaceutical compositions for transdermal administration includes acrylates copolymer, acrylic acid-isooctyl acrylate copolymer, acrylic adhesive 788, adcote 72a103, aerotex resin 3730, alcohol, alcohol, dehydrated, aluminum polyester, bentonite, butylated hydroxytoluene, butylene glycol, butyric acid, caprylic/capric triglyceride, carbomer 1342, carbomer 940, carbomer 980, carrageenan, cetylpyridinium chloride, citric acid, crospovidone, daubert 1-5 pestr (matte) 164z, diethylene glycol monoethyl ether, diethylhexyl phthalate, dimethicone copolyol, dimethicone mdx4-4210, dimethicone medical fluid 360, dimethylaminoethyl methacrylate-butyl methacrylate-methyl methacrylate copolymer, dipropylene glycol, duro-tak 280-2516, duro-tak 387-2516, duro-tak 80-1196, duro-tak 87-2070, duro-tak 87-2194, duro-tak 87-2287, duro-tak 87-2296, duro-tak 87-2888, duro-tak 87-2979, edetate disodium, ethyl acetate, ethyl oleate, ethylcelluloses, ethylene vinyl acetate copolymer, ethylene-propylene copolymer, fatty acid esters, gelva 737, glycerin, glyceryl laurate, glyceryl oleate, heptane, high density polyethylene, hydrochloric acid, hydrogenated polybutene 635-690, hydroxyethyl cellulose, hydroxypropyl cellulose, isopropyl myristate, isopropyl palmitate, lactose, lanolin anhydrous, lauryl lactate, lecithin, levulinic acid, light mineral oil, medical adhesive modified s-15, methyl alcohol, methyl laurate, mineral oil, nitrogen, octisalate, octyldodecanol, oleic acid, oleyl alcohol, oleyl oleate, pentadecalactone, petrolatum, white, polacrilin, polyacrylic acid (250000 mw), polybutene (1400 mw), polyester, polyester polyamine copolymer, polyester rayon, polyethylene terephthalates, polyisobutylene, polyisobutylene (1100000 mw), polyisobutylene (35000 mw), polyisobutylene 178-236, polyisobutylene 241-294, polyisobutylene 35-39, polyisobutylene low molecular weight, polyisobutylene medium molecular weight, polyisobutylene/polybutene adhesive, polypropylene, polyvinyl acetate, polyvinyl alcohol, polyvinyl chloride, polyvinyl chloride-polyvinyl acetate copolymer, polyvinylpyridine, povidone k29/32, povidones, propylene glycol, propylene glycol monolaurate, ra-2397, ra-3011, silicon, silicon dioxide, colloidal, silicone, silicone adhesive 4102, silicone adhesive 4502, silicone adhesive bio-psa q7-4201, silicone adhesive bio-psa q7-4301, silicone/polyester film strip, sodium chloride, sodium citrate, sodium hydroxide, sorbitan monooleate, stearalkonium hectorite/propylene carbonate, titanium dioxide, triacetin, trolamine, tromethamine, union 76 amsco-res 6038 and viscose/cotton.

A pharmaceutical nucleic acid vaccine composition for intradermal administration may comprise at least one inactive ingredient. A non-exhaustive list of inactive ingredients for use in pharmaceutical compositions for intradermal administration includes benzalkonium chloride, benzyl alcohol, carboxymethylcellulose sodium, creatinine, edetate disodium, glycerin, hydrochloric acid, metacresol, methylparaben, phenol, polysorbate 80, protamine sulfate, sodium acetate, sodium bisulfite, sodium chloride, sodium hydroxide, sodium phosphate, sodium phosphate, dibasic, sodium phosphate, dibasic, heptahydrate, sodium phosphate, monobasic, anhydrous and zinc chloride.

### Depot Administration

As described herein, in some embodiments, the composition is formulated in depots for extended release. Generally, a specific organ or tissue (a “target tissue”) is targeted for administration.

In some aspects, the nucleic acid vaccines are spatially retained within or proximal to a target tissue. Provided are method of providing a composition to a target tissue of a mammalian subject by contacting the target tissue (which contains one or more target cells) with the composition under conditions such that the composition, in particular the nucleic acid component(s) of the composition, is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the amount of the nucleic acid present in the composition that enters one or more target cells. For example, at least 1, 5, 10, 20, 30, 40, 50, 60, 70, 80, 85, 90, 95, 96, 97, 98, 99, 99.9, 99.99 or greater than 99.99% of the nucleic acids administered to the subject are present intracellularly at a period of time following administration. For example, intramuscular injection to a ruminant subject is performed using an aqueous composition containing a ribonucleic acid and a transfection reagent, and retention of the composition is determined by measuring the amount of the ribonucleic acid present in the muscle cells.

Aspects of the invention are directed to methods of providing a composition to a target tissue of a ruminant subject, by contacting the target tissue (comprising one or more target cells) with the composition under conditions such that the composition is substantially retained in the target tissue. The composition contains an effective amount of a polynucleotides such that the polypeptide of interest is produced in at least one target cell. The compositions generally contain a cell penetration agent, although “naked” nucleic acid vaccine (such as nucleic acids without a cell penetration agent or other agent) is also contemplated, and a pharmaceutically acceptable carrier.

In some circumstances, the amount of a protein produced by cells in a tissue is desirably increased. Preferably, this increase in protein production is spatially restricted to cells within the target tissue. Thus, provided herein are methods of increasing production of a protein of interest in a tissue of a ruminant subject. A composition is provided that contains polynucleotides characterized in that a unit quantity of composition has been determined to produce the polypeptide of interest in a substantial percentage of cells contained within a predetermined volume of the target tissue.

In some embodiments, the nucleic acid vaccine composition includes a plurality of different polynucleotides, where one or more than one of the polynucleotides encodes a polypeptide of interest. Optionally, the composition also comprises a cell penetration agent to assist in the intracellular delivery of the composition. A determination is made of the dose of the composition required to produce the polypeptide of interest in a substantial percentage of cells contained within the predetermined volume of the target tissue (generally, without inducing significant production of the polypeptide of interest in tissue adjacent to the predetermined volume, or distally to the target tissue). Subsequent to this determination, the determined dose is introduced directly into the tissue of the mammalian subject. In one embodiment, the invention provides for the nucleic acid vaccines to be delivered in more than one injection or by split dose injections.

In some embodiments, the compositions may be retained near target tissue using a small disposable drug reservoir, patch pump or osmotic pump. Non-limiting examples of patch pumps include those manufactured and/or sold by BD® (Franklin Lakes, NJ), Insulet Corporation (Bedford, MA), SteadyMed Therapeutics (San Francisco, CA), Medtronic (Minneapolis, MN) (e.g., MiniMed), UniLife (York, PA), Valeritas (Bridgewater, NJ), and SpringLeaf Therapeutics (Boston, MA). A non-limiting example of an osmotic pump include those manufactured by DURECT® (Cupertino, CA) (e.g., DUROS® and ALZET®).

### Pulmonary Administration

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient). As a non-limiting example, the nucleic acid vaccines described herein may be formulated for pulmonary delivery by the methods described in U.S. Pat. No. 8,257,685; herein incorporated by reference in its entirety.

Pharmaceutical nucleic acid vaccine compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

The compositions and formulations provided herein which may be used for pulmonary delivery may further comprise one or more surfactants. Suitable surfactants or surfactant components for enhancing the uptake of the compositions of the invention include synthetic and natural as well as full and truncated forms of surfactant protein A, surfactant protein B, surfactant protein C, surfactant protein D and surfactant Protein E, di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine; phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamellar bodies which are the natural carrier vehicles for the components of surfactant, omega-3 fatty acids, polyenic acid, polyenoic acid, lecithin, palmitinic acid, non-ionic block copolymers of ethylene or propylene oxides, polyoxypropylene, monomeric and polymeric, polyoxyethylene, monomelic and polymeric, poly(vinyl amine) with dextran and/or alkanoyl side chains, Brij 35, Triton X-100 and synthetic surfactants ALEC, Exosurf, Survan and Atovaquone, among others. These surfactants can be used either as single or part of a multiple component surfactant in a formulation, or as covalently bound additions to the 5′ and/or 3′ ends of the nucleic acid component of a pharmaceutical composition herein.

### Intranasal, Nasal and Buccal Administration

Formulations described herein as being useful for pulmonary delivery are useful for intranasal delivery of a nucleic acid vaccine pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical nucleic acid vaccine composition for inhalation (respiratory) administration may comprise at least one inactive ingredient. A non-exhaustive list of inactive ingredients for use in pharmaceutical compositions for inhalation (respiratory) administration includes acetone sodium bisulfite, acetylcysteine, alcohol, alcohol, dehydrated, ammonia, apaflurane, ascorbic acid, benzalkonium chloride, calcium carbonate, carbon dioxide, cetylpyridinium chloride, chlorobutanol, citric acid, d&c yellow no. 10, dichlorodifluoromethane, dichlorotetrafluoroethane, edetate disodium, edetate sodium, fd&c yellow no. 6, fluorochlorohydrocarbons, gelatin, glycerin, glycine, hydrochloric acid, hydrochloric acid, diluted, lactose, lactose monohydrate, lecithin, lecithin, hydrogenated soy, lecithin, soybean, lysine monohydrate, mannitol, menthol, methylparaben, nitric acid, nitrogen, norflurane, oleic acid, polyethylene glycol 1000, povidone k25, propylene glycol, propylparaben, saccharin, saccharin sodium, silicon dioxide, colloidal, sodium bisulfate, sodium bisulfite, sodium chloride, sodium citrate, sodium hydroxide, sodium lauryl sulfate, sodium metabisulfite, sodium sulfate anhydrous, sodium sulfite, sorbitan trioleate, sulfuric acid, thymol, titanium dioxide, trichloromonofluoromethane, tromethamine and zinc oxide.

A pharmaceutical nucleic acid vaccine composition for nasal administration may comprise at least one inactive ingredient. Any or none of the inactive ingredients used may have been approved by the US Food and Drug Administration (FDA). A non-exhaustive list of inactive ingredients for use in pharmaceutical compositions for nasal administration includes acetic acid, alcohol, dehydrated, allyl.alpha.-ionone, anhydrous dextrose, anhydrous trisodium citrate, benzalkonium chloride, benzethonium chloride, benzyl alcohol, butylated hydroxyanisole, butylated hydroxytoluene, caffeine, carbon dioxide, carboxymethylcellulose sodium, cellulose, microcrystalline, chlorobutanol, citric acid, citric acid monohydrate, dextrose, dichlorodifluoromethane, dichlorotetrafluoroethane, edetate disodium, glycerin, glycerol ester of hydrogenated rosin, hydrochloric acid, hypromellose 2910 (15000 mpa·s), methylcelluloses, methylparaben, nitrogen, norflurane, oleic acid, petrolatum, white, phenylethyl alcohol, polyethylene glycol 3350, polyethylene glycol 400, polyoxyl 400 stearate, polysorbate 20, polysorbate 80, potassium phosphate, monobasic, potassium sorbate, propylene glycol, propylparaben, sodium acetate, sodium chloride, sodium citrate, sodium hydroxide, sodium phosphate, sodium phosphate, dibasic, sodium phosphate, dibasic, anhydrous, sodium phosphate, dibasic, dihydrate, sodium phosphate, dibasic, dodecahydrate, sodium phosphate, dibasic, heptahydrate, sodium phosphate, monobasic, anhydrous, sodium phosphate, monobasic, dihydrate, sorbitan trioleate, sorbitol, sorbitol solution, sucralose, sulfuric acid, trichloromonofluoromethane and trisodium citrate dihydrate.

### Ophthalmic and Auricular (Otic) Administration

A pharmaceutical nucleic acid vaccine composition may be prepared, packaged, and/or sold in a formulation suitable for delivery to and/or around the eye and/or delivery to the ear (e.g., auricular (otic) administration). Non-limiting examples of route of administration for delivery to and/or around the eye include retrobulbar, conjuctival, intracorneal, intraocular, intravitreal, ophthlamic and subconjuctiva. Such formulations may, for example, be in the form of eye drops or ear drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this invention. A multilayer thin film device may be prepared to contain a pharmaceutical composition for delivery to the eye and/or surrounding tissue.

A pharmaceutical nucleic acid vaccine composition for ophthalmic administration may comprise at least one inactive ingredient. Any or none of the inactive ingredients used may have been approved by the US Food and Drug Administration (FDA). A non-exhaustive list of inactive ingredients for use in pharmaceutical compositions for ophthalmic administration includes acetic acid, alcohol, alcohol, dehydrated, alginic acid, amerchol-cab, ammonium hydroxide, anhydrous trisodium citrate, antipyrine, benzalkonium chloride, benzethonium chloride, benzododecinium bromide, boric acid, caffeine, calcium chloride, carbomer 1342, carbomer 934p, carbomer 940, carbomer homopolymer type b (allyl pentaerythritol crosslinked), carboxymethylcellulose sodium, castor oil, cetyl alcohol, chlorobutanol, chlorobutanol, anhydrous, cholesterol, citric acid, citric acid monohydrate, creatinine, diethanolamine, diethylhexyl phthalate, divinylbenzene styrene copolymer, edetate disodium, edetate disodium anhydrous, edetate sodium, ethylene vinyl acetate copolymer, gellan gum (low acyl), glycerin, glyceryl stearate, high density polyethylene, hydrocarbon gel, plasticized, hydrochloric acid, hydrochloric acid, diluted, hydroxyethyl cellulose, hydroxypropyl methylcellulose 2906, hypromellose 2910 (15000 mpa·s), hypromelloses, jelene, lanolin, lanolin alcohols, lanolin anhydrous, lanolin nonionic derivatives, lauralkonium chloride, lauroyl sarcosine, light mineral oil, magnesium chloride, mannitol, methylcellulose (4000 mpa·s), methylcelluloses, methylparaben, mineral oil, nitric acid, nitrogen, nonoxynol-9, octoxynol-40, octylphenol polymethylene, petrolatum, petrolatum, white, phenylethyl alcohol, phenylmercuric acetate, phenylmercuric nitrate, phosphoric acid, polidronium chloride, poloxamer 188, poloxamer 407, polycarbophil, polyethylene glycol 300, polyethylene glycol 400, polyethylene glycol 8000, polyoxyethylene-polyoxypropylene 1800, polyoxyl 35 castor oil, polyoxyl 40 hydrogenated castor oil, polyoxyl 40 stearate, polypropylene glycol, polysorbate 20, polysorbate 60, polysorbate 80, polyvinyl alcohol, potassium acetate, potassium chloride, potassium phosphate, monobasic, potassium sorbate, povidone k29/32, povidone k30, povidone k90, povidones, propylene glycol, propylparaben, soda ash, sodium acetate, sodium bisulfate, sodium bisulfite, sodium borate, sodium borate decahydrate, sodium carbonate, sodium carbonate monohydrate, sodium chloride, sodium citrate, sodium hydroxide, sodium metabisulfite, sodium nitrate, sodium phosphate, sodium phosphate dihydrate, sodium phosphate, dibasic, sodium phosphate, dibasic, anhydrous, sodium phosphate, dibasic, dihydrate, sodium phosphate, dibasic, heptahydrate, sodium phosphate, monobasic, sodium phosphate, monobasic, anhydrous, sodium phosphate, monobasic, dihydrate, sodium phosphate, monobasic, monohydrate, sodium sulfate, sodium sulfate anhydrous, sodium sulfate decahydrate, sodium sulfite, sodium thiosulfate, sorbic acid, sorbitan monolaurate, sorbitol, sorbitol solution, stabilized oxychloro complex, sulfuric acid, thimerosal, titanium dioxide, tocophersolan, trisodium citrate dihydrate, triton 720, tromethamine, tyloxapol and zinc chloride.

A pharmaceutical nucleic acid vaccine composition for retrobulbar administration may comprise at least one inactive ingredient. A non-exhaustive list of inactive ingredients for use in pharmaceutical compositions for retrobulbar administration includes hydrochloric acid and sodium hydroxide.

A pharmaceutical nucleic acid vaccine composition for intraocular administration may comprise at least one inactive ingredient. A non-exhaustive list of inactive ingredients for use in pharmaceutical compositions for intraocular administration includes benzalkonium chloride, calcium chloride, citric acid monohydrate, hydrochloric acid, magnesium chloride, polyvinyl alcohol, potassium chloride, sodium acetate, sodium chloride, sodium citrate and sodium hydroxide.

A pharmaceutical nucleic acid vaccine composition for intravitreal administration may comprise at least one inactive ingredient. A non-exhaustive list of inactive ingredients for use in pharmaceutical compositions for intravitreal administration includes calcium chloride, carboxymethylcellulose sodium, cellulose, microcrystalline, hyaluronate sodium, hydrochloric acid, magnesium chloride, magnesium stearate, polysorbate 80, polyvinyl alcohol, potassium chloride, sodium acetate, sodium bicarbonate, sodium carbonate, sodium chloride, sodium hydroxide, sodium phosphate dibasic heptahydrate, sodium phosphate monobasic monohydrate and trisodium citrate dehydrate.

A pharmaceutical nucleic acid vaccine composition for subconjunctival administration may comprise at least one inactive ingredient. A non-exhaustive list of inactive ingredients for use in pharmaceutical compositions for subconjunctival administration includes benzyl alcohol, hydrochloric acid and sodium hydroxide.

A pharmaceutical nucleic acid vaccine composition for auricular administration may comprise at least one inactive ingredient. A non-exhaustive list of inactive ingredients for use in pharmaceutical compositions for auricular administration includes acetic acid, aluminum acetate, aluminum sulfate anhydrous, benzalkonium chloride, benzethonium chloride, benzyl alcohol, boric acid, calcium carbonate, cetyl alcohol, chlorobutanol, chloroxylenol, citric acid, creatinine, cupric sulfate, cupric sulfate anhydrous, edetate disodium, edetic acid, glycerin, glyceryl stearate, hydrochloric acid, hydrocortisone, hydroxyethyl cellulose, isopropyl myristate, lactic acid, lecithin, hydrogenated, methylparaben, mineral oil, petrolatum, petrolatum, white, phenylethyl alcohol, polyoxyl 40 stearate, polyoxyl stearate, polysorbate 20, polysorbate 80, polyvinyl alcohol, potassium metabisulfite, potassium phosphate, monobasic, povidone k90f, povidones, propylene glycol, propylene glycol diacetate, propylparaben, sodium acetate, sodium bisulfite, sodium borate, sodium chloride, sodium citrate, sodium hydroxide, sodium phosphate, dibasic, anhydrous, sodium phosphate, dibasic, heptahydrate, sodium phosphate, monobasic, anhydrous, sodium sulfite, sulfuric acid and thimerosal.

### Dosing

Provided herein are methods comprising administering nucleic acid vaccines to a ruminant subject. The specific dose level for any particular ruminant will depend upon a variety of factors including the species, the activity of the specific compound employed; the specific composition employed; the age, body weight, general health, sex, and diet of the ruminant; the time of administration, route of administration, and rate of excretion of the specific compound employed; drugs used in combination or coincidental with the specific compound employed; and like factors well known in the arts. Compositions in accordance with the present disclosure are typically formulated in dosage unit form for ease of administration and uniformity of dosage. It will be understood, however, that the total daily usage of the compositions of the present disclosure may be decided by the attending administrator within the scope of sound judgment.

In certain embodiments, compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, twenty, thirty, forty, or more administrations). When multiple administrations are employed, split dosing regimens such as those described herein may be used.

According to the present disclosure, nucleic acid vaccines may be administered in split-dose regimens. As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In some embodiments, the nucleic acid vaccines of the present disclosure are administered to a ruminant subject in split doses. The nucleic acid vaccines may be formulated in buffer only or in a formulation described herein.

### Dosage Forms

Vaccines, antibodies, milk, animal feed, agents (e.g., an agent that reduces methane production in a ruminant, a probiotic bacterial strain, etc.), or other compositions of the present disclosure may be formulated into a dosage form described herein, such as an intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S. P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the nucleic acid vaccines then depends upon its rate of dissolution which, in turn, may depend upon crystal size and crystalline form. Alternatively, delayed absorption of a parenterally administered nucleic acid vaccine may be accomplished by dissolving or suspending the nucleic acid vaccines in an oil vehicle. Injectable depot forms are made by forming microencapsule matrices of the nucleic acid vaccines in biodegradable polymers such as polylactide-polyglycolide. Depending upon the ratio of nucleic acid vaccines to polymer and the nature of the particular polymer employed, the rate of polynucleotides release can be controlled. Examples of other biodegradable polymers include, but are not limited to, poly(orthoesters) and poly(anhydrides). Depot injectable formulations may be prepared by entrapping the nucleic acid vaccines in liposomes or microemulsions which are compatible with body tissues.

### Pulmonary Formulation

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 μm to 500 μm. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Multi-Dose and Repeat-Dose Administration

In some embodiments, vaccines, antibodies, milk, animal feed, agents (e.g., an agent that reduces methane production in a ruminant, a probiotic bacterial strain, a small molecule inhibitor, etc.), or other compositions of the present disclosure may be administered in two or more doses (referred to herein as “multi-dose administration”). Such doses may comprise the same components or may comprise components not included in a previous dose. Such doses may comprise the same mass and/or volume of components or an altered mass and/or volume of components in comparison to a previous dose. In some embodiments, multi-dose administration may comprise repeat-dose administration. As used herein, the term “repeat-dose administration” refers to two or more doses administered consecutively or within a regimen of repeat doses comprising same or different components. In some embodiments, the repeat dose may comprise substantially the same components provided at substantially the same mass and/or volume. In some embodiments, subjects may display a repeat-dose response. As used herein, the term “repeat-dose response” refers to a response in a subject to a repeat-dose that differs from that of another dose administered within a repeat-dose administration regimen. In some embodiments, such a response may be the expression of a protein in response to a repeat-dose comprising a nucleic acid vaccine composition. In such embodiments, protein expression may be elevated in comparison to another dose administered within a repeat-dose administration regimen or protein expression may be reduced in comparison to another dose administered within a repeat-dose administration regimen. Alteration of protein expression may be from about 1% to about 20%, from about 5% to about 50% from about 10% to about 60%, from about 25% to about 75%, from about 40% to about 100% and/or at least 100%. A reduction in expression of mRNA administered as part of a repeat-dose regimen, wherein the level of protein translated from the administered RNA is reduced by more than 40% in comparison to another dose within the repeat-dose regimen is referred to herein as “repeat-dose resistance.”

### Adjuvant

Adjuvants or immune potentiators, may also be administered with or in combination with one or more vaccines (e.g., protein vaccine, nucleic acid vaccine).

The term “adjuvant” refers to an agent that when administered in conjunction with or as part of a composition described herein augments, enhances, and/or boosts the immune response to the protein encoded by the nucleic acid vaccine (e.g., at least one cell surface protein or fragment thereof of at least one methanogen), but when the agent is administered alone does not generate an immune response. In some embodiments, the adjuvant generates an immune response to the protein encoded by the nucleic acid vaccine and does not produce an allergy or other adverse reaction. Adjuvants can enhance an immune response by several mechanisms including, e.g., lymphocyte recruitment, stimulation of B and/or T cells, and stimulation of macrophages.

In some embodiments, an adjuvant acts as a co-signal to prime T-cells and/or B-cells and/or NK cells as to the existence of the protein encoded by the nucleci acid vaccine.

Advantages of adjuvants include the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccines. These may be co-administered by any route, e.g., intramusculary, subcutaneous, IV or intradermal injections. Adjuvants useful in the present invention may include, but are not limited to, natural or synthetic. They may be organic or inorganic.

When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (Glaxo SmithKline), AS04 (Glaxo SmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al, in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al, N. Engl. J. Med. 336, 86-91 (1997)).

Aduvants may be selected from any of the classes (1) mineral salts, e.g., aluminium hydroxide and aluminium or calcium phosphate gels; (2) emulsions including: oil emulsions and surfactant based formulations, e.g., microfluidised detergent stabilised oil-in-water emulsion, purified saponin, oil-in-water emulsion, stabilised water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensoactive compounds; (9) carbohydrates; or combinations thereof. Adjuvants for DNA nucleic acid vaccines (DNA) have been disclosed in, for example, Kobiyama, et al Vaccines, 2013, 1(3), 278-292, the contents of which are incorporated herein by reference in their entirety. Any of the adjuvants disclosed by Kobiyama may be used in the vaccines of the present disclosure.

Other adjuvants which may be utilized in the vaccines of the present disclosure include any of those listed on the web-based vaccine adjuvant database, Vaxjo; World Wide Web at violinet.org/vaxjo/ and described in for example Sayers, et al., J. Biomedicine and Biotechnology, volume 2012 (2012), Article ID 831486, 13 pages, the content of which is incorporated herein by reference in its entirety.

Selection of appropriate adjuvants will be evident to one of ordinary skill in the art. Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derb/ed P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hlFN-gamma/Interferon-g, Interleukin-4p, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(LC), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant El 12K of Cholera Toxin mCT-El 12K, and/or Matrix-S. Other adjuvants which may be co-administered with the nucleic acid vaccines of the invention include, but are not limited to interferons, TNF-alpha, TNF-beta, chemokines such as CCL21, eotaxin, HMGB1, SA100-8alpha, GCSF, GMCSF, granulysin, lactoferrin, ovalbumin, CD-40L, CD28 agonists, PD-1, soluble PD1, LI or L2, or interleukins such as IL-1, IL-2, IL-4, IL-6, IL-7, IL-10, IL-12, IL-13, IL-21, IL-23, IL-15, IL-17, and IL-18. These may be administered with the nucleic acid vaccine on the same encoded polynucleotide, e.g., polycistronic, or as separate mRNA encoding the adjuvant and antigen.

In some embodiments, the adjuvant comprises Glucopyranosyl Lipid Adjuvant (GLA), CpG oligodeoxynucleotides (e.g., Class A or B), poly(LC), aluminum hydroxide, or Pam3CSK4.

In some embodiments, the adjuvant comprises: (a) (±)-N-(3-aminopropyl)-N,N-dimethyl-2,3-bis(syn-9-tetradeceneyloxy)-1-propanaminium bromide (GAP-DMORIE) and a neutral lipid; (b) a cytokine; (c) mono-phosphoryl lipid A and trehalosedicorynomycolateAF (MPL+TDM); (d) a solubilized mono-phosphoryl lipid A formulation; and/or (e) CRL1005/BAK.

In some embodiments, the neutral lipid in (a) comprises (a) 1,2-dioleoyl-sn-glycero-3-phosphoethanolamine (DOPE); (b) 1,2-diphytanoyl-sn-glycero-3-phosphoethanolamine (DPyPE); and/or (c) 1,2-dimyristoyl-glycer-3-phosphoethanolamine (DMPE).

In some embodiments, the adjuvant comprises saponin, Montanide ISA61, a chitosan thermogel, a lipid nanoparticle/cationic liposome adjuvant, or any combination thereof. In preferred embodiments, the adjuvant comprises Montanide ISA61.

In some embodiments, the pharmaceutical composition of the present disclosure comprises a transfection facilitating compound. In some embodiments, the pharmaceutical composition of the present disclosure comprises an adjuvant and a transfection facilitating compound. In some embodiments, the transfection facilitating compound comprises (±)-N-(2-hydroxyethyl)-N,N-dimethyl-2,3-bis(tetradecyloxy)-1-propanaminium bromide) (DMRIE).

### Antibody

Unless otherwise specified here within, the terms “antibody” and “antibodies” broadly encompass naturally-occurring forms of antibodies (e.g. IgG, IgA, IgM, IgE); and recombinant antibodies, such as single-chain antibodies, chimeric antibodies, and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., at least one cell surface protein or fragment thereof of at least one methanogen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CHi domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to ruminant immunoglobulin constant region cDNA or genomic sequences, in order to generate expression vectors encoding complete IgG polypeptides or other isotypes (e.g., IgGA). VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. chimeric, etc.). Antibodies may also be fully ruminant. The terms “monoclonal antibodies” and “monoclonal antibody composition,” as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The present disclosure includes a monoclonal antibody that works particularly well in binding and neutralizing at least one methanogen. Upon immunizing a ruminant population (e.g., of milk-producing ruminant), the milk comprising the antibody can be screened for antibodies with superior activity (e.g., specific binding, neutralizing at least one methanogen, etc.). The amino acid sequence of such antibodies can be determined (e.g., mass spec-based sequencing, Next Gen Sequencing, or other methods known in the art), their expressing DNA vectors can be synthesized, and monoclonal antibodies can be produced. One or a combination of at least two or more antibodies can be added to the drinking water and/or animal feed, and be given to a ruminant population.

Alternatively, such monoclonal antibodies can be generated by immunizing a vehicle animal (e.g., mouse, rabbit, etc.), and hybridomas expressing the animals can be recovered. Standard hybridoma methods for producing antibodies are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, NY (2001)). Antibodies produced by hybridomas can be screened and utilized according to the methods described above and herein.

In some embodiments, the antibody is a polyclonal antibody. In some embodiments, the antibody is a monoclonal antibody. In some embodiments, the antibody is an IgG or IgA. In some embodiments, the antibody is IgA. The IgA isoform, at least in cattle, may be more stable in the rumen. For example, IgA levels in cattle saliva were reduced by only 40% after 8 h exposure to rumen contents while IgG levels were reduced by 80%.

In some embodiments, the antibody is lyophilized. In some embodiments, the antibody is in a pharmaceutical composition of the present disclosure or those known in the art. In some embodiments, a composition comprising an antibody further comprises at least one excipient and/or carrier. In some embodiments, the antibody is in the animal feed. In some embodiments, the antibody is in the solid animal feed. In other embodiments, the antibody is in the liquid animal feed. In some embodiments, the antibody is in the drinking water or milk. In preferred embodiments, the antibody is orally consumed by a ruminant such that the antibody comes in contact with at least one methanogen present in the gut of the ruminant. In preferred embodiments, oral administration of the antibody reduces the number and/or type of at least one methanogen.

### Methods for Detection of Antibody

In certain embodiments, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In some embodiments, the antibody titer is produced or reached by 10 days following vaccination, by 20 days following vaccination, by 30 days following vaccination, by 40 days following vaccination, or by 50 or more days following vaccination. In some embodiments, the titer is produced or reached following a single dose of vaccine administered to the subject. In other embodiments, the titer is produced or reached following multiple doses, e.g., following a first and a second dose (e.g., a repeat dose.)

In certain aspects, antigen-specific antibodies are measured in units of pg/ml or are measured in units of IU/L (International Units per liter) or mIU/ml (milli International Units per ml). In some embodiments of the invention, an efficacious vaccine produces >0.5 pg/ml, >0.1 pg/ml, >0.2 pg/ml, >0.35 pg/ml, >0.5 pg/ml, >1 pg/ml, >2 pg/ml, >5 pg/ml or >10 pg/ml. In some embodiments, an efficacious vaccine produces >10 mIU/ml, >20 mIU/ml, >50 mIU/ml, >100 mIU/ml, >200 mIU/ml, >500 mIU/ml or >1000 mIU/ml.

Methods of detecting the presence of antibodies are well known in the art.

In some embodiments, antibody level or concentration is determined or measured by neutralization assay, e.g., neutralization of at least one methanogen.

Other exemplary methods include, but are not limited to, immunodiffusion, immunoelectrophoresis, radioimmunoassay (RIA), enzyme-linked immunosorbent assays (ELISAs; including variants such as competitive ELISA, sandwich ELISA, etc.), immunofluorescent assays, Western blotting, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference).

For example, ELISA and RIA procedures may be conducted such that a desired protein standard (e.g., an extracellular domain of at least one cell surface protein or a fragment thereof of at least one methanogen) is labeled (with a radioisotope such as ¹²⁵I or 3sS, or an assayable enzyme, such as horseradish peroxidase or alkaline phosphatase), and is brought into contact with a sample comprising the antibody, whereon the amount of the labeled protein standard bound to the antibody is measured.

Enzymatic and radiolabeling of a protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzymes are non-specific (such as using formaldehyde), and will only yield a proportion of active enzyme.

It may be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

### Milk

In certain aspects, provided herein are milk and derivatives thereof. Milk produced by vaccinated female ruminants (e.g., dairy cows) comprises antibodies that bind at least one cell surface protein or a fragment thereof of at least one methanogen. Such milk can be orally consumed by ruminants such that the antibodies therein can come in contact with at least one methanogen present in the gut of the ruminants. Upon contact, the antibodies in the milk can neutralize the at least one methanogen and contribute to reducing methane production by the ruminants.

In some embodiments, the milk is pasteurized and/or homogenized. In some embodiments, the milk is lyophilized, filtered, concentrated, evaporated, or processed to form dry milk powder (e.g., boiling at low pressure at low temperature). In some embodiments, said processing may allow longer shelf life of the milk/milk product and the antibodies present therein. In some embodiments, the fat content is removed/reduced from the milk. Processing of milk and/or preparation of derivatives of milk are well known in the art.

Appropriate care is taken to preserve the structural and functional (e.g., binding a methanogen) aspects of the antibodies. For example, in some embodiments, high pressure (˜200 MPa) and low temperature (−4° C.) are used throughout the process as described at least by Kim et al. (2008) Journal of Dairy Science, 91:4176-4182. In other embodiments, milk may be pasteurized at low-temperature of 60° C. for 10 minutes at standard pressure. These conditions may pasteurize milk without significantly altering the antibody function.

Alternatively, milk can be filtered to remove microorganisms instead of pasteurizing. Microfiltration is a process that replaces pasteurization and produces milk with fewer microorganisms and longer shelf life without a change in the quality of the milk. In this process, cream is separated from the skimmed milk and the skimmed milk is forced through ceramic microfilters that trap 99.9% of microorganisms in the milk (as compared to 99.999% killing of microorganisms in standard high temperature short time pasteurization).

Ultrafiltration uses finer filters than microfiltration, which allow lactose and water to pass through while retaining fats, calcium and protein. As with microfiltration, the fat may be removed before filtration and added back in afterwards. Ultrafiltered milk is used widely in the industry in cheesemaking.

### Animal Feed

Provided herein are animal feeds that are useful in reducing methane production by a ruminant. Such animal feed may be used in combination with any one of vaccines, antibodies, milk, agents (e.g., an agent that reduces methane production in a ruminant, a probiotic bacterial strain, a small molecule inhibitor, etc.), or other compositions of the present disclosure (e.g., those reducing methane production in a ruminant). Animal feed comprises at least one feed additive, which reduces the methane production in a ruminant.

In some embodiments, the animal feed comprises an antibody that binds at least one cell surface antigen or a fragment thereof of at least one methanogen.

In some embodiments, the animal feed comprises a composition comprising an antibody that binds at least one cell surface antigen or a fragment thereof of at least one methanogen. For example, the animal feed may comprise milk or derivatives thereof comprising said antibody.

In some embodiments, the animal feed is liquid (e.g., drinking water, milk). An antibody that binds at least one cell surface antigen or a fragment thereof of at least one methanogen or a composition comprising same may be added to the liquid animal feed (e.g., drinking water) before being given to the ruminant. In some embodiments, said antibody or a composition comprising same may be added to the drinking water. In other embodiments, the milk or derivatives thereof comprising said antibody may be given directly to the ruminant, or added to other liquid animal feed (e.g., drinking water).

In other embodiments, the animal feed is solid. In some such embodiments, the animal feed may comprise hay, straw, silage, compressed and pelleted feeds, oils and mixed rations, and sprouted grains and legumes. An antibody that binds at least one cell surface antigen or a fragment thereof of at least one methanogen or a composition comprising same may be added to the solid animal feed before being given to the ruminant.

In some embodiments, an animal feed may comprise fats and fatty acids that further aid in reducing methane production in ruminants. Based on a meta-analysis, fat supplementation reduced CH₄ by 3.77% in cattle and 4.30% in sheep per 1% dietary fats. Fat decreases CH₄ production (expressed as g/kg digestible dry matter (DM)) more from sheep than from cattle, which was attributed to the comparatively lower depression of DM digestion together with numerically larger depression of CH₄ production (g/kg DM) by fat in sheep. Among fatty acids, C12:0, C18:3 and other polyunsaturated fatty acids (PUFA) are more potent than saturated fatty acids. The CH4-suppressing efficacy of fats generally persists, with persistent suppression being noted for 72 days and longer in cattle.

Fats supplemented up to 6% of the diet (DM) can also improve milk production while appreciably decreasing CH₄ emissions (15%) in cattle, but higher concentrations decreased production efficiency due to a reduction of feed digestion and fermentation. Medium-chain fatty acids (MCFA) and PUFA can lower abundance and metabolic activities of rumen methanogens and change their species composition. PUFA can also directly inhibit protozoa and serve as hydrogen sink through biohydrogenation. Both MCFA and PUFA appear to damage the cell membrane, thereby abolishing the selective permeability of cell membrane, which is required for survival and growth of methanogens and other microbes. The inhibitory effect of fat on methanogenesis is more pronounced in cattle fed concentrate-based diets than in cattle fed forage-based diets. Because C12: and C14:0 is more inhibitory to M. ruminantium at pH 5 than at pH 7, the concentrate level-dependent anti-methanogenic efficacy of MCFA and PUFA is probably attributed to the lower pH associated with high-concentrate diets.

In some embodiments, the animal feed comprises fat and/or fatty acid. In some embodiments, the animal feed comprises fat and/or fatty acid that is at least about 1%, 2%, 3%, 4%, 5%, or 6% of the diet (e.g., diet based on dry matter).

Numerous animal feed and feed additives are known in the art. Any agent that reduce methane production in a ruminant (e.g., small molecule inhibitors, e.g., Table 8B, probiotic bacterial strain, etc.; see below) described herein or those known in the art may be used as a feed additive. Certain exemplary feed additives include: berberine, nitrate, eucalyptus oil, alliin, diallyl disulfide (DADS), flavanone glycoside (e.g., neohesperidin, isonaringin, poncirin, hesperidin), 3-nitrooxypropanol, rac-4-Phenylbutane-1,2-diyl dinitrate, 2-(hydroxymethyl)-2-(nitrooxymethyl)-1,3-propanediol, N-ethyl-3-nitro-oxy-propionic sulfonyl amide, 5-nitrooxy-pentanenitrile, 5-nitrooxy-pentane, 3-nitro-oxy-propyl propionate, 1,3-bis-nitrooxypropane, 1,4-bis-nitrooxybutane, 1,5-bis-nitrooxypentane, 3-nitro-oxy-propyl benzoate, 3-nitro-oxy-propyl hexanoate, 3-nitro-oxy-propyl 5-nitro-oxy-hexanoate, benzylnitrate, isosorbid-dinitrate, N-[2-(nitrooxy)ethyl]-3-pyridinecarboxamide, 3-nitrooxy propionic acid, methyl-3-nitrooxy propionate, ethyl-3-nitrooxy propionate, ethyl-4-nitrooxy butanoate, ethyl-3-nitrooxy butanoate, 5-nitrooxy pentanoic acid, ethyl-5-nitrooxy pentanoate, 6-nitrooxy hexanoic acid, ethyl-6-nitrooxy hexanoate, ethyl-4-nitrooxy-cyclohexylcarboxylate, 8-nitrooxy octanoic acid, ethyl-8-nitrooxy octanoate, 11-nitrooxy undecanoic acid, ethyl-11-nitrooxy undecanoate, 5-nitrooxy-pentanoic amide, 5-nitrooxy-N-methyl-pentanoic amide, lauric acid, and haloform (e.g., bromoform, chloroform, iodoform).

Agents that Reduce Methane Production in Ruminants

### Combination Treatment

Vaccines, antibodies, milk, animal feed, and agents (e.g., an agent that reduces methane production in a ruminant, a probiotic bacterial strain, etc.) may be administered to a ruminant in any combination. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent (vaccines, antibodies, milk, animal feed, agents that reduce methane production in a ruminant) will be administered at a dose and/or on a time schedule determined for that agent. In some embodiments, the present disclosure encompasses the delivery of combinations that may improve immune response against at least one methanogen, and/or reduce methane production by a ruminant.

The combinations can conveniently be presented for use in the form of a pharmaceutical formulation and thus pharmaceutical compositions comprising a combination as defined above together with a pharmaceutically acceptable diluent or carrier represent a further aspect of the invention.

The individual compounds of such combinations can be administered either sequentially or simultaneously in separate or combined pharmaceutical formulations.

It will further be appreciated that (vaccines, antibodies, milk, animal feed, agents that reduce methane production in a ruminant) in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually. In some embodiments, the combinations, each or together may be administered according to the split dosing regimens described herein.

The term “conjoint” or “combination” administration, as used herein, refers to the administration of two or more agents that aid in reducing methane production in a ruminant. The different agents comprising the combination may be administered concomitant with, prior to, or following the administration of one or more agents.

In certain embodiments, combination administration can demonstrate synergisms between the two or more agents resulting in a greater methane reduction in ruminant than either agent alone.

Synergistic effects, also known as synergy, refer to the phenomenon where the combined effect of two or more factors or components is greater than the sum of their individual effects. In other words, when these factors or components interact, they create an amplified or enhanced effect that is greater than what would be expected based on their individual contributions.

In certain cases, the agents may have different modes of action or mechanisms by which they exert their effects, for example targeting different methanogens or different methanogen enzymes. When these agents are combined, their actions can complement each other, targeting different aspects of a problem or working on multiple pathways simultaneously. This complementary action enhances their overall effectiveness, resulting in a better outcome than either agent could achieve alone.

In certain cases, synergism can significantly enhance the efficacy of the agents involved. For example, the agents may interact in a way that enhances their absorption, distribution, or bioavailability, increasing their effectiveness in treating a particular condition.

In certain cases, one agent may enhance the effects of the other without contributing much individually. This is known as potentiation. The presence of one agent can increase the uptake, binding affinity, or sensitivity of the other, making it more potent and effective. The combined effect is greater than what would be achieved by either agent on its own.

In certain cases, one or more agents may have inherent weaknesses or face resistance from target organisms or systems. By combining them with another agent, the synergistic interaction can bypass or counteract these obstacles, leading to a more effective outcome. For example, antibodies generated via vaccination of a first vaccine may face resistance to ruminal proteases, thus, combining with protease inhibitors or one or more additional vaccine towards certain ruminal proteases may reduce the resistance of the first vaccine.

In certain cases, combining two agents can amplify the positive effects or benefits they provide individually. For example, combining a vaccine for a methanogen with a vaccine towards a separate microorganism that is syntropic with the methanogen can amplify the positive effects or benefits of the vaccine by further reducing the fitness of the methanogen.

The level of synergism achieved when combining agents depends on numerous factors and is typically assessed through experimental studies or empirical observations specific to the agents and desired outcomes. Generally, combination of agents can lead to different degrees of synergisms ranging from No synergism to Supra-additive synergism.

No Synergism: In some cases, the combined effect of two or more agents may simply be additive or even less than additive. This means that the combined effect is equal to the sum of their individual effects or even lower. In such instances, no synergism is observed, and the agents may not interact in a way that amplifies their effects.

Mild to Moderate Synergism: A common outcome when combining agents is a mild to moderate level of synergism. This implies that the combined effect is greater than the sum of their individual effects, but not dramatically so. The degree of synergism may vary depending on the specific agents and the conditions of their interaction.

Strong Synergism: In some cases, the combination of agents can lead to a strong synergistic effect. This means that the combined effect is significantly greater than the sum of their individual effects. Strong synergism often results in an amplified and more potent effect, exceeding what would be expected based on the additive effects of the individual agents.

Supra-additive Synergism: In rare instances, the combined effect of two or more agents can be supra-additive, meaning it surpasses even strong synergism. Supra-additive synergism results in an exceptionally powerful effect that far exceeds the sum of the individual effects. Such cases are usually considered highly beneficial, as they can provide remarkable outcomes in terms of efficacy, efficiency, or other desired parameters.

Any suitable degree of synergism (%_{Synergism}) can be demonstrated with a combination therapy comprising a nucleic acid vaccine and another agent, such as an improvement of at least 5, 10, 20, 30, 40, 50, 60, 70, 80, 90, 100, 200, 300, or 400% and/or not more than 10, 20, 30, 40, 50, 60, 70, 80, 90, 100, 200, 300, 400, 500, or 1000%, for example 5-1000%, preferably 10-500%, more preferably 30-300%. In certain embodiments, the improvement is measured in the amount of methane reduced when administered a combination therapy as compared to either agent alone, for example % _{Synergism}=Methane_{combo}*(Methane_{vaccine})⁻¹. In certain embodiments, the % _{Synergism} is measured in a herd of at least 10, 20, 30, 40, 50, 60, 70, 80, 90, 100, 500, or 1000 animals and/or not more than 20, 30, 40, 50, 60, 70, 80, 90, 100, 500, 1000, or 2000 animals and the statistical significance, e.g., coefficient of variation of % _{Synergism} within the herd is at least 50, 60, 70, 75, 80, 85, 90, 95, 99, 99.5, or 100%.

In certain embodiments, the synergism resulting from the combinatorial therapy results in a prolonged efficacy of the treatment as compared to either alone. In certain embodiments, the combinatorial therapy is effective for at least 1, 2, 3, 4, 5, 6, 7, 8, 9, 10, 11, 12, 16, or 20 months and/or not more than 2, 3, 4, 5, 6, 7, 8, 9, 10, 11, 12, 16, 20, or 24 months, for example 1-24 months. In certain embodiments, the length of efficacy of the treatment is at least 5, 10, 20, 30, 40, 50, 60, 70, 80, 90, 100, 200, 300, or 400% and/or not more than 10, 20, 30, 40, 50, 60, 70, 80, 90, 100, 200, 300, 400, 500, or 1000% greater than either agent alone, for example 5-1000%, preferably 10-500%, more preferably 30-300%.

An exemplary combinatorial therapy includes vaccination with a first vaccine encoding one or more methanogen surface proteins in combination with at least one additional vaccine encoding one or more of different methanogen surface proteins.

Another exemplary combinatorial therapy includes vaccination with a vaccine encoding one or more methanogen surface proteins in combination with administration of a small molecule inhibitor of methanogenesis. Without wishing to be bound to theory, it is hypothesized that the small molecule inhibitor of methanogenesis may remove the plurality of ruminal methanogens, and antibodies generated from the vaccination prevent new methanogens for colonizing the methanogen-deficient rumen.

Accordingly, in certain aspects, a vaccine of the present disclosure is administered to a ruminant conjointly or in a combination with at least one inhibitor of methane production described herein or those known in the art. In some embodiments, the at least one inhibitor is selected from Table 8B.

In some embodiments, the at least one inhibitor is administered to a ruminant at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times. In some embodiments, the at least one inhibitor is administered to a ruminant daily, semiweekly, weekly, biweekly (every 2 weeks), monthly, semiannually, or annually. In some embodiments, the at least one inhibitor is administered to a ruminant every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the at least one inhibitor is administered to a ruminant for a duration of at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks. In some embodiments, the at least one inhibitor is administered to a ruminant for at least 1 week but no more than 1 month. In some embodiments, the at least one inhibitor is administered to a ruminant orally, intravenously, intramuscularly, or subcutaneously. In preferred embodiments, the at least one inhibitor is administered to a ruminant orally. In some embodiments, the at least one inhibitor is administered to a ruminant as a feed additive.

In some embodiments, the at least one inhibitor is administered to a ruminant concomitant with, prior to, or following the vaccination with a vaccine of the present disclosure. In some embodiments, the at least one inhibitor is administered to a ruminant on the same day as the ruminant is vaccinated. In some embodiments, a ruminant is administered with the at least one inhibitor one or more times to reduce the methane production by the ruminant, and said ruminant is vaccinated as a maintenance regimen.

In some embodiments, the at least one inhibitor comprises 3-NOP. In some embodiments, a ruminant is administered at least about or no more than 0.1, 0.2, 0.3, 0.4, 0.5, 0.6, 0.7, 0.8, 0.9, 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 grams (g) of 3-NOP per dose. In some embodiments, 3-NOP is administered to a ruminant every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days.

In some embodiments, at least about 0.5 g but no more than 25 g of 3-NOP is administered to a ruminant in a given day. In preferred embodiments, about 2.5 g of 3-NOP is administered to a ruminant in a given day. In some embodiments, about 2.5 g of 3-NOP is administered to a ruminant per day for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

TABLE 8B
Exemplary methanogenesis inhibitor

| Chemical | Reference |
|---|---|
| 1,3-bis-nitrooxypropane | US10154981B2 |
| 1,3-bis-nitrooxypropane | US9266814B2 |
| 1,4-bis-nitrooxybutane | US10806706B2 |
| 1,4-bis-nitrooxybutane | US10154981B2 |
| 1,4-bis-nitrooxybutane | US9266814B2 |
| 1,5-bis-nitrooxypentane | US10806706B2 |
| 1,5-bis-nitrooxypentane | US10154981B2 |
| 1,5-bis-nitrooxypentane | US9266814B2 |
| 11-nitrooxy undecanoic acid | US9365489B2 |
| 11-nitrooxy undecanoic acid | US10154981B2 |
| 2 mercaptoethanesulfonate | EP2747181A1 |
| 2-bromoethane sulfonic acid (2-BESA) | EP2747181A1 |
| 2-bromoethane sulphonate | US6251879B1 |
| 2-chloroethanesulfonate | EP2747181A1 |
| 2-nitropropanol | EP2747181A1 |
| 3-nitro-oxy-propyl 5-nitro-oxy-hexanoate | US10154981B2 |
| 3-nitro-oxy-propyl benzoate | US10154981B2 |
| 3-nitro-oxy-propyl hexanoate | US10154981B2 |
| 3-nitro-oxy-propyl propionate | US10154981B2 |
| 3-nitrooxy propionic acid | US9365489B2 |
| 3-nitrooxy propionic acid | US10154981B2 |
| 3-nitrooxy-propyl 5-nitrooxy-hexanoate | US9266814B2 |
| 3-nitrooxy-propyl benzoate | US9266814B2 |
| 3-nitrooxy-propyl hexanoate | US9266814B2 |
| 3-nitrooxy-propyl propionate | US9266814B2 |
| 3-nitrooxypropanol | US10806706B2 |
| 3-nitrooxypropanol | US10154981B2 |
| 3-nitrooxypropanol | US9266814B2 |
| 5-nitrooxy hexanoic acid | US10806706B2 |
| 5-nitrooxy pentanoic acid | US10806706B2 |
| 5-nitrooxy pentanoic acid | US9365489B2 |
| 5-nitrooxy pentanoic acid | US10154981B2 |
| 5-nitrooxy-N-methyl-pentanoic amide | US9365489B2 |
| 5-nitrooxy-N-methyl-pentanoic amide | US10154981B2 |
| 5-nitrooxy-N-pentanoic amide | US9365489B2 |
| 5-nitrooxy-pentane | US10154981B2 |
| 5-nitrooxy-pentane | US9266814B2 |
| 5-nitrooxy-pentanenitrile | US10154981B2 |
| 5-nitrooxy-pentanenitrile | US9266814B2 |
| 5-nitrooxy-pentanoic amide | US10154981B2 |
| 6-nitrooxy hexanoic acid | US9365489B2 |
| 6-nitrooxy hexanoic acid | US10154981B2 |
| 8-nitrooxy octanoic acid | US9365489B2 |
| 8-nitrooxy octanoic acid | US10154981B2 |
| 9-nitrooxynonanol | US10806706B2 |
| abietic acid | US10799544B2 |
| acetylene | EP2747181A1 |
| ajoene | US20220256890A1 |
| Alcaligenes faecalis | US20140072535A1 |
| allicin | US20220256890A1 |
| alliin | US20220256890A1 |
| allylpropyl disulfide | US20220256890A1 |
| Asparagopsis armata | US10881697B2 |
| Bacillus amyloliquefaciens | WO2021211548A1 |
| Bacillus strain 300 (NRRL No. B-50943) | US20210360945A1 |
| Bacillus strain 86 (NRRL No. B-50944) | US20210360945A1 |
| Bacillus, Lactobacillus, Strptococcus, Candida and Pichia | WO2007072935A1 |
| bambermycin | AU2018229465A1 |
| benzylnitrate | US10154981B2 |
| berberine, nitrate, and eucalyptus oil | US10440975B2 |
| bis-(2-nitrooxyethyl) ether | US9266814B2 |
| bis(2-hydroxyethyl)amine dinitrate | US10806706B2 |
| bromochloromethane | US6251879B1 |
| Bromoforms | US20220175670A1 |
| chloroform | EP2747181A1 |
| Chloroforms | US20220175670A1 |
| Cysteine | US5843498A |
| dehydoabietic acid | US10799544B2 |
| diallyl disulfide | US20180093308A1 |
| diallyl disulphide | US20220256890A1 |
| diallyl trisulfide | US20180093308A1 |
| diallyl trisulfide | US20220256890A1 |
| egg powder from immunizing hens with methane-producing M. | US7820171B2 |
| stadtmaniae methanogens | |
| Eggs laid by hens innoculated with methanogen antigens | US20200121730A1 |
| essential oil | US20180001358A1 |
| ethyl 2 butynoate | EP2747181A1 |
| ethyl propionate | US20180093308A1 |
| ethyl-11-nitrooxy undecanoate | US9365489B2 |
| ethyl-11-nitrooxy undecanoate | US10154981B2 |
| ethyl-3-nitrooxy butanoate | US10154981B2 |
| ethyl-3-nitrooxy propionate | US9365489B2 |
| ethyl-3-nitrooxy propionate | US10154981B2 |
| ethyl-3-nitrooxy-butanoate | US9365489B2 |
| ethyl-4-nitrooxy butanoate | US10154981B2 |
| ethyl-4-nitrooxy-butanoate | US9365489B2 |
| ethyl-4-nitrooxy-cyclohexylcarboxylate | US9365489B2 |
| ethyl-4-nitrooxy-cyclohexylcarboxylate | US10154981B2 |
| ethyl-5-nitrooxy pentanoate | US9365489B2 |
| ethyl-5-nitrooxy pentanoate | US10154981B2 |
| ethyl-6-nitrooxy hexanoate | US9365489B2 |
| ethyl-6-nitrooxy hexanoate | US10154981B2 |
| ethyl-8-nitrooxy octanoate | US9365489B2 |
| ethyl-8-nitrooxy octanoate | US10154981B2 |
| ethylene | EP2747181A1 |
| eugenol | US7763273B2 |
| fluoroacetate | EP2747181A1 |
| fluvastatin | EP2747181A1 |
| halothane | US6251879B1 |
| hesperidin | EP2838376B1 |
| hop acid | US8197863B2 |
| hydrogen peroxide | WO2010071222A1 |
| hydroxycinnamic acid | WO2021038832A1 |
| Impatiens balsamina | NZ562783A |
| Iodoforms | US20220175670A1 |
| isonaringin | EP2838376B1 |
| isopimaric acid | US10799544B2 |
| isosorbid-dinitrate | US10154981B2 |
| isosorbid-dinitrate | US9266814B2 |
| laidlomycin | AU2018229465A1 |
| lasalocid | AU2018229465A1 |
| limonene | US7763273B2 |
| lovastatin | EP2747181A1 |
| lumazine | EP2747181A1 |
| medium and long chain fatty acids | EP2747181A1 |
| Megasphaera sp. & Coprococcus catus | US10961559B2 |
| methyl chloride | EP2747181A1 |
| methyl fluoride | EP2747181A1 |
| methyl-3-nitrooxy propionate | US9365489B2 |
| methyl-3-nitrooxy propionate | US10154981B2 |
| mevastatin | EP2747181A1 |
| Monascus sp. | US20130011384A1 |
| monensin | AU2018229465A1 |
| N-[2-(nitrooxy)ethyl]-3-pyridinecarboxamide | US10154981B2 |
| N-[2-(nitrooxy)ethyl]-3-pyridinecarboxamide | US9266814B2 |
| N-ethyl-3-nitro-oxy-propionic sulfonyl amide | US10154981B2 |
| neoabietic acid | US10799544B2 |
| neohesperidin | EP2838376B1 |
| nitrate | EP2747181A1 |
| nitrite | EP2747181A1 |
| nitroethane | EP2747181A1 |
| palustric acid | US10799544B2 |
| Peroxide source, peroxidase | US20210227850A1 |
| phosphate | EP2747181A1 |
| pimaric acid | US10799544B2 |
| poncirin | EP2838376B1 |
| Propionibacterium | US20120276058A1 |
| propynoic acid | EP2747181A1 |
| protease-resistant bacteriocin | EP1673983A1 |
| R(OC2H4)n(OH); R = C12-15; n = 7 | AU2013288441B2 |
| R1(CH2)nONO2; n = 1-15; R1 = H, C1-C6alkyl, phenyl, —OH, | US10806706B2 |
| —NH2, —CN, —COOH, —O(C═O)R8, —NHC(═O)R8, | |
| SO2NHR8, or —ONO2; R8 = C1-C6alkyl, phenyl, pyridyl; when | |
| n is >3 the hydrocarbon chain may be interrupted by —O— or | |
| —NH— | |
| rac-4-Phenylbutane-1,2-diyl dinitrate, 2-(hydroxymethyl)-2- | US10154981B2 |
| (nitrooxymethyl)-1,3-propanediol | |
| red yeast rice | US20140322798A1 |
| s-allyleysteine | US20220256890A1 |
| saponin | US20180001358A1 |
| Saponin & medium chain fatty acid | US10695393B2 |
| sodium bromoethenesulfonate (BES) | EP2747181A1 |
| vinyldithiines | US20220256890A1 |
| ω-nitrooxyC3-10alkane-1-ol | US20210392922A1 |
| 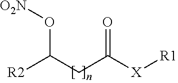 | US9365489B2 |
| 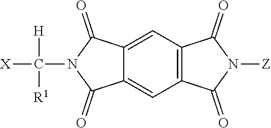 | WO2022103280A1 |
| 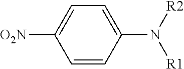 | US20160183564A1 |
| 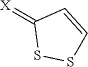 | EP3628169A1 |
| 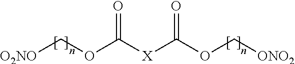 | US20210163397A1 |
| 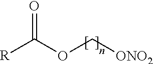 | WO2020221805A1 |
| 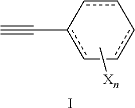 | AU2018383221A1 |
| 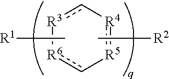 | US20030219467A1 |
| Asparagopsis taxiformis | USPP34607P3 |
| Lauric acid | |

*Each of the references in Table 8B is incorporated herein by reference.

Other exemplary inhibitors of methane production are discussed below.
Probiotics that Reduce Methane Production in Ruminants

Probiotics are a class of beneficial active microorganisms or their cultures. Probiotics are useful in reducing CH4 emissions in ruminants (Table 8C). There are many types of probiotics, and different strains have different inhibitory effects on CH4 emissions. For example, the GA03 strain of Acetobacter is more effective at inhibiting CH4 production than other isolated strains. Most probiotics reduce CH4 production by influencing the activities of ruminal microorganisms, with no adverse effects on animals. In addition, probiotics enhance ruminal fermentation.

Lactic acid bacteria, which have been used as feed additives for a long time, not only reduce CH4 emissions per unit volatile fatty acid (VFA) output, but also improve the fermentation quality and fiber digestibility of silage. In addition, the denitrifying bacterium Bacillus 79R4 could prevent NO-2-N poisoning and microbial ecosystems from impairing fermentation efficiency. Furthermore, Bacillus licheniformis reduces CH4 production and increases feed energy and protein utilization.

TABLE 8C
Exemplary probiotic bacteria that reduce methane production in ruminants

| | | Addition amount; maximum | |
|---|---|---|---|
| Types of probiotics | Inhibitory effect | suppression methane amount | Inhibition machanism |
| Propionic acid bacillus | * (Most propionic bacteria) | 100 μL of the propionic acid | Unknown |
| | *** (P. jensenii LMGT282 and | bacteria culture (2 × 10⁸ to 4 × 10⁸ | |
| | P. thoenii LMGT2827 or T159) | colony forming units), | |
| | | Propionibacterium thoenii T159; 20% | |
| Lactic acid bacteria | *** | 5.3 lg cfu/g fresh weight, | Hydrogen consumption |
| | | Lactobacillus plantarum, 8.8 ml/g(72 h) | |
| Acetic acid bacteria | *** | 1% Proteiniphilum acetatigenes | Reduced the number of |
| | | GA03; — | methanogens |
| Enterococcus faecium | *** | 0.19%; 2.08 mM/mL | Alters microbial flora |
| SROD | | | |
| Probiotic products | *** | 2 g probiotic products in | Reduce the number of |
| of Ruminococcus | | powder; 1.2 ml/g of dry matter | rumen protozoa |
| flavefaciens | | 10 ml probiotic products in liquid; | |
| | | 1.2 ml/g of dry matter | |
| Bacillus licheniformis | *** | 2.5 × 10⁹; 2.7 L/d | Unknown |
| Saccharomyces | ** (Pedraza-Hernandez et al., 2019) | — | Affects rumen microbes |
| cerevisiae | * (Darobigone et al., 2019) | | |

Prebiotics that Reduce Methane Production in Ruminants

Prebiotics are substances that are not easily digested or absorbed by the host. They selectively stimulate the growth and activity of one or several ruminal microorganisms with a positive effect on ruminal fermentation. Prebiotics suppress ruminal CH4 production in ruminants. Prebiotics mainly reduce rumen CH4 production by altering the bacterial community structure, influencing the permeability of the cell walls of methanogenic archaea, and stimulating other bacteria to compete with methanogens for H2 (Table 9).

The prebiotic chitosan can influence bacterial community structures by altering microbial population compositions, for example, by replacing fibrinolytic enzyme-producing microbes (Firmicutes and Fibrobacteres) with amylolytic enzyme-producing microbes (Bacteroides and Proteus); in turn, reducing CH4 production. Chitosan could influence the ruminal fennentation process by altering VFA distributions and increasing propionic acid concentrations, which reduces CH4 production in turn. However, the reduction in CH4 is associated with the degree of chitosan deacetylation, which could alter the penneability of the methanogen cell wall. In addition, various yeast products could reduce CH4 emissions by stimulating acetic acid-producing bacteria to compete with methanogens or metabolize hydrogen.

TABLE 9
Exemplary prebiotic bacteria that reduce methane production in ruminants

| | | Addition amount; maximum | |
|---|---|---|---|
| Types of prebiotics | Inhibitory effects | methane suppression amount | Inhibition machanism |
| Chitosan | *** | 3000 (molecular weights) | (1) Alters microbial |
| | | dry matter; 22.9% | community structure |
| | | ml/day (Tong et al., 2020) | (Tong et al., 2020) |
| | | 2% Chitosan + 21% of crude | (2) Alters fermentation |
| | | glycerin; 53.67% (Seankamsorn, | pathway (Seankamsorn, |
| | | Cherdthong & Wanapat, 2020) | Cherdthong & |
| | | | Wanapat, 2020) |
| | | | (3) Influences |
| | | | methanogenic bacteria |
| | | | cell wall permeability |
| | | | (Zanferari et al., 2018) |
| Yeast products | *** | 4 mg/l g dry matter; — | Indirect consumption of |
| | | | hydrogen |

Other Agents that Reduce Methane in Ruminants

Among the CH4 mitigation options, inhibiting the growth or the metabolic activity of methanogens is the most effective approach. Another strategy is to modulate rumen microbiome so that fermentation is shifted toward decreased H₂ production and increased production of reduced VFA (e.g., propionate). Provided herein are exemplary and non-exhaustive descriptions of anti-methanogenic compounds evaluated with a focus on their impact rumen methanogens.

### Coenzyme M Analogs

Methyl-CoM reductase (Mcr) mediates the final step of all the methanogenesis pathways and CoM (2-mercaptoethanesulfonic acid) is an essential cofactor serving as the methyl group carrier. Mcr reduces methyl-CoM to CH₄. CoM is found in all known methanogens but not in other archaea or bacteria. Several halogenated sulfonated compounds, including 2-bromoethanesulfonate (BES), 2-chloroethanesulfonate (CES), and 3-bromopropanesulfonate (BPS), are structural analogs of CoM, and they can competitively and specifically inhibit Mcr activity, lowering CH₄ production at relatively low concentrations. Different species of methanogens vary in sensitivity to these inhibitors. Of three species tested on BES, Mbb. Ruminantium was the most sensitive, while Methanosarcina mazei was the least sensitive, with Methanomicrobium mobile being intermediate. All three species appeared to be resistant to BPS up to 250 μmol/L in pure cultures. The different sensitivity to these CoM analogs has been attributed to varying ability to uptake these inhibitors into the cells. Methanogens able to synthesize their own CoM are less dependent on external CoM and are thus less sensitive. Mbb. Ruminantium is the only ruminal methanogen that requires CoM synthesized by other methanogens.

Halogenated Aliphatic C₁-C₂ Hydrocarbon

Halogenated aliphatic compounds with 1 or 2 carbons, such as chloroform, bromochloromethane (BCM), bromoform, bromodichloromethane, dibromochloromethane, carbon tetrachloride, trichloroacetamide, and trichloroethyladipate, can lower ruminal CH₄ production. These halogenated compounds block the function of corrinoid enzymes and inhibit cobamide-dependent methyl group transfer in methanogenesis. These halogenated compounds also competitively inhibit CH₄ production by serving as terminal electron (e) acceptors. Drenching chloroform to cattle inhibited methanogenesis substantially for up to 32 days without affecting feed digestion or basic rumen function. The addition of BCM depressed CH₄ production both in vitro and in vivo. In steers fed grain-based diets, BCM decreased CH₄ production by 50 to 60% with no signs of toxicity or residues in meat. It was also reported that the abundance of total bacteria and protozoa was not changed, but methanogenesis and growth of methanogens were drastically inhibited by BCM in both batch cultures and continuous fermenters. While the commercial use of chloroform, a recognized carcinogen, is not practical, it provides validation for the class of BCM compounds in reducing methane production.

Some marine plants such as red seaweed, and algae, lichen, and fungi can contain high concentrations of organobromine compounds such as bromomethane and bromoform. A recent in vitro study showed that red seaweed Asparagopsis taxiformis lowered CH₄ production by 99% at a dose of 2% of organic matter substrate. No adverse effect on feed digestion or fermentation was noted at <5% (of dry matter) inclusion. Thus, red seaweed, and probably other organobromine-rich plants, may offer a potentially practical natural approach to mitigate CH₄ emission.

### Nitrooxy Compounds

3-Nitrooxypropanol (3NOP) and ethyl-3NOP, two new synthetic compounds, have been shown to have specific anti-methanogenic properties. 3NOP appears to inactive Mcr by competitively binding to the Mcr active site and then oxidizing the Ni¹⁺ that is required for Mcr activity. Feeding of 3NOP at a dose rate of 2.5 g/day/cow mixed in diets decreased CH₄ emission by 60% per kg of DM intake. In a study using beef cattle, 3NOP fed at 2.0 g/day/cow decreased CH₄ yield by 59%, and the inhibition persisted for up to 112 days without much effect on feed intake, nutrient digestibility or total VFA concentrations. In one recent study, 3NOP fed at 40-80 mg/kg feed DM in dairy cows decreased CH₄ production by about 30% persistently for up to 84 days. Similarly, 3NOP fed at 2.5 g/day/cow decreased CH₄ yield by 37% in dairy cows. In sheep, 3NOP at 0.5 g/day also decreased CH₄ production by 29% without adverse effect on digestion or rumen fermentation. However, when 3NOP was directly added to the rumen through rumen cannula at a daily dose of 0.50 or 2.5 g per cow (equivalent to 25 to 125 mg/kg feed dry matter), the degree of CH₄ suppression declined to 7-10%. The later study suggests that 3NOP needs to be fed together with the diet to achieve higher efficacy. Thus, 3NOP could be used to lower CH₄ emission from cows and sheep without adverse effects on nutrient utilization or animal performance. It has been demonstrated that 3NOP indeed decreased methanogen abundance while increasing the abundance of protozoa.

TABLE 10
Exemplary nitrogen-containing compounds that reduce methane production in ruminants

| Types of | Inhibitory | Addition amount; maximum | |
|---|---|---|---|
| nitrogenous compounds | effect | methane suppression amount | Inhibition mechanism |
| Nitrate | *** | 20 mg/g dry matter; 21% | (1) Hydrogen consumption; |
| | | (Alvarez-Hess et al., 2019) | (2) Inhibits the proliferation |
| | | 5 mmol/L; 32.92% | of mathanogens; reduces their |
| | | (Wu et al., 2019) | activity and abundance |
| | | 5 mM; 43.26% Liu et al., 2017) | |
| Encapsulated nitrate (EN) | ** | 70 g/100 kg of body | Reduces methane reducing |
| | | weight; 18.5% CH₄/kg of | bacteria |
| | | forage dry matter intake | |
| | | (Granja-Salcedo et al., 2019) | |
| | | 2.5%; 9.37 mM/d | |
| | | (Capelari et al., 2018) | |
| | | 2.5%; 2.8 g/kg Dry matter intake | |
| | | (Alemu et al., 2019) | |
| Urea and nitrate mixture | ** | 34 g/kg straw dry matter + 6 g/kg | Indirect consumption of hydrogen |
| | | dry matter of ammonium | |
| | | nitrate; 10.2% (Zhang et al., 2019) | |
| | | Urea + ammonium nitrate (34 + | |
| | | 6 g/kg of dry matter, respectively); | |
| | | 3.1 mL/g dry matter (Zhang et al., | |
| | | 2018) | |
| Nitrocthane (NE), 2-Nitroethanol | *** | 10 mmol/L; 96.7% (NE), 96.7% | (1) Inhibits the activity |
| (NEOH), 2-Nitro-1-Propanol | | (NEOH), 41.7% (NPOH) | of methanogens; |
| (NPOH) | | | (2) Inhibits methyl- |
| | | | coenzyme M gene expression; |
| | | | (3) Reduces the content of |
| | | | coenzyme F420 and F430 |
| 3-Nitrooxypropanol (NOP) | *** | 0.08 mg/g dry matter; 44% | Inhibits methyl-Coenzyme M |
| | | (Alvarez-Hess et al., 2019) | activity |
| | | 2.5 g/animal/day; 38%/kg | |
| | | dry matter intake (Martinez- | |
| | | Fernandez et al., 2018) | |
| | | 60 mg/kg of feed dry matter; | |
| | | 26%/day (Melgar et al., 2020) | |
| | | 1.6 g; 28%(roughage), 23% | |
| | | (concentrate pellet) (Van | |
| | | Wesemael et al., 2019) | |

### Pterin Compounds

Pterin is a group of structural analogs of deazaflavin (F₄₂₀), which is a coenzyme involved in two steps of the hydrogenotrophic methanogenesis pathway. Therefore, pterin compounds can competitively inhibit CH₄ production. In one study, CH₄ production by Mbb. Ruminantium, Ms. Mazei, and Mm. mobile was significantly decreased by lumazin (2,4-pteridinedione) in a dose-dependent manner from 0.06 to 0.24 mmol/L. As expected, pterin is much less efficacious in mixed rumen cultures than in pure methanogen cultures. It was suggested that lumazine could be degraded or transformed by some microbes in mixed cultures or adsorbed to solid particles where it becomes unavailable to methanogens. Some N-substituted derivatives of p-aminobenzoic acid, which are inhibitors of methanopterin synthesis in methanogens, decreased methanogenesis in ruminal cultures without inhibiting VFA production.

### Hydroxymethylglutaryl-CoA (HMG-S-CoA) Reductase Inhibitors

All archaea contain long-chain isoprenoid alcohols as the major component of their cell membrane. Isoprenoid alcohols are unique to archaea. They are synthesized from mevalonate that is formed by reduction of 3-hydroxy-3-methylglutaryl coenzyme A (HMG-S-CoA) catalyzed by HMG-S-CoA reductase. This enzyme is also used for the synthesis of the same precursor ultimately used in cholesterol synthesis in humans. As an inhibitor of HMG-S-CoA reductase, statins (e.g., lovastatin and mevastatin) can inhibit the growth of methanogens by inhibiting the synthesis of mevalonate. Lovastatin and mevastatin may also act as a potential inhibitor of F₄₂₀-dependent NADP oxidoreductase as shown in the model structure of that enzyme. In the earliest reported study, mevastatin at 5.6 μmol/L inhibited the growth of all three strains of rumen Methanobrevibacter, but not rumen bacteria in vitro. In studies using a rumen simulation technique (Rusitec), lovastatin (150 mg/L) reduced CH₄ production by approximately 42% without altering bacterial counts or nutrient fermentation.

The high cost of pure statins promoted a search for natural sources of statins as agents to mitigate CH₄ production. Lovastatin is a secondary metabolite of idiophase of several fungal species (e.g., Penicillium spp., Aspergillus terreus, Monascus purpureus, and Pleurotus ostreatus), and it can reach a concentration up to 2.8% of the dry weight of P. ostreatus (oyster mushrooms) and 261 mg/kg fermented rice straw. Lovastatin extracted from fermented rice straw significantly reduced total CH₄ production by rumen methanogens by nearly 28% after 48 h in vitro incubation. Extract from A. terreus-fermented rice straw containing lovastatin (97 mg/g dry mass) also significantly reduced CH₄ production and abundance of methanogens, especially Methanobacteriales, and aerobic fungi, but increased several fiber-degrading bacteria. Lovastatin also altered the morphology of M. smithii significantly, resulting in abnormal membrane formation and asymmetric cell divisions and increased HMG-S-CoA reductase gene expression. Fermented rice straw extract also modulated expression of several genes associated with methanogenesis, increasing expression of mtr, mta, and mcr while decreasing expression of hmd and fno. Supernatant fractions containing statins produced by Mortierella wolfii also appeared promising to inhibit methanogenesis without reducing overall fermentation. In another study using sheep, fermented rice straw containing metabolites (possibly pravastatin and mevastatin) produced by Monascus spp. Decreased CH₄ emission (by 30%), the abundance of methanogens, and ruminal acetate: propionate ratio compared with the unfermented rice straw.

Diallyl disulfide, the main ingredient of garlic oil, is known to inhibit HMG-S-CoA reductase. Garlic oil (300 mg/L) was more effective than lovastatin as an inhibitor of CH₄ production (by up to 91% reduction); however, garlic oil also inhibited bacterial growth, which likely reduces the availability of methanogenesis substrates. Garlic oil lowered CH₄ production in vitro and growth of methanogens, altered community structure of methanogens after 24 h incubation. Moreover, interestingly, the anti-methanogenic efficacy increased over time up to 18 days of incubation.

### Plant Secondary Metabolites

Plants secondary metabolites (PSM), such as saponins, tannins, flavonoids, organosulphur compounds, and essential oils, have anti-microbial activities against several types of microorganisms. Numerous PSM extracts have been recognized as potential inhibitors of rumen methanogens and CH₄ production. Some forage plants rich in tannins and saponins have also shown promise at mitigating CH₄ emission from ruminants.

TABLE 11
Exemplary plant extracts that reduce methane production in ruminants

| | | Inhibitory | Addition amount; maximum | |
|---|---|---|---|---|
| Types of probiotics | | effects | suppression methane amount | Inhibition mechanisms |
| Plant extracts | Corymbia citriodora leaf | *** | 10 ml/calf/day; — | (1) Protozoa number |
| | extract | | | reduced (1.84 × 10⁵/ml) |
| | | | | (2) Ratios of volatile fatty |
| | | | | acids altered |
| | Aloe vera, Carica | *** | 25 mg/L and 50 mg/L Azadirachta | Unknown |
| | papaya, Azadirachta | | indica, Carica papaya, Tithonia | |
| | indica, Moringa oleifera, | | diversifolia; 15%, Jatropha | |
| | Tithonia diversifolia, | | curcas and Moringa oleifera pods; | |
| | Jatropha curcas, and | | 30% (Akanema & Hassen, 2018) | |
| | Moringa oleifera pod | | Eragrostis substrate, 4 ml plant | |
| | extracts | | extracts; 50% reduction in methane/ | |
| | | | Total gas production (Akanema, | |
| | | | Hassen & Adejoro, 2020) | |
| | Pomegranate peel | *** | 2% of dry matter intake, | Unknown |
| | extract and Desert teak | | Punica granatum; 46% | |
| | extract | | Tecomella undulata; 42% | |
| | Rhus succedanea extract | *** | 50 mg/L; lowest | Unknown |
| | Areca catechu and Acacia | ** | 2% dry matter basis, Areca | Unknown |
| | nilotica extract | | Catechu; 21%; Acacia nilotica; | |
| | | | 23% | |
| | Asparagopsis armata | *** | 1.0%; 67.2% (Roque et al., 2019) | Unknown |
| | Garlic extract | *** | 0.5%; —(Kim et al., 2018b) | Decreased abundance of |
| | | | 1 g the experimental mixture; 6.9 ± | methanogenic archaea |
| | | | 10.7 ml/d (Eger et al., 2018) | |
| | Plant extracts reservatrol | *** | 25 mg, high-forage diets; 60%; | Decreased abundance of |
| | | | high-concentrate diets; 41% | Methanobacter |
| | Plant extracts caffeic | *** | 12 mM, Caffeic acid; 37.58%; | Unknown |
| | acid and p-coumaric | | p-coumaric acid; 28.33% | |
| | acid | | | |

### 1. Tannins

Tannins decrease CH₄ production by directly inhibiting methanogens and indirectly decreasing H₂ production as a result of decreased fiber digestion and protozoal population in the rumen. The inhibitory activity of tannins extracted from Lotus pedunculatus was demonstrated on pure cultures of methanogens. The inhibition of methanogen populations was also shown by tannins in the rumen of goats fed diets containing tannins. Studies on structure-activity relationships have shown that types and molecular weights of tannins are important in determining their potency in lowering CH₄ production and abundance and diversity of rumen methanogens, with high molecular weight condensed tannins (CT) being more potent. Such structure-activity relationships have been demonstrated using members of Methanobacteriales including Methanobrevibacter.

### 2. Flavonoids

Flavonoids have not been extensively evaluated with respect to rumen methanogenesis. It was reported that inclusion of flavone, myricetin, naringin, rutin, quercetin, or kaempferol decreased in-vitro CH₄ production by 5 to 9 mL/g DM. Their potency ranked as follows: myricetin≥kaempferol≥flavone≥quercetin≥naringin≥rutin≥catechin. Catechin decreased CH₄ production both in vitro and in vivo. All the flavonoids, when fed at 0.2 g/kg DM, noticeably decreased relative abundances of hydrogenotrophic methanogens, and citrus (Citrus aurantium) extract rich in mixed flavonoids and its pure flavonoid components, neohesperidin and naringin, appeared to result in the greatest inhibition. Methanosarcina spp. Were also inhibited by poncirin, neohesperidin, naringin and their mixture. Flavonoids directly inhibit methanogens and also likely acts as H₂ sinks via cleavage of ring structures (e.g., catechin) and reductive dihydroxylation.

### 3. Saponins

The effects of saponins on rumen fermentation, rumen microbial populations, and ruminant productivity have been examined extensively. Quillaja saponin at 1.2 g/L, but not at 0.6 g/L, lowered CH₄ production in vitro and the abundance of methanogens (by 0.2-0.3 log) and altered their composition. Ivy fruit saponin decreased CH₄ production by 40%, modified the structure of the methanogen community, and decreased its diversity. Saponins from Saponaria officinalis decreased CH₄ and abundance of both methanogens and protozoa in vitro. It is hypothesized that saponins lower H2 production, thereby reduce CH4 production.

### 4. Essential Oils

The effects, mostly beneficial, of essential oils (EO) on rumen fermentation, microbial populations, and ruminant productivity have frequently been reviewed. Several EO compounds, either in pure form or in mixtures, are anti-methanogenic. The effects of EO on CH₄ production and methanogens are variable depending on dose, types, and diet. Five EO (clove, eucalyptus, peppermint, origanum, and garlic oil) that have different chemical structures in vitro at three different doses (0.25, 0.50 and 1.0 g/L) were tested for their effect on CH₄ production and archaeal abundance and diversity. Overall, all these EO suppressed CH₄ production and abundance of archaea and protozoa in a dose-dependent manner, but they differed in potency. Thyme oil or cinnamon oil fed to Holstein steers at 0.5 g/day decreased the relative abundance of total protozoa and methanogens. However, feeding beef cattle a blend of EO (CRINA®) did not affect CH₄ production, methanogen abundance or its diversity. Overall, methanogens may be directly inhibited or indirectly inhibited by Eos via inhibition of protozoa and H₂-producing bacteria in the rumen.

### Alternative Hydrogen Sinks

Compounds with a redox potential higher than CO₂ can thermodynamically outcompete CO₂ for reducing equivalents produced during rumen fermentation. These compounds, thus, can be used as alternative e acceptors to redirect e flux away from methanogenesis. The commonly evaluated alternative e acceptors are discussed below.

### 1. Nitrate and Sulfate

Nitrate (NO₃¹⁻) decreased CH₄ production both in vitro and in vivo. Mechanistically, nitrate decreases CH₄ production by outcompeting CO₂ as an e⁻ acceptor, and its reduction intermediates, nitrite (NO₂¹⁻) and nitrous oxide (N₂O), also directly inhibit methanogens as well as some H₂ producers. Sulfate also lowers CH₄ production, but much less effectively than nitrate. Archaeal abundance declined in goats receiving nitrate. While nitrate is not toxic to methanogens, it is toxic to protozoa, fungi and to a lesser extent to select bacterial species, suggesting a more general toxicity of nitrate. Nitrate can replace a portion of the dietary nitrogen as it is reduced to ammonia.

### 2. Nitrocompounds

A few organic nitrocompounds have been evaluated for their efficacy to decrease methanogens and CH₄ production. These compounds can serve as e acceptors by some bacteria competing with methanogens for reducing equivalents. This is demonstrated by nitroethane that can be used as a terminal e acceptor by Dentitrobacterium detoxificans, thereby indirectly decreasing CH₄ production. Nitrocompounds may also inhibit methanogenesis by directly inhibiting the activity of formate dehydrogenase/formate hydrogen lyase and hydrogenase, all of which are involved in the early step(s) of the hydrogenotrophic methanogenesis pathway, or inhibiting e transfer between ferredoxin and hydrogenase.

Nitrocompounds generally are quite effective in lowering CH₄ production, with 3-nitro-propionate, 2-nitropropanol, 2-nitroethanol and nitroethane being able to decrease CH₄ production by 57 to 98% in vitro. Using sheep, it was shown that nitroethane decreased CH₄ production by up to 45% and 69%, respectively, when orally administrated at 24 and 72 mg/kg body weight daily for 5 days. Although less effective than nitroethane, 2-nitropropanol also significantly lowered CH₄ production (by 37%) in steers.

### 3. Propionate and Butyrate Enhancers

Malate, acrylate, oxaloacetate, and fumarate are intermediates of carbohydrate fermentation. They can be converted to propionate or used in anabolism for the synthesis of amino acids or other molecules. They can accept reducing equivalents and thus stoichiometrically lower H₂ available for CH₄ production. When added at a concentration of 3.5 g/L, fumarate decreased CH₄ production by 38% in continuous fermenters with forages as a substrate. Types of forages and their combinations appeared to affect the anti-methanogenic efficacy of fumarate, ranging from 6 to 27% inhibition at 10 mmol/L. Acrylate also depresses CH₄ production in the rumen, but to a lesser extent than an equimolar level of fumarate. Malate was found to decrease CH₄ production by beef cattle in a dose-dependent manner, with a 16% decrease being noted when fed at 7.5% of DM intake, which corresponds to a 9% reduction per unit of DM intake. Different studies reported different anti-methanogenic potencies of this type of e-acceptors. Fumarate fed to goats at 10 g/day/goat was found to decrease the abundance of methanogens and CH₄ production only by 11.9% while increasing concentrations of total VFA, acetate and propionate. Some of the intermediates of pyruvate conversion to butyrate can act as e acceptors, which could also decrease CH₄ production.

### 4. Unsaturated Organic Acids

Unsaturated fatty acids can act as hydrogen sinks during their biohydrogenation and thereby lower CH₄ production. Propynoic acid (an unsaturated analog of propionic acid), 3-butenoic acid and 2-butynoic acid (both unsaturated analogs of butyric acid), and ethyl 2-butynoate each at 6 to 18 mmol/L have been evaluated as alternative e sinks to lower methanogenesis in vitro. Only propynoic acid and ethyl 2-butynoate markedly lowered CH₄ production, by 65 to 76% and 24 to 79%, respectively. In another study, propynoic acid lowered CH₄ production by 67% and 78% at 6 and 12 mmol/L, respectively and decreased methanogen abundance. Propynoic acid and ethyl 2-butynoate are directly toxic to methanogens, and species of methanogens vary in their sensitivity to these two inhibitors, with Mbb. Ruminantium being most sensitive, Ms. Mazei least sensitive, and Mm. mobile intermediate.

### Inhibitors to Hydrogen-Producing Bacteria

### 1. Ionophores

Ionophores, such as monensin and lasalocid, are commonly used to improve rumen microbial metabolism. Being highly lipophilic ion carriers, they pass through the cell wall of Gram-positive bacteria and penetrate into the cell membrane. Therein, they serve as H⁺/Na⁺ and H⁺/K⁺ antiporters, dissipating ion gradients that are needed for ATP synthesis, nutrient transport, and other essential cellular activities and ultimately resulting in delayed cell division and even cell death. Ionophores preferentially inhibit Gram-positive bacteria, including members of class Clostridia, including Ruminococcus species that produce acetate and H₂. Ionophores can also inhibit some Gram-negative rumen bacteria, including bacteria that produce formate and H₂. Therefore, ionophores may lower CH₄ emission by decreasing H₂ production. For examples, monensin fed at 24-35 mg/kg diet lowered CH₄ production by up to 10% (g/kg DM intake), though no CH₄ suppression was observed at 10-15 ppm. In a recent in vivo study, however, monensin at 60 mg/day/cow did not lower CH₄ production by tropical cattle, though it decreased CH₄ production by about 30% when fed at 250 mg/day/cow. As repeatedly noted, at such high supplementation level, DM intake was lowered, which explains most of the observed decrease in CH₄ emission. Ionophores are not known to directly inhibit methanogens, but they can change the population dynamics of methanogen species. For example, monensin decreased the population of Methanomicrobium spp. While increasing that of Methanobrevibacter spp. Total methanogens were also decreased in cattle fed monensin. These can be explained by reduced availability of H₂ and differences in affinity for H₂ and growth kinetics among methanogen species.

### 2. Bacteriocins

Bacteriocins are proteins or peptides produced by bacteria and inhibit select microbial species in the rumen and other habitats. There are only a few studies investigating the effect of bacteriocins on CH₄ emission. Bovicin HC5, a bacteriocin produced by Streptococcus spp. From the rumen, was reported to suppress CH₄ by 50% in vitro. Nisin, a bacteriocin produced by Lactobacillus lactis subsp. Lactis, has also been shown to decrease CH₄ production in vitro by up to 40% depending upon its concentration. Similar to monensin, bacteriocins probably modulate rumen fermentation leading towards increased propionate, thereby decreasing CH₄ production.

Additional Agents that Reduce Methane in Ruminants

### Introduction

Climate change is a global problem requiring innovative solutions to reduce production of and/or increase the sequestration of deleterious atmospheric gases and/or precursors thereof. The present invention relates to compositions, methods, and/or kits that reduce global deleterious atmospheric gases and/or precursors thereof. In preferred embodiments, provided herein are compositions, methods, and/or kits that reduce the production of deleterious atmospheric gases and/or precursors thereof. The deleterious atmospheric gases and/or precursors thereof to be reduced can be generated from any suitable production source, such as flooded ecosystems and/or agriculturally relevant ecosystems, for example, farming sites. In certain embodiments, provided herein are compositions, methods, and/or kits comprising one or more small molecules that affect the production of one or more deleterious atmospheric gases and/or precursors thereof. The one or more small molecules can be formulated in any suitable manner, for example, as a solid or liquid. In certain embodiments, the solid and/or liquid formulations are delivered to the site of use by human and/or by machine. In preferred embodiments, the one or more small molecules that affect the production of one or more deleterious atmospheric gases and/or precursors thereof target microorganisms.

### Ecosystems

A. Flooded Ecosystems

In certain embodiments, provided herein are compositions, methods, and/or kits for use in flooded ecosystems. As used herein, the term “flooded ecosystem” includes an area where water is present at or near the surface of the soil for varying periods of time during the year. In certain cases, a flooded ecosystem comprises water at or near the surface of the soil for at least a portion of the growing season. The flooded ecosystem can be any suitable flood ecosystem, such as a wetland, for example a lagoon, wet meadow, marsh, swamp, peatland, mire, bog, fen, mangrove forest, carr, pocosin, floodplain, vernal pool, paddy field, an agricultural field, or a combination thereof. In certain embodiments, the wetland is tidal or non-tidal. In certain embodiments, the wetland comprises freshwater, brackish water, or salt water. In a preferred embodiment, the flooded ecosystem comprises a paddy field, more preferably a rice paddy.

Typically, the flooded ecosystem comprises hydric soil, i.e., soil formed under conditions of saturation, flooding or ponding for a period of time during the growing season. One of many characteristics of hydric soil includes the presence of hypoxic and/or anaerobic conditions in the soil, wherein an environment with little to no oxygen provides favorable conditions for anaerobic organisms. For example, the anaerobic environment generated from rice farming, e.g., a rice paddy, promotes the growth of methanogenic microorganisms whose activity results in the generation of one or more deleterious atmospheric gases and/or precursors thereof, including methane. It is estimated that methane from rice production contributes to 1.5% of the total global deleterious atmospheric gas emissions. Provided herein, are compositions, methods, and/or kits for reducing the production of one or more deleterious atmospheric gases and/or precursors thereof from flooded ecosystems.

B. Agriculturally Relevant Ecosystems

In certain embodiments, provided herein are compositions, methods, and/or kits for use in suitable agriculturally relevant ecosystems. In certain embodiments, the agriculturally relevant ecosystem comprises arable land and/or pastureland, such crop land, meadows, pastures, and/or forests. Provided herein, are compositions, methods, and/or kits for reducing the production of one or more deleterious atmospheric gases and/or precursors thereof from an agriculturally relevant ecosystem.

Microbial Sources of Deleterious Atmospheric Gases and/or Precursors Thereof

A. Methanogens

In certain embodiments, wherein the one or more deleterious atmospheric gases and/or precursors thereof are microbially derived, the microorganism can be any suitable microorganism, such as a methanogen, for example a Methanopyrales, Methanococcales, Methanobacteriales, Methanosarcinales, Methanomicrobiales, Methanocellales, Methanomassiliicoccales, Halobacteriales, Thermoplasmataltes, or a combination thereof. In certain embodiments, the methanogen comprises an archaea. In preferred embodiments, the microorganism comprises a methanogen.

B. Biochemical Pathways

In certain embodiments, the one or more deleterious atmospheric gases and/or precursors thereof are microbially derived through one or more biosynthetic pathway. The deleterious atmospheric gas can be any suitable deleterious atmosphere gas, such as carbon dioxide, methane, nitrous oxide, or a combination thereof. The deleterious atmospheric gas precursor can be any suitable precursor, such as acetate, hydrogen, carbon, methanol, monomethylamine, dimethylamine, trimethylamine, nitric oxide, or a combination thereof. In preferred embodiments, the deleterious atmosphere gas comprises carbon dioxide, hydrogen, or methane more preferably methane. In certain embodiments, wherein the resultant deleterious atmospheric gas comprises methane, the one or more biosynthetic pathways include the acetoclastic, hydrogenotrophic, and methylotrophic pathways, which differ based on the starting substrates, i.e., precursor, (FIG. 1), more preferably the acetoclastic or hydrogenotrophic pathways, even more preferably the acetoclastic pathway.

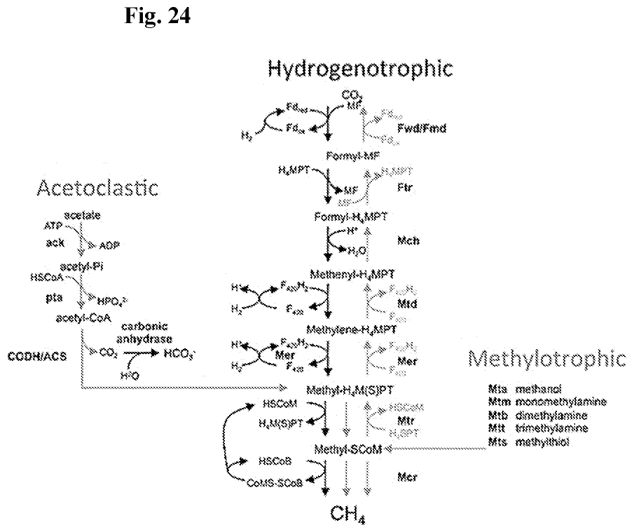

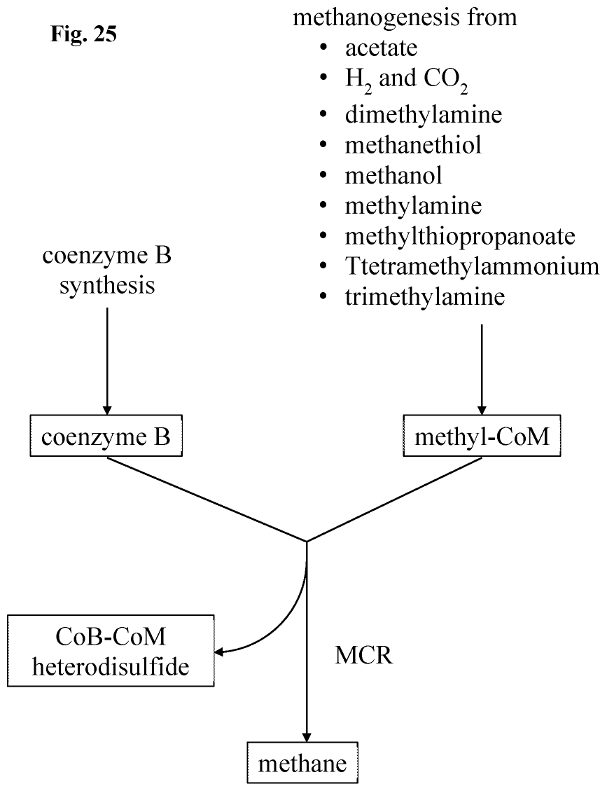

The acetoclastic pathway comprises a series of enzymes that convert the precursor acetate through a series of enzymatic conversions to methane. Starting from acetate, (1) acetate is converted to acetyl phosphate by acetate kinase (ack); (2) acetyl phosphate is converted to acetyl-CoA by phosphotransacetylase (pta); (3) the acetyl group from acetyla-CoA is transferred to a protein intermediate by acetyl-CoA decarbonylase; (4) the acetyl group is then transferred to tetrhydrosarcinapterin to form 5-methyl-tetrahydrosarcinapterin by methyltetrahydrosarcinapterin methyltransferase; (5) 5-methyl-tetrahydrosarcinapterin is converted to methyl-CoM by methyl-H₄SPT:CoM methyltransferase (Mtr); and (6) methyl-CoM is reduced to methane by methyl-CoM reductase (Mcr) (FIG. 24 and FIG. 25).

The hydrogenotrophic pathway comprises a series of enzymes that convert the precursors hydrogen and carbon dioxide to methane. Starting from carbon dioxide and hydrogen, (1) a formylmethanofuran dehydrogenase (Fwd/Fmd) produces a formylmethanofuran, (2) which is further converted into 5-formyl-tetrahydromethanopterin by a formylmethanofuran:H₄MPT formylatransfer (Ftr); (3) 5-formyl-tetrahydromethanopterin is further converted into 5,10-methenyltetrahydromethanopterin by methyl-H₄MPT cyclohydrolase (Mch); (4) 5,10-methenyltetrahydromethanopterin is converted to N6-methyltetrahydromethanopterin by F₄₂₀-dependent methylene-H₄MPT reductase (Mer); (5) N6-methyltetrahydromethanopterin is converted to methyl-CoM by methyl-H₄MPT:coenzyme M methyltransferase (Mtr); and (6) methyl-CoM is reduced to methane by methyl-CoM reductase (Mcr) (FIG. 24 and FIG. 25).

The methylotrophic pathway comprises a series of enzymes that convert one or more of dimethylamine, methanethiol, methanol, methylamine, methylthiopropanoate, tetramethylammonium, and/or trimethylamine into methyl-CoM, wherein methyl-CoM is reduced to methane by methyl-CoM reductase (Mcr) (FIG. 24 and FIG. 25).

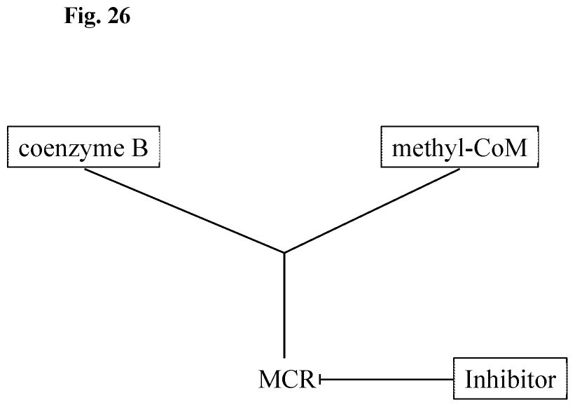

In certain embodiments, provided herein are compositions, methods, and/or kits comprising one or more small molecules that reduce the activity of one or more enzymes in one or more methane biosynthetic pathways. The enzyme can be any suitable enzyme, such as 3-(methylthio)propanoate:coenzyme M methyltransferase, acetate kinase, acetyl-CoA decarbonylase, acetyl-CoA decarbonylase/synthase complex α₂ε₂, acetyl-CoA decarbonylase/synthase complex J, acetyl-CoA decarbonylase/synthase complex γδ, acetyl-CoA synthase, carbon monoxide dehydrogenase, carbonic anhydrase, Co-methyltransferase, coenzyme M reductase, cyclohydrolase, dehydrogenase, dimethylamine-[corrinoid protein] Co-methyltransferase, F₄₂₀-dependent methylene-H₄MPT reductase, F₄₂₀-dependent methylene-H₄SPT dehydrogenase, formylmethanofuran dehydrogenase, formylmethanofuran:H₄MPT formyltransferase, formylmethanofuran:H₄SPT formyltransferase, formyltransferase, H₂-forming methylene-H₄MPT dehydrogenase, methanol-5-hydroxybenzimidazolylcobamide Co-methyltransferase, methenyl-H₄MPT cyclohydrolase, methyl-coenzyme M reductase, methyl-H₄SPT:CoM methyltransferase, methylated [methylamine-specific corrinoid protein]:coenzyme M methyltransferase, methylcobamide:CoM methyltransferase, methylthiol:coenzyme M methyltransferase, methyltransferase, MtaC protein:coenzyme M methyltransferase, phosphotransacetylase, tetrahydromethanopterin S-methyltransferase, tetramethylammonium methyltransferase, trimethylamine-corrinoid protein Co-methyltransferase, or a combination thereof. In a preferred embodiment, the enzyme comprises methyl-CoM reductase (Mcr) (FIG. 26).

Compositions for Reducing Production of Deleterious Atmospheric Gases and/or Precursors Thereof

In certain embodiments provided herein are compositions. In certain embodiments, provided herein are compositions comprising one or more small molecules. In preferred embodiments, provided herein are compositions comprising one or more small molecules that reduce the production of one or more deleterious atmospheric gases and/or precursors thereof. The small molecule can be any suitable small molecule for reducing the production of one or more greenhouse gases and/or precursors thereof, for example a small molecule that interferes with the uptake and/or conversion of acetate, hydrogen, carbon dioxide, methanol, monomethylamine, dimethylamine, trimethylamine, nitric oxide, or a combination thereof, and/or a small molecule that interfere with the production of carbon dioxide, hydrogen nitrous oxide, or a combination thereof. In preferred embodiments, the small molecule interferes with the uptake and/or conversion of acetate, hydrogen and/or carbon dioxide and/or the production of carbon dioxide or methane, more preferably with the production of methane.

A. Small Molecules that Affect Production of Deleterious Atmospheric Gases and/or Precursors Thereof

In certain embodiments, provided herein is a composition for reducing emissions of deleterious atmospheric gasses and/or precursors thereof from a flooded ecosystem comprising: one or more small molecules that reduce the production of one or more deleterious atmospheric gasses and/or precursors thereof. The one or more small molecules that reduce the production of one or more deleterious atmospheric gasses and/or precursors can be any suitable molecule.

In certain embodiments, the one or more small molecules that reduce the production of one or more deleterious atmospheric gasses and/or precursors comprises a compound with the formula the formula

R¹—[CH₂]n-ONO₂
wherein
n is an integer from 1 to 15;
R¹ is selected from the group consisting of H, C₁-C₆alkyl, phenyl, —OH, —NH₂, —CN, —COOH, —O(C═O)R³, —NHC(═O)R³, SO₂NHR³, or —ONO₂, —SH and R³ is C₁-C₆alkyl, phenyl, pyridyl;
with the proviso that when n is >3 the hydrocarbon chain may be interrupted by —O— or —NH—.

In some embodiments, the one or more small molecules that reduce the production of one or more deleterious atmospheric gases and/or precursors comprises 3-nitrooxypropanol, 9-nitrooxynonanol, 5-nitrooxy pentanoic acid, 6-nitrooxy hexanoic acid, bis(2-hydroxyethyl)amine dinitrate, 1,4-bis-nitrooxybutane, 1,5-bis-nitrooxypentane, or any combination thereof. Preferably, the one or more small molecules is 3-nitrooxypropanol (3-NOP).

In some embodiments, the composition comprises about 1 to about 25% by weight of the small molecule, about 5 to about 20% by weight of the small molecule, or about 5 to about 15% by weight of the small molecule.

### Solid Carriers

In certain embodiments, the composition further comprises one or more solid carriers. As used herein, the term “solid carrier” includes additives commonly used in the preparation of powderous formulations such as thickeners, for example gums or cellulose derivatives such as xanthan gum, karaya gum and/or ethylcellulose. The one or more solid carriers can be any agriculturally suitable carrier, such as attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

In certain embodiments, the one or more solid carriers comprises any carrier suitable for ingestion, such as a saccharide comprising cellulose, xantham gum, karaya gum, ethylcellulose, inositol, galactose, arabinose, lactose, lactulose, mannitol, mannose, sorbose, turanose, platinose, or a combination thereof.

In some embodiments, the carrier comprises attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

In other embodiments, the carrier comprises attapulgite, kaolinite, fuller's earth, calcium carbonate, perlite, diatomaceous earth, calcium silicate, fly ash, a polysaccharide, a disaccharide, a monosaccharide, a gum, silica, propylene glycol, hemp protein, biochar, montmorillonite, activated charcoal, lignin, wood flour, hemp protein, pea protein, soy protein, gelatin, casein, chitosan, talc, calcium phosphate, arginine, lysine, calcium carbonate, carbon black, glutamine, betaine, bismuth phosphate, bismuth citrate, iron phosphate, or any combination thereof.

In some embodiments, the carrier comprises the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, or a combination thereof.

In some embodiments, the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, carrageenan, cellulose acetate, hydroxypropyl cellulose, cellulose acteate phthalate, maltrodextran, dextran, inulin, corn starch, amylopectin, sodium startch glycolate, pentaerthritol, cyclodextrin, or a combination thereof.

In certain preferred embodiments, the the solid carrier comprises silica and ethylcellulose, more particularly about 10% to about 50% by weight of the silica and about 50 to about 90% by weight of the ethylcelluose.

In other preferred embodiments, the solid carrier comprises silica and activated charcoal, particularly about 10% to about 90% by weight of the silica and about 10% to about 90% by weight of the activated charcoal.

In certain embodiments, the binder further comprises arginine, lysine, or both arginine and lysine. While not being bound by theory, it is believed that arginine and lysine are capable of forming hydrogen bonds with the small molecule, such as 3-NOP, thereby altering the release rate into the flooded ecosystem.

In other preferred embodiments, the carrier comprises activated charcoal and ethylcellulose, particularly about 10% to about 50% by weight of the activated charcoal and about 40 to about 90% by weight of the ethylcellulose.

In some embodiments, the carrier further comprises about 1 to about 10% by weight of sodium lignosulfate. While not being bound by theory, it is believed that sodium lignosulfate improves coating adhesion to the tablet resulting in a reduction in release rate of the small molecule.

In other embodiments, the carrier comprises arginine and polycaprolactone, such as about 10 to about 60% by weight of the arginine and about 30 to about 90% by weight of the polycaprolactone.

In other preferred embodiments, the carrier comprises 25% silica, 66% polycaprolactone, such as about 10 to about 60% by weight of the silica and about 30 to about 90% by weight of the polycaprolactone.

In certain embodiments, the composition comprises a granular shape. The composition may comprise any suitable shape, such as a spherical-, square-, rectangular-, capsular-, cylindrical-, conical-, ovular-, triangular-, diamond-, disk-like shape, or a combination thereof. In certain embodiments, the shape of the particle affects the rate of dissolution of the particle.

The granular particle can comprise any suitable texture, for example hard or soft. In certain embodiments, the texture of the particle affects the rate of dissolution of the particle. In certain embodiments, the composition comprises a combination of differently textured pellets each of which release the small molecule at different rates.

In certain embodiments, the granular particles comprise a uniform size distribution, for example about ±20%, ±15%, ±10%, ±5%, ±2%, or ±1% size distribution in the median particle size. In certain embodiments, the granular particles comprise a non-uniform size distribution, for example greater than about ±20%. In certain embodiments, the granular particles comprise a plurality of differently sized populations of granular particles each of which comprise a uniform size distribution.

In certain embodiments, the one or more solid carrier dissolves and thereby releases the one or more small molecules that reduce the production of greenhouse gases and/or precursors thereof. In a preferred embodiment, the one or more solid carriers will dissolve in water.

C. Extended and Delayed Release

It may be necessary to vary the rate of dissolution of the composition. For example, one may want to produce an extended-release formulation, wherein the composition releases the one or more small molecules over a period of time to maintain a suitable environmental concentration of the one or more small molecules. This can be beneficial to reduce the frequency of applications, for example to reduce labor costs and/or applications in rural and/or hard to reach environments. In certain embodiments, complete dissolution of the composition and full release of the one or more small molecules occurs over at least about 1, 2, 3, 4, 5, 6, 7, 14, 21, 28, 35, 42, 49, 56, 63, 70, 77, 84, 91, 98, 105, 112, 119, 126, 133, 140, and/or nor more than about 2, 3, 4, 5, 6, 7, 14, 21, 28, 35, 42, 49, 56, 63, 70, 77, 84, 91, 98, 105, 112, 119, 126, 133, 140, or 147 days, for example about 1 to about 147 days, preferably about 7 to about 63 days, more preferably about 7 to about 42 days even more preferably about 14 to about 42 days yet even more preferably about 14 to about 28 days.

In certain embodiments, the extended-release formula may comprise any suitable extended-release formula. In certain embodiments, the extended-release formula comprises one or more additives that reduce the rate of dissolution of the composition, one or more additives that reduce the rate of dissolution of the small molecule from the composition, one or more additives that comprise a membrane that dissolves over time, wherein the rate of dissolution of the membrane controls the rate of release of the one or more small molecules, a suitable alternative, or a combination thereof.

In certain embodiments, the population of granular particles comprises a plurality of populations of granular particles wherein each population comprises a different formulation and/or shape. In certain embodiments, the population of granular particles comprises a first population and a second population. In certain embodiments, the population of granular particles further comprises at least 1, 2, 3, 4, 5, 5, 6, 8, or 9 and/or no more than 4, 5, 6, 7, 8, 9, or 10 additional populations, for example a total of 3-10 additional populations, preferably 3-7 additional populations, more preferable 3-5 additional populations. In a preferred embodiment, each of the additional populations comprises a different formulation than the others.

In certain embodiments, the rate of dissolution of the granular particles is modulated by the size of the granular particle. In certain embodiments, smaller granular particles dissolve faster than larger granular particles, such that each successive larger population in the plurality of populations of differently size particles provides a delayed release compared to the smaller populations of particles. In certain embodiments, an increased proportion of larger to smaller granular particles in a population of granular particles results in slower rates of dissolution of the population of granular particles.

In certain embodiments, the first population of particles comprises an immediate release formulation. In certain embodiments, the second population comprises a delayed release formulation, wherein the second population dissolves and/or releases the one or more small molecules that reduce the production of one or more deleterious atmospheric gasses and/or precursors after the first population. In certain embodiments, each additional population comprises a delayed release formulation, wherein each population dissolves and/or releases the one or more small molecules that reduce the production of one or more deleterious atmospheric gasses and/or precursors at a different time than each of the other populations.

In some embodiments, an immediate release formulation releases the one or more small molecules that reduce the production of one or more deleterious atmospheric gases with about 1 day, 2 days, 3 days, 4 days, 5 days, or 6 days. In some embodiments, a delayed release formulation releases the one or small molecules after about 1 week or more, for example up to about 21 weeks.

In some embodiments, the first population of particles provides has a half-life for the one or more small molecules (such as about about 1 to 12 hours), the second population has a longer half-life (such as about 24 or more hours), each additional population has a longer half-life than the previous population such that an effective amount of the one or more small molecules is maintained for weeks or months. Thus, the present compositions advantageously do not, in such embodiments, require repeated, frequent applications to the flooded ecosystem.

In certain embodiments, the extended release happens within the first 10% of the half-life and then the 1st delayed happens with 1-2 half lives, then the next with 1-2 of the delayed release.

In some embodiments, the particles have a size ranging from about 1 mm to about 20 mm, about 1 to about 15 mm, about 1 to about 10 mm, about 5 to about 20 mm, about 5 to about 15 mm, or about 5 to about 10 mm.

Coatings

In certain embodiments, the composition may comprise a coating, for examples particles or tablet having a coating. The coating can comprise any suitable coating, such as a wax, a fat, or a synthetic polymer. In certain embodiments, the wax comprises organic compounds consisting of long alkyl chains, natural waxes (plant, animal) which are typically esters of fatty acids and long chain alcohols as well as synthetic waxes, which are long-chain hydrocarbons lacking functional groups. In certain embodiments, the fat comprises a wide group of compounds which are soluble in organic solvents and largely insoluble in water such as hydrogenated fats (or saturated fats) which are generally triesters of glycerol and fatty acids. Suitable fats can have natural or synthetic origin. In certain embodiment, the fat comprises glycerine monostearate, carnauba wax, candelilla wax, sugarcane wax, palmitic acid, stearic acid hydrogenated cottonseed oil, hydrogenated palm oil and hydrogenated rapeseed oil, or combinations thereof. Any suitable synthetic polymer can be used, such as poly-L-glutamic acid (PGA) and polylactic acid (PLA). In preferred embodiments, the synthetic polymer is at least partially water soluble.

The coating may be single layer or multiple layers, preferably two layers.

In some embodiments, the coating is selected from cellulose acetate phlalate, ethyl cellulose, hydroxypropyl cellulose, polycaprolactone, alginate, chitosan, polyethylene glycol, cellulose actate, triacetin, propylene glycol, n-methyl-2-pyrollidone, and any combination thereof.

In certain preferred embodiments, the coating comprises two or more polyelectrolytes, such as polystyrene sulfonate, polyethyleneimine, sodium lignosulfate, polyglutamic acid and poly-L-lysine, poly-L-arginine, polyallylamine hydrochloride, polyacrylic acid, or any combination thereof.

In some preferred embodiments, the polyelectrolytes comprise polyallylamine hydrochloride and sodium lignosulfate.

In some preferred embodiments, the polyelectrolytes comprise comprise polyallylamine hydrochloride and polystyrene suylfonate.

In other preferred embodiments, the polyelectrolytes comprise sodium lignosulfate and one of polyglutamic acid and poly-L-lysine, or poly-L-arginine, and sodium lignosulfate.

In still other preferred embodiments, the polyelectrolytes comprise polystyrene sulfonate and one of polyglutamic acid and poly-L-lysine, or poly-L-arginine.

The polyelectrolytes may, in certain embodiments, be chemically cross-linked with a cross-linking agent.

In certain embodiments, the composition comprises one or more coatings applied with minimal to no bubbles. Additionally or alternatively, the composition comprises one or more coatings that comprise a foam or a plurality of air bubbles. In certain cases, the foamed coating can temporarily alter the buoyancy of the composition. One such example includes a composition comprising a foamed coating that floats when initially applied to a flooded ecosystem, then, after a period of time, the air pockets in the foamed coating fill with water resulting in the composition sinking to the bottom of the flooded ecosystem. This may be advantageous when the composition must be distributed from a centralized water source to a plurality of downstream flood ecosystems. Additionally or alternative, this may be advantageous to allow a composition applied to a small portion of a large flooded ecosystem to more evenly distribute across the entirety of the surface before sinking to the floor of the flood ecosystem.

D. Additives with a Density Greater than Water

In certain embodiments, the composition further comprises one or more additives with a density greater than water. For example, the additive may have a density greater than 1.1, preferably about 1.1 mg/mL to about 3 mg/mL, about 1.5 to about 3 mg/mL, about 1.5 to about 2.5 mg/mL, or about 1.5 to about 2 mg/mL. Suitable additives include silica, attapulgite, kaolinite, fuller's earth, calcium carbonate, perlite, diatomaceous earth, calcium silicate, fly ash, or a combination thereof. In certain embodiments, the one or more additives with a density greater than water result in the composition sinking below the water surface when applied to the flooded ecosystem. In certain embodiments, the one or more additives with a density greater than water result in the composition partially or completely sinking to the bottom of the flooded ecosystem. In preferred embodiments, the composition completely sinks to the bottom of the flooded ecosystem. In certain embodiments, the composition comprising the additive with a density greater than water has a density of at least 1.1, preferably about 1.1 mg/mL to about 3 mg/mL, about 1.5 to about 3 mg/mL, about 1.5 to about 2.5 mg/mL, or about 1.5 to about 2 mg/mL.

E. Agriculturally Beneficial Additives

In certain embodiments, the composition further comprises one or more agriculturally beneficial additives. The agriculturally beneficial additive can be any suitable additive depending on the application, such a vitamin, a nutrient, an antibiotic, a fungicide, an herbicide, a fertilizer, or a combination thereof.

In certain embodiments, the additive includes one or more suitable components that reduce methanogenesis by methanogens, such as, seaweed (e.g., Asparagopsis taxiformis), kelp, 3-nitrooxypropanol, anthraquinones, ionophores (e.g., monensin and/or lasalocid), polyphenols (e.g., saponins, tannins), organosulfurs (e.g., garlic extract), flavonoids (e.g., quercetin, rutin, kaempferol, naringin, and anthocyanidins; bioflavonoids from green citrus fruits, rose hips and black currants), carboxylic acid, terpenes (e.g., D-limonene, pinene and citrus extracts), or a combination thereof.

Methods for Reducing Production of Deleterious Atmospheric Gases and/or Precursors Thereof

In certain embodiments provided herein are methods. In certain embodiments, provided herein are methods for using one or more small molecules that reduce the production of one or more deleterious atmospheric gases and/or precursors thereof. In certain embodiments, provided herein are methods for applying one or more small molecules that reduce the production of one or more deleterious atmospheric gases and/or precursors thereof to any suitable environment. The suitable environment can comprise any suitable environment as described in the Ecosystems section above. In preferred embodiments, the suitable environment comprises a flooded ecosystem, such as a rice paddy.

In certain embodiments, the method for reducing emissions of deleterious atmospheric gasses and/or precursors thereof from a flooded ecosystem comprises applying a composition comprising one or more small molecules that reduce the production of the deleterious atmospheric gasses and/or precursors thereof to the flooded ecosystem. The composition can comprise any suitable composition. In a preferred embodiment, the composition comprises any one of the compositions as described in the Compositions for reducing production of deleterious atmospheric gases and/or precursors thereof section above. In a more preferred embodiments, the composition comprises 3-NOP.

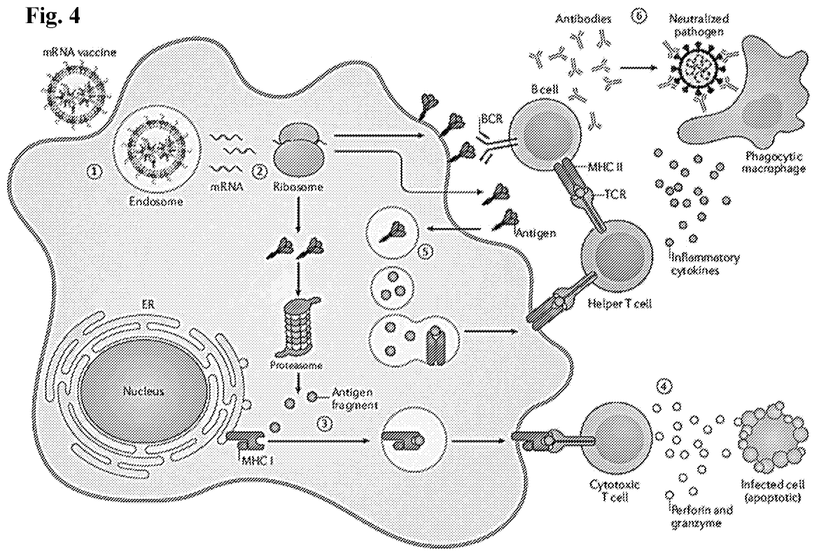

An exemplary method is shown in FIG. 4. In FIG. 4, the flood environment comprises a rice paddy (401) comprising one or more rice plants (402), wherein there is an oxygen-rich, anaerobic zone near the surface of the water (403) and an oxygen-deprived, anaerobic zone below the surface of the water (404). To the rice paddy, one or more compositions comprising one or more small molecules that reduce the production of one or more deleterious gases and/or precursors thereof (405) is applied, wherein the composition sinks to the paddy floor (406), wherein the dissolution of the composition reduces the production of the one or more deleterious gases and/or precursors thereof by one or more microorganisms.

In certain cases, the composition needs to be reapplied periodically to maintain a suitable concentration of the one or more small molecules. In certain embodiments, the method further comprises reapplying after a period of time a composition comprising one or more small molecules that reduce the production of the deleterious atmospheric gasses and/or precursors thereof to the flooded ecosystem. In certain embodiments, the method further comprises, reapplying again after a period of time a composition comprising one or more small molecules that reduce the production of the deleterious atmospheric gasses and/or precursors thereof to the flooded ecosystem. Any suitable number of reapplications may be performed as needed to maintain a an effective amount of the one or more small molecules. In some embodiments, the composition is reapplied after about 7 to about 28 days, about 7 to 46 days, about 7 to 92 days or about 7 to 147 days.

In certain embodiments, the composition is delivered to the one or more flooded ecosystems. Any suitable delivery method can be used, such as delivery with or without human intervention, for example aerial delivery, e.g., by drone.

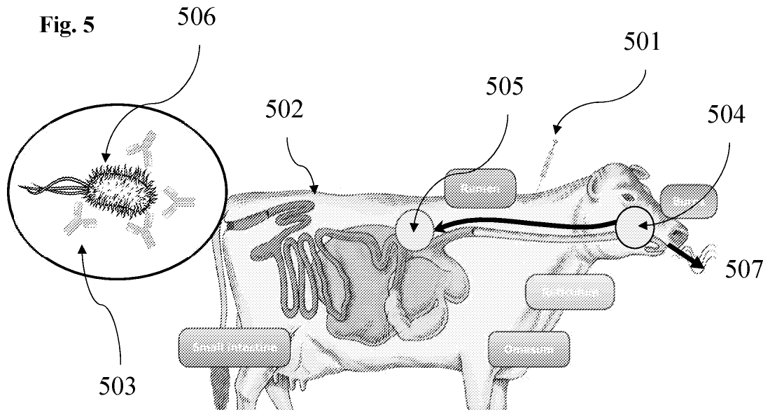

An exemplary method of delivery is shown in FIG. 5. In FIG. 5, one or more compositions comprising one or more small molecules that reduce the production of one or more deleterious gases and/or precursors thereof is loaded into an aerial vehicle, e.g., a drone, (501), wherein the aerial vehicle distributes (502) the composition to the flooded ecosystem (503).

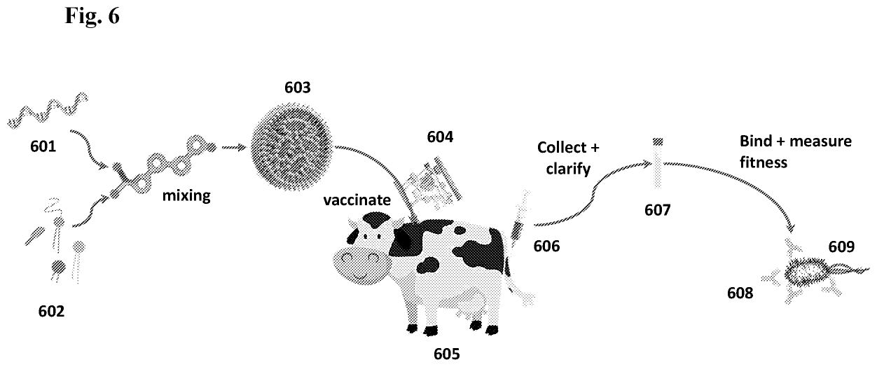

The composition can be applied at any suitable gestational period for the agricultural crop. For example, the composition can be applied during Vegetation, Reproductive, and/or Ripening stage during rice production in a paddy field as shown in FIG. 6.

In certain embodiments, the composition is delivered to one or more water sources, whereby the composition is delivered to the one or more flooded ecosystems upon use of the water from the source. For example, the water source can be a pool used to irrigate a cropland, e.g., a paddy field, wherein the composition is distributed to the cropland as the water is pulled from the water source.

In certain cases, the concentration of the one or more small molecules can be measured to ensure the presence of a suitable concentration of the one or more small molecules. Any suitable method may be used to measure the concentration, such as a strip test, liquid chromatography, or thin layer chromatography. The method can be performed with or without human intervention.

In some embodiments, the amount of the small molecule (e.g., 3-NOP) applied is from about 0.1 ppm to about 100 ppm per acre of flooded ecosystem. In other embodiments, the amount is about 0.5 ppm to about 50 ppm per acre. In still other embodiments, the amount is about 0.5, 1, 5, 10, 15, 20, 25, 30, 40, 45 or 50 pp per acre.

In some embodiments, the amount of the small molecule applied is from about 0.5 ppm to about 50 ppm per acre.

Kits for Reducing Production of Deleterious Atmospheric Gases and/or Precursors Thereof

In certain embodiments, provided herein are kits. In certain embodiments, the kit comprises any one of the compositions as described in the Compositions for reducing production of deleterious atmospheric gases and/or precursors thereof section. In certain embodiments, the kit further comprises a suitable container for shipping.

### Methods of Using Vaccines, Animal Feed, Antibodies, Milk

Provided herein are methods of using the vaccines, antibodies, milk, animal feed, agents (e.g., an agent that reduces methane production in a ruminant, a probiotic bacterial strain, etc.), or any combination thereof.

In certain aspects, provided herein are methods of inducing an immune response against at least one methanogen in a ruminant, the method comprising administering to the ruminant the vaccines or pharmaceutical compositions of the present disclosure.

In some embodiments, the immune response comprises a B cell response (e.g., to produce the antibodies). The antibodies produced in response to the vaccine are transferred to the saliva of the ruminants, which are swallowed by the ruminants to enter the rumen. Once in the rumen, the antibodies come in contact with at least one methanogen to bind/neutralize said methanogen.

As used herein, the term “neutralization of a methanogen” encompasses any reduction in one or more activities that are normally carried out by the methanogen in the absence of the antibodies that bind the methanogen.

In some embodiments, the activity of a methanogen includes but is not limited to, the activity that aids in producing methane gas. For example, binding of the antibodies to the methanogen may reduce the ability of the methanogen to carry out biochemical reactions that are necessary to produce methane, e.g., reduce the ability to convert hydrogen (H2) and carbon dioxide (CO₂) or acetate into methane (CH4) and ATP. In some embodiments, the reduced ability to produce methane may lower the fitness of methanogen in the rumen.

In some embodiments, the activity of a methanogen includes but is not limited to, the activity that aids in forming a granular colony with other bacteria. In some embodiments, such activity may be disrupted physically—e.g., antibodies binding to the methanogen would prevent physical association and/or film formation of the granular colony of bacteria. In some embodiments, a reduction in the activity of forming a granular colony may lead to the reduced ability of a methanogen to remain in the rumen. In some embodiments, such reduced ability may result in the reduction of the total number of methanogens inside the rumen.

In certain aspects, provided herein are methods of reducing the activity, number, and/or type of methanogens in the gut of a ruminant, the method comprising administering to the ruminant the vaccines or pharmaceutical compositions of the present disclosure.

In certain aspects, provided herein are methods of reducing the amount of methane produced by a ruminant, the method comprising administering to the ruminant the vaccines or pharmaceutical compositions of the present disclosure.

In preferred embodiments, any one of the methods produces an antibody against at least one methanogen. In some embodiments, the antibody is an IgG or an IgA. In preferred embodiments, the antibody is an IgA. The IgA isoform, at least in cattle, may be more stable in the rumen. For example, IgA levels in cattle saliva were reduced by only 40% after 8 h exposure to rumen contents while IgG levels were reduced by 80%.

In some embodiments, the antibody is produced in an amount sufficient to: (a) carry the antibody to the gut; (b) reduce the number and/or type of methanogens in the gut; and/or (c) reduce the amount of methane produced by the ruminant.

In some embodiments, the method reduces the methane production by the ruminant by at least about 1%, 5%, 10%, 20%, 30%, 40%, 50%, 60%, 70%, 80%, 90%, or 95% as compared with a control.

In some embodiments, the control is an accepted reference, or the amount of methane production in a ruminant that has not been vaccinated.

Notably, methane emission or production of methane by a ruminant can occur at any part of its intestinal track, which includes, e.g., a rumen and a lower bowel (lower intestinal track). The rumen accounts for 90% of all methane production. The rumen has no adaptive immune response. Thus, to be effective in reducing the level of methane production, the rumen or a methanogen therein must be exposed to neutralizing antibodies that bind and inactivate the methanogen. By contrast, the lower bowel, which accounts for 10% of all methane production, has adaptive immune response such that any immune response due to a vaccine can be amplified in the lower bowel. Accordingly, in preferred embodiments, the result of immune response from a vaccine is exposed to the lower bowel of a ruminant, which then further amplifies the effect of the vaccine. In other words, in preferred embodiments, a vaccine of the present disclosure or the immune response it elicits is exposed to a methanogen in a lower bowel of the ruminant. Thus, the present disclosure encompasses a method of reducing (i) methane production and/or (ii) activity, number, and/or type of methanogens in the lower intestinal track of a ruminant, the method comprising administering to the ruminant a nucleic acid vaccine comprising a nucleic acid encoding at least one methanogen cell surface protein or a fragment thereof.

As described herein, in some embodiments, the vaccine or the pharmaceutical composition is administered to the ruminant via a route selected from intramuscular administration, intradermal administration, subcutaneous administration, and nasal administration.

In some embodiments, the ruminant is administered with at least one dose of the vaccine or pharmaceutical composition.

In some embodiments, the ruminant is administered with at least one or two repeat doses of the vaccine or pharmaceutical composition (e.g., booster dose).

In some embodiments, the ruminant is administered with the repeat dose of the vaccine or pharmaceutical composition after at least about 2 weeks, 1 month, 6 months, or 12 months from the time the ruminant is administered with the preceding dose of the vaccine.

In some embodiments, the ruminant is administered with the repeat dose of the vaccine or pharmaceutical composition no more than about 3 months, 6 months, 12 months, or 24 months from the time the ruminant is administered with the preceding dose of the vaccine.

In some embodiments, the ruminant receives the repeat dose of the vaccine after at least about 2 weeks and no more than about 18 months from the time the ruminant is administered with the preceding dose of the vaccine.

In some embodiments, the ruminant receives a repeat dose of the vaccine after at least about 4 weeks and no more than about 12 months from the time the ruminant is administered with the preceding dose of the vaccine.

In certain embodiments, the ruminant is administered with a dosage of between 1 ug/kg and 400 ug/kg of the nucleic acid vaccine.

In some embodiments, the ruminant is administered with a dosage of the nucleic acid comprising at least about 25 ug, 50 ug, 100 ug, 150 ug, 200 ug, 250 ug, or 300 ug of the RNA.

In some embodiments, the ruminant is administered with a dosage of the nucleic acid comprising at least about 100 ug.

In some embodiments, the ruminant is administered with a dosage of the nucleic acid comprising at least about 200 ug.

In some embodiments, the ruminant is administered with a dosage of the nucleic acid comprising at least about 300 ug.

In certain embodiments, the methods of the present disclosure further comprises administering to the ruminant at least one agent (e.g., at least one additional agent) that reduces the level of methane produced by the ruminant.

In some embodiments, the at least one agent is selected from 3-Nitrooxypropanol (3-NOP), ethyl-3NOP, 2-bromoethanesulfonate (BES), 2-chloroethanesulfonate (CES), 3-bromopropanesulfonate (BPS), bromochloromethane (BCM), bromoform, bromodichloromethane, dibromochloromethane, carbon tetrachloride, trichloroacetamide, trichloroethyladipate, lumazin (2,4-pteridinedione), p-aminobenzoic acid, lovastatin, mevastatin, pravastatin, diallyl disulfide, garlic oil, saponins, tannins, flavonoids, nitrate, nitroethane, -nitro-propionate, 2-nitropropanol, 2-nitroethanol, malate, acrylate, oxaloacetate, fumarate, propynoic acid, 3-butenoic acid, 2-butynoic acid, ethyl 2-butynoate, monensin, lasalocid, bovicin HC5, nisin, and any combination thereof.

In preferred embodiments, the agent is 3-NOP or ethyl-3-NOP.

In certain aspects, provided herein are methods of reducing methane production in a ruminant, the method comprising orally administering to and/or feeding the ruminant the antibody, the milk and/or the derivatives thereof, and/or the animal feed of the present disclosure.

In certain aspects, the methods of the present disclosure relate to a ruminant. In some embodiments, the ruminant is selected from a cow, a bull, a bison, a yak, a buffalo, an antelope, a goat, a sheep, a deer, a giraffe, a caribou, a gazelle, a macropod, a llama, a camel, and an alpaca.

In some embodiments, the ruminant is an offspring (e.g., calf) of the vaccinated female ruminant that received the milk comprising an antibody that binds at least one methanogen.

In some embodiments, the ruminant is an adult ruminant. In other embodiments, the ruminant is a young ruminant (e.g., a calf). In some embodiments, a young ruminant includes a ruminant from birth to weaning. In some embodiments, a young ruminant includes a ruminant from birth up to two years of age, such from birth up to 3, 4, 5, 6, 7, 8, 9, 10, 11, or 12 months. Accordingly, in some embodiments, a young ruminant may be at least, about, or no more than 1, 2, 3, 4, 5, 6, 7, 8, 9, 10, 11, 12, 13, 14, 15, 16, 17, 18, 19, 20, 21, 22, 23, or 24 months in age. In preferred embodiments, a young ruminant is administered with a vaccine, antibodies, milk, animal feed, agent (e.g., an agent that reduces methane production in a ruminant, a probiotic bacterial strain, a small molecule inhibitor, etc.), or other composition of the present disclosure prior to establishment of methanogens. In some such embodiments, a lower dose of vaccine or other agents may be required.

In some embodiments, a ruminant is a ruminant born from a vaccinated parent(s). In some embodiments, a ruminant is a ruminant born from a vaccinated mother such that the ruminant received a high level of methanogen-neutralizing antibodies in the colostrum and milk fed to the ruminant at birth. Such a ruminant or a ruminant who received an early treatment may have low initial methanogen establishment, thereby enhancing a long term performance of the vaccines, antibodies, milk, animal feed, agents (e.g., an agent that reduces methane production in a ruminant, a probiotic bacterial strain, a small molecule inhibitor, etc.), or other compositions of the present disclosure.

In some embodiments, a ruminant is vaccinated with each change in hands and/or environment, such as from one owner to another, one ranch to another, or one geographical area to another. Typical ranch operations will vaccinate a newly arrived animal to manage disease, and vaccinating with a vaccine of the present disclosure (e.g., a vaccine that targets at least one methanogen cell surface protein) will fall into this standard agronomic practice.

In some embodiments, a ruminant is vaccinated with a vaccine of the present disclosure (e.g., a vaccine that targets at least one methanogen cell surface protein) at a time when the ruminant is subject to at least one other vaccination. For example, a ruminant (e.g., a domesticated ruminant, a dairy cow, a beef cow) is subject to vaccination against infectious bovine rhinotracheitis (IBR), bovine virus diarrhea (BVD), paranfluenza-3 (PI3), bovine respiratory syncytial virus (BRSV), clostridia, E. Coli mastitis, leptospirosis, mannheimia hemolytica, brucella, vibriosis, campylobacter, trichomonas, trichomoniasis, rotavirus, coronavirus, and/or respiratory disease. An exemplary vaccination regimen for a ruminant throughout the life cycle is shown in Table 8A.

TABLE 8A
Vaccination program for a ruminant

| Animal | Life Stage | Exemplary Vaccinations |
|---|---|---|
| Dairy | Adult | 4-way viral- |
| | Dry-off (e.g., 45-60 days | IBR/BVD/PI3/BRSV |
| | pre-calving) | Leptospirosis (5-way) |
| | | Clostridial (7-way) |
| | | J-5 (mastitis) |
| Dairy | Adult | 4-way viral- |
| | Pre-breeding (e.g., 30 days | IBR/BVD/PI3/BRSV |
| | prior to breeding season) | Leptospirosis (5-way) |
| Dairy | Calf | 4-way viral- |
| | Weaning (e.g., 6-12 weeks) | IBR/BVD/PI3/BRSV |
| | | Leptospirosis (5-way) |
| | | Clostridial (7-way) |
| | | Mannheimia hemolytica |
| Dairy | Calf | 4-way viral- |
| | Yearling (i.e., 12-24 mo) | IBR/BVD/PI3/BRSV |
| | | Leptospirosis (5-way) |
| | | Clostridial (7-way) |
| | | Mannheimia hemolytica |
| | | Brucella |
| Beef | Adult | 4-way viral- |
| | Pre-breeding (e.g., 30 days | IBR/BVD/PI3/BRSV |
| | prior to breeding season) | Leptospirosis (5-way) |
| | | Vibriosis |
| | | (Campylobacter)- |
| | | natural mating |
| | | Trichomonas-natural |
| | | mating |
| Beef | Adult | 4-way viral- |
| | Pregnancy examination (e.g., 30-45 | IBR/BVD/PI3/BRSV |
| | days post-breeding season) | Leptospirosis (5-way) |
| | | Clostridial (7-way) |
| | | Scour vaccines-E. coli, |
| | | possibly combination |
| | | with rota and coronavirus |
| Beef | Calf | Clostridial vaccines |
| | Newborn (e.g., 0-3 mo) | Possibly respiratory |
| | | vaccines, if there is a |
| | | history of disease in very |
| | | young calves |
| Beef | Calf | 4-way viral- |
| | Weaning (e.g., 6-8 mo) | IBR/BVD/PI3/BRSV |
| | | Leptospirosis (5-way) |
| | | Clostridial (7-way) |
| | | Mannheimia hemolytica |
| | | Brucella |
| Beef | Adult | 4-way viral- |
| | Arrival at stocker | IBR/BVD/PI3/BRSV |
| | operation (e.g., 6-8 mo) | Mannheimia hemolytica |
| Beef | Adult | 4-way viral- |
| | 14-21 d post-arrival at | IBR/BVD/PI3/BRSV |
| | stocker operation | Clostridial (7-way) |
| | | Mannheimia hemolytica |
| | | Leptospirosis (5-way) |
| | | (optional) |
| Beef | Adult | 4-way viral- |
| | Arrival at feedlot | IBR/BVD/PI3/BRSV |
| | operation (e.g., 9-11 mo) | Mannheimia hemolytica |
| Beef | Adult | 4-way viral- |
| | 14-21 d post-arrival at | IBR/BVD/PI3/BRSV |
| | feedlot operation | Clostridial (7-way) |
| | | Mannheimia hemolytica |
| | | Leptospirosis (5-way) |
| | | (optional |

Vaccinating a large number of ruminants (e.g., in a ranch, e.g., herding ruminants) is a challenging process. Thus, in preferred embodiments, the administration of a vaccine of the present disclosure is incorporated into a pre-existing vaccination program that a ruminant is already subject to. Such a method reduces the cost, time, and efforts in administering a vaccine of the present disclosure to a ruminant.

### Method of Producing the RNA Vaccine

Methods of producing the RNA vaccine are well known in the art. For example, preparation of IVT mRNA is described at least in Sahin et al. (2014) Nature Reviews Drug Discovery 13:759-780; and Vlatkovic (2021) Biomedicines 9(5):530, each of which is incorporated herein by reference).

In some embodiments, the mRNA is synthesized in a cell-free system by in vitro transcription from a DNA template, such as a linearized plasmid or a PCR product. With the exception of the 5′ cap, this DNA template encodes all the structural elements of a functional mRNA. In vitro transcription is performed with T7 or SP6 RNA polymerase in the presence of nucleotides or analogs thereof and thereafter the mRNA is capped enzymatically. The template DNA is then digested by dNases and the mRNA is purified by conventionally used methods for isolating nucleic acids.

In some embodiments, the nucleic acid vaccines described herein may be formulated in a nanoparticle to be delivered by a parenteral route as described in U.S. Pub. No. US20120207845; the contents of which are herein incorporated by reference in its entirety. In some embodiments, the nucleic acid vaccines may be formulated in a lipid nanoparticle made by the methods described in US Patent Publication No US20130156845 or International Publication No WO2013093648 or WO2012024526, each of which is herein incorporated by reference in its entirety.

The lipid nanoparticles described “erei” may be made in a sterile environment by the system and/or methods described in US Patent Publication No. US20130164400, herein incorporated by reference in its entirety.

In some embodiments, the LNP formulation may be formulated by the methods described in International Publication Nos. WO2011127255 or WO2008103276, the contents of each of which are herein incorporated by reference in their entirety.

In some embodiments, such LNPs are synthesized using methods comprising microfluidic mixers. Exemplary microfluidic mixers may include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. I:e37; Chen, D. et al., Rapid discovery of potent siRNA-containing lipid nanoparticles enabled by controlled microfluidic formulation. J Am Chem Soc. 2012. 134(16):6948-51; each of which is herein incorporated by reference in its entirety). In some embodiments, methods of LNP generation comprising SHM, further comprise the mixing of at least two input streams wherein mixing occurs by micro structure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, each of which is expressly incorporated herein by reference in their entirety. In one embodiment, the nucleic acid vaccine of the present invention may be formulated in lipid nanoparticles created using a micromixer such as, but not limited to, a Slit Interdigital Micro structured Mixer (SIMM-V2) or a Standard Slit Interdigital Micro Mixer (SSIMM) or Caterpillar (CPMM) or Impinging-jet (IJMM) from the Institut fiir Mikrotechnik Mainz GmbH, Mainz Germany).

In some embodiments, the nucleic acid vaccines of the present invention may be formulated in lipid nanoparticles created using microfluidic technology (see Whitesides, George M. The Origins and the Future of Microfluidics. Nature, 2006 442: 368-373; and Abraham et al. Chaotic Mixer for Microchannels. Science, 2002 295: 647-651; each of which is herein incorporated by reference in its entirety). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295: 647-651; which is herein incorporated by reference in its entirety).

In some embodiments, the nucleic acid vaccines of the present invention may be formulated in lipid nanoparticles created using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

### Methods for Detection of Methane Production

Various methods for detecting the level of methane produced by ruminants are known in the art and can be applied to the methods of the present disclosure.

In some embodiments, portable respiration hoods for tethered and non-tethered animals (Garnsworthy et al. (2012) J. Dairy Sci. 95:3166-3180; Garnsworthy et al. (2019) Animals 9:837; Zimmerman and Zimmerman WO2011130538; each of which is incorporated herein by reference) directly measure the gas concentration of incoming and exhaust air from individual animals.

In some embodiments, tracer-ratio gas releases from the animal (Johnson et al. (1994) Environ. Sci. Technol., 28, 359-362, which is incorporated herein by reference), such as Sulfur hexafluoride (SF6) (Grainger et al. (2007) J. Dairy Sci., 90:2755-2766; Vechi et al. (2022) Agriculture, Ecosystems and Environment 330:107885; each of which is incorporated herein by reference), assumes that the tracer gas and the emitted CH4 have similar transport paths, so that a tracer measurement can establish the CH4 emission rate.

In some embodiments, micrometeorological techniques are typically considered a herd-scale measurement, where the emission rate is calculated from the measurement of enhanced gas concentrations downwind of an animal herd (Harper et al. (2011) Anim. Feed Sci. Tech., 166-167, 227-239, which is incorporated herein by reference), and these include the mass balance technique (Laubach et al. (2008) Aust. J. Exp. Agr., 48:132-137; Lockyer and Jarvis (1995) Environ. Pollut. 90:383-390; each of which is incorporated by reference), eddy covariance (Dengel et al. (2011) Glob. Change Biol., 17:3524-3533; Felber et al. (2015) Biogeosciences, 12:3925-3940; each of which is incorporated herein by reference), and inverse dispersion techniques (Flesch et al., (2005) Atmos. Environ., 39:4863-4874; Todd et al. (2014) J. Environ. Qual., 43:1125-1130; Bai et al. (2021) Atmos. Meas. Tech., 14:3469-3479; each of which is incorporated herein by reference). The main advantage of micrometeorological techniques is that they do not interfere with the animals or the environment.

There are also devices that measure the level of methane (see e.g., Rey et al. (2019) Animals 9:563, Mapfumo et al. (2018) Pastoralism: Research, Policy and Practice 8:15; each of which is incorporated herein by reference). For example, the laser methane detector (LMD) is a hand held open path laser measuring device (e.g., LaserMethaneMini (Tokyo Gas Engineering Co., Ltd. Anritsu Devices Co., Ltd., Tokyo, Japan)). The principle of the LMD measuring technology is described (Chagunda et al. (2013) Animal, 7:394-400; Garnsworthy et al. (2012) J. Dairy Sci. 95:3166-3180; and Chagunda et al. (2009) Comput. Electron. Agric. 68:157-160; each of which is incorporated herein by reference). Briefly, this device is based on infrared absorption spectroscopy using a semiconductor laser for CH4 detection. The device must be pointed towards the nostrils of the cow from a fixed distance. Then, the LMD measures the density of the air column between the device and the animal's nostrils. The reflected laser beam is detected by the device, and its signal is processed and converted to the cumulative CH4 concentration along the laser path in ppm-m. The LMD is connected to a tablet (Samsung Galaxy Tab A6, New Jersey, USA) running GasViewer app (Tokyo Gas Engineering Solutions, Tokyo, Japan) via Bluetooth connection for exporting and storing the data in real time at 0.5 s intervals. The effect of atmospheric ambient CH4 concentration from the measurements is discounted using the offset function of the LMD.

The non-dispersive infrared analyzer CH4 analyzer (NDIR) (Guardian NG Edinburg Instruments Ltd., Livinstong, UK) is one of the so-called sniffer methods that measure CH4 concentration (ppm) in breath or exhaled air. These methods have been previously used (e.g., by Garnsworthy et al. (2012) J. Dairy Sci. 95:3166-3180) to assess the CH4 production of dairy cows at commercial farms. Briefly, a gas sampling tube from the front of a cow's head to a gas analyzer to continuously measure CH4 concentration in the cow's breath is used. Then, air is drawn through the instrument by an integral pump between the gas inlet port and analyzer. The device can have a range of 0 to 10,000 ppm, and air can be sampled continuously at a rate of 1 L/min through an 8 mm polyamide tube, using approximately 2 m of tube from the analyzer to cow's nostrils. Methane concentration can be recorded at 1 s intervals and stored in a datalogger (Data Recorder SRD-99; Simex Sp. Z o.o, Gdansk, Poland). Baseline or ambient CH4 concentration can be calculated as mean CH4 concentration before starting the measurements and subtracted from the measured data. Each day before starting measurements, the NDIR analyzer should be verified using standard mixtures of CH4 in nitrogen (0.0%, 0.25%, 0.50%, 0.75% and 1.0%; MESA International Technologies INC, Santa Ana, CA, USA).

Certain methods and devices are described further below and in Table 12.

TABLE 12
Features of exemplary methods for measuring methane output by individual animals

| | Purchase | Running | | | Behaviour | |
|---|---|---|---|---|---|---|
| Method | Cost ² | Cost ² | Labour ² | Repeatability | Alteration ³ | Throughput |
| Respiration chamber | High | High | High | High | High | Low |
| SF₆ technique | Medium | High | High | Medium | Medium | Medium |
| Breath sampling during | Low ⁴ | Low | Low | Medium | None | High |
| milking and feeding | | | | | | |
| GreenFeed | Medium | Medium | Low | Medium | Low | Medium |
| Laser methane detector | Low | Low | High | Low | Low-Medium | Medium |

As indicated above and shown in Table 12, exemplary methods include respiration chambers, the sulfuur hexafluoride (SF6) tracer technique, breath sampling during milking or feeding, the GreenFeed system, and the laser methane detector. Each method measures different components of methane output. Only respiration chambers measure total emissions from the animal via the oral, nasal and anal routes; all other methods ignore emissions via the anus and only measure methane emitted in breath. Breath measurements are justified because 99% of methane is emitted from the mouth and nostrils, and only 1% via the anus. The SF6 technique samples breath over 24 h, whereas other techniques use spot samples of breath over periods of minutes throughout the day, so diurnal variation has to be considered. The majority of methane (87%) is released by eructation, which provides a clear signal for sample processing.

### Respiration Chamber

Respiration chambers for open- or closed-circuit indirect calorimetry are considered the ‘Gold Standard’, and were used extensively in nutrition studies when establishing the Metabolisable Energy system. A single animal (or occasionally more) is confined in a chamber for between 2 and 7 days. Concentration of methane (and other gases if required) is measured at the air inlet and outlet vents of the chamber. The difference between outlet and inlet concentrations is multiplied by airflow to indicate methane emissions rate. In most installations, a single gas analyser is used to measure both inlet and outlet concentrations, often for two or more chambers. This involves switching the analyser between sampling points at set intervals, so concentrations are actually measured for only a fraction of the day.

Respiration chambers vary in construction materials, size of chamber, gas analysis equipment and airflow rate, all of which can influence results. Validation of 22 chambers at six UK research sites revealed an uncertainty of 25.7% between facilities, which was reduced to 2.1% when correction factors were applied to trace each facility to the international standard for methane. The main sources of uncertainty were stability and measurement of airflow, which are crucial for measuring methane emission rate. It was concluded, however, that chambers were accurate for comparing animals measured at the same site. It is an added challenge, when benchmarking alternative methods against respiration chambers, that the respiration chambers themselves have not been benchmarked against respiration chambers at other facilities.

For large-scale evaluation of methane emissions by individual animals, respiration chambers are challenging, with only a single study in growing Angus steers and heifers exceeding 1000 animals, which found methane production to be moderately heritable h²=0.27±0.07. Installation costs and running costs are high, and only one animal can be measured at a time. If the monitoring time is three days per animal, and chambers are run continuously, then maximum throughput would be approximately 100 animals per chamber per year. In practice, throughput is likely to be 30 to 50 animals per year. Cows are social animals, and confinement in a chamber may ultimately influence their feeding behaviour, resulting in less feed being consumed and in a different meal pattern compared with farm conditions. Altered feeding patterns or levels is not a problem for metabolic studies evaluating feeds, but can be a problem when evaluating individual animals. Furthermore, the representativeness of respiration chambers to grazing systems has been called into question. However, promising developments have led to more animal friendly respiration chambers constructed from cheaper, transparent materials. These lower the cost and reduce the stress of confinement with minimal disruptions to accuracy, precision and no drop in feed intake of the cows (Hellwing et al. (2012) J. Dairy Sci. 95:6077-6085, which is incorporated herein by reference).

### The SF6 Method

The SF6 tracer gas technique was developed in an attempt to measure methane emissions by animals without confinement in respiration chambers. Air is sampled near the animal's nostrils through a tube attached to a halter and connected to an evacuated canister worn around the animal's neck or on its back. A capillary tube or orifice plate is used to restrict airflow through the tube so that the canister is between 50 and 70% full after approximately 24 h. A permeation tube containing SF6 is placed into the rumen of each animal. The pre-determined release rate of SF6 is multiplied by the ratio of methane to SF6 concentrations in the canister to calculate methane emission rate.

Many research centres have used the SF6 technique with variations in design of sampling and collection equipment, permeation tubes, and gas analysis. Reliable results depend on following standard protocols, with greatest variation coming from accuracy of determining SF6 release rate from permeation tubes and control of sampling rate. With capillary tubes, sampling rate decreases as pressure in the canister increases, whereas an orifice plate gives a steadier sampling rate over 24 h. A source of error that has not been evaluated is that animals might interact and share methane emissions when the sampling tube of one animal is near the head of another animal. There is good agreement between methane emissions measured by the SF6 technique and respiration chambers, although results from the SF6 technique are more variable. For large-scale evaluation of methane emissions by individual animals, the SF6 technique is more useful than respiration chambers. Animal behaviour and intake might be affected by wearing the apparatus, and by daily handling to exchange canisters, but the technique is considerably less intrusive than respiration chambers, because cows remain in the herd. Labour and monetary costs for changing canisters each day and for lab analysis are high. Throughput is limited by the number of sets of apparatus available, handling facilities, labour, and the capacity of the lab for gas analysis. Animals need to be measured for 5 to 7 days, and it is recommended that group size should be less than 15 animals, so maximum throughput would be about 750 animals per year. Heritability has been estimated for methane production in grazing Holstein cows at h²=0.33±0.15.

### Breath Sampling During Milking and Feeding

Several research groups have developed methods to measure methane concentration in breath of cows during milking and/or feeding. These are often referred to as ‘sniffer methods’ because they use devices originally designed to detect dangerous gas leaks. Air is sampled near the animal's nostrils through a tube fixed in a feed bin and connected directly to a gas analyser. The feed bin might be in an automatic milking station or in a concentrate feeding station. Different research centres use different gas analysers (Nondispersive Infrared (NDIR), Fourier-transform infrared (FTIR) or photoacoustic infrared (PAIR)) and different sampling intervals (1, 5, 20 or 90-120 s). Methane concentration during a sampling visit of typically between 3 and 10 min may be specified as the overall mean, or the mean of eructation peaks. Some centres use CO2 as a tracer gas and calculate daily methane output according to ratio of methane to CO2 and daily CO2 output predicted from performance of the cow. Repeatability and rank correlations were higher for eructation peaks than for mean concentrations, and were higher for eructation peaks than for methane to CO2 ratio. However, all methods show good repeatability.

For large-scale evaluation of methane emissions by individual animals, breath-sampling methods have significant advantages compared with other methods. Breath-sampling methods are non-invasive because, once installed, animals are unaware of the equipment and are in their normal environment. Animals follow their normal routine, which includes milking and feeding, so no training of animals, handling, or change of diet is required. Equipment is relatively cheap, although more expensive gas analysers are available, and running costs are negligible.

The compromise for non-invasiveness of breath-sampling is that concentrations of gasses in the sampled air are influenced by cow head position relative to the sampling tube. The use of head position sensors and data filtering algorithms can remove the effects when the cow's head is completely out of the feed bin, but not within the feed bin. Consequently, sniffer measurements are more variable than flux methods, with factors like variable air flow in the barn increasing measurement error (imprecision), and head position, a highly repeatable characteristic, inflating between-cow variability.

Using CO2 as a tracer gas partly addresses the issue but, because CO2 arises from metabolism as well as rumen fermentation, variability of CO2 emissions has to be considered. A further consideration is diurnal variation in breath concentrations of methane and CO2 because animals are spot-sampled at different times of day and night. Diurnal variation can be accounted for either by fitting a model derived from the whole group of animals, or by including time of measurement in the statistical model.

The number of observations per analyser is limited only by number of cows assigned to one automatic milking station or concentrate feeding station and length of time equipment is installed. Typically, each analyser will record 40 to 70 animals 2 to 7 times per day for 7 to 10 days, although the number of sampling stations per analyser can be increased by using an automatic switching system. Throughput per analyser is likely to be 2000 to 3000 animals per year. Estimates of heritability for methane production measured using this method range from h²=0.12 to 0.45 over multiple studies.

### GreenFeed

GreenFeed (C-Lock Inc., Rapid City, SD, USA) is a sophisticated sniffer system where breath samples are provided when animals visit a bait station. As with other sniffer systems, GreenFeed samples breath from individual animals several times per day for short periods (3 to 7 min). GreenFeed is a portable standalone system used in barn and pasture applications, and incorporates an extractor fan to ensure active airflow and head position sensing for representative breath sampling. Measurements are pre-processed by the manufacturer, and data are available in real time through a web-based data management system. As GreenFeed captures a high proportion of emitted air and measures airflow, which can be calibrated using a tracer gas, methane emission is estimated as a flux at each visit. Providing visits occur throughout the 24 h, methane emission can be estimated directly as g/day.

A limitation of the GreenFeed system is that animals require training to use the system, although animals which have been trained to use the system will readily use it again. However, some animals will not use the system or will use it infrequently, and frequency of visits is affected by diet. This can be a challenge when screening commercial herds for methane emission under genetic evaluation.

The manufacturer recommends 15 to 25 animals per GreenFeed unit, and recordings are made typically for 7 days. If all animals visit the unit adequately, throughput per unit is likely to be 750 to 1250 animals per year.

### Laser Methane Detector (LMD)

The laser methane detector (LMD) is a highly responsive, hand-held device that is pointed at an animal's nostrils and measures methane column density along the length of the laser beam (ppm·m). In the first implementation of LMD on a farm, measurements for each cow were taken over periods of 15 to 25 s between eructation events, and could detect methane emitted each time the animal breathed out. In a later study with sheep and beef cattle, monitoring periods of 2 to 4 min allowed authors to separate breathing cycles from eructation events. Typically, animals are restrained either manually or in head yokes at a feed fence for the required length of time. The operator has to stand at the same distance (1 to 3 m) from each animal every time and must be careful to keep the laser pointed at the animal's nostrils throughout the measurement period.

The LMD can be used in the animal's normal environment, although for consistency restraint is required during measurement. Because the LMD measures methane in the plume originating from the animal's nostrils, results can be a_ected by factors such as: distance from the animal; pointing angle; animal's head orientation and head movement; air movement and temperature in the barn; adjacent animals; and operator variation. Operator variation is likely to be one of the biggest factors, because the operator controls distance and pointing angle, and is responsible for ensuring that the laser remains on target. The structure of the barn and the resulting ventilation conditions and wind speed at the location of the measurement are also considerable sources of variation in recorded methane.

Assuming operator fatigue does not limit measurements, each LMD could record up to 10 animals per hour. If each animal is recorded 3 times (on 3 consecutive days, for example), throughput is likely to be up to 1000 animals per year.

Methods for Producing of Recombinant Protein and/or Antibodies

The terms“expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, gPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a cell which has been transfected, infected or transformed by a nucleic acid and/or a vector according to the invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids may be used to produce a recombinant polypeptide of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing an antibody or a polypeptide of the invention according to the invention, said method comprising the steps consisting of (i) introducing in vitro or ex vivo a recombinant nucleic acid or a vector as described herein into a competent host cell, (ii) culturing in vitro or ex vivo the recombinant host cell obtained and (iii), optionally, selecting the cells which express and/or secrete said antibody or polypeptide. Such recombinant host cells can be used for the production of antibodies and polypeptides of the invention.

Antibodies and fragments thereof, immunoglobulins, and polypeptides of the present invention may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides of the present invention can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

In particular, the present invention further relates to a method of producing an antibody or a polypeptide of the invention, which method comprises the steps consisting of: (i) culturing a transformed host cell according to the invention under conditions suitable to allow expression of said antibody or polypeptide; and (ii) recovering the expressed antibody or polypeptide.

Antibodies and other polypeptides of the present invention are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-ruminant chimeras, or one ruminant-another ruminant (e.g., goat-cow) chimeras) of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class. Chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, methods for producing antibody fragments are well-known. For example, Fab fragments of the present invention can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′)2 fragments of the present invention can be obtained treating an antibody which specifically reacts with a ganglioside with a protease, pepsin. Also, the F(ab′)2 fragment can be produced by binding Fab′ described below via a thioether bond or a disulfide bond.

Fab′ fragments of the present invention can be obtained treating F(ab′)2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv.

### Kits

The present invention also encompasses kits. For example, the kit can comprise a vaccine of the present disclosure, any one of pharmaceutical compositions described herein, at least one additional agent that reduces methane production in a ruminant described herein, or any combination thereof, packaged in a suitable container and can further comprise instructions for using such reagents. The kit may also contain other components, such as administration tools packaged in the same or separate container.

### Exemplary Embodiments

1. A vaccine comprising at least one nucleic acid encoding at least one cell surface protein or a fragment thereof (e.g., an antigenic fragment, e.g., a fragment comprising an epitope, e.g., a fragment comprising an extracellular domain or a portion thereof) of at least one methanogen.

2. The vaccine of 1, wherein the at least one methanogen is of a family Methanobacteriaceae.

3. The vaccine of 1 or 2, wherein the at least one methanogen is of a genus selected from: Methanobrevibacter, Methanosphaera, Methanomicrobium, Methanobacterium, Methanocorpusulum, Methanosaeta, Methanoculleus, Methanosarcina, and Thermoplasmatales, optionally Methanobrevibacter, Methanomicrobium, and Methanosarcina.

4. The vaccine of any one of 1-3, wherein the at least one methanogen comprises Methanobacterium formicicum, Methanobacterium bryantii, Methanobrevibacter ruminantium, Methanobrevibacter millerae, Methanobrevibacter olleyae, Methanomicrobium mobile, Methanoculleus olentangyi, Methanosarcina barkeri, Methanobrevibacter boviskoreani, Methanobacterium beijingense, Methanoculleus marisnigri, Methanoculleus bourgensis, Methanosarcina mazei, Thermoplasmatales archaeon BRNA1, Methanobrevibacter gottschalkii, Methanobrevibacter thaueri, Methanobrevibacter smithii, Methanosphaera stadtmanae, Methanococcoides burtonii, Methanolobus psychrophilus R15, Methanobacterium paludism, Methanohalobium evestigatum, Methanomethylovorans hollandica, Methanothrix soehngenii, Methanocaldococcus vulcanius, Methanosalsum zhilinae, Methanocorpusculum labreanum, Methanoregula formicica, Methanoculleus marisnigri, Methanocella arvoryzae, Methanoculleus bourgensis, Methanolacinia petrolearia, Methanospirillum hungatei, Methanoplanus limicola, Methanohalophilus mahii, Methanococcus aeolicus, Methanosphaerula palustris, Methanocaldococcus fervens, Methanocaldococcus jannaschii, Methanocaldococcus sp. FS406-22, Methanoregula boonei, Methanobrevibacter sp. AbM4, Methanobrevibacter ruminantium, Methanosphaera, Methanobacterium formicicum, Methanocaldococcus villosus, Methanosarcina barkeri, Methanobacterium lacus, Methanotorris igneus, Methanotorris formicicus, Methanocaldococcus infernus, Methanofollis liminatans, Methanothermococcus okinawensis, Methanobrevibacter smithii, Methanobrevibacter, Methanocella conradii, Methanothermococcus thermolithotrophicus, Methanococcus maripaludis, Methanococcus maripaludis, Methanococcus vannielii, Methanothermus fervidus, Methanosarcina acetivorans, Methanosarcina mazei, Methanosaeta harundinacea 6Ac, Methanococcus maripaludis, Methanococcus voltae, Methanolinea tarda, Methanolobus psychrophilus, Methanosaeta harundinacea, or any combination thereof.

5. The vaccine of any one of 1-4, wherein the at least one methanogen comprises Methanobrevibacter ruminantium and/or Methanobrevibacter gottschalkii.

6. The vaccine of any one of 1-5, wherein the vaccine is monovalent.

7. The vaccine of any one of 1-5, wherein the vaccine is multivalent, optionally wherein the vaccine comprises at least one nucleic acid encoding at least one cell surface protein or a fragment thereof of Methanobrevibacter ruminantium and Methanobrevibacter gottschalkii.

8. The vaccine of 7, wherein the vaccine comprises at least one nucleic acid encoding at least 2, 3, 4, 5, 6, 7, 8, 9 or 10 polypeptides (e.g., of one cell surface protein or of multiple cell surface proteins; or fragments thereof).

9. The vaccine of 7 or 8, wherein the vaccine comprises at least one nucleic acid encoding at least 15, 20, 30, 40, 50, or 100 polypeptides (e.g., of one cell surface protein or of multiple cell surface proteins; or fragments thereof).

10. The vaccine of 8 or 9, wherein the polypeptides are of at least 2, 3, 4, 5, 6, 7, 8, 9 or 10 methanogens.

11. The vaccine any one of 1-10, wherein at least one nucleic acid encodes a concatemeric polypeptide.

12. The vaccine of any one of 1-11, wherein the at least one cell surface protein or a fragment thereof comprises an adhesin-like protein, adhesin-like protein with cysteine protease domain, tetrahydromethanopterin S-methyltransferase subunit, a fragment thereof, and/or any combination thereof.

13. The vaccine of any one of 1-12, wherein the at least one cell surface protein or a fragment thereof comprises at least 90%, 91%, 92%, 93%, 94%, 95%, 96%, 97% 98%, 99%, or 100% sequence identity to the amino acid sequence set forth in Table 2A, Table 2B, Table 3, Table 19, Table 20, Table 21, or a fragment thereof.

14. The vaccine of any one of 1-13, wherein the at least one nucleic acid comprises at least 90%, 91%, 92%, 93%, 94%, 95%, 96%, 97% 98%, 99%, or 100% sequence identity to the nucleotide sequence set forth in Table 2A, Table 2B, Table 3, Table 19, Table 20, Table 21, or a fragment thereof.

15. The vaccine of any one of 1-14, wherein the at least one nucleic acid is codon-optimized for expression in a ruminant, optionally codon-optimized for expression in Bos taurus.

16. The vaccine of any one of 1-15, wherein the at least one nucleic acid is a deoxyribonucleic acid (DNA) or a ribonucleic acid (RNA).

17. The vaccine of any one of 1-16, wherein the at least one nucleic acid is DNA.

18. The vaccine of 17, wherein the DNA comprises coding and non-coding sequences.

19. The vaccine of 17 or 18, wherein the DNA is operably linked to a promoter.

20. The vaccine of 19, wherein the promoter is selected form CMV promoter, CAG promoter, SCP promoter, CMVe-SCP, CMVmax, JET, PGK, EF-1a, AHSP promoter, MND promoter, Wiskott-Aldrich promoter, and PKLR promoter.

21. The vaccine of any one of 17-20, wherein the DNA comprises:
(a) a transcription regulatory element (e.g., an enhancer, a transcription termination sequence, a proximal promoter element, a locus control region); and/or
(b) a translation regulatory element (e.g., Kozak sequence, an untranslated region (5′ UTR or 3′ UTR), a polyadenylation signal sequence).

22. The vaccine of any one of 17-21, wherein the DNA further comprises
(a) a sequence encoding a signal peptide (e.g., Table 5A and Table 5C); and/or
(b) a sequence encoding a transmembrane domain, optionally further comprising a cytoplasmic domain (e.g., Table 5B and Table 5C).

23. The vaccine of any one of 17-22, wherein the DNA is linear or circular.

24. The vaccine of any one of 17-23, wherein the DNA is double-stranded or single-stranded.

25. The vaccine of any one of 17-24, wherein the DNA is single-stranded and comprises at least one hairpin.

26. The vaccine of any one of 17-24, wherein the DNA is double-stranded and comprises a telomeric sequence (e.g., a closed linear DNA, e.g., dbDNA™)

27. The vaccine of any one of 17-26, wherein the DNA comprises at least one chemical modification.

28. The vaccine of 27, wherein the at least one chemical modification is a terminal modification, which is present at 5′ end and/or 3′ end.

29. The vaccine of 27 or 28, wherein the at least one chemical modification comprises phosphorothioate, triethylene glycol (TEG), Locked Nucleic Acid (LNA, a 2′-oxygen-4′-carbon methylene linkage), hexaethylene glycol (Sp18), 1,3-propanediol (SpC3), 2′-O-methoxyethyl (MOE) ribonucleotides, 2′-O-methyl ribonucleotides (2′-Ome), 2′-fluoro (2′-F) nucleotides, or any combination thereof.

30. The vaccine of 29, wherein the at least one chemical modification comprises at least five consecutive phosphorothioate bonds.

31. The vaccine of 29 or 30, wherein the at least one chemical modification comprises at least three consecutive 2′-O-methyl nucleosides and/or 2′-O-methoxyethyl nucleosides.

32. The vaccine of any one of 17-31, wherein the DNA is in a vector.

33. The vaccine of 32, wherein the vector is a plasmid.

34. The vaccine of any one of 17-33, wherein the DNA is packaged in a virus, e.g., AAV, e.g., bovine AAV (e.g., for transduction to a subject).

35. The vaccine of any one of 1-16, wherein the at least one nucleic acid is RNA (e.g., mRNA).

36. The vaccine of 35, wherein the RNA comprises a 5′ cap.

37. The vaccine of 35 or 36, wherein the RNA comprises the 5′ cap with at least one chemical modification.

38. The vaccine of any one of 35-37, wherein the 5′ terminal cap is selected from:
(a) (Cap 0) m7G(5′)pppN1pN2p;
(b) (Cap 1) m7G(5′)pppN1mpNp;
© (Cap 2) m7G(5′)pppN1mpN2mp;
(d) (Cap 4) m7Gpppm(3)(6,6,2′)Apm(2′)Apm(2′)Cpm(2)(3,2′)Up; and
© Anti-reverse Cap Analog (ARCA).

39. The vaccine of any one of 35-38, wherein the RNA comprises 5′ UTR.

40. The vaccine of 39, wherein the 5′ UTR comprises a secondary structure.

41. The vaccine of 39, wherein the 5′ UTR does not comprise a secondary structure.

42. The vaccine of any one of 39-41, wherein the 5′ UTR comprises a Kozak sequence and/or at least one translational enhancer element (TEE).

43. The vaccine of 42, wherein the TEE comprises internal ribosome entry site (IRES).

44. The vaccine of any one of 39-43, wherein the 5′ UTR comprises a nucleic acid sequence that is at least 60%, 65%, 70%, 75%, 80%, 85%, 90%, 95%, or 100% identical to:
(a) the 5′ UTR or a fragment thereof of a gene that is highly expressed in muscle (e.g., MyoD, Myosin, Myoglobin, Myogenin, Herculin), endothelial cells (e.g., Tie-1, CD36), or lung epithelial cells (e.g., SP-A/B/C/D); and/or
(b) any one of the 5′ UTRs or a fragment thereof listed in Table 4A.

45. The vaccine of any one of 35-44, wherein the RNA comprises a 3′ UTR.

46. The vaccine of 45, wherein the 3′ UTR comprises a secondary structure.

47. The vaccine of 45, wherein the 3′ UTR does not comprise a secondary structure.

48. The vaccine of any one of 45-47, wherein the 3′ UTR comprises at least one translational enhancer element (TEE) and/or a stem loop (e.g., a histone stem loop).

49. The vaccine of any one of 45-48, wherein the 3′ UTR comprises a nucleic acid sequence that is at least 60%, 65%, 70%, 75%, 80%, 85%, 90%, 95%, or 100% identical to any one of the 3′ UTRs listed in Table 4B.

50. The vaccine of any one of 35-49, wherein the RNA comprises a polyA tail or a polyadenylation signal.

51. The vaccine of 50, wherein the polyA tail is at least 50, 60, 70, 80, 90, 100, 110, 120, 130, 140, or 150 bps long.

52. The vaccine of 50 or 51, wherein the polyA tail is about 100-150 bps long.

53. The vaccine of any one of 50-52, wherein the polyA tail comprises a linker (e.g., that disrupts the polyA tail).

54. The vaccine of 53, wherein the linker comprises the sequence UGC or a plurality thereof (e.g., multiple copies of the UGC linker).

55. The vaccine of any one of 35-54, wherein the RNA comprises a sequence encoding a signal peptide, optionally wherein the sequence encodes a signal peptide selected from the signal peptides listed in Table 5A and Table 5C.

56. The vaccine of any one of 35-55, wherein the RNA comprises a sequence encoding a transmembrane © domain and/or a sequence encoding cytoplasmic domain, optionally wherein the sequence encodes a transmembrane domain and/or a cytoplasmic domain selected from those listed in Table 5B and Table 5C.

57. The vaccine of any one of 35-56, wherein the RNA comprises:
(a) a 5′ cap, a 5′ untranslated region (UTR), a sequence encoding at least one cell surface protein or a fragment thereof of at least one methanogen, a 3′ UTR, and a polyA tail;
(b) a 5′ cap, a 5′ UTR, a sequence encoding a signal peptide, a sequence encoding at least one cell surface protein or a fragment thereof of at least one methanogen, a 3′ UTR, and a poly A tail; or
© a 5′ cap, a 5′ UTR, a sequence encoding a signal peptide, a sequence encoding at least one cell surface protein or a fragment thereof of at least one methanogen, a sequence encoding a transmembrane domain, optionally further comprising a sequence encoding a cytoplasmic domain, 3′ UTR, and a poly A tail.

58. The vaccine of any one of 35-57, wherein the at least one cell surface protein or a fragment thereof of at least one methanogen is expressed as a cytoplasmic protein, as a secreted protein, or as a membrane-tethered protein.

59. The vaccine of any one of 35-58, wherein the RNA comprises at least one chemical modification.

60. The vaccine of 59, wherein the chemical modification is in the sequence encoding at least one cell surface protein or a fragment thereof of at least one methanogen.

61. The vaccine of any one of 35-60, wherein the RNA comprises at least one chemical modification selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and any combination thereof.

62. The vaccine of any one of 35-61, wherein the RNA comprises a chemical modification in at least 75%, 80%, 85%, 90%, 95%, 99%, or 100% of the uracils in the sequence encoding at least one cell surface protein or a fragment thereof of at least one methanogen.

63. The vaccine of 62, wherein the chemical modification is in the 5-position of the uracil.

64. The vaccine of 62 or 63, wherein the at least one chemical modification is selected from the group consisting of pseudouridine, N1-methyl pseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methxyuridine, and 2′-O-methyl uridine.

65. The vaccine of any one of 62-64, wherein the chemical modification comprises N1-methyl pseudouridine.

66. A pharmaceutical composition comprising the vaccine of any one of 1-65, and at least one carrier and/or at least one excipient.

67. The pharmaceutical composition of 66, wherein the vaccine is formulated in lipid or saline.

68. The pharmaceutical composition of 66 or 67, wherein the vaccine is formulated in lipid.

69. The pharmaceutical composition of 67 or 68, wherein the nucleic acid (e.g., DNA or RNA) to total lipid ratio is at least about 0.05 (wt/wt).

70. The pharmaceutical composition of any one of 67-69, wherein the vaccine is formulated in a liposome, a lipoplex, or a lipid nanoparticle.

71. The pharmaceutical composition of 70, wherein the vaccine is formulated in a lipid nanoparticle.

72. The pharmaceutical composition of 70 or 71, wherein the vaccine is formulated in a lipid nanoparticle comprising an ionizable lipid, a helper lipid, a PEGylated lipid, a structural lipid (e.g., sterol), or any combination thereof.

73. The pharmaceutical composition of any one of 70-72, wherein the lipid nanoparticle comprises an ionizable lipid, a helper lipid, a PEGylated lipid, and a structural lipid (e.g., sterol).

74. The pharmaceutical composition of 72 or 73, wherein:
(a) the ionizable lipid is an ionizable cationic lipid; and/or
(b) the helper lipid is a neutral lipid.

75. The pharmaceutical composition of any one of 70-74, wherein the lipid nanoparticle has a molar ratio of about 20-60% ionizable lipid: about 5-25% helper lipid: about 25-55% structural lipid; and about 0.5-15% PEGylated lipid.

76. The pharmaceutical composition of any one of 70-75, wherein the lipid nanoparticle has a molar ratio of about 50% ionizable lipid: about 10% helper lipid: about 38.5% structural lipid; and about 1.5% PEGylated lipid.

77. The pharmaceutical composition of any one of 72-76, wherein the ionizable lipid comprises 2,2-dilinoleyl-4-dimethylaminoethyl-[1,3]-dioxolane (Dlin-KC2-DMA), dilinoleyl-methyl-4-dimethylaminobutyrate (Dlin-MC3-DMA), di((Z)-non-2-en-1-yl) 9-((4-(dimethylamino)butanoyl)oxy)heptadecanedioate (L319), 4-hydroxybutyl)azanediyl)bis(hexane-6,1-diyl)bis(2-hexyldecanoate (ALC-0315), SM-102, A18-Iso5-2DC18, A6, 3060i10, or any combination thereof.

78. The pharmaceutical composition of any one of 72-77, wherein the PEGylated lipid comprises (2-[(polyethylene glycol)-2000]-N,N-ditetradecylacetamide (PEG2000-DMA (also called ALC-0159)); and/or polyethylene glycol 2000 dimyristoyl glycerol (PEG2000-DMG).

79. The pharmaceutical composition of any one of 72-78, wherein the helper lipid comprises 1,2-distearoyl-sn-glycero-3-phosphocholine (DSPC) and/or 1,2-dioleoyl-sn-glycero-3-phosphoethanolamine (DOPE).

80. The pharmaceutical composition of any one of 72-79, wherein the structural lipid comprises: cholesterol, β-Sitosterol, 20α-Hydroxycholesterol, sterol, or any combination thereof.

81. The pharmaceutical composition of any one of 70-80, wherein the lipid nanoparticle comprises SM-102, polyethylene glycol 2000 dimyristoyl glycerol (PEG2000-DMG), 1,2-distearoyl-sn-glycero-3-phosphocholine (DSPC), and cholesterol.

82. The pharmaceutical composition of any one of 70-80, wherein the lipid nanoparticle comprises ((4-hydroxybutyl)azanediyl)bis(hexane-6,1-diyl)bis(2-hexyldecanoate, 2-(polyethylene glycol 2000)-N,N-ditetradecylacetamide (PEG2000-DMA), 1,2-distearoyl-sn-glycero-3-phosphocholine (DSPC), and cholesterol

83. The pharmaceutical composition of any one of 66-82, further comprising potassium chloride, monobasic potassium phosphate, sodium chloride, dibasic sodium phosphate dihydrate, sucrose, or any combination thereof.

84. The pharmaceutical composition of any one of 66-82, further comprising tromethamine, tromethamine hydrochloride, sucrose, or any combination thereof.

85. The pharmaceutical composition of any one of 70-84, wherein the nanoparticle has:
(a) a mean diameter of about 50 nm to about 200 nm, optionally about 80 nm to about 100 nm;
(b) a polydispersity index (PDI) of less than 0.4; and/or
© a net neutral charge at a neutral pH.

86. The pharmaceutical composition of any one of 66-85, further comprising an adjuvant.

87. The pharmaceutical composition of 86, wherein the adjuvant comprises saponin, Montanide ISA61, a chitosan thermogel, a lipid nanoparticle/cationic liposome adjuvant, or any combination thereof.

88. The pharmaceutical composition of 86 or 87, wherein the adjuvant comprises Montanide ISA61.

89. The pharmaceutical composition of any one of 66-88, further comprising a transfection facilitating compound.

90. The pharmaceutical composition of 89, wherein the transfection facilitating compound comprises (±)-N-(2-hydroxyethyl)-N,N-dimethyl-2,3-bis(tetradecyloxy)-1-propanaminium bromide) (DMRIE).

91. A method of inducing an immune response against at least one methanogen in a ruminant, the method comprising administering to the ruminant the vaccine of any one of 1-62, or the pharmaceutical composition of any one of 66-90.

92. The method of 91, wherein the immune response comprises a B cell response and/or a T cell response.

93. A method of reducing the activity, number, and/or type of methanogens in the gut of a ruminant, the method comprising administering to the ruminant the vaccine of any one of 1-65, or the pharmaceutical composition of any one of 66-90.

94. A method of reducing the amount of methane produced by a ruminant, the method comprising administering to the ruminant the vaccine of any one of 1-65, or the pharmaceutical composition of any one of 66-93.

95. The method of any one of 91-94, wherein the ruminant produces an antibody against at least one methanogen.

96. The method of 95, wherein the antibody is an IgG or an IgA, preferably an IgA.

97. The method of 95 or 96, wherein the antibody is produced in an amount sufficient to:
(a) carry the antibody to the gut;
(b) reduce the number and/or type of methanogens in the gut; and/or
© reduce the amount of methane produced by the ruminant.

98. The method of any one of 91-97, wherein the method reduces the methane production by the ruminant by at least about 1%, 5%, 10%, 20%, 30%, 40%, 50%, 60%, 70%, 80%, 90%, or 95% as compared with a control.

99. The method of 98, wherein the control is an accepted reference, or the amount of methane production in a ruminant that has not been vaccinated.

100. The method of any one of 91-99, wherein the vaccine or the pharmaceutical composition is administered to the ruminant via a route selected from intramuscular administration, intradermal administration, subcutaneous administration, and nasal administration.

101. The method of any one of 91-100, wherein the ruminant is administered with at least one dose of the vaccine or pharmaceutical composition.

102. The method of any one of 91-101, wherein the ruminant is administered with at least one or two repeat doses of the vaccine or pharmaceutical composition (e.g., booster dose).

103. The method of 102, wherein the ruminant is administered with the repeat dose of the vaccine or pharmaceutical composition after at least about 2 weeks, 1 month, 6 months, or 12 months from the time the ruminant is administered with the preceding dose of the vaccine.

104. The method of 102 or 103, wherein the ruminant is administered with the repeat dose of the vaccine or pharmaceutical composition no more than about 3 months, 6 months, 12 months, or 24 months from the time the ruminant is administered with the preceding dose of the vaccine.

105. The method of any one of 102-104, wherein the ruminant receives the repeat dose of the vaccine after at least about 2 weeks and no more than about 18 months from the time the ruminant is administered with the preceding dose of the vaccine.

106. The method of any one of 102-105, wherein the ruminant receives a repeat dose of the vaccine after at least about 4 weeks and no more than about 12 months from the time the ruminant is administered with the preceding dose of the vaccine.

107. The method of any one of 91-106, wherein the ruminant is administered with a dosage of between 1 ug/kg and 400 ug/kg of the nucleic acid vaccine.

108. The method of any one of 91-107, wherein the ruminant is administered with a dosage of the nucleic acid comprising at least about 25 ug, 50 ug, 100 ug, 150 ug, 200 ug, 250 ug, or 300 ug of the RNA.

109. The method of any one of 91-108, wherein the ruminant is administered with a dosage of the nucleic acid comprising at least about 100 ug.

110. The method of any one of 91-109, wherein the ruminant is administered with a dosage of the nucleic acid comprising at least about 200 ug.

111. The method of any one of 91-110, wherein the ruminant is administered with a dosage of the nucleic acid comprising at least about 300 ug.

112. The method of any one of 91-111, further comprising administering to the ruminant at least one agent that reduces the level of methane produced by the ruminant.

113. The method of 112, wherein the at least one agent is administered to a ruminant concomitant with, prior to, or after the vaccination.

114. The method of 112 or 113, wherein the at least one agent is administered to a ruminant after the vaccination.

115. The method of any one of 112-114, wherein the at least one agent is administered to a ruminant daily, semiweekly, weekly, biweekly (every two weeks), or monthly.

116. The method of any one of 112-115, wherein the at least one agent is administered to a ruminant for a duration of at least 1 week but no more than 1 month.

117. The method of any one of 112-116, wherein the at least one agent is selected from:
(a) the agents listed in Table 8B; and/or
(b) 3-Nitrooxypropanol (3-NOP), ethyl-3NOP, 2-bromoethanesulfonate (BES), 2-chloroethanesulfonate (CES), 3-bromopropanesulfonate (BPS), bromochloromethane (BCM), bromoform, bromodichloromethane, dibromochloromethane, carbon tetrachloride, trichloroacetamide, trichloroethyladipate, lumazin (2,4-pteridinedione), p-aminobenzoic acid, lovastatin, mevastatin, pravastatin, diallyl disulfide, garlic oil, saponins, tannins, flavonoids, nitrate, nitroethane, -nitro-propionate, 2-nitropropanol, 2-nitroethanol, malate, acrylate, oxaloacetate, fumarate, propynoic acid, 3-butenoic acid, 2-butynoic acid, ethyl 2-butynoate, monensin, lasalocid, bovicin HC5, nisin, and any combination thereof.

118. The method of any one of 112-117, wherein the at least one agent is 3-NOP or ethyl-3NOP.

119. The method of 118, wherein the ruminant is administered with at least about 0.5 g but no more than 25 g of 3-NOP per day.

120. The method of 118 or 119, wherein the ruminant is administered with at least about 1 g but no more than 5 g of 3-NOP per day.

121. The method of any one of 118-120, wherein the ruminant is administered with about 2.5 g of 3-NOP per day.

122. The method of any one of 118-121, wherein the ruminant is administered with 3-NOP for a duration of at least 1 week but no more than 1 month.

123. The method of any one of 112-122, wherein the at least one agent is formulated in animal feed.

124. The method of 112, wherein the at least one agent is a composition comprising one or more small molecules that reduce the production of one or more deleterious atmospheric gases and/or precursors thereof and one or more agriculturally suitable carriers.

125. The method of 124, wherein the one or more agriculturally suitable carriers comprises a solid carrier.

126. The method of 125, wherein the one or more solid carriers comprises attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

127. The method of 125, wherein the one or more solid carriers comprises attapulgite, kaolinite, fuller's earth, calcium carbonate, perlite, diatomaceous earth, calcium silicate, fly ash, a polysaccharide, a disaccharide, a monosaccharide, a gum, silica, propylene glycol, hemp protein, biochar, montmorillonite, activated charcoal, lignin, wood flour, hemp protein, pea protein, soy protein, gelatin, casein, chotsan, talc, calcium phosphate, arginine, lysine, calcium carbonate, carbon black, glutamine, betaine, bismuth phosphate, bismuth citrate, iron phosphate, or any combination thereof.

128. The method of any one of 125-127, wherein the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, or a combination thereof.

129. The method of any one of 125-127, wherein the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, carrageenan, cellulose acetate, hydroxypropyl cellulose, cellulose acteate phthalate, maltrodextran, dextran, inulin, corn starch, amylopectin, sodium startch glycolate, pentaerthritol, cyclodextrin, or a combination thereof.

130. The method of any one of 124-129, wherein the solid carrier comprises silica and ethylcellulose.

131. The method of 130, wherein the carrier comprises about 10% to about 50% by weight of the silica and about 50 to about 90% by weight of the ethylcelluose.

132. The method of any one of 124-129, wherein the carrier comprises silica and activated charcoal.

133. The method of any one of 124-129, wherein the carrier comprises about 10% to about 90% by weight of the silica and about 10% to about 90% by weight of the activated charcoal.

134. The method of any one of 133, wherein the carrier further comprises arginine, lysine, or both arginine and lysine.

135. The method of any one of 124-129, wherein the carrier comprises activated charcoal and ethylcellulose.

136. The method of 135, wherein the carrier comprises about 10% to about 50% by weight of the activated charcoal and about 40 to about 90% by weight of the ethylcellulose.

137. The method of 135 or 136, wherein the carrier further comprises about 1 to about 10% by weight of sodium lignosulfate or about 1 to about 10% by weight of hydroxyethyl cellulose.

138. The method of any one of 124-129, wherein the carrier comprises arginine and polycaprolactone.

139. The method of 138, wherein the carrier comprises about 10 to about 60% by weight of the arginine and about 30 to about 90% by weight of the polycaprolactone.

140. The method of any one of 124-129, wherein the carrier comprises silica and polycaprolactone.

141. The method of 139, wherein the carrier comprises about 10 to about 60% by weight of the silica and about 30 to about 90% by weight of the polycaprolactone.

142. The method of any one of 125-141, wherein the one or more solid carriers is inert.

143. The method of any one of 125-142, wherein the one or more solid carriers is water soluble.

144. The method of any one of 124-143, further comprising one or more additives with a density greater than water and/or one or more additives that reduces the rate of dissolution of the composition in water.

145. The method of 144, wherein the composition has a density of at least 1.1, preferably about 1.1 mg/mL to about 3 mg/mL, about 1.5 to about 3 mg/mL, about 1.5 to about 2.5 mg/mL, or about 1.5 to about 2 mg/mL.

146. The method of 144 or 145, wherein the one or more additives with a density greater than water comprises silica, attapulgite, kaolinite, fuller's earth, calcium carbonate, perlite, diatomaceous earth, calcium silicate, fly ash, or any combination thereof.

147. The method of any one of 144-146, wherein the one or more additives that reduces the rate of dissolution of the composition further reduces a rate of release of the one or more small molecules into water.

148. The method of 147, wherein the composition dissolves over at least about 1, 2, 3, 4, 5, 6, 7, 14, 21, 28, 35, 42, 49, 56, 63, 70, 77, 84, 91, 98, 105, 112, 119, 126, 133, 140, and/or nor more than about 2, 3, 4, 5, 6, 7, 14, 21, 28, 35, 42, 49, 56, 63, 70, 77, 84, 91, 98, 105, 112, 119, 126, 133, 140, or 147 days, about 1 to about 147 days, more preferably 7-63 days, more preferably about 7-42 days, even more preferably 14-42 days yet even more preferably 14-28 days.

149. The method of any one of 124-148, wherein about 40 to about 80% of the small molecules that reduce the production of one or more deleterious atmospheric gases and/or precursors is thereof is released in water after 15 days.

150. The method of any one of 124-149, wherein the composition comprises particles having a uniform size distribution.

151. The method of any one of 124-150, wherein the composition comprises particles having a non-uniform size distribution.

152. The method of 150 or 151, wherein the particles comprise a spherical-, square-, rectangular-, capsular-, cylindrical-, conical-, ovular-, triangular-, diamond-, or disk-like shape.

153. The method of any one of 124-152, wherein the particles have a size ranging from about 1 mm to about 20 mm, about 1 to about 15 mm, about 1 to about 10 mm, about 5 to about 20 mm, about 5 to about 15 mm, or about 5 to about 10 mm.

154. The method of any one of 124-153, wherein the particles further comprises a coating.

155. The method of 154, wherein the coating comprises at least two layers.

156. The method of 154 or 155, wherein the coating is selected from cellulose acetate phlalate, ethyl cellulose, hydroxypropyl cellulose, polycaprolactone, alginate, chitosan, polyethylene glycol, cellulose actate, triacetin, propylene glycol, n-methyl-2-pyrollidone, and any combination thereof.

157. The method of 154 or 155, wherein the coating comprises two or more polyelectrolytes.

158. The method of 157, wherein the polyelelctrolytes comprise polystyrene sulfonate, polyethyleneimine, sodium lignosulfate, polyglutamic acid and poly-L-lysine, poly-L-arginine, polyallylamine hydrochloride, polyacrylic acid, or any combination thereof.

159. The method of 158, wherein the polyelectrolytes comprise polyallylamine hydrochloride and sodium lignosulfate.

160. The method of 158, wherein the polyelectrolytes comprise polyallylamine hydrochloride and polystyrene suylfonate.

161. The method of 158, wherein the polyelectrolytes comprise sodium lignosulfate and one of polyglutamic acid and poly-L-lysine, or poly-L-arginine, and sodium lignosulfate.

162. The method of 158, wherein the polyelectrolytes comprise polystyrene sulfonate and one of polyglutamic acid and poly-L-lysine, or poly-L-arginine.

163. The method of any one of 157-162, wherein the two or more polyelectrolytes are crosslinked.

164. The method of any one of 124-163, wherein the one or more small molecules comprise a molecule that interferes with the uptake and/or conversion of acetate, H₂, CO₂, methanol, monomethylamine, dimethylamine, trimethylamine, nitric oxide, or a combination thereof.

165. The method of any one of 124-164, wherein the one or more small molecules that reduce the production of one or more deleterious atmospheric gases and/or precursors interact with an enzyme selected from the group consisting of 3-(methylthio)propanoate:coenzyme M methyltransferase, acetate kinase, acetyl-CoA decarbonylase, acetyl-CoA decarbonylase/synthase complex α₂ε₂, acetyl-CoA decarbonylase/synthase complex 3, acetyl-CoA decarbonylase/synthase complex γδ, acetyl-CoA synthase, carbon monoxide dehydrogenase, carbonic anhydrase, Co-methyltransferase, coenzyme M reductase, cyclohydrolase, dehydrogenase, dimethylamine-[corrinoid protein] Co-methyltransferase, F₄₂₀-dependent methylene-H₄MPT reductase, F₄₂₀-dependent methylene-H₄SPT dehydrogenase, formylmethanofuran dehydrogenase, formylmethanofuran:H₄MPT formyltransferase, formylmethanofuran:H₄SPT formyltransferase, formyltransferase, H₂-forming methylene-H₄MPT dehydrogenase, methanol-5-hydroxybenzimidazolylcobamide Co-methyltransferase, methenyl-H₄MPT cyclohydrolase, methyl-coenzyme M reductase, methyl-H₄SPT:CoM methyltransferase, methylated [methylamine-specific corrinoid protein]:coenzyme M methyltransferase, methylcobamide:CoM methyltransferase, methylthiol:coenzyme M methyltransferase, methyltransferase, MtaC protein:coenzyme M methyltransferase, phosphotransacetylase, tetrahydromethanopterin S-methyltransferase, tetramethylammonium methyltransferase, trimethylamine-corrinoid protein Co-methyltransferase, and any combination thereof.

166. The method of 165, wherein the one or more small molecules that reduce the production of one or more deleterious atmospheric gases and/or precursors interact with methyl-coenzyme M reductase (MCR).

167. The method of any one of 124-166, wherein the one or more small molecules that reduce the production of one or more deleterious atmospheric gases and/or precursors comprise a compound having the formula

R¹—[CH₂]ₙ—ONO₂ wherein
n is an integer from 1 to 15;
R¹ is selected from the group consisting of H, C₁-C₆alkyl, phenyl, —OH, —NH₂, —CN, —COH, —O(C═O)R³, —NHC(═O)R³, —SO₂NHR³, or —ONO₂, —SH and R³ is C₁-C₆alkyl, phenyl, pyridyl;
   with the proviso that when n is >3 the hydrocarbon chain may be interrupted by —O— or —NH—.

168. The method of 167, wherein the one or more small molecules that reduce the production of one or more deleterious atmospheric gases and/or precursors is selected from 3-nitrooxypropanol, 9-nitrooxynonanol, 5-nitrooxy pentanoic acid, 6-nitrooxy hexanoic acid, bis(2-hydroxyethyl)amine dinitrate, 1,4-bis-nitrooxybutane, 1,5-bis-nitrooxypentane, and any combination thereof.

169. The method of 167, wherein the one or more small molecules that reduce the production of one or more deleterious atmospheric gases and/or precursors comprises 3-nitrooxypropanol (3-NOP).

170. The method of any one of 124-169, wherein the composition comprises about 1 to about 25% by weight of the small molecule, about 5 to about 20% by weight of the small molecule, or about 5 to about 15% by weight of the small molecule.

171. The method of any one of 124-170, wherein the composition comprises a plurality of populations of particles, wherein each population or particles comprises a different formulation, a different shape, and/or a different size distribution.

172. The method of 171, wherein the plurality of populations of particles comprises a first population and a second population.

173. The method of 172, wherein the population of granular particles further comprises at least 1, 2, 3, 4, 5, 5, 6, 8, or 9 and/or no more than 4, 5, 6, 7, 8, 9, or 10 additional populations, for example 3-10 additional populations, preferably 3-7 additional populations, more preferably 3-5 additional populations.

174. The method of 172 or 173, wherein the first population comprises an immediate release formulation.

175. The method of any one of 172-174, wherein the second population comprises a delayed release formulation.

176. The method of any one of 173-175, wherein each additional population comprises a delayed release formulation, wherein each population dissolves and/or releases the one or more small molecules that reduce the production of one or more deleterious atmospheric gases and/or precursors at a different time than each of the other populations.

177. An antibody produced by the method of any one of 91-176, or a fragment thereof.

178. The antibody of 177, wherein the antibody is a monoclonal antibody.

179. The antibody of 177 or 178, wherein the antibody is an IgG or an IgA, preferably an IgA.

180. The antibody of any one of 177-179, wherein the antibody is lyophilized.

181. The antibody of any one of 177-180, wherein the antibody is in a pharmaceutical composition comprising at least one excipient and/or carrier.

182. Milk and/or a derivative thereof produced by the ruminant of any one of 91-176.

183. The milk and/or a derivative thereof of 182, wherein the milk and/or derivatives thereof comprises an antibody that binds at least one methanogen.

184. The milk and/or a derivative thereof of 182 or 183, wherein the milk and/or derivatives thereof is pasteurized and/or homogenized.

185. The milk and/or a derivative thereof of any one of 182-184, wherein the milk and/or derivatives thereof is lyophilized or evaporated to form dry milk powder (e.g., boiling at low pressure at low temperature).

186. The milk and/or a derivative thereof of any one of 182-185, further comprising at least one agent that reduces methane production in a ruminant, optionally wherein the at least one agent is selected from the agents in Table 8B.

187. An animal feed comprising:
(a) the antibody of any one of 182-181;
(b) at least one agent that reduces methane production in a ruminant, optionally wherein the at least one agent is selected from the agents in Table 8B;
© the milk of any one of 182-186; or
(d) any combination of two or more of (a)-(c).

188. The animal feed of 187, wherein the animal feed is liquid (e.g., drinking water, milk) or solid (e.g., fodder).

189. The animal feed of 187 or 188, wherein the animal feed comprises fat and/or fatty acid, optionally wherein the animal feed comprises fat and/or fatty acid that is at least about 1%, 2%, 3%, 4%, 5%, or 6% of the diet.

190. A method of reducing methane production in a ruminant, the method comprising orally administering to and/or feeding the ruminant the antibody of any one of 177-181, the milk and/or a derivative thereof of any one of 182-186, the animal feed of any one of 187-189, or any combination of two or more thereof.

191. The method of 190, further comprising administering at least one agent that reduces methane production in a ruminant, optionally wherein the at least one agent is selected from the agents in Table 8B.

192. The method of 190 or 191, further comprising administering the ruminant with at least one vaccine of any one of 1-65 or at least one pharmaceutical composition of any one of 66-90.

193. The method of any one of 91-176 and 190-192, wherein the ruminant is selected from a cow, a bull, a bison, a yak, a buffalo, an antelope, a goat, a sheep, a deer, a giraffe, a caribou, a gazelle, a macropod, a llama, a camel, and an alpaca.

194. The method of any one of 91-176 and 190-193, wherein the ruminant is an adult ruminant.

195. The method of any one of 91-176 and 190-193, wherein the ruminant is a young ruminant (e.g., before weaning or below 2 years of age).

196. The method of any one of 91-176 and 190-193, wherein the ruminant is a pregnant female ruminant.

197. The method of any one of 91-176 and 190-193, wherein the ruminant is an offspring (e.g., calf) of the vaccinated female ruminant that received the milk comprising an antibody that binds at least one methanogen.

198. The method of any one of 91-176 and 190-197, wherein the vaccine is administered to a ruminant as a part of a pre-existing vaccination program to which the ruminant is subject (e.g., Table 8A).

199. The method of any one of 91-176 and 190-198, wherein the vaccine is administered to a ruminant when the ruminant is subject to or receives at least one other vaccine, wherein the at least one other vaccine is against against infectious bovine rhinotracheitis (IBR), bovine virus diarrhea (BVD), parainfluenza-3 (PI3), bovine respiratory syncytial virus (BRSV), clostridia, E. Coli mastitis, leptospirosis, mannheimia hemolytica, brucella, vibriosis, campylobacter, trichomonas, trichomoniasis, rotavirus, coronavirus, and/or respiratory disease.

200. The method of any one of 91-176 and 190-197, wherein the vaccine is administered to a ruminant when the ruminant changes in hands and/or a changes in environment.

201a. The method of any one of 91-176 and 190-200, wherein the vaccine or the immune response it elicits is exposed to a methanogen in a lower bowel of the ruminant.

201b. The method of any one of 91-176 and 190-200, wherein the vaccine reduces methane production in the lower intestinal track (lower bowel) of the ruminant, optionally wherein the method results in at least about 1%, 2%, 3%, 4%, 5%, 6%, 7%, 8%, 9%, 10%, 11%, 12%, 13%, 14%, 15%, 16%, 17%, 18%, 19%, or 20% reduction in the level of methane produced by the ruminant, optionally wherein the reduction in the level of methane is compared to an untreated ruminant. In some embodiments, the ruminant methane emissions are measured using a respiratory chamber.

201c. A method of reducing the activity, number, and/or type of methanogens in the lower intestinal track of a ruminant, the method comprising administering to the ruminant the vaccine of any one of 1-65, or the pharmaceutical composition of any one of 66-90.
201d. A method of reducing the amount of methane produced by a ruminant in the lower intestinal track, the method comprising administering to the ruminant the vaccine of any one of 1-65, or the pharmaceutical composition of any one of 66-93.
201e. A method of inducing an immune response against at least one methanogen in the lower intestinal track of a ruminant, the method comprising administering to the ruminant the vaccine of any one of 1-62, or the pharmaceutical composition of any one of 66-90.

### EXAMPLES

### Example 1: DNA Vaccine Preparation

### Construction of Expression Vectors

Plasmid constructs comprising the coding regions encoding at least one cell surface protein or a fragment (e.g., extracellular domain) thereof of at least one methanogen (e.g., those listed in Table 2A, Table 2B, Table 3, Table 19, Table 20, or Table 21) are isolated from methanogen RNA by RT PCR, or prepared by direct synthesis since the sequence is known, by standard methods and are inserted into a vector via standard restriction sites, by standard methods. In this example, the DNA encoding an extracellular domain of an mtr polypeptide is cloned.

The codon-optimized coding regions are generated using the method described herein or the methods known in the art. The coding regions or codon optimized coding regions are constructed using standard PCR methods, or are ordered commercially. The coding regions or codon-optimized coding regions are inserted into the vector via standard restriction sites, by standard methods.

Plasmids constructed as above are propagated in Escherichia coli and purified by the alkaline lysis method (Sambrook, J., et al, Molecular Cloning: A Laboratory Manual, Cold Spring Harbor Laboratory Press, Cold Spring Harbor, NY, ed. 2 (1989)). CsCl-banded DNA are ethanol precipitated and resuspended in 0.9% saline to a final concentration of 2 mg/ml for injection. Alternately, plasmids are purified using any of a variety of commercial kits, or by other known procedures involving differential precipitation and/or chromatographic purification.

Expression is tested by formulating each of the plasmids in DMRIE/DOPE and transfecting ruminant cell lines, e.g., Bos Taurus cell lines CPAE (endothelial cell), EBTr (NBL-4) (trachea cell), FB2.K (kidney cell), LB9.Bm (lymph node cell), BT (nasal cell), FBHE (heart endothelial cell), CPA47 (pulmonary artery endothelial cell), BCE C/D-1b (eye endothelial cell), BL3.1 (B lymphocyte), EJG (adrenal gland endothelial cell), BL-3 (B lymphocyte), MDBK (NBL-1) (kidney cell), LB9.D (LB9.Sk) (skin cell), SBAC (fibroblast), and BEND (endometrium cell line), all of which are available commercially from ATCC.org. Appropriate culture media and conditions for the above-described host cells are known in the art.

The supernatants are collected and the protein production tested by Western blot or ELISA. The relative expression of the wild type and codon optimized constructs are compared.

A single plasmid may comprise a nucleic acid encoding a single cell surface protein or a fragment thereof.

Alternatively, more than two proteins or fragments may be placed in a single plasmid as a polycistronic construct. For example, a polycistronic construct, where two or more polypeptides or fragments are transcribed as a single transcript (under a single promoter) in cells may be constructed by separating the various coding regions with IRES sequences (Jang et al. J. Virol. 62: 2636-43 (1988); Jang et al. Enzyme 44:292-309(1990)).

Still alternatively, fragments of a single cell surface protein can be placed in multiple plasmids, which can then be co-formulated.

Alternatively, two or more coding regions may be inserted into a single plasmid, each with their own promoter sequence (i.e., not a polycistron).

### Preparation of Vaccine Formulations

Plasmid constructs comprising codon-optimized and non-codon-optimized coding regions encoding at least one cell surface protein of a methanogen or a fragment thereof are formulated with Montanide ISA61 with or without addition of immunomodulatory monophosphoryl lipid A (MPL). Montanide ISA61 (Seppic, France) was used at a ratio of 6.0/4.0 w/w adjuvant/DNA. MPL (Sigma-Aldrich, USA) was used at a concentration of 0.2 mg per dose.

### Example 2. RNA Vaccine Preparation

The methods of preparing RNA vaccine are well known in the art. In some cases, the methods described in WO2012/135805 (incorporated herein by reference) is used.

### DNA Construct

A DNA construct comprising the gene encoding an mtr or a fragment thereof is constructed using the standard methods. The open reading frame (ORF) encoding the extracellular domain of mtr may be flanked by a 5′ untranslated region (UTR) which may contain a strong Kozak translational initiation signal and/or an alpha-globin 3′ UTR which may include an oligo(dT) sequence for templated addition of a poly-A tail. The mRNAs may be modified to reduce the cellular innate immune response. The modifications to reduce the cellular response may include pseudoundine (W) and 5-methyl-cytidine (5meC or m⁵C). (see, Kariko K et al. Immunity 23: 165-75 (2005), Kariko K et al. Mol Ther 16: 1833-40 (2008), Anderson B R et al. NAR (2010); herein incorporated by reference).

The ORF may also include various upstream or downstream additions (such as, but not limited to, β-globin, tags, etc.) may be ordered from a service such as, but limited to, DNA2.0 (Menlo Park, CA) and may contain multiple cloning sites which may have Xbal recognition. Upon receipt of the construct, it may be reconstituted and transformed into chemically competent E. coli. In this example, NEB DH5-alpha Competent E. coli are used. Transformations are performed according to NEB instructions using 100 ng of plasmid. The protocol includes co-incubation of the DNA construct with the E. Coli, followed by heat shock at 42° C. for 30 seconds. Mix in SOC media, shake, then plate the transformed E. Coli. Incubate at 37° C.

To isolate the plasmid (up to 850 μg), a maxi prep is performed using the Invitrogen PURELIN™ HiPure Maxiprep Kit (Carlsbad, CA), following the manufacturer's instructions.

IVT mRNA Preparation

In order to generate cDNA for In Vitro Transcription (IVT), the plasmid is first linearized using a restriction enzyme such as Xbal. A typical restriction digest with Xbal will comprise the following: Plasmid 1.0 μg; 10× Buffer 1.0 μl Xbal 1.5 μl; water up to 10 μl; incubated at 37° C. for 1 hr. If performing at lab scale (<5 μg), the reaction is cleaned up using Invitrogen's PURELINK™ PCR Micro Kit (Carlsbad, CA) per manufacturer's instructions. Larger scale purifications may need to be done with a product that has a larger load capacity such as Invitrogen's standard PURELINK™ PCR Kit (Carlsbad, CA). Following the cleanup, the linearized vector is quantified using the NanoDrop and analyzed to confirm linearization using agarose gel electrophoresis.

The in vitro transcription reaction generates mRNA containing modified nucleotides or modified RNA. The input nucleotide triphosphate (NTP) mix is made in-house using natural and un-natural NTPs.

A typical in vitro transcription reaction includes the following:
1. Template cDNA 1.0 μg
2. 10× transcription buffer (400 mM Tris-HCl pH 8.0, 190 mM MgCl₂, 50 mM DTT, 10 mM Spermidine) 2.0 μl
3. Custom NTPs (25 mM each) 7.2 μl
4. RNase Inhibitor 20 U
5. T7 RNA polymerase 3000 U
6. dH₂O Up to 20.0 μl and
7. Incubation at 37° C. for 3 hr-5 hrs.

The crude IVT mix may be stored at 4° C. overnight for cleanup the next day. 1 u of RNase-free DNase is then used to digest the original template. After 15 minutes of incubation at 37° C., the mRNA is purified using Ambion's MEGACLEAR™ Kit (Austin, TX) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

Enzymatic Capping of mRNA

Capping of the mRNA is performed as follows where the mixture includes: IVT RNA 60 g-180 g and dH₂O up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM Cl, 12.5 mM MgCl₂) (10.0 μl); 20 mM GTP (5.0 l); 20 mM S-Adenosyl Methionine (2.5 μl); RNase Inhibitor (100 U); 2′-O-Methyltransferase (400U); Vaccinia capping enzyme (Guanylyl transferase) (40 U); dH₂O (Up to 28 μl); and incubation at 37° C. for 30 minutes for 60 g RNA or up to 2 hours for 180 μg of RNA.

The mRNA is then purified using Ambion's MEGACLEAR™ Kit (Austin, TX) following the manufacturer's instructions. Following the cleanup, the RNA is quantified using the NANODROP™ (ThermoFisher, Waltham, MA) and analyzed by agarose gel electrophoresis to confirm the RNA is the proper size and that no degradation of the RNA has occurred. The RNA product may also be sequenced by running a reverse-transcription-PCR to generate the cDNA for sequencing.

5′-capping of modified RNA may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-0 methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes are preferably derived from a recombinant source.

Cap structure can be analyzed for capping reaction efficiency by LC-MS after capped mRNA nuclease treatment. Nuclease treatment of capped mRNAs would yield a mixture of free nucleotides and the capped 5′-5-triphosphate cap structure detectable by LC-MS. The amount of capped product on the LC-MS spectra can be expressed as a percent of total mRNA from the reaction and would correspond to capping reaction efficiency. The cap structure with a higher capping reaction efficiency would have a higher amount of capped product by LC-MS.

### PolyA Tailing Reaction

Without a poly-T in the cDNA, a poly-A tailing reaction must be performed before cleaning the final product. This is done by mixing Capped IVT RNA (100 al); RNase Inhibitor (20 U); 10× Tailing Buffer (0.5 M Tris-HCl (pH 8.0), 2.5 M NaCl, 100 mM MgCl₂)(12.0 al); 20 mM, ATP (6.0 al); Poly-A Polymerase (20 U); dH₂O up to 123.5 μl and incubation at 37° C. for 30 min. If the poly-A tail is already in the transcript, then the tailing reaction may be skipped and proceed directly to cleanup with Ambion's MEGACLEAR™ kit (Austin, TX) (up to 500 μg). Poly-A Polymerase is preferably a recombinant enzyme expressed in yeast.

For studies performed and described herein, the poly-A tail is encoded in the IVT template to comprise 160 nucleotides in length. However, it should be understood that the processivity or integrity of the Poly-A tailing reaction may not always result in exactly 160 nucleotides. Hence Poly-A tails of approximately 160 nucleotides, e.g, about 150-165, 155, 156, 157, 158, 159, 160, 161, 162, 163, 164 or 165 are within the scope of the invention.

### Protein Expression Assay

IVT mRNAs encoding an mtr or a fragment thereof containing the ARCA (3′ 0-Me-m7G(5′)ppp(5′)G) cap analog or the CapI structure can be transfected into any one of ruminant cell lines, e.g., Bos Taurus cell lines CPAE (endothelial cell), EBTr (NBL-4) (trachea cell), FB2.K (kidney cell), LB9.Bm (lymph node cell), BT (nasal cell), FBHE (heart endothelial cell), CPA47 (pulmonary artery endothelial cell), BCE C/D-1b (eye endothelial cell), BL3.1 (B lymphocyte), EJG (adrenal gland endothelial cell), BL-3 (B lymphocyte), MDBK (NBL-1) (kidney cell), LB9.D (LB9.Sk) (skin cell), SBAC (fibroblast), and BEND (endometrium cell line), all of which are available commercially from ATCC.org. Appropriate culture media and conditions for the above-described host cells are known in the art.

6, 12, 24 and 36 hours post-transfection, the amount of mtr polypeptide expressed can be assayed by ELISA or Western blot. IVT mRNAs that expresses higher levels of mtr would correspond to the IVT mRNA with a higher translationally-competent Cap structure.

When transfected into mammalian cells, the modified mRNAs have a stability of between 12-18 hours or more than 18 hours, e.g., 24, 36, 48, 60, 72 or greater than 72 hours.

Formulation of IVT mRNA Using Lipidoids

IVT mRNA is formulated for in vitro experiments by mixing the IVT mRNA with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for siRNA-lipidoid formulations is used as a starting point. Initial IVT mRNA-lipidoid formulations may consist of particles composed of 42% lipidoid, 48% cholesterol and 10% PEG, with further optimization of ratios possible. After formation of the particle, IVT mRNA is added and allowed to integrate with the complex. The encapsulation efficiency is determined using a standard dye exclusion assays.

Formulation of IVT mRNA Nanoparticles

Solutions of synthesized lipid, 1,2-distearoyl-3-phosphatidylcholine (DSPC) (Avanti Polar Lipids, Alabaster, AL), cholesterol (Sigma-Aldrich, Taufkirchen, Germany), and PEG-c-DOMG (NOF, Bouwelven, Belgium) are prepared at concentrations of 50 mM in ethanol and stored at −20° C. The lipids are combined to yield molar ratio of 50:10:38.5: 1.5 (Lipid: DSPC: Cholesterol: PEG-c-DOMG) and diluted with ethanol to a final lipid concentration of 25 mM. Solutions of modified mRNA at a concentration of 1-2 mg/mL in water are diluted in 50 mM sodium citrate buffer at a pH of 3 to form a stock modified mRNA solution. Formulations of the lipid and modified mRNA are prepared by combining the synthesized lipid solution with the modified mRNA solution at total lipid to IVT mRNA weight ratio of 10:1, 15:1, 20:1 and 30:1. The lipid ethanolic solution is rapidly injected into aqueous modified mRNA solution to afford a suspension containing 33% ethanol. The solutions are injected either manually (MI) or by the aid of a syringe pump (SP) (Harvard Pump 33 Dual Syringe Pump Harvard Apparatus Holliston, MA).

To remove the ethanol and to achieve the buffer exchange, the formulations are dialyzed twice against phosphate buffered saline (PBS), pH 7.4 at volumes 200-times of the primary product using a Slide-A-Lyzer cassettes (Thermo Fisher Scientific Inc. Rockford, IL) with a molecular weight cutoff (MWCO) of 10 kD. The first dialysis was carried at room temperature for 3 hours and then the formulations were dialyzed overnight at 4° C. The resulting nanoparticle suspension was filtered through 0.2 μm sterile filter (Sarstedt, Numbrecht, Germany) into glass vials and sealed with a crimp closure.

### Characterization of Formulations

A Zetasizer Nano ZS (Malvern Instruments Ltd, Malvern, Worcestershire, UK) is used to determine the particle size, the polydispersity index (PDI) and the zeta potential of the RNA nanoparticles in 1×PBS in determining particle size and 15 mM PBS in determining zeta potential.

Ultraviolet-visible spectroscopy is used to determine the concentration of modified mRNA nanoparticle formulation. 100 μl of the diluted formulation in 1×PBS is added to 900 μl of a 4: 1 (v/v) mixture of methanol and chloroform. After mixing, the absorbance spectrum of the solution is recorded between 230 nm and 330 nm on a DU 800 spectrophotometer (Beckman Coulter, Beckman Coulter, Inc., Brea, CA). The RNA concentration in the nanoparicle formulation is calculated based on the extinction coefficient of the RNA used in the formulation and on the difference between the absorbance at a wavelength of 260 nm and the baseline value at a wavelength of 330 nm.

QUANT-IT™ RIBOGREEN® RNA assay (Invitrogen Corporation Carlsbad, CA) is used to evaluate the encapsulation of RNA by the nanoparticle. The samples are diluted to a concentration of approximately 5 μg/mL in TE buffer (10 mM Tris-HCl, 1 mM EDTA, pH 7.5). 50 uL of the diluted samples are transferred to a polystyrene 96 well plate, then either 50 μl of TE buffer or 50 ul of a 2% Triton X-100 solution is added. The plate is incubated at a temperature of 37° C. for 15 minutes. The RIBOGREEN® reagent is diluted 1: 100 in TE buffer, 100 ul of this solution is added to each well. The fluorescence intensity is measured using a fluorescence plate reader (Wallac Victor 1420 Multilablel Counter; Perkin Elmer, Waltham, MA) at an excitation wavelength of −480 nm and an emission wavelength of −520 nm. The fluorescence values of the reagent blank are subtracted from that of each of the samples and the percentage of free modified RNA is determined by dividing the fluorescence intensity of the intact sample (without addition of Triton X-100) by the fluorescence value of the disrupted sample (caused by the addition of Triton X-100).

### In Vitro Incubation—Testing the RNA Vaccine In Vitro

Cells of the cow cell lines (described above) are seeded on 96-well plates (Greiner Bio-one GmbH, Frickenhausen, Germany) at a density of 35,000 cells per well in 100 μl cell culture medium. Formulations containing the RNA nanoparticles are added in quadruplicates directly after seeding the cells and incubated. Cells were trypsinized with Trypsin/EDTA (Biochrom AG, Berlin, Germany). Samples are then analyzed by ELISA or Western blot for expression of the mtr polypeptide encoded by the IVT mRNA encapsulated in the lipid nanoparticles. This tests whether the lipid nanoparticle formulation is effective in delivering the IVT mRNA to the cells.

### Example 3: Ruminant Immunization

Eighteen 5-month-old male Holestein-Friesian calves are used. All animals are grazed on pasture with water ad libitum.

Eighteen calves were allocated randomly into 3 groups. The nucleic acid vaccine (DNA or RNA) with an adjuvant Montanide ISA61 (n=6) is administered subcutaneously in the anterior region of the neck. A second group receives the nucleic acid vaccine without adjuvant (n=6) administered in a similar manner. A control group (n=6) is not vaccinated. Animals are re-vaccinated at 3 weeks after the first vaccination with the same vaccine. Blood samples are collected by jugular venepuncture, with serum separated and stored at −20-C. Saliva samples are collected using a cotton swab placed in mouth. The swab is then placed in a salivette saliva collection tube (Sarstedt, Germany), centrifuged at 2000×g for 10 min and the flow-through material harvested is stored at −20-C. Rumen content samples are collected using a stomach tube, centrifuged at 10,000×g for 15 min and the supernatants (rumen fluid) used for measurement of antibody. Blood, saliva and rumen content samples are collected at weeks 0, 3, 6, and 8. Week 0 samples (n=18) are used to determine level of total bovine Ig in serum, saliva and rumen fluid samples.

### Example 4: Measurement of Antibodies Using ELISA

### Total Bovine IgA and IgG ELISA

ELISA plates (Maxisorp, Nunc™, Thermo Fisher Scien-tific, Denmark) are coated overnight at 4° C. with sheep anti-bovine IgA (AbD-serotec, UK) for total IgA or sheep anti-bovine IgG (AbD-serotec, UK) for total IgG at a protein concentration of 1 ug/mL in PBS. The plates are washed three times with PBS containing 0.05% w/v Tween-20 (PBST) and then blocked for 2 h at room temperature with 150 uL/well of normal sheep serum in PBS (0.1% w/v for IgA and 1% w/v for IgG). 100 uL/well of saliva (dilution range 1:102-1:105), serum (dilution range 1:103-1:107), rumen fluid (dilution range 1:10-1:103) or 2-fold serial dilutions of standards in PBS are added to wells induplicate and plates are incubated for 2 h at room temperature. The plates are washed three times in PBST and incubated for 1 h at room temperature with 100 uL/well of either horseradish peroxidase (HRP)-conjugated sheep anti-bovine IgA (AbD-serotec, UK) diluted 1:10,000 or HRP-conjugated sheep anti-bovine IgG (AbD-serotec, UK) diluted 1:30,000 in blocking buffer for IgA and IgG determination, respectively. The plates are washed three times in PBST and incubated for 10 min at room temperature in the dark with 100 uL/well of tetramethyl benzidine (TMB) substrate (BD OptEIA™, BD Biosciences, USA). The reactions are stopped by addition of 50 uL/well of 0.05M H₂SO₄ and read at 450 nm. A linear standard curve is fitted to the 2-fold serial dilution of the standards (100 ng/mL to 1.56 ng/mL) and used for the calculation of antibody concentration (ug/mL). Bovine IgA standard is obtained from purification of cattle saliva using anion-exchange chromatography followed by size exclusion gel filtration whereas bovine IgG standard was obtained commercially(Bethyl Laboratories, USA).

### Antigen-Specific IgA and IgG ELISA

ELISA assays are developed to monitor a mtr-specific IgA and mtr-specific IgG responses in serum, saliva and rumen content. ELISA plates are coated overnight with 50 uL/well of mtr (4 ug/mL) in PBS at 4° C. The plates are washed three times in PBST and then blocked for 1 h at room temperature with 150 uL/well of 1% casein in PBS. Then, 100 uL/well of saliva (dilution range 1:10-1:104), serum (dilution range 1:10-1:105), rumen fluid (dilution range 1:2-1:20) or 2-fold serial dilutions of the positive serum control in PBS are added to duplicate wells and plates incubated for 2 h at room temperature. The plates are washed three times in PBST and incubated for 1 h at room temperature with 100 uL/well of either HRP-conjugated sheep anti-bovine IgA (AbD-serotec, UK) or HRP-conjugated sheep anti-bovine IgG (AbD-serotec, UK) diluted 1:5000 in blocking buffer for mtr-IgA and mtr-IgG determination, respectively. The plates are washed three times in PBST and incubated for 10 min at room temperature in the dark with 100 uL/well of TMB substrate. The reactions are stopped by addition of 50 uL/well of 0.05 M H₂SO₄ and read at 450 nm. Mtr specific IgA and IgG levels of the unknown samples are assigned a unit value based on a positive control (serum obtained from the animal with the highest antibody responses at week 8). A linear curve is fitted to the 2-fold serial dilution of the positive serum control (1:100 to 1:6400 for mtr-IgA and 1:100,000 to 1:6,400,000 for mtr-IgG) and then used for the calculation of antibody concentration (Units/mL) on every ELISA plate. Based on the curves, the mtr-specific IgA concentration in the positive serum control (1:100 dilution) is assigned a value of 6400 Units/mL, while the mtr-specific IgG concentration in the positive serum (1:100,000 dilu-tion) is assigned a value of 6,400,000 Units/mL. For saliva and rumen fluid samples, mtr-specific IgA and mtr-specific IgG are standardised by total IgA or total IgG to control for animal variation in saliva flow rates and rumen content compositions. Data are presented as units mtr-specific IgA/mg of total IgA or mtr-specific IgG/mg of total IgG.

### Example 5: Measurement of Methane Produced by Cows

The LMD hand-held open path laser measuring device is used. The NDIR, a sniffer method that measures CH4 concentration (ppm) in breath or exhaled air, is also used.

The records are measured in 29 Holstein and Brown Swiss dairy cows. Cows are 18.5% of the 1st parity, 52% of the 2nd parity, 18.5% of the 3rd parity and 11% of the 4th parity. Average days in milk at the beginning of the experiment is 102±88 d. Cows were offered a partial mixed ration consisting of corn silage, grass silage and straw ad libitum, and a mean of 5 kg of concentrate supplied in the automated milking systems (AMS).

Breath CH4 concentration is measured on six different days during four months. Measurements are performed after morning unified distribution between 10:00 and 14:00. Animals are restrained and CH4 is measured simultaneously with the two devices during a 5 min sampling period, obtaining a total of 164 paired measurements.

An operator pointed the LMD at a cow's nostril at a fixed distance of 1 m and trying to maintain the angle from which the LMD is pointed to the cow. Data are recorded every 0.5 s in a tablet. Another operator simultaneously places NDIR sampling tube on the cow's nostrils in order to measure CH4 concentration in exhaled air with the NDIR and data are recorded every is in a datalogger.

### Example 6: Comparison of Intramuscular (IM) and Subcutaneous (SQ) Vaccination Protocol and Antigen Presentation Strategies

This example demonstrates total and specific immune response to ovalbumin (OVA) using both subcutaneous (SQ) and intramuscular (IM) vaccination. Bovine nucleic acid vaccination protocols (SQ and IM) were assessed in vivo in the dairy cows through the quantification of the adaptive immune response following inoculation with ovalbumin (OVA). Immune response was assessed by quantifying total IgG and OVA-specific IgG over a 28-day period after vaccination. Both SQ and IM vaccination provided a robust immune response, with IM vaccination providing a higher total immune response than SQ vaccination.

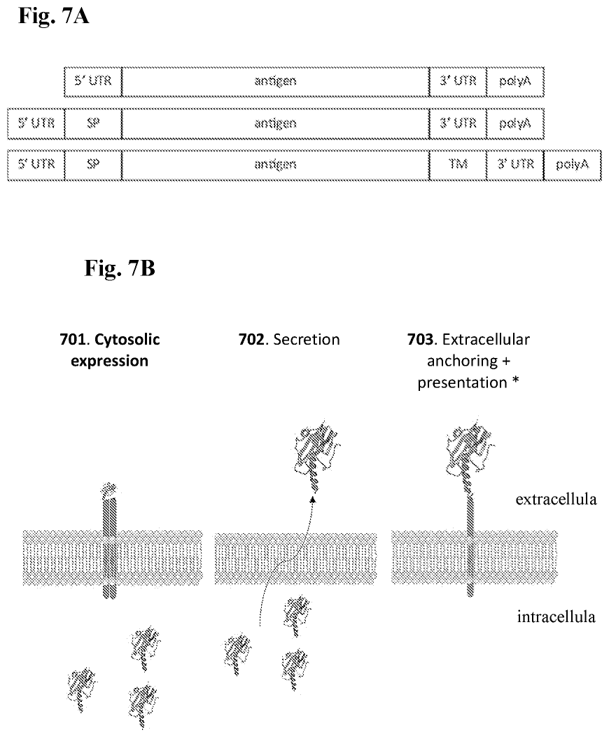

Further, the example demonstrates total and specific immune response to various OVA expression strategies. As shown in FIG. 7A, three OVA constructs (Table 13) were generated: ARK015 (top) comprises a 5′ UTR, a start codon, a sequence encoding the OVA antigen, a 3′ UTR, and a poly A tail; ARK014 (middle) comprises a 5′ UTR, a start codon, a signal peptide (SP), a sequence encoding the OVA antigen, a 3′ UTR, and a poly A tail; ARK011 (bottom) comprises a 5′ UTR, a start codon, a signal peptide (SP), a sequence encoding the OVA antigen, a transmembrane (TM) domain, a 3′ UTR, and a poly A tail. The three OVA constructs result in three different expressions of the OVA protein in the cell and display of the OVA protein to the immune system as shown in FIG. 7B. Specifically, ARK015 results in cytosolic expression (701) of the OVA protein. Without being bound by theory, the OVA protein is then processed and displayed on the cell surface via the major histocompatibility complex. ARK014 is expressed and secreted (702) from the cell for display to the immune system. A portion of the protein may also be processed and displayed on the cell surface via the major histocompatibility complex. ARK011 is expressed and anchored on the cell surface via the transmembrane domain (703) for display to the immune system. A portion of the protein may also be processed and displayed on the cell surface via the major histocompatibility complex. All three expression strategies resulted in a total and specific immune response in dairy cows.

mRNA Synthesis and LNP Preparation

N1-pseudouridine-modified mRNA for the three ova constructs were synthesized at TriLink with a 5′ UTR sequence of 5′ AGGAAATAAGAGAGAAAAGAAGAGTAAGAAGAAATATAAGAGCCACC 3′(SEQ ID NO: 16965), a 3′ UTR sequence of 5′ GCTGCCTTCTGCGGGGCTTGCCTTCTGGCCATGCCCTTCTTCTCTCCCTTGCACCTGT ACCTCTTGGTCTTTGAATAAAGCCTGAGTAGGAAG 3′ (SEQ ID NO: 16966), and a 120A poly A tail (SEQ ID NO: 16988). The mRNAs were purified into 1 mm sodium citrate buffer, pH 6.4 and shipped on dry ice. All mRNAs were stored at −80° C. until use.

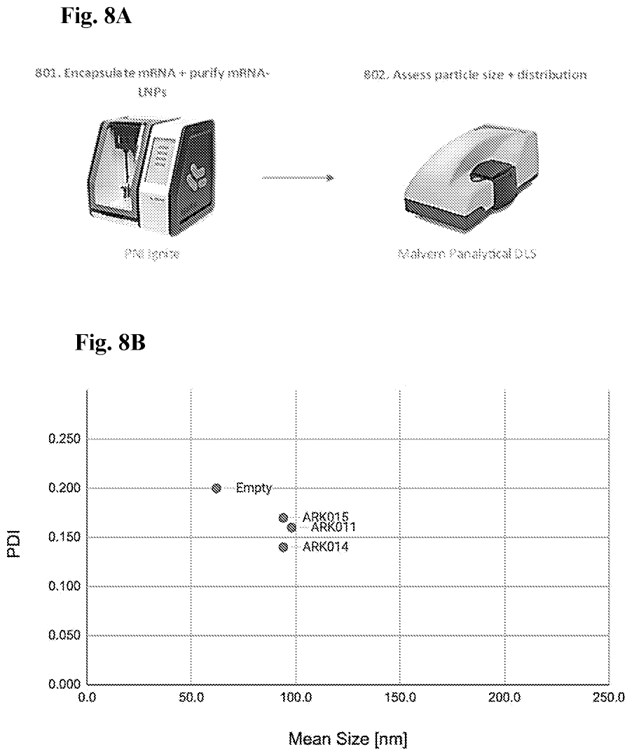

To prepare lipid nanoparticles (LNPs), the mRNA stocks were thawed on ice. Nucleic acid concentrations were validated by absorbance at 260 nm using a NanoDrop. The mRNAs were then diluted to a final concentration of 0.35 mg/mL mRNA in Precision Nanosystem's dilution buffer. 10 mL of the dilute mRNAs were loaded into separate Luer lock syringes and all bubbles were removed by gentle tapping. A NanoAssemblr Ignite NxGen Cartridge was loaded into the cartridge slot of the Ignite instrument (FIG. 8A, 801) and one mRNA syringe was attached to the aqueous inlet of the cartridge. 3.3 mL of Genvoy-ILM reagent was loaded into a separate Luer lock syringes and all bubbles were removed by gentle tapping. The Genvoy-ILM syringe was then attached to the organic inlet of the cartridge. LNPs were then generated and collected into a 15 mL conical tube. The process was repeated for the remaining two mRNAs and for a buffer only LNP control (empty). Immediately after LNPs were generated, the samples were loaded into 10K dialysis cartridges, submerged into 1×DPBS (Ca²⁺ and Mg²⁺-free) to reduce the ethanol concentration to <0.5%. After dialysis the LNPs were recovered from the dialysis cartridges and sterile filtered at 0.2 um. Size and polydispersity (FIG. 8A, 802) were measured using a Malvern Panalytical DLS by diluting 40 uL of LNPs into 360 uL of 1×DPBS. Size (x-axis) and polydispersity (y-axis) of the resultant LNPs are shown in FIG. 8B and Table 14. The resulting LNPs had a mean particle size of ˜95 nm and a PDI of <0.18. Encapsulation efficiency was measured using a RiboGreen assay following Precision Nanosystems recommended protocol using a BioTek Synergy H1 Plate Reader. The resultant encapsulation efficiencies are shown in Table 14, with an average of ˜85%.

### Table 13. Nucleic Acid Sequences

See SEQ ID NO: 16673 to SEQ ID NO: 16675 for the nucleic acid sequence of ARK011, ARK014, and ARK015, respectively.

* When expressed as RNA the T may be replaced with U or pseudo Uridine.

TABLE 14
LNP size, PDI, and encapsulation efficiency measurements

| mRNA ID | Z-avg. (d · nm) | PDI | Encapsulation efficiency |
|---|---|---|---|
| ARK011 | 97.79 | 0.1665 | 83% |
| ARK014 | 94.06 | 0.140 | 87% |
| ARK015 | 96.66 | 0.1705 | 84% |
| Empty | 62.095 | 0.2145 | N/A |

### Vaccination and Sample Collection

Dairy cows were dried off for a period of eight days prior to vaccination. Each vaccine formulation was administered either by IM or SQ injection to three animals. In total, 15 animals were vaccinated with LNPs containing mRNA and 6 animals were vaccinated with empty LNPs. Intramuscular injections were delivered into the cow's right neck muscle by veterinary-trained staff using an 18-gauge 1-1.5″ needle. Subcutaneous injections were delivered into the cow's right shoulder by veterinary-trained staff using an 18-gauge 0.5-0.75″ needle. Total blood samples (˜20 mL) were collected by veterinary-trained staff. Samples were collected into 10 mL vacutainer tubes (2×10 mL) from the coccygeal vein (tail vein) by needle puncture. Harvested blood samples were then processed via ultracentrifugation for serum collection. Isolated serum samples were aliquoted, labeled and stored in cryogenic tubes at −20° C. until use.

### Sera IgG Measurements

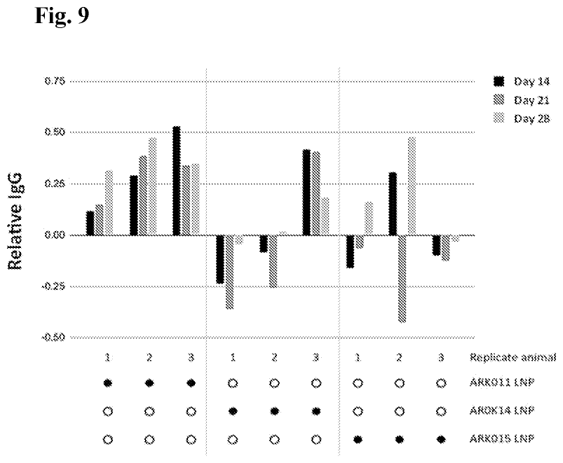

Sera samples were thawed on ice and total IgG was quantified using Triple J Farms' Ig Radial Immunodiffusion (RID) kits (Cat No. 728411) following the manufacturers recommended protocol. Sera IgG measurements are shown in Table 15. While, there was substantial biological variability between animals, each mRNA vaccinated animal demonstrated a maintained immune response over the 28-day test period while the empty vaccinated animals demonstrated a reduction in total sera IgG. In particular, when the treated groups were normalized against the empty groups, the ARK011 construct demonstrated a robust and prolonged immune response across all 3 animals (FIG. 9). The ARK014 and ARK015 constructs demonstrated a detectable immune response in at least one of the treated animals.

TABLE 15
Sera IgG measurements

| | | Ani- | | | | | |
|---|---|---|---|---|---|---|---|
| | | mal | | | | | |
| | Treat- | repli- | Dose | Serum IgG Concentration (mg/dL) | | | |
| Construct | ment | cate | [ug] | Day 0 | Day 14 | Day 21 | Day 28 |
| ARK011 | IM | 1 | 353 | 5,261 | 4,902 | 4,994 | 3,702 |
| ARK011 | IM | 2 | 353 | 5,203 | 5,664 | 6,017 | 4,145 |
| ARK011 | IM | 3 | 353 | 6,150 | 6,716 | 5,823 | 3,794 |
| ARK011 | SQ | 1 | 353 | 4,964 | 6,139 | 7,379 | 3,730 |
| ARK011 | SQ | 2 | 353 | 4,956 | 4,815 | 5,722 | 4,877 |
| ARK011 | SQ | 3 | 353 | 5,599 | 4,777 | 4,034 | 4,344 |
| ARK014 | IM | 1 | 348 | 3,031 | 3,349 | 2,773 | 2,691 |
| ARK014 | IM | 2 | 348 | 3,011 | 4,027 | 3,231 | 2,861 |
| ARK014 | IM | 3 | 348 | 3,804 | 6,213 | 6,100 | 3,326 |
| ARK015 | IM | 1 | 468 | 3,658 | 3,692 | 4,070 | 3,272 |
| ARK015 | IM | 2 | 468 | 4,086 | 5,735 | 2,488 | 4,153 |
| ARK015 | IM | 3 | 468 | 3,167 | 3,947 | 3,797 | 2,723 |
| ARK015 | SQ | 1 | 468 | 4,875 | 5,972 | 5,722 | 4,015 |
| ARK015 | SQ | 2 | 468 | 3,680 | 3,257 | 3,149 | 3,009 |
| ARK015 | SQ | 3 | 468 | 2,964 | 3,894 | 3,981 | 2,221 |
| empty | IM | 1 | — | 5,186 | 5,204 | 5,226 | 3,509 |
| empty | IM | 2 | — | 3,167 | 4,494 | 4,514 | 2,422 |
| empty | IM | 3 | — | 2,670 | 3,459 | 3,280 | 2,507 |
| empty | SQ | 1 | — | 7,813 | 4,834 | 7,246 | 5,065 |
| empty | SQ | 2 | — | 2,670 | 3,273 | 3,745 | 3,009 |
| empty | SQ | 3 | — | 5,286 | 8,496 | 8,165 | 4,577 |

### VA-Specific Ig Measurements

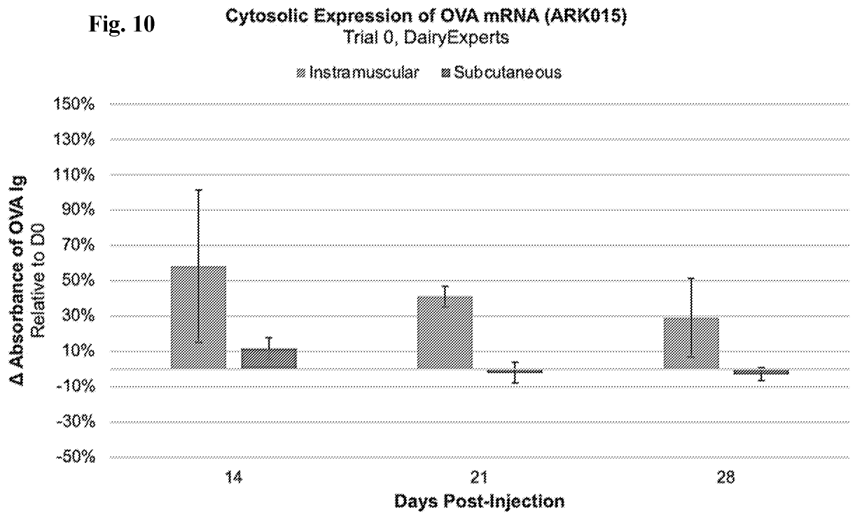

Sera samples were thawed on ice and total IgG was quantified using NovateinBio's Bovine OVA sIgG Antibody ELISA Kit (Cat No. BG-BVN11731) following the manufacturer's recommended protocol. Ova-specific IgG measurements are shown in Table 16. Each of the animals vaccinated with a construct show a demonstrable OVA-specific IgG response over the 28-day test period. Interestingly, intramuscular injection of ARK015 LNPs demonstrated ˜4-fold increased OVA-specific IgG response as compared to subcutaneous injection (FIG. 10).

TABLE 16
OVA-specific IgG measurements

| | | Animal | Absorbance (OD₄₅₀) | | | |
|---|---|---|---|---|---|---|
| Construct | Treatment | replicate | Day 0 | Day 14 | Day 21 | Day 28 |
| ARK011 | IM | 1 | 0.327 | 0.64 | 0.757 | 0.626 |
| ARK011 | IM | 2 | 0.591 | 0.616 | 0.513 | 0.603 |
| ARK011 | IM | 3 | 1.937 | 1.817 | 2.024 | 1.663 |
| ARK011 | SQ | 1 | 0.756 | 1.085 | 1.063 | 0.704 |
| ARK011 | SQ | 2 | 1.004 | 1.16 | 1.036 | 0.916 |
| ARK011 | SQ | 3 | 0.952 | 0.955 | 0.7 | 0.68 |
| ARK014 | IM | 1 | 0.475 | 0.615 | 0.571 | 0.483 |
| ARK014 | IM | 2 | 0.837 | 0.88 | 0.833 | 0.986 |
| ARK014 | IM | 3 | 2.199 | 1.838 | 1.381 | 2.025 |
| ARK015 | IM | 1 | 0.842 | 1.653 | 1.23 | 1.097 |
| ARK015 | IM | 2 | 0.938 | 1.048 | 1.261 | 0.999 |
| ARK015 | IM | 3 | 0.308 | 0.513 | 0.443 | 0.465 |
| ARK015 | SQ | 1 | 1.394 | 1.287 | 1.504 | 1.368 |
| ARK015 | SQ | 2 | 1.327 | 1.175 | 1.277 | 1.066 |
| ARK015 | SQ | 3 | 3.178 | 2.478 | 1.985 | 1.691 |
| empty | IM | 1 | 1.427 | 1.19 | 1.058 | 0.939 |
| empty | IM | 2 | 1.217 | 1.301 | 1.18 | 1.176 |
| empty | IM | 3 | 0.512 | 0.566 | 0.571 | 0.476 |
| empty | SQ | 1 | 1.128 | 1.093 | 0.918 | 0.848 |
| empty | SQ | 2 | 0.735 | 0.786 | 0.729 | 0.769 |
| empty | SQ | 3 | 1.172 | 1.119 | 1.147 | 1.085 |

This example demonstrates total and OVA-specific immune response in dairy cattle following vaccination with various mRNA LNP vaccines. Further, this example demonstrates that expression strategy and immune presentation may affect both total and OVA-specific immune response.

### Example 7: Metagenomic Analysis of Ruminal Microbiome

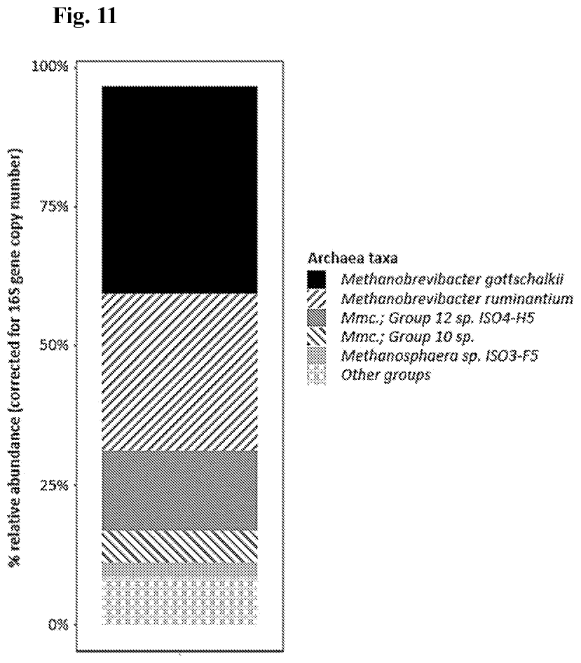

This example demonstrates the that two methanogen strains, Methanobrevibacter ruminantium and Methanobrevibacter gottschalkii, comprise ˜65% of the methanogen load in the ruminal microbiome, and, thus, are high value targets for vaccine development. Ruminal 16S sequencing datasets were used to analyze the microbial community composition of rumen samples from 394 cattle across 26 countries (Henderson (2015) Scientific Reports). The average relative abundance value of each archaeal species was pulled from the dataset and corrected for 16S gene copy number (GCN). Methanogen genomes can contain multiple copies of the 16S rRNA gene, so correcting for the GCN enables the capture of a more accurate picture of archaeal community structure. The 16S GCN values for each taxon was determined using the rrnDB, which is a database that documents rRNA operon copy number variation in Bacteria and Archaea. All taxa within the dataset were represented in the rrnDB2. The resulting abundance analysis is shown in FIG. 11. Specifically, FIG. 11 shows the relative abundance of the archaeal species that composed >1% of the 16S GCN-corrected data. The methanogenic taxa Methanobrevibacter, Methanosphaera, and Methanomethylophiceae compose the vast majority of the archaeal community. Methanobrevibacter gottschalkii and Methanobrevibacter ruminantium are the dominant species, composing ˜65% of the ruminal microbiome across all 394 cattle when combined. In addition, the study found that within the rumen microbial community, the archaeal community in general is stable across host species, diet, and geographic region, as bacterial taxa are the main drivers of most observed variation. This knowledge builds confidence that the mRNA constructs from the two dominant methanogen species will be relevant on a global scale.

### Example 8: Immunization of Cattle with Nucleic Acid Vaccines Encoding Methanogen Cell Surface Proteins

This example demonstrates reduction in methane production by methanogens when treated with sera samples from animals vaccinated with nucleic acid vaccines encoding methanogen cell surface proteins. Beef cattle were vaccinated with mRNA vaccines comprising a sequence as shown in Table 17C formulated as LNPs or an empty LNP control. Immune response was assessed by quantifying total IgG over a 35-day period after vaccination and reduction in methane production was quantified after sera addition to methanogen monocultures.

mRNA Synthesis and LNP Preparation

N1-pseudouridine-modified mRNA for the constructs shown in Tables 17A-C were synthesized at TriLink with a 5′ UTR sequence of 5′ AGGAAATAAGAGAGAAAAGAAGAGTAAGAAGAAATATAAGAGCCACC 3′ (SEQ ID NO: 16967), a 3′ UTR sequence of 5′ GCTGCCTTCTGCGGGGCTTGCCTTCTGGCCATGCCCTTCTTCTCTCCCTTGCACCTGT ACCTCTTGGTCTTTGAATAAAGCCTGAGTAGGAAG 3′ (SEQ ID NO: 16968), and a 120A poly A tail (SEQ ID NO: 16988). The mRNAs were purified into 1 mM sodium citrate buffer, pH 6.4 and shipped on dry ice. All mRNAs were stored at −80° C. until use.

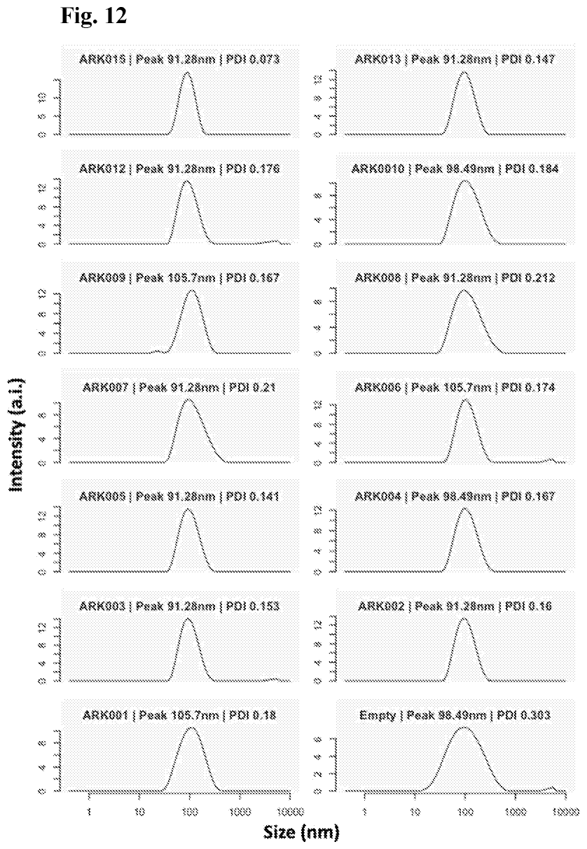

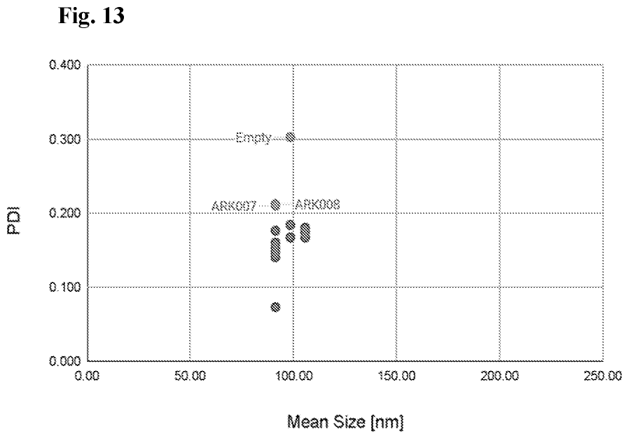

To prepare lipid nanoparticles (LNPs), the mRNA stocks were thawed on ice. Nucleic acid concentrations were validated by absorbance at 260 nm using a NanoDrop. The mRNAs were then diluted to a final concentration of 0.35 mg/mL mRNA in Precision Nanosystem's dilution buffer. 10 mL of the dilute mRNAs were loaded into separate Luer lock syringes and all bubbles were removed by gentle tapping. A NanoAssemblr Ignite NxGen Cartridge was loaded into the cartridge slot of the Ignite instrument and one mRNA syringe was attached to the aqueous inlet of the cartridge. 3.3 mL of Genvoy-ILM reagent was loaded into a separate Luer lock syringes and all bubbles were removed by gentle tapping. The Genvoy-LM syringe was then attached to the organic inlet of the cartridge. LNPs were then generated and collected into a 15 mL conical tube. The process was repeated for the remaining mRNAs and for a buffer only LNP control (empty). Immediately after LNPs were generated, the samples were loaded into 10K dialysis cartridges, submerged into 1×DPBS (Ca²⁺ and Mg²⁺-free) to reduce the ethanol concentration to <0.5%. After dialysis the LNPs were recovered from the dialysis cartridges and sterile filtered at 0.2 um. Size and polydispersity (FIG. 12 and FIG. 13) were measured using a Malvern Panalytical DLS by diluting 40 uL of LNPs into 360 uL of 1×DPBS. Size (x-axis) and polydispersity (y-axis) of the resultant LNPs are shown in FIGS. 12-13 and Table 18. The resulting LNPs had a mean particle size of ˜90 nm and a PDI of <0.2. Encapsulation efficiency was measured using a RiboGreen assay following Precision Nanosystems recommended protocol using a BioTek Synergy Hi Plate Reader. The resultant encapsulation efficiencies are shown in Table 18, with an average of ˜60%.

TABLE 17A
Genes and Bos taurus codon-optimized sequence of the genes

| | | Bos taurus optimized |
|---|---|---|
| Name | Species | sequence (SEQ ID NO) |
| WP_004031278.1 | Methanobacterium | SEQ ID NO: 16969 |
| | formicicum | |
| WP_012956721.1 | Methanobrevibacter | SEQ ID NO: 16970 |
| | ruminantium | |
| WP_019264904.1 | Methanobrevibacter | SEQ ID NO: 16971 |
| | smithii | |
| WP_016359091.1 | Methanobrevibacter | SEQ ID NO: 16972 |
| | sp. AbM4 | |
| WP_011033493.1 | Methanosarcina | SEQ ID NO: 16973 |
| | mazei | |
| mru0143 | Methanobrevibacter | SEQ ID NO: 16974 |
| | ruminantium | |
| mru0842 | Methanobrevibacter | SEQ ID NO: 16975 |
| | ruminantium | |
| mru2048 | Methanobrevibacter | SEQ ID NO: 16976 |
| | ruminantium | |
| mru1222 | Methanobrevibacter | SEQ ID NO: 16977 |
| | ruminantium | |
| mru2047 | Methanobrevibacter | SEQ ID NO: 16978 |
| | ruminantium | |
| Ova | Gallus gallus | SEQ ID NO: 16979 |
| Signal | | SEQ ID NO: 16980 |
| peptide | | |
| TM/C | | SEQ ID NO: 16981 |
| domains | | |

TABLE 17B
Constructs

| Name | Note |
|---|---|
| Ark_001 | SP + WP_004031278.1 + TM/C |
| Ark_002 | SP + WP_012956721.1 + TM/C |
| Ark_003 | SP + WP_019264904.1 + TM/C |
| Ark_004 | SP + WP_016359091.1 + TM/C |
| Ark_005 | SP + WP_011033493.1 + TM/C |
| Ark_006 | SP + mru0143 + TM/C |
| Ark_007 | SP + mru0842 + TM/C |
| Ark_008 | SP + mru2048 + TM/C |
| Ark_009 | SP + mru1222 + TM/C |
| Ark_010 | SP + mru2047 + TM/C |
| Ark_011 | SP + Ova + TM/C |
| Ark_012 | SP + WP_012956721.1 + STOP |
| Ark_013 | SP + mru0143 + STOP |
| Ark_014 | SP + OVA + STOP |
| Ark 015 | OVA + STOP |

*SP: signal peptide; OVA: ovalbumin; TM/C: transmembrane domain and cytoplasmic domain

TABLE 17C
Nucleic acid sequences

See SEQ ID NO: 16676 to SEQ ID NO: 16688 for the nucleic
acid sequence of ARK001-ARK010, ARK012, ARK013, and
ARK015, respectively.
* When expressed as RNA the T may be replaced with U or pseudo Uridine.

TABLE 18
LNP size, PDI, and encapsulation efficiency measurements

| mRNA ID | Z-avg. (d · nm) | PDI | Encapsulation efficiency |
|---|---|---|---|
| ARK001 | 78.82 | 0.086 | 51% |
| ARK002 | 78.82 | 0.108 | 51% |
| ARK003 | 78.82 | 0.118 | 40% |
| ARK004 | 78.82 | 0.108 | 51% |
| ARK005 | 78.82 | 0.064 | 56% |
| ARK006 | 91.28 | 0.141 | 73% |
| ARK007 | 78.82 | 0.137 | 57% |
| ARK008 | 78.82 | 0.166 | 66% |
| ARK009 | 68.06 | 0.160 | 59% |
| ARK010 | 78.82 | 0.183 | 51% |
| ARK012 | 78.82 | 0.100 | 49% |
| ARK013 | 78.82 | 0.1841 | 60% |
| ARK015 | 68.06 | 0.130 | 62% |
| Empty | 68.06 | 0.228 | N/A |

### Denaturing Agarose Gel Electrophoresis of LNPs

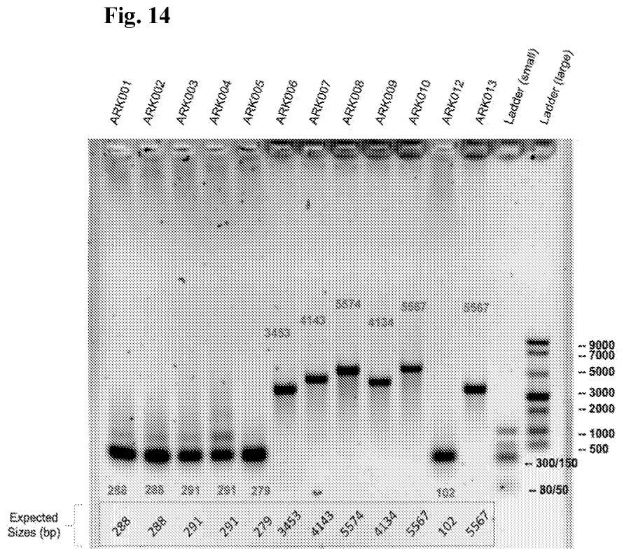

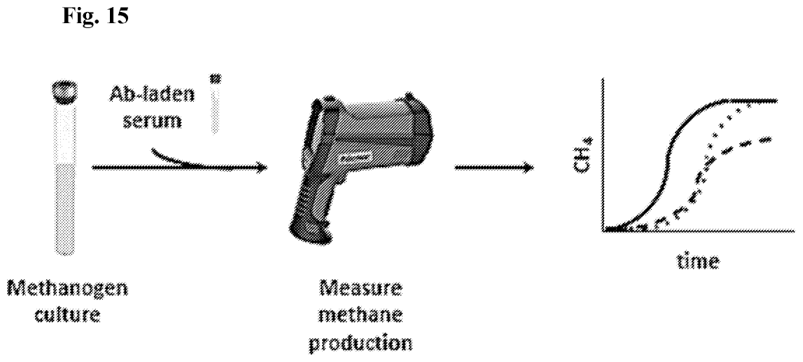

After LNP formulation, LNPs were broken and the recovered mRNA was analyzed by denaturing agarose gel electrophoresis. Briefly, 10 uL of LNPs were combined with 2×RNA loading dye (Thermofisher) and heated at 70° C. for 10 minutes. The denatured mRNAs were then loaded onto a 1% agarose gel and run at 120 V constant for 45 minutes in 1×TAE buffer. The gel was stained with SybrSafe and then imaged on a BioRad GelDoc. The resulting image demonstrating the integrity and correct size of the mRNA after LNP formulation is shown in FIG. 14.

### Vaccination and Sample Collection

Healthy, weaned, Angus cross steers were utilized for this study. All calves were weaned for a minimum of sixty days and received pre-weaned vaccinations a minimum of sixty days prior to initiation of the study. Animals were transferred to the study site fourteen days prior to vaccination to allow for acclimation to the study diet and environment. Each vaccine formulation was administered by IM injection to three animals. In total, 39 animals were vaccinated with LNPs containing ˜250 ug mRNA and 3 animals were vaccinated with empty LNPs. Intramuscular injections were delivered into the cow's right neck muscle by veterinary-trained staff using an 18-gauge 1-1.5″ needle. Animals were boosted with a second vaccine administration on day 21. Total blood samples (˜100 mL) were collected from the jugular vein by veterinary-trained staff and processed by ultracentrifugation within 24 hours of sample collection. Isolated serum samples were aliquoted, labeled and stored in cryogenic tubes at −20° C. until use.

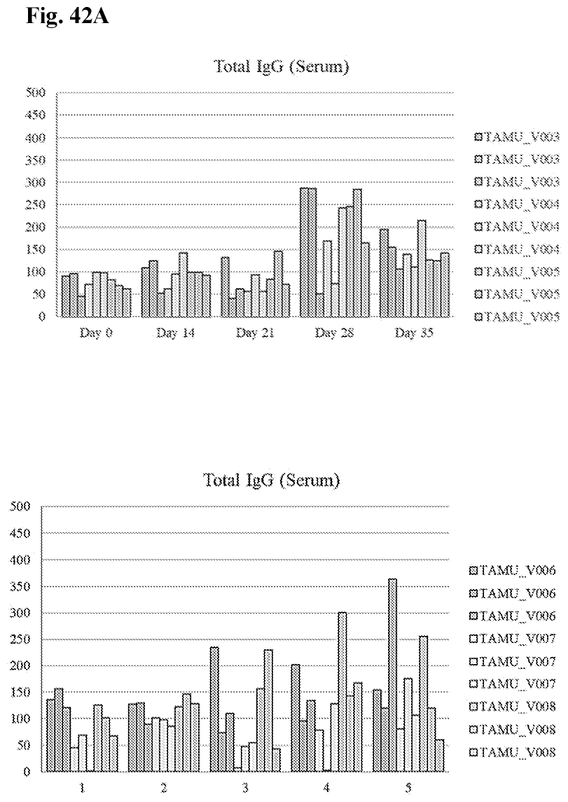

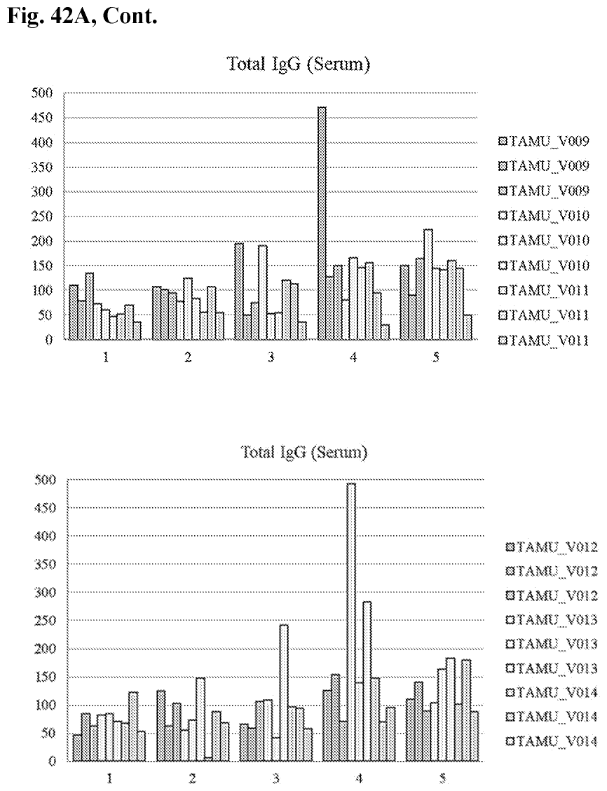

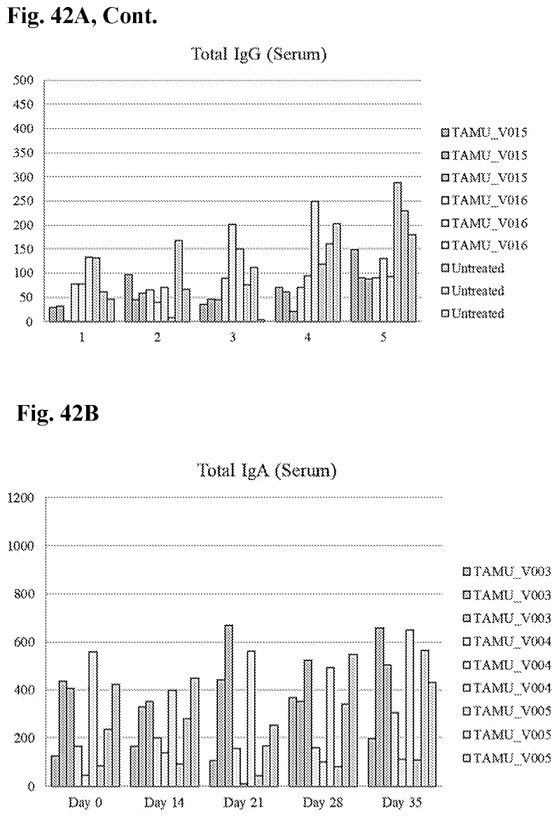

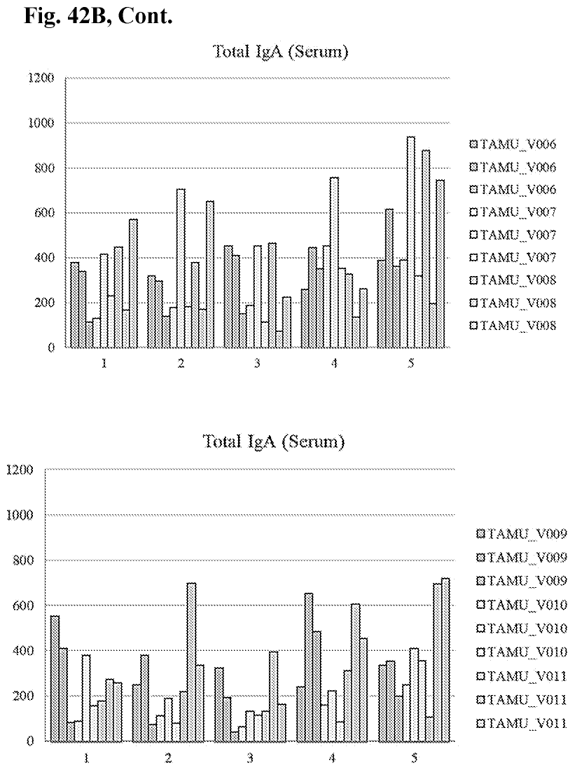

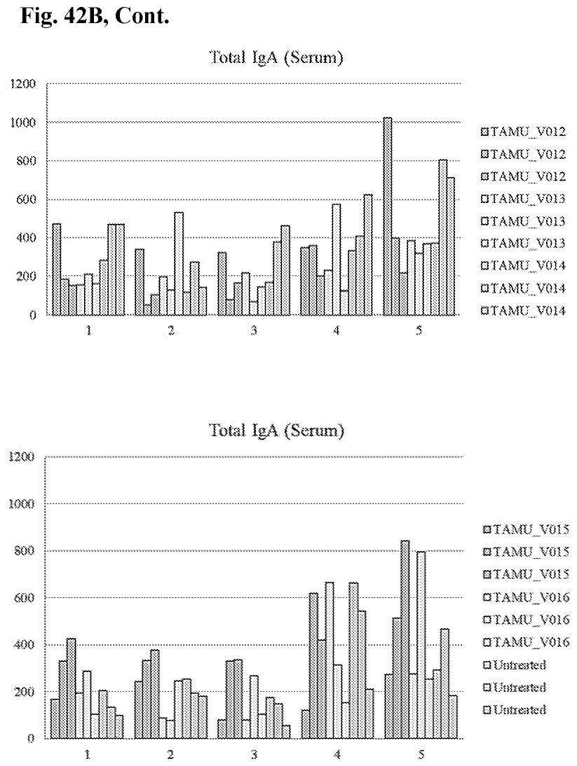

Using the isolated serum samples, ELISA assays were performed to detect total IgG (FIG. 42A) and total IgA (FIG. 42B) levels in the cows' serums.

Methane Production Measurements for Methanogen Monocultures Treated with Sera

200 uL of serum from vaccinated animals were added to sterile anaerobic culture tubes. Open plasma containing tubes were placed into an anaerobic chamber containing 95% nitrogen and 5% hydrogen and allowed to stand 1 hr at ambient temperature. A single seeded culture of methanogen (M. gottschalkii or M. ruminantium) was prepared in BY medium at a volume sufficient to distribute 2.5 mL to each plasma sample and control tube. 2.5 mL of methanogen culture was added to each tube containing the serum, a septa was inserted and crimped into place. Sealed tubes were removed from the anaerobic chamber and each was pressurized with 25 PSI 80% H₂, 20% CO₂ gas via a hypodermic needle through the septum. Tubes were placed into a 38° C. shaking incubator and periodically removed for methane measurements. Methane measurements in the tube headspace were made with a handheld remote methane detector using a jig to hold the instrument and tube during measurement.

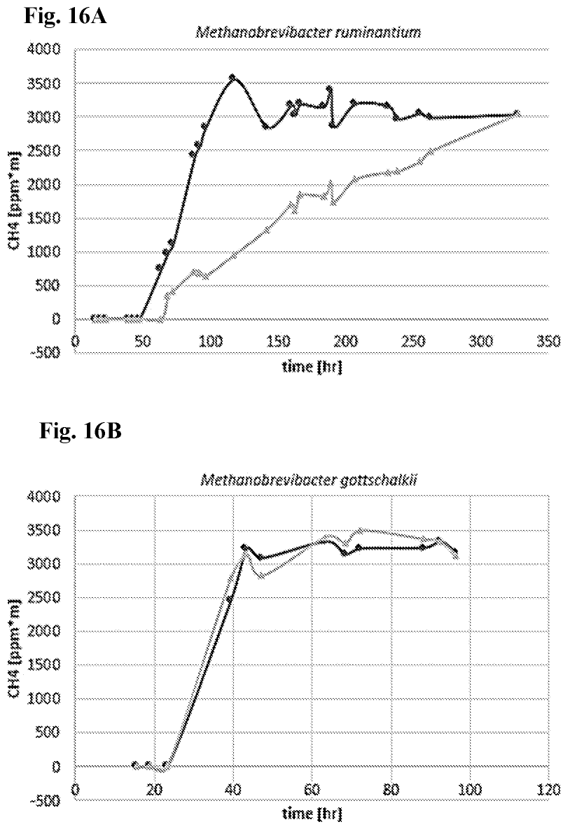

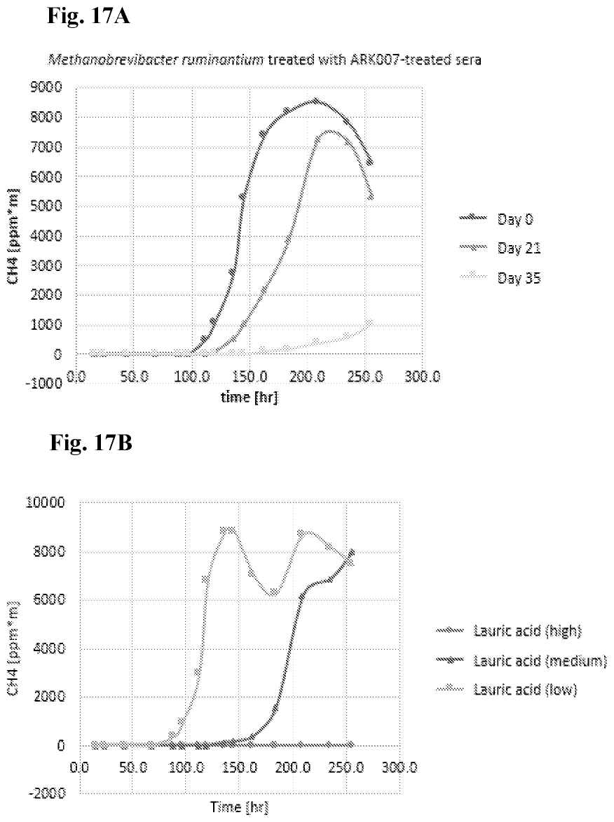

Methane measurements for select sera are shown in FIG. 16 and FIG. 17.

FIG. 16 shows species selective reduction in methane production when treated with vaccinated sera. Briefly, animals were vaccinated with ARK007, a nucleic acid vaccine encoding a protein present in M. ruminantium but not M. gottschalkii. Methane production (y-axis) over time (x-axis) is plotted in FIG. 16A for a culture of Methanobrevibacter ruminantium combined with a serum sample collected at day 14 after vaccination with ARK007 (light gray, triangles) compared to the same animal pre-vaccination (dark gray, circles). Methane production rate is reduced by 3× in the sample treated with the day 14 serum. Methane reaches maximal production at −300 hrs in the sample treated with the day 14 serum as compared to −100 hrs when treated with pre-vaccination serum. FIG. 16B shows methane production over time for a culture of Methanobrevibacter gottschalkii treated with the same sera samples. There is no notable reduction in methane production for M. gottschalkii when treated with either the day 14 or pre-vaccination sera. This demonstrates the ability to generate highly specific and selective antibodies by vaccination with nucleic acids encoding methanogen cell surface proteins.

FIG. 17 shows a comparison of the effect of serum from animals vaccinated with ARK007 compared to a common small molecule inhibitor or methanogenesis. FIG. 17A shows increased reduction in methane production by monocultures of Methanobrevibacter ruminantium when treated with sera collected pre-vaccination, after a first injection on day 0, and after a boost injection on day 21. Methane production rate is reduced ˜2× when treated with serum collected after a single vaccination (day 21) and ˜20× when treated with serum collected after a boost vaccination (day 35). FIG. 17B shows reduction is methane production rate when treated with a small molecule inhibitor of methanogenesis (lauric acid) at 3 different concentrations. This demonstrates that the vaccinated sera samples are preforming with similar efficacy to known small molecule inhibitors of methanogenesis.

This example demonstrates the ability to significantly reduce methane production by vaccination with nucleic acid vaccine encoding methanogen cell surface proteins. Vaccination resulted sera antibodies that when combined with methanogens in monoculture result in ˜3-20× reduction in methane production rate in vitro.

### Example 9: Informatic Analysis of Antigen Targets and Vaccination

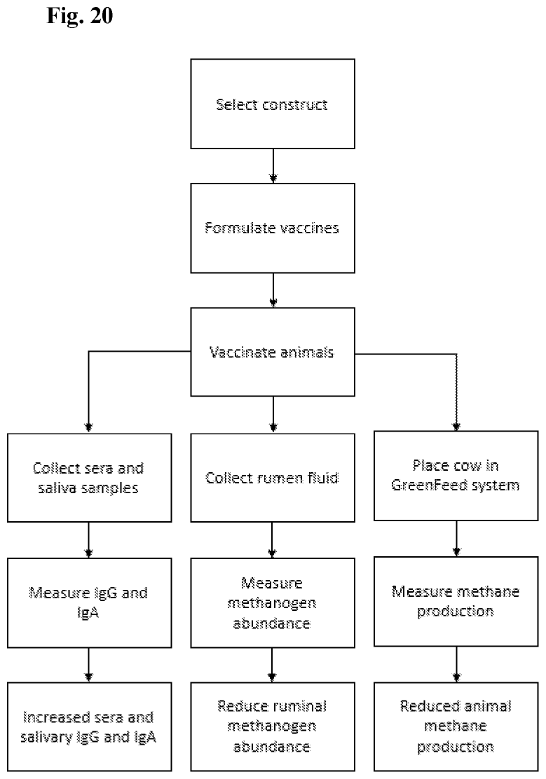

This example demonstrates informatic selection of 40 methanogen cell surface proteins, generation of nucleic acid vaccines encoding those cell surface proteins, and subsequent methods for analyzing the efficacy of those antigens as nucleic acid vaccines for the reduction of methane production. An exemplary methodology for testing selected constructs is shown in FIG. 20.

### Cell Surface Protein Selection

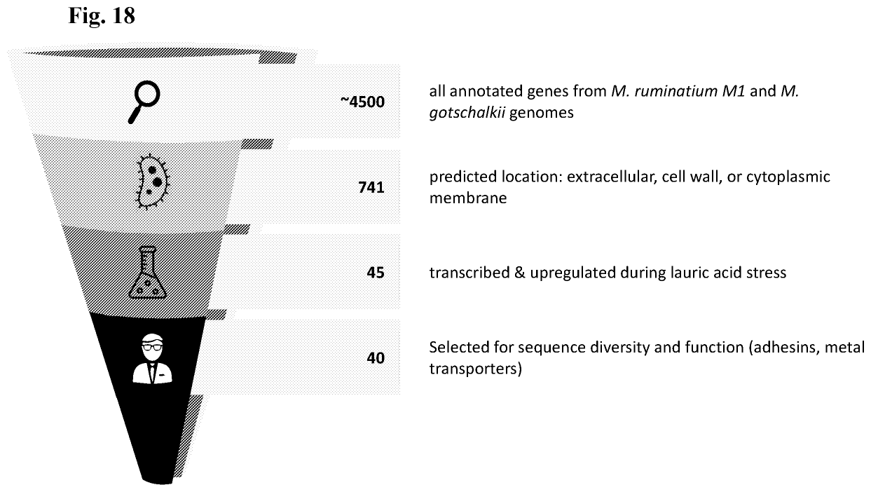

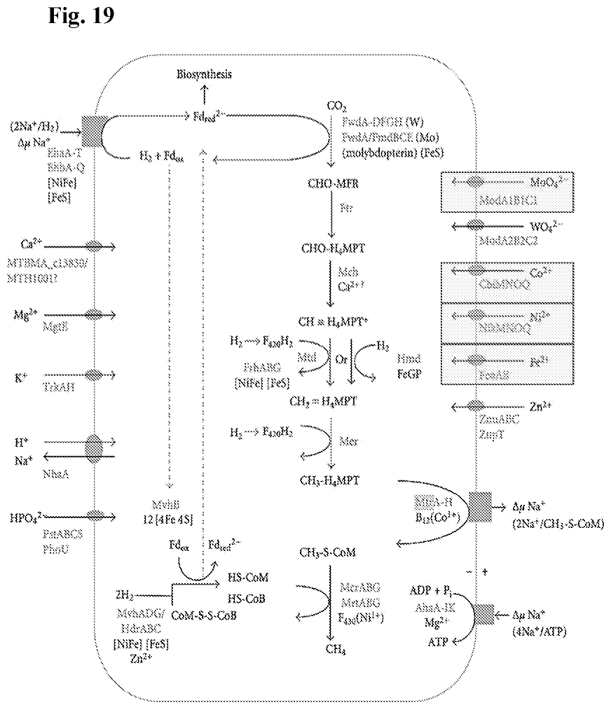

The cell surface protein selection pipeline used to curate 40 proteins for vaccine production is illustrated in FIG. 18. Briefly, Methanobrevibacter ruminantium and Methanobrevibacter gottschalkii were identified as the two most prevalent ruminal methanogens in the abundance analysis (Example 7). Each open reading frame (ORF) from their published genomes were curated first database comprising ˜4500 ORFS. Subcellular localization for each ORF was predicted using pSORTdb (Lau (2020) Nucleic Acids Research) and the predicted localization was appended to the first database. The 741 predicted non-intracellular proteins were then ranked based on published gene expression changes in response to lauric acid stress (Zhou (2018) BMC Research Notes) resulting in a second database comprising 45 proteins. The DNA sequences of the proteins were collected from NCBI and were used to search a subset of rumen transcriptomic studies in the Sequence Read Archive (SRA). Proteins were given additional emphasis for inclusion in the final set of candidates based on if there was evidence that either the exact nucleotide sequence or a highly similar sequence was found in transcriptomic data. From examination of the combination of all of these datasets, as well as information based on the predicted functions of the proteins (adhesin or metal binding for example), a final list of sequences was output for additional studies comprising 40 sequences. The protein IDs were used to download the protein sequences from NCBI. As an additional step prior to ordering constructs, the proteins were analyzed using a signal peptide predictor and signal peptides were removed from the final designs. In addition, some of the proteins were too long for synthesis of the template for mRNA production. In this case, the proteins were examined and truncated versions of the protein were chosen based on predicted domain boundaries. Sequences underwent codon optimization using the Bos taurus codon usage table and uridine depletion in order to be used for mRNA production. This final list of sequences (Table 19, ARK016-055) encompasses a list of proteins from two methanogens commonly found in the rumen of cattle. As these proteins are predicted to be outside of the cells and there is evidence indicating they are expressed, they serve as a set of promising leads to be used in future vaccine studies to reduce methane production within the rumen.

### Table 19. Selected Methanogen Cell-Surface Proteins

See SEQ ID NO: 16689 to SEQ ID NO: 16728 for the native nucleic acid sequences of selected methanogen cell-surface proteins (ARK016-ARK055, respectively). See SEQ ID NO: 16729 to SEQ ID NO: 16768 for the Bos Taurus codon-optimized and uridine-depleted nucleic acid sequences of selected methanogen cell-surface proteins (ARK016-ARK055, respectively). See SEQ ID NO: 16769 to SEQ ID NO: 16808 for the amino acid sequences of selected methanogen cell-surface proteins (ARK016-ARK055, respectively).

mRNA Synthesis and LNP Preparation

N1-pseudouridine-modified mRNA for the constructs shown in Table 19 are synthesized at TriLink with a 5′ UTR sequence of 5′ AGGAAATAAGAGAGAAAAGAAGAGTAAGAAGAAATATAAGAGCCACC 3′ (SEQ ID NO: 16982), a 3′ UTR sequence of 5′ GCTGCCTTCTGCGGGGCTTGCCTTCTGGCCATGCCCTTCTTCTCTCCCTTGCACCTGT ACCTCTTGGTCTTTGAATAAAGCCTGAGTAGGAAG 3′ (SEQ ID NO: 16983), and a 120A poly A tail (SEQ ID NO: 16988). The constructs were ordered with a signal peptide (5′ ATGTTCGTGTTCCTCGTGCTCCTGCCCCTCGTCTCCAGCCAGTGCGTG 3′ (SEQ ID NO: 16984)) and/or transmembrane domain (5′ CCCTGGTACATCTGGCTGGGCTTCATCGCAGGACTGATCGCCATCGTCATGGTCACC ATAATGCTCTGTTGCATGACCTCCTGCTGTAGCTGCCTGAAAGGCTGTTGCAGCTGC GGGAGCTGCTGCAAATTCGACGAAGACGATAGCGAACCTGTGCTCAAAGGGGTGAA GTTGCATTACACATAGTGA (SEQ ID NO: 16985)). The mRNAs are purified into 1 mM sodium citrate buffer, pH 6.4 and shipped on dry ice. All mRNAs are stored at −80° C. until use.

To prepare lipid nanoparticles (LNPs), the mRNA stocks are thawed on ice. Nucleic acid concentrations are validated by absorbance at 260 nm using a NanoDrop. The mRNAs are then diluted to a final concentration of 0.35 mg/mL mRNA in Precision Nanosystem's dilution buffer. 10 mL of the dilute mRNAs are loaded into separate Luer lock syringes and all bubbles are removed by gentle tapping. A NanoAssemblr Ignite NxGen Cartridge is loaded into the cartridge slot of the Ignite instrument and one mRNA syringe is attached to the aqueous inlet of the cartridge. 3.3 mL of Genvoy-ILM reagent is loaded into a separate Luer lock syringes and all bubbles are removed by gentle tapping. The Genvoy-ILM syringe is then attached to the organic inlet of the cartridge. LNPs are then generated and collected into a 15 mL conical tube. The process is repeated for the remaining mRNAs and for a buffer only LNP control (empty). Immediately after LNPs are generated, the samples are loaded into 10K dialysis cartridges, submerged into 1×DPBS (Ca²⁺ and Mg²⁺-free) to reduce the ethanol concentration to <0.5%. After dialysis the LNPs are recovered from the dialysis cartridges and sterile filtered at 0.2 um. Size and polydispersity are measured using a Malvern Panalytical DLS by diluting 40 uL of LNPs into 360 uL of 1×DPBS. Encapsulation efficiency is measured using a RiboGreen assay following Precision Nanosystems recommended protocol using a BioTek Synergy H1 Plate Reader.

### Vaccination and Sample Collection

Healthy, weaned, Angus cross steers are weaned for a minimum of sixty days and received pre-weaned vaccinations a minimum of sixty days prior to initiation of the study. Animals are transferred to the study site fourteen days prior to vaccination to allow for acclimation to the study diet and environment. Each vaccine formulation is administered by IM injection to three animals. In total, 120 animals are vaccinated with LNPs containing ˜500 ug mRNA and 3 animals are vaccinated with empty LNPs. Intramuscular injections are delivered into the cow's right neck muscle by veterinary-trained staff using an 18-gauge 1-1.5″ needle. Animals are boosted with a second vaccine administration on day 21. Total blood samples (˜100 mL) are collected from the jugular vein by veterinary-trained staff and processed by ultracentrifugation within 24 hours of sample collection. Isolated serum samples are aliquoted, labeled and stored in cryogenic tubes at −20° C. until use.

### Ruminal Microbiome Measurements

˜50 mL of fresh rumen samples are collected by esophageal tubing and retained in conical vials. Samples are strained through three layers of cheesecloth, with the liquid phase aliquoted into 50 mL conical vials. All samples re snap-frozen in liquid N2 and stored at −20° C. for subsequent processing. Samples are processed within twenty-four hours of collection. Sample aliquots are labeled with animal number, collection method, collection date and time.

Isolation of DNA from the rumen follows the methods defined in Henderson (Henderson, G. et al. Effect of DNA Extraction Methods and Sampling Techniques on the Apparent Structure of Cow and Sheep Rumen Microbial Communities. PLOS One 8, e74787 (2013), which is incorporated herein by reference), with preference given to methods involving both phenol-chloroform and mechanical lysis steps (PCQI, PCBB, PCSA). PCR amplification of the hypervariable V6-V8 regions of the 16S rRNA gene is performed using the archaea-specific Ar915aF/Ar1386R primer set with Illumina adapters as defined in Table 1 of Kittelmann 2015 (Kittelmann et al. Buccal swabbing as a noninvasive method to determine bacterial, archaeal, and eukaryotic microbial community structures in the rumen. Appl Environ Microbiol 81:7470-7483 (2015), which is incorporated herein by reference). and PCR cycle conditions as defined in Kittelmann 2013(Kittelmann et al. Simultaneous Amplicon Sequencing to Explore CoOccurrence Patterns of Bacterial, Archaeal and Eukaryotic Microorganisms in Rumen Microbial Communities. PLOS One 8(2): e47879 (2013), which is incorporated herein by reference). PCR products are stored at the appropriate conditions until subsequent use. The PCR products are then purified, quality checked, prepared into sequencing libraries, and analyzed for 16S rRNA sequencing within three weeks of the final rumen sample collection (Day 90). Libraries are generated using the PerkinElmer NextFlex DNA-Seq kit. The 16S rRNA amplicon libraries are sequenced on the Illumina MiSeq v3 600-cycle (2×300 bp) platform. Following the completion of 16S rRNA sequencing, the resulting data is analyzed for methanogen abundance. Methanogen abundance is reduced by ˜10-80% after treatment with nucleic acid vaccines encoding methanogen cell surface proteins as compared to pre-vaccination.

### Methane Production Measurements

Cow enteric methane production is monitored using GreenFeed Systems (C-Lock, Inc., Rapid City, SD). Briefly, feed intake is recorded daily throughout the study period using GrowSafe Systems. Animals are fed once daily in the morning and have access to a water source at all times. Body weight is recorded periodically, at the time of total blood draws. Enteric methane, hydrogen, and carbon dioxide emissions are measured daily throughout the study period using GreenFeed Systems (C-Lock, Inc., Rapid City, SD). Animals have free access to the GreenFeed System throughout the study period; animals are coerced to use the system. Methane yield and intensity is calculated using dry matter intake for each measurement period separately. Methane production is reduced by ˜10-80% when treated with nucleic acid vaccines encoding methanogen cell surface proteins as compared to pre-vaccination.

This example demonstrates informatic selection of methanogen cell surface proteins for formulation into nucleic acid vaccines, vaccination into ruminants, and subsequent reductions in methane in vivo.

### Example 10: Ruminal Metagenomic Mining of Methanogen Cell Surface Proteins

This example demonstrates informatic selection of candidate methanogen cell surface proteins, generation of nucleic acid vaccines encoding those cell surface proteins, and subsequent methods for analyzing the efficacy of those antigens as nucleic acid vaccines for the reduction of methane production. An exemplary methodology for testing selected constructs is shown in FIG. 20.

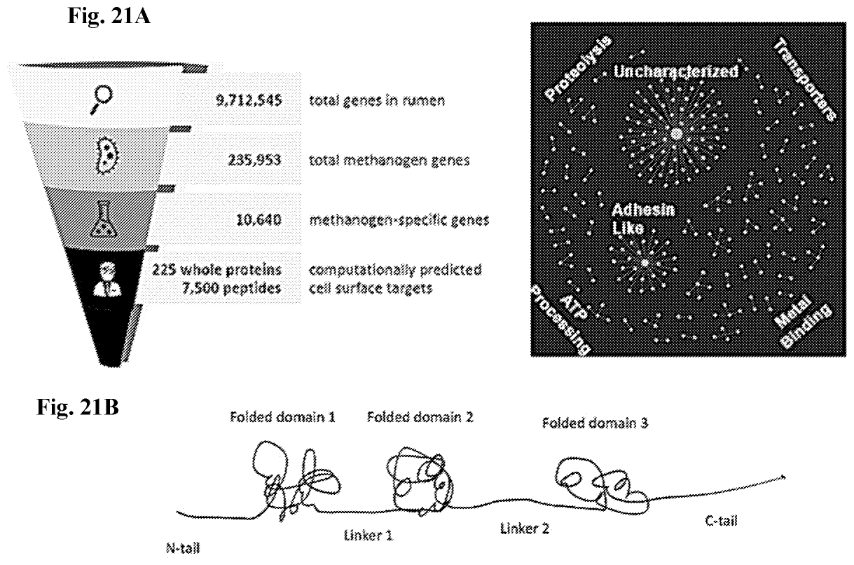

To identify candidate methanogen cell surface proteins, a comparative genomics analysis of ruminal methanogen genomes from rumen samples from 283 beef cattle was used (Stewart (2019) Nature Biotechnology, which is incorporated herein by reference). In total, the analysis comprised 4941 genomes were assembled, of which 126 were archaea and 111 were Methanobrevibacter. Genes were annotated in the 4941 genomes using DIAMOND, resulting in first database comprising 9,712,545 total open reading frames. Methanogen genes were identified by narrowing the total 4941 genomes to those that were archaeal genomes comprising one or more methanogenesis metabolic pathway genes. From those, a second database comprising 235,935 methanogen open reading frames was generated. UniRef100 gene's Gene Onotology terms were obtained and GO term enrichment analysis was performed on each of the ORFs in the second database. The p-values for the enrichment analysis were used as input to a machine learning classification model to classify genomes as methanogen or non-methanogen. This resulted in a third database comprising 10,640 methanogen-specific genes. GO terms most specific to methanogens were used to select candidate genes for localization prediction. pSORTb and TMbed was used to predict subcellular localization and selected whole genes and domains predicted to be accessible to antibodies (e.g., localized to the cell membrane or extracellularly). Based on the subcellular localization, 225 whole proteins and ˜7500 peptide fragments were selected for vaccine production. The bioinformatic selection is shown in FIG. 21A (left). FIG. 21A (right) further shows a network map of the enriched cell surface protein functions, with key methanogen enriched cell surface proteins having functions related to adhesin-like proteins, metal binding, ATP processing, proteolysis, and transport. Many of the adhesin-like proteins comprise cysteine protease domains that are important for cell health, survival, and membrane remodeling.

### Table 20. Select Non-Methanobrevibacter Genes

See SEQ ID NO: 16809 to SEQ ID NO: 16832 for the native nucleic acid sequences of select non-methanobrevibacter genes. See SEQ ID NO: 16833 to SEQ ID NO: 16856 for the bovine codon-optimized nucleic acid sequences of select non-methanobrevibacter genes. See SEQ ID NO: 16857 to SEQ ID NO: 16880 for the amino acid sequences of select non-methanobrevibacter genes.

### Table 21. Select Protein Fragment Sequences

Table 21 presents the sequences for the fragments of the proteins in Table 20. These fragments are isolated functional domains based on predicted computation folding. For example, a hypothetical protein in FIG. 21B comprising 3 folded domains would yield 3 fragments, each of which is a stable domain on its own and can be used as an antigen for vaccine.

See SEQ ID NO: 16681 to SEQ ID NO: 16904 for the native nucleic acid sequences encoding the select protein fragments. See SEQ ID NO: 16905 to SEQ ID NO: 16928 for the bovine codon-optimized nucleic acid sequences encoding the select protein fragments. See SEQ ID NO: 16929 to SEQ ID NO: 16952 for the amino acid sequences of the select protein fragments.

mRNA Synthesis and LNP Preparation

N1-pseudouridine-modified mRNA for the constructs shown in Table 20 and Table 21 are synthesized at TriLink with a 5′ UTR sequence of 5′ AGGAAATAAGAGAGAAAAGAAGAGTAAGAAGAAATATAAGAGCCACC 3′ (SEQ ID NO: 16986), a 3′ UTR sequence of 5′ GCTGCCTTCTGCGGGGCTTGCCTTCTGGCCATGCCCTTCTTCTCTCCCTTGCACCTGT ACCTCTTGGTCTTTGAATAAAGCCTGAGTAGGAAG 3′ (SEQ ID NO: 16987), and a 120A poly A tail (SEQ ID NO: 16988). The constructs were ordered with a signal peptide (5′ ATGTTCGTGTTCCTCGTGCTCCTGCCCCTCGTCTCCAGCCAGTGCGTG 3′ (SEQ ID NO: 16989)) and/or transmembrane domain (5′ CCCTGGTACATCTGGCTGGGCTTCATCGCAGGACTGATCGCCATCGTCATGGTCACC ATAATGCTCTGTTGCATGACCTCCTGCTGTAGCTGCCTGAAAGGCTGTTGCAGCTGC GGGAGCTGCTGCAAATTCGACGAAGACGATAGCGAACCTGTGCTCAAAGGGGTGAA GTTGCATTACACATAGTGA (SEQ ID NO: 16990)). The mRNAs are purified into 1 mM sodium citrate buffer, pH 6.4 and shipped on dry ice. All mRNAs are stored at −80° C. until use.

To prepare lipid nanoparticles (LNPs), the mRNA stocks are thawed on ice. Nucleic acid concentrations are validated by absorbance at 260 nm using a NanoDrop. The mRNAs are then diluted to a final concentration of 0.35 mg/mL mRNA in Precision Nanosystem's dilution buffer. 10 mL of the dilute mRNAs are loaded into separate Luer lock syringes and all bubbles are removed by gentle tapping. A NanoAssemblr Ignite NxGen Cartridge is loaded into the cartridge slot of the Ignite instrument and one mRNA syringe is attached to the aqueous inlet of the cartridge. 3.3 mL of Genvoy-ILM reagent is loaded into a separate Luer lock syringes and all bubbles are removed by gentle tapping. The Genvoy-ILM syringe is then attached to the organic inlet of the cartridge. LNPs are then generated and collected into a 15 mL conical tube. The process is repeated for the remaining mRNAs and for a buffer only LNP control (empty). Immediately after LNPs are generated, the samples are loaded into 10K dialysis cartridges, submerged into 1×DPBS (Ca²⁺ and Mg²⁺-free) to reduce the ethanol concentration to <0.5%. After dialysis the LNPs are recovered from the dialysis cartridges and sterile filtered at 0.2 um. Size and polydispersity are measured using a Malvern Panalytical DLS by diluting 40 uL of LNPs into 360 uL of 1×DPBS. Encapsulation efficiency is measured using a RiboGreen assay following Precision Nanosystems recommended protocol using a BioTek Synergy H1 Plate Reader.

### Vaccination and Sample Collection

Healthy, weaned, Angus cross steers are weaned for a minimum of sixty days and received pre-weaned vaccinations a minimum of sixty days prior to initiation of the study. Animals are transferred to the study site fourteen days prior to vaccination to allow for acclimation to the study diet and environment. Each vaccine formulation is administered by IM injection to three animals. In total, 120 animals are vaccinated with LNPs containing ˜500 ug mRNA and 3 animals are vaccinated with empty LNPs. Intramuscular injections are delivered into the cow's right neck muscle by veterinary-trained staff using an 18-gauge 1-1.5″ needle. Animals are boosted with a second vaccine administration on day 21. Total blood samples (˜100 mL) are collected from the jugular vein by veterinary-trained staff and processed by ultracentrifugation within 24 hours of sample collection. Isolated serum samples are aliquoted, labeled and stored in cryogenic tubes at −20° C. until use.

### Ruminal Microbiome Measurements

˜50 mL Of fresh rumen samples are collected by esophageal tubing and retained in conical vials. Samples are strained through three layers of cheesecloth, with the liquid phase aliquoted into 50 mL conical vials. All samples re snap-frozen in liquid N₂ and stored at −20° C. for subsequent processing. Samples are processed within twenty-four hours of collection. Sample aliquots are labeled with animal number, collection method, collection date and time.

Isolation of DNA from the rumen follows the methods defined in Henderson (Henderson, G. et al. Effect of DNA Extraction Methods and Sampling Techniques on the Apparent Structure of Cow and Sheep Rumen Microbial Communities. PLOS One 8, e74787 (2013), which is incorporated herein by reference), with preference given to methods involving both phenol-chloroform and mechanical lysis steps (PCQI, PCBB, PCSA). PCR amplification of the hypervariable V6-V8 regions of the 16S rRNA gene is performed using the archaea-specific Ar915aF/Ar1386R primer set with Illumina adapters as defined in Table 1 of Kittelmann 2015 (Kittelmann et al. Buccal swabbing as a noninvasive method to determine bacterial, archaeal, and eukaryotic microbial community structures in the rumen. Appl Environ Microbiol 81:7470-7483 (2015), which is incorporated herein by reference). and PCR cycle conditions as defined in Kittelmann 2013(Kittelmann et al. Simultaneous Amplicon Sequencing to Explore CoOccurrence Patterns of Bacterial, Archaeal and Eukaryotic Microorganisms in Rumen Microbial Communities. PLOS One 8(2): e47879 (2013), which is incorporated herein by reference). PCR products are stored at the appropriate conditions until subsequent use. The PCR products are then purified, quality checked, prepared into sequencing libraries, and analyzed for 16S rRNA sequencing within three weeks of the final rumen sample collection (Day 90). Libraries are generated using the PerkinElmer NextFlex DNA-Seq kit. The 16S rRNA amplicon libraries are sequenced on the Illumina MiSeq v3 600-cycle (2×300 bp) platform. Following the completion of 16S rRNA sequencing, the resulting data is analyzed for methanogen abundance. Methanogen abundance is reduced by ˜10-80% after treatment with nucleic acid vaccines encoding methanogen cell surface proteins as compared to pre-vaccination.

### Methane Production Measurements

Cow enteric methane production is monitored using GreenFeed Systems (C-Lock, Inc., Rapid City, SD). Briefly, feed intake is recorded daily throughout the study period using GrowSafe Systems. Animals are fed once daily in the morning and have access to a water source at all times. Body weight is recorded periodically, at the time of total blood draws. Enteric methane, hydrogen, and carbon dioxide emissions are measured daily throughout the study period using GreenFeed Systems (C-Lock, Inc., Rapid City, SD). Animals have free access to the GreenFeed System throughout the study period; animals are coerced to use the system. Methane yield and intensity is calculated using dry matter intake for each measurement period separately. Methane production is reduced by ˜10-80% when treated with nucleic acid vaccines encoding methanogen cell surface proteins as compared to pre-vaccination.

This example demonstrates informatic selection of methanogen cell surface proteins for formulation into nucleic acid vaccines, vaccination into ruminants, and subsequent reductions in methane in vivo.

### Example 11: Pregnant and Postpartum Animal Vaccination

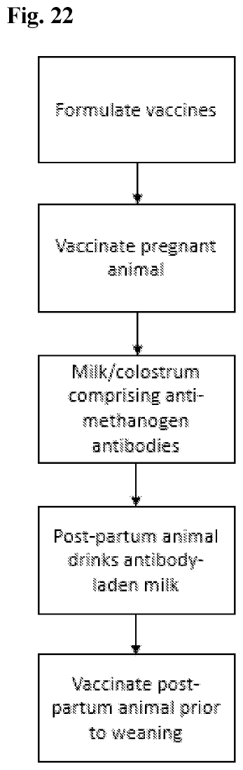

FIG. 22 shows an exemplary vaccination protocol. At least one vaccine (e.g., a nucleic acid vaccine comprising a nucleic acid sequence encoding at least one methanogen surface antigen) is prepared. The at least one vaccine is used to vaccinate a pregnant animal (e.g., ruminant). The unborn fetus is exposed to the antibodies against methanogen generated by the mother. After birth, the child ruminant receives milk laden with the antibodies against the at least one methanogen surface antigen. The child ruminant is vaccinated with at least one vaccine of the present disclosure prior to weaning. The child ruminant may be vaccinated with a vaccine encoding the same or different methanogen surface antigen.

### Example 12: Combinatorial Therapies

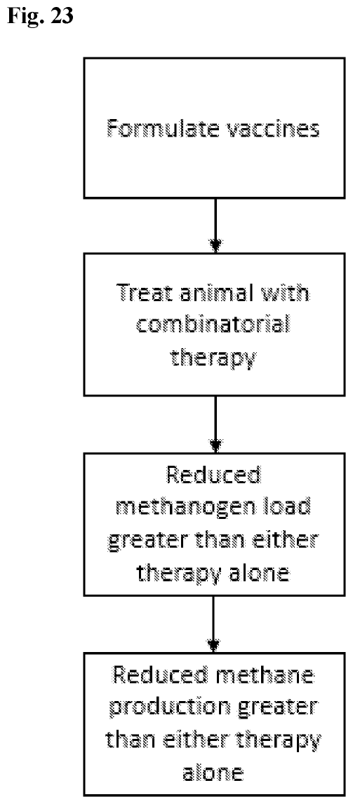

FIG. 23 shows another exemplary vaccination protocol. At least one vaccine (e.g., a nucleic acid vaccine comprising a nucleic acid sequence encoding at least one methanogen surface antigen) is prepared. A ruminant is treated with a combinatory therapy, which comprises any two or more selected from a vaccine, antibodies, milk, animal feed, an agent (e.g., an agent that reduces methane production in a ruminant, a probiotic bacterial strain, a small molecule inhibitor, etc.), and other composition of the present disclosure (e.g., those reducing methane production in a ruminant). Here, a reduction in methane production that is greater than the use of a single therapy alone is achieved.

### Example 13: Synthesis of 3-NOP

Adapted from from “Structure-Based Design, Synthesis, and Biological Evaluation of Indomethacin Derivatives as Cyclooxygenase-2 Inhibiting Nitric Oxide Donors” (J. Med. Chem. 2007, 50, 6367-6382).

All reagents purchased from Sigma-Aldrich and Fisher Scientific and used as received.

3-Bromopropanol (78.0 g, 0.56 mol) in acetonitrile (300 mL) was added dropwise within minutes to a solution of silver nitrate (145.9 g, 0.86 mol) in acetonitrile (600 mL) and stirred at room temperature for 24 hr. The solution was protected from light by being covered with aluminum foil. After 24 hr, 5:1 excess of brine was added to the reaction mixture and stirred for 1 hr. Silver halide was filtered through Celite and filtrate was extracted with diethyl ether (300 mL×3). The organic layer was washed with brine (300 mL×3). Dried over sodium sulfate, and concentrated (59 g, 86% yield). Product confirmed via NMR (>97% purity based on HPLC).

3-NOP pH and Temperature Stability: Confirmed via HPLC over a 1-month time period at temperature between 4-30C and pH 4-10.

3-NOP Volatility:500 mg of 3-NOP in a 5 dram vial was left open to air at ˜18-24C at 15-30% RH over the course of 6 days. Average mass loss is ˜0.25% per day.

Compaction Study

The viability of different binders for densification were examined. 0.5 g of powder was tableted under 20 kN in a 13 mm pellet die. Potential binders are summarized in Table 22.

TABLE 22
Potential binders ranked based on densification.

| | | | 2: compacts, |
|---|---|---|---|
| | | | no strength |
| 5: Unbreakable | | 3: powdery break, | 1: compacts, |
| tablet | 4: clean braking, | retains moderate | disintegrates |
| by hand | no dust | strength | 0: no compaction |
| Xanthan gum | Lignin | Pea protein | Chitosan |
| k-carrageenan | Inulin | Soy protein | Hydroxyethyl |
| | | | cellulose |
| Sodium alginate | Wood flour | Gelatin | Talc |
| Cellulose acetate | Corn Starch | Casein | Sodium starch |
| | | | glycolate |
| Ethyl cellulose | Amylopectin | | Pentaerythritol |
| Hydroxypropyl | Hemp protein | | Calcium phosphate |
| cellulose | | | |
| Cellulose acetate | | | Diatomaceous earth |
| phthalate | | | (Celite) |
| Maltodextrin | | | Biochar |
| Dextran | | | |

Tablets for Encapsulation of 3-NOP

A multilayer encapsulation system was employed for extended-release formulations consisting of 3 main parts:
1) Adsorbent—inert, non-toxic, solid-support for 3-NOP (ie. silica, biochar, activated charcoal, talc, arginine, lysine, calcium carbonate, carbon black, cyclodextrin, calcium phosphate, celite (diatomaceous earth), glutamine, betaine, bismuth phosphate, bismuth citrate, iron phosphate, etc)
2) Binder/matrix—allow for granulation of adsorbent to form solid core (ie, starches, flours, sugars, proteins, gums, gelatin, cellulose and derivatives, etc)
3) Coating—single to multilayer coatings to decrease water penetration and modify release kinetics

All materials purchased from Sigma-Aldrich and Fisher Scientific and used as received unless otherwise specified.

Tablet Preparation

3-NOP was adsorbed onto the corresponding adsorbent at a given weight percent (noted in formulations) by dropwise addition to adsorbent stirring on a hot plate. The mixture was left to stir for more than 30 minutes until a free-flowing powder was obtained. The resulting mixture was speed mixed with the corresponding binder for 1 minute followed by compaction of in a carver press using an 8-13 mm pellet die under various forces of 10-60 kN.

Coated Tablet Preparation

Selected coating materials were dissolved in various solvents at 5-20 wt %. Tablets were then dip coated multiple times allowing for drying in between to give the desired coating weight.

3-NOP Release Quantification and Corresponding Tablet Formulations

13 mm, 0.5 g tablets were placed in a sealed 5 dram vial with 10 mL of distilled water and incubated at 70 F until full dissolution of 3-NOP. 1 mL aliquots were taken every few days and refreshed with 1 mL of distilled water. Aliquots were filtered through a 0.22 um PVDF filter before being quantified via HPLC.

Uncoated Tablets

All formulation percentages are based on weight. 2 replicates of 13 mm disc-shaped, 0.5 g tablets were compacted under 20 kN. The uncoated formulations had the following compositions:
AH-01-7.5% 3-NOP, 12.5% propylene glycol, 60% Cargill hemp protein
AH-02-8.57% 3-NOP, 22.86% Celite, 68.57% hemp protein
AH-03-8.57% 3-NOP, 22.86% Wakefield Biochar, 68.57% Cargill hemp protein
AH-04-8.57% 3-NOP, 22.86% K10 montmorillonite, 68.57% Cargill hemp protein
AH-05-8.57% 3-NOP, 22.86% activated charcoal, 68.57% Cargill hemp protein

The 3-NOP release of the uncoated tablets after 12 hours is shown in Table 23.

| | Total Average | |
|---|---|---|
| | Concentration | Standard |
| | of 3-NOP | deviation |
| Formula | (mM) | (mM) |
| AH-01 | 24.56 | 2.09 |
| AH-02 | 24.50 | 1.76 |
| AH-03 | 19.61 | 0.17 |
| AH-04 | 33.10 | 0.10 |
| AH-05 | 18.58 | 0.07 |

Coated Tablets

Tablets for coating experiments consist of 10% 3-NOP, 20% Wakefield biochar, and 70% cellulose acetate (Mn: 50,000). Tablets were then coated with ˜0.2 wt % of the corresponding coating material, as shown in the table in Table 24. The release profile over periods of 4, 6 and 11 days for each formula is also shown.

TABLE 24
3-NOP release profile of coated tablets over 4 days.

| | | Total Average | Standard |
|---|---|---|---|
| | Time | Concentration | deviation |
| | (days) | (mM) | (mM) |
| Cellulose Acetate | 4 | 34.21 | 2.23 |
| Phthalate (CAP) | | | |
| CAP | 6 | 30.65 | 0.04 |
| CAP | 11 | 32.38 | 0.24 |
| Ethyl cellulose (EC) | 4 | 29.92 | 0.06 |
| EC | 6 | 31.07 | 0.01 |
| EC | 11 | 31.50 | 0.26 |
| Hydroxy propyl | 4 | 31.70 | 0.05 |
| cellulose (HPC) | | | |
| HPC | 6 | 30.35 | 0.01 |
| HPC | 11 | 32.53 | 0.4 |
| Polycaprolactone | 4 | 32.08 | 0.29 |
| (CAPA 6800) | | | |
| CAPA 6800 | 6 | 31.00 | 0.14 |
| CAPA 6800 | 11 | 31.99 | 0.23 |
| Alignate (layer 1) | 4 | 30.30 | 0.17 |
| Chitosan (layer 2) | | | |
| Alignate (layer 1) | 6 | 30.22 | 0.17 |
| Chitosan (layer 2) | | | |
| Alignate (layer 1) | 11 | 31.90 | 0.10 |
| Chitosan (layer 2) | | | |
| Polycaprolactone | 4 | 33.68 | 0.01 |
| Mn: 2000 | | | |
| Polycaprolactone | 6 | 32.25 | 0.18 |
| Mn: 2000 | | | |
| Polycaprolactone | 11 | 33.23 | 0.09 |
| Mn: 2000 | | | |
| PEG Mn: 1500 | 4 | 32.74 | 0.22 |
| PEG Mn: 1500 | 6 | 31.88 | 1.30 |
| PEG Mn: 1500 | 11 | 33.48 | 0.29 |

Further formulations were prepared and tested. 3 replicates of 13 mm disc-shaped, 0.5 g tablets were compacted under 60 kN. All formulas consist of 23.8% 3-NOP, 28.6% silica, and 47.6% ethyl cellulose. Tablets were then coated with 60-80 wt % of the corresponding coating material. Control tablet (CTRL) is uncoated. “R” denotes replicate number. CAP coating solution was 18 wt % in 70% ethanol/water. Cellulose acetate (CA) coating was 6 wt % in 70% ethanol/ethyl acetate with an additional 5 wt % triethyl citrate, 5 wt % propylene glycol, and 5 wt % N-methyl-2-pyrollidone. The release profiles were analyzed at 1, 4, 8, 11, and 15 days, as summarized in Table 25.

TABLE 25
3-NOP release profile of coated tablets over 15 days.
Total concentration of 3-NOP is listed in mM.

| | | | | | |
|---|---|---|---|---|---|
| CAP R1 | 25.74 | 47.79 | 59.39 | 61.05 | 59.68 |
| CAP R2 | 18.72 | 43.71 | 52.56 | 53.74 | 53.37 |
| CAP R3 | 23.12 | 50.66 | 57.23 | 59.23 | 58.75 |
| AVG | 22.53 | 47.39 | 56.39 | 58.01 | 57.26 |
| STD | 3.54 | 3.49 | 3.49 | 3.81 | 3.41 |
| CA R1 | 50.90 | 75.98 | 73.80 | 66.97 | 61.48 |
| CA R2 | 71.29 | 78.41 | 73.11 | 66.66 | 60.18 |
| CA R3 | 63.86 | 69.38 | 65.56 | 61.28 | 57.00 |
| AVG | 62.01 | 74.59 | 70.82 | 64.97 | 59.55 |
| STD | 10.32 | 4.67 | 4.57 | 3.20 | 2.31 |
| CTRL R1 | 45.80 | 80.64 | 80.33 | 73.64 | |
| CTRL R2 | 45.80 | 84.41 | 83.39 | 76.22 | 69.25 |
| CTRL R3 | 45.80 | 77.15 | 75.92 | 70.11 | 62.74 |
| AVG | 45.80 | 80.73 | 79.88 | 73.32 | 66.00 |
| STD | 0.00 | 3.63 | 3.76 | 3.07 | 4.60 |

Coated Tablets

All formulation percentages are based on weight. 3 replicates of 13 mm disc-shaped, 0.5 g tablets were compacted under 60 kN. All formulas consist of 23% 3-NOP, 30% silica, and 47% ethyl cellulose. Tablets were then coated with 60-80 wt % of the corresponding coating material. Control tablet (CTRL) is uncoated. V1 coating solution was 20 wt % cellulose acetate Mn: 50000 in 70% acetone/ethyl acetate with BYK coating additives. V2 coating solution was 20 wt % in 70% acetone/ethyl acetate cellulose acetate Mn: 50000 with BYK coating additives. Ethyl cellulose coating solution was 20 wt % in 70% ethanol/water with 15 wt % triacetin. CAP coating solution was 18 wt % in 70% ethanol/water with 10 wt % propylene glycol. The 3-NOP release profile of the coated tablets over 18 days is shown in Table 26.

TABLE 26
3-NOP release profile of coated tablets over 18 days.
Total concentration of 3-NOP is listed in mM.

| Time | 1 | 5 | 8 | 12 | 18 |
|---|---|---|---|---|---|
| V1 R1 | 33.04 | 70.32 | 70.57 | 66.04 | 60.88 |
| V1 R2 | 57.54 | 77.54 | 77.42 | 72.65 | 67.44 |
| V1 R3 | 61.42 | 71.21 | 70.08 | 65.71 | 61.19 |
| AVG | 50.67 | 73.02 | 72.69 | 68.13 | 63.17 |
| STD | 15.39 | 3.93 | 4.10 | 3.91 | 3.70 |
| V2 R1 | 31.15 | 66.47 | 65.62 | 61.03 | 56.78 |
| V2 R2 | 32.55 | 66.81 | 67.61 | 63.67 | 59.55 |
| V2 R3 | 53.59 | 69.37 | 71.28 | 67.53 | 63.01 |
| AVG | 39.10 | 67.55 | 68.17 | 64.08 | 59.78 |
| STD | 12.57 | 1.59 | 2.87 | 3.27 | 3.12 |
| EC R1 | 17.99 | 39.67 | 45.39 | 47.12 | 50.00 |
| EC R2 | 15.84 | 38.15 | 43.53 | 45.83 | 49.27 |
| EC R3 | 21.21 | 52.28 | 58.64 | 60.42 | 63.56 |
| AVG | 18.35 | 43.37 | 49.19 | 51.13 | 54.28 |
| STD | 2.70 | 7.75 | 8.24 | 8.08 | 8.05 |
| CAP R1 | 24.07 | 57.62 | 63.10 | 69.28 | 68.61 |
| CAP R2 | 23.24 | 56.30 | 61.85 | 60.67 | 59.42 |
| CAP R3 | 37.58 | 63.57 | 71.80 | 69.89 | 70.48 |
| AVG | 28.30 | 59.17 | 65.58 | 66.61 | 66.17 |
| STD | 8.05 | 3.87 | 5.42 | 5.15 | 5.92 |
| CTRL R1 | 61.62 | 97.29 | 89.52 | 81.26 | 74.20 |
| CTRL R2 | 45.40 | 82.69 | 79.42 | 73.16 | 67.07 |
| CTRL R3 | 53.75 | 83.30 | 76.77 | 70.18 | 64.12 |
| AVG | 53.59 | 87.76 | 81.90 | 74.87 | 68.47 |
| STD | 8.11 | 8.26 | 6.73 | 5.74 | 5.18 |

Bilayer Coated Tablets

All formulation percentages are based on weight. 3 replicates of 13 mm disc-shaped, 0.5 g tablets were compacted under 60 kN. All formulas consist of 15% 3-NOP, 20% silica, and 65% ethyl cellulose. Bilayer coatings were applied with ˜10 wt % of the first coating material and ˜20 wt % of the second coating material. CAP/EC denoted first and second coating material. The 3-NOP release profile of bilayer coated tablets over 27 days is shown in Table 27.

TABLE 27
3-NOP release profile of bilayer coated tablets over 27
days. Total concentration of 3-NOP is listed in mM.

| | Time (days) | | | | | | | | |
|---|---|---|---|---|---|---|---|---|---|
| | 1.25 | 2.5 | 5.5 | 8 | 10 | 14 | 17 | 21 | 27 |
| CAP/ | 6.27 | 8.80 | 15.97 | 18.49 | 20.41 | 24.15 | 24.59 | 24.46 | 26.21 |
| EC R1 | | | | | | | | | |
| CAP/ | 4.39 | 7.77 | 15.50 | 17.78 | 19.54 | 22.90 | 23.44 | 23.74 | 25.94 |
| EC R2 | | | | | | | | | |
| CAP/ | 5.63 | 8.43 | 15.44 | 17.84 | 19.29 | 20.36 | 20.94 | 21.18 | 22.65 |
| EC R3 | | | | | | | | | |
| AVG | 5.43 | 8.33 | 15.64 | 18.04 | 19.75 | 22.47 | 22.99 | 23.13 | 24.93 |
| STD | 0.96 | 0.52 | 0.29 | 0.39 | 0.59 | 1.93 | 1.87 | 1.73 | 1.98 |
| EC/ | 9.75 | 14.58 | 22.35 | 23.09 | 23.76 | 26.40 | 26.04 | 26.12 | 26.82 |
| CAP R1 | | | | | | | | | |
| EC/ | 10.72 | 17.13 | 24.89 | 26.10 | 26.78 | 29.51 | 28.89 | 28.52 | 29.31 |
| CAP R2 | | | | | | | | | |
| EC/ | 9.84 | 15.34 | 22.33 | 24.08 | 25.21 | 28.03 | 27.91 | 27.56 | 28.17 |
| CAP R3 | | | | | | | | | |
| AVG | 10.10 | 15.68 | 23.19 | 24.42 | 25.25 | 27.98 | 27.61 | 27.40 | 28.10 |
| STD | 0.54 | 1.31 | 1.47 | 1.54 | 1.51 | 1.56 | 1.45 | 1.21 | 1.25 |
| CTRL | 26.11 | 28.76 | 36.26 | 37.49 | 36.75 | 35.77 | 33.69 | 31.26 | 32.66 |
| R1 | | | | | | | | | |
| CTRL | 26.12 | 28.43 | 38.45 | 39.57 | 38.58 | 37.45 | 34.91 | 32.16 | 33.30 |
| R2 | | | | | | | | | |
| CTRL | 25.23 | 29.94 | 40.65 | 41.23 | 45.02 | 42.91 | 39.10 | 27.92 | −2.04 |
| R3 | | | | | | | | | |
| AVG | 25.82 | 29.05 | 38.45 | 39.43 | 40.12 | 38.71 | 35.90 | 30.45 | 21.31 |
| STD | 0.51 | 0.79 | 2.19 | 1.87 | 4.34 | 3.73 | 2.84 | 2.24 | 20.22 |

Adsorbent Study

Various adsorbents were assessed for potential binding affinity and adsorptive capacity of 3-NOP. Activated carbon/charcoal and silica were identified as the most suitable adsorbents based on their high binding affinity and adsorptive capacity.

Briefly, 0.2 g of adsorbent was added to 4 mL of 2.5% 3-NOP/H2O stock solution or 10 mL of 1% 3-NOP/H2O stock solution. The solution was then placed in an ultrasonic bath for ˜4 hr and then allowed to equilibrate over 24 h. All materials were purchased from Sigma-Aldrich and used as received. After 24 h, adsorbents were tested for their binding affinity and adsorptive capacity by looking at the reduction of 3-NOP concentration via HPLC at 230 nm.

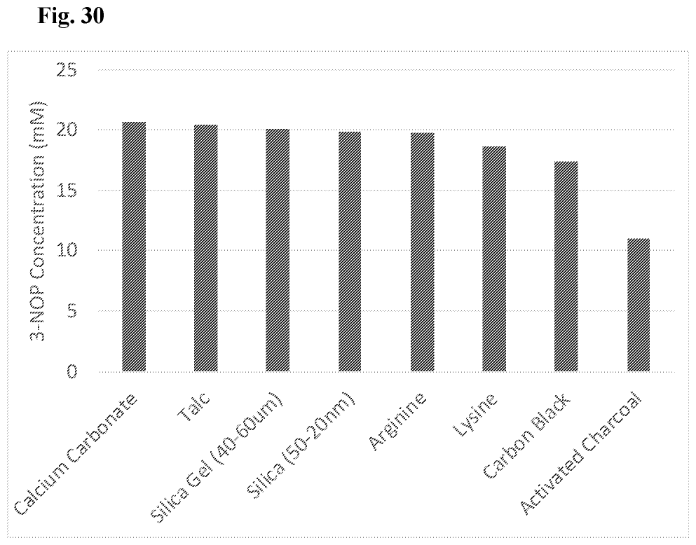

The following adsorbents were tested and integrated areas were converted to mM concentrations of 3-NOP based on calibration standards of known 3-NOP concentration in distilled water, as seen in Table 28. FIG. 30 is graph showing the concentration of 3-NOP (mM) v. adsorbent (20 mM stock solution).

TABLE 28
Change in concentration
v. control of 0.2 g of
adsorbent in 4 mL
of 2.5% 3-NOP/H2O.

| | 3-NOP |
|---|---|
| | concentration |
| Adsorbent | (mM) |
| Control | 20.649 |
| Calcium Carbonate | 20.649 |
| Talc | 20.463 |
| Silica Gel (40-60 um) | 20.043 |
| Silica (5-20 nm) | 19.920 |
| Arginine | 19.782 |
| Lysine | 18.583 |
| Carbon Black | 17.422 |
| Activated Charcoal | 11.063 |

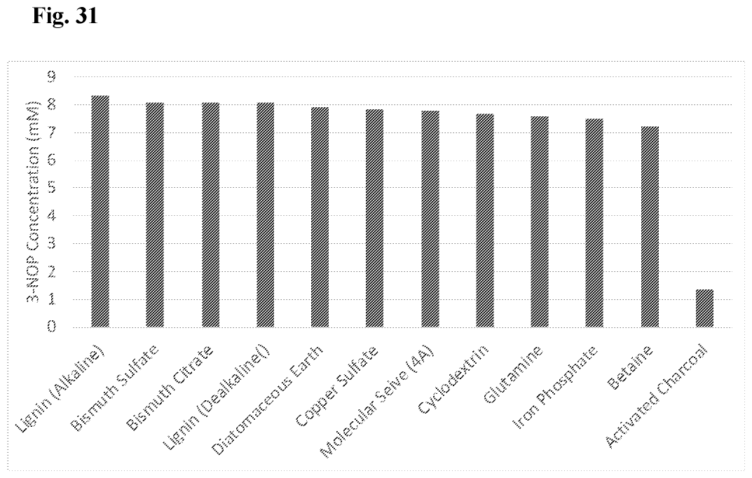

Table 29 shows the change in concentration v. control of 0.2 g of adsorbent in 10 mL of 1% 3-NOP/H2O, and FIG. 31 shows 3-NOP concentration (mM) v. adsorbent (˜8 mM stock solution).

TABLE 29
Change in concentration
v. control of 0.2 g of
adsorbent in 10 mL
of 1% 3-NOP/H2O.

| | 3-NOP |
|---|---|
| | concentration |
| Adsorbent | (mM) |
| Control | 8.258 |
| Lignin (Alkaline) | 8.326 |
| Bismuth Sulfate | 8.079 |
| Bismuth Citrate | 8.072 |
| Lignin (Dealkaline) | 8.056 |
| Diatomaceous Earth | 7.895 |
| Copper Sulfate | 7.814 |
| Molecular Sieve (4Å) | 7.771 |
| Cyclodextrin | 7.676 |
| Glutamine | 7.588 |
| Iron Phosphate | 7.503 |
| Betaine | 7.202 |
| Activated Charcoal | 1.358 |

Activated carbon shows strong binding affinity for 3-NOP filtering ˜45 wt % in concentrated (20 mM) conditions with ˜80 wt % in more dilute concentrations (8 mM). It appears that the adsorbent pore size should be greater than 4 Å for adsorption of 3-NOP. Mesoporous adsorbents with high surface area such as silica and activated carbon show high adsorptive capacity with no visible wetting seen until ˜75 wt %. Layer-by-Layer

Polyelectrolytes Coatings

Formulated tablets containing 3-NOP, an adsorbent, and a binder can be coated with sequential layers of oppositely charged polyelectrolytes. While not being bound by theory, it is believed that ionic crosslinking between the layers can result in a controlled release of 3-NOP out of the tablets. Examples of polyelectrolyte pairs that can be used include polyglutamic acid and polylysine, polyallylamine hydrochloride and polyacrylic acid, and polyallylamine hydrochloride and polystyrene sulfonate. An exemplary formulated tablet includes an adsorbent of silica, arginine, lysine, and activated charcoal (30-45%) and a binder of ethyl cellulose and hydroxypropyl cellulose (40-55%).

TABLE 30
Formulation examples of 3-NOP tablets.
Tablet weight varied from 0.2-0.5 g.
Tablet Formulation

| | Part per | | Part per |
|---|---|---|---|
| | formulation | | formulation |
| Adsorbent | (wt %) | Binder | (wt %) |
| Silica, Arginine, | 30-45 | Ethyl Cellulose, | 40-55 |
| Lysine, Activated | | Hydroxypropyl | |
| Charcoal | | Cellulose | |

The release data of 3-NOP tablets coated with multilayers of some polyelectrolytes is shown in Table 31.

TABLE 31
Release (%) of 3-NOP out of tablets formulated with
15% 3-NOP, 30% silica, and 55% ethyl cellulose. Weight
of tablets ~0.5 g. Calculations are based on a
comparison to control tablets with no coating.

| | Release Compared to Control Tablets (%) | | | | | | | |
|---|---|---|---|---|---|---|---|---|
| | Day | Day | Day | Day | Day | Day | Day | Day |
| PE Pair | 1 | 3 | 6 | 8 | 14 | 16 | 21 | 30 |
| PLK20 + | 58.1 | 56.6 | 61.3 | 62.6 | 64.7 | 63.6 | 63.8 | 63.2 |
| PRE20 | | | | | | | | |
| PAH + PAA | 61.5 | 62.5 | 67.1 | 70.2 | 70.4 | 71.4 | 71.7 | 71.6 |
| PAH + PSS | 61 | 66.5 | 74.7 | 76 | 77.5 | 76.6 | 76.7 | 74.8 |

3-NOP to Adsorbent Ratio Study

Optimal loading of 3-NOP onto adsorbents was determined by varying ratios of 3-NOP to adsorbent. 3-NOP and adsorbent were speed mixed at 2000 rpm for 1 min at the following ratios by weight: 1:1.5, 1:2, 1:2.5. The adsorbed 3-NOP powder mixture was then placed in distilled water at 1% 3-NOP loading. The solution was allowed to equilibrate for 24 hr and then an aliquot was taken for HPLC to determine the reduction in 3-NOP concentration. Table 32 shows the average integrates are of 3-NOP in solution at various adsorbent ratios.

TABLE 32
Average integrated area of 3-NOP in solution at varying 3-NOP:adsorbent ratios.

| | 3-NOP:Adsorbent | | | | | |
|---|---|---|---|---|---|---|
| | 1:1.5 | | 1:2 | | 1:2.5 | |
| | Average | | Average | | Average | |
| | Integrated | Standard | Integrated | Standard | Integrated | Standard |
| | Area | Deviation | Area | Deviation | Area | Deviation |
| 3-NOP | 2566.4 | 51.3 | 2566.4 | 51.3 | 2566.4 | 51.3 |
| Silica | 2499.4 | 293.2 | 2321.9 | 99.3 | 2340.9 | 30.3 |
| Activated | 1751.2 | 29.6 | 1581.9 | 8.5 | 1403.7 | 14.5 |
| charcoal | | | | | | |
| Lysine | 2336.2 | 61.7 | 2308.9 | 23.9 | 2266.6 | 56.1 |
| Arginine | 2406.0 | 113.2 | 2372.8 | 61.2 | 2435.2 | 230.2 |
| Glutamic | 2445.9 | 149.9 | 2452.5 | 78.9 | 2446.3 | 85.0 |
| acid | | | | | | |

Higher ratios of activated carbon to 3-NOP result in higher adsorptive capacity. At ratio of 1:2.5 an adsorption capacity of ˜45% is observed and at a ratio of 1:1.5 an adsorption capacity of ˜30% is observed.

Activated Carbon Tablets

Based on the 3-NOP:adsorbent ratio study, the same ratios were tested for tablet integrity, friability, porosity, etc to determine the optimal 3-NOP loading. 500 mg, 13 mm tablets were compacted under 60 kN.

High ratios of activated carbon (1:2.5 3-NOP:adsorbent) resulted in tablets that absorb more water and coating solutions. This water/solvent absorption of non-wetted charcoal causes difficulty in producing uniform, defect free coatings. High loadings of activated charcoal >25% in tablets result in poor tablet integrity characterized by loss in hardness, increased porosity, tablet spilling, and poor shape retention. The preferred loading for tablets with poor disintegration was 1:1.5 3-NOP:adsorbent.

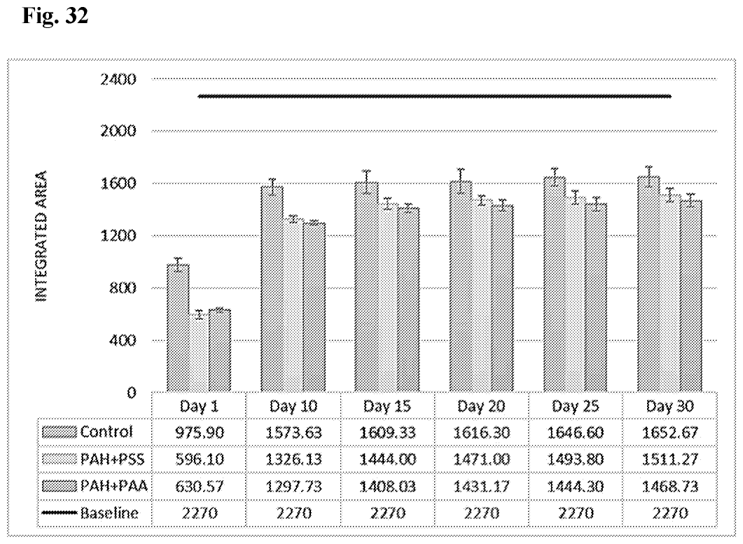

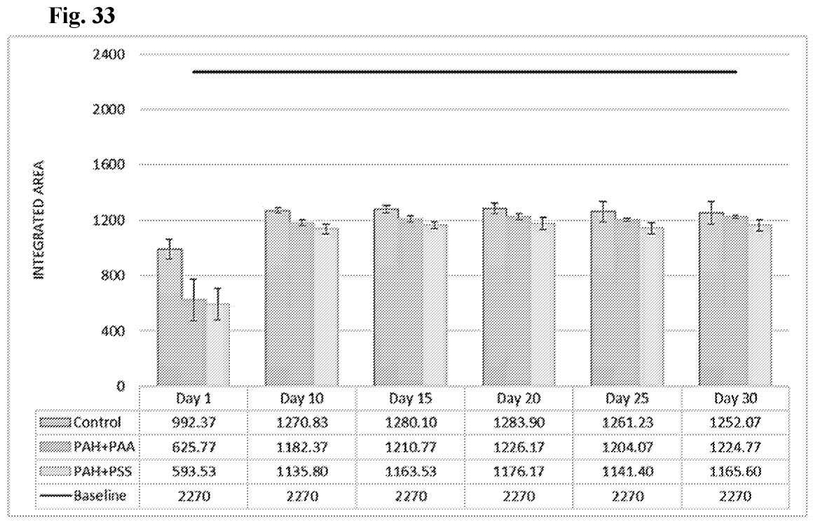

FIG. 32 shows a multilayer tablet formulation above consisting of 10 wt % 3-NOP, 15 wt % activated carbon, and 75 wt % ethyl cellulose. The first day release of coated tablets is ˜40% less than uncoated tablets and after 30 days, ˜30% 3-NOP remains adsorbed on activated carbon.

FIG. 33 shows a multilayer tablet formulation consisting of 10 wt % 3-NOP, 25 wt % activated carbon, and 75 wt % ethyl cellulose. After 30 days, ˜50% 3-NOP remains adsorbed on activated carbon. These adsorptive capacities are in-line with what was observed during the 3-NOP:adsorbent ratio study.

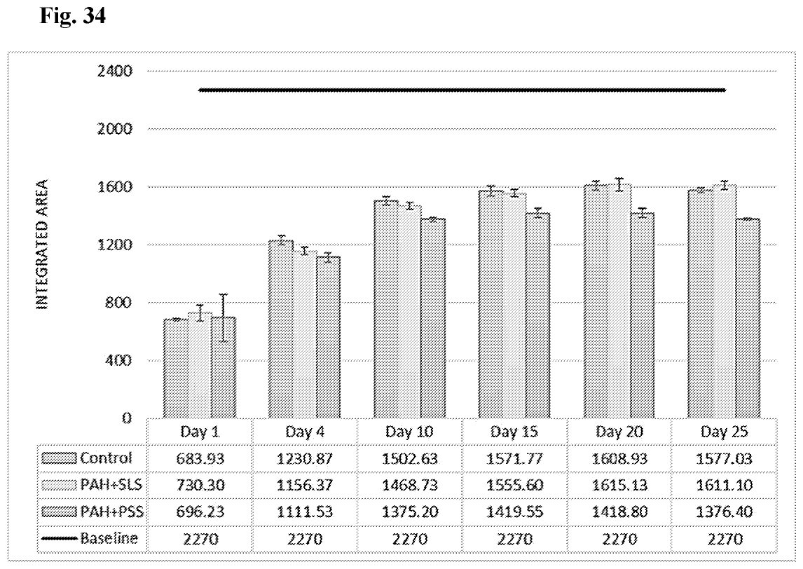

FIG. 34 shows a multilayer formulation consisting of 10 wt % 3-NOP, 15 wt % activated carbon, 5 wt % sodium lignosulfonate, and 70 wt % ethyl cellulose. The addition of 5 wt % sodium lignosulfonate improves coating adhesion to the tablet resulting in ˜30% reduction in the first day 3-NOP release. Over time, the release rate remains similar to previous formulations with a residual 30% 3-NOP remaining adsorbed to activated carbon.

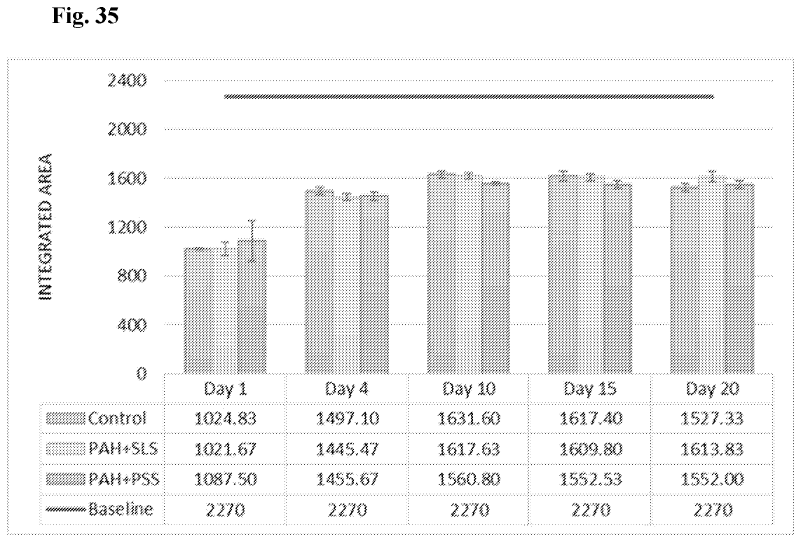

FIG. 35 shows a multilayer formulation consisting of 10 wt % 3-NOP, 15 wt % activated carbon, 5 wt % hydroxypropyl cellulose, and 70 wt % ethyl cellulose. The addition of 5 wt % hydroxypropyl cellulose, a hydrophilic water-soluble binder, results in increased release between day 4 and day 10. After 30 days, ˜30% 3-NOP remains adsorbed to activated carbon.

Silica v. Activated Carbon Controls to Assess Binding Affinity of 3-NOP

Various control experiments were conducted to confirm the adsorbed 3-NOP to activated carbon after 30 days. Activated carbon was compared to silica as preferred adsorbents.

500 mg of 1:1.5 3-NOP:adsorbent were speed mixed at 2000 rpm for 1 min. The resulting powder mixture was placed in 10 mL of distilled water and 10 mL of acetonitrile (ACN) separately. The solutions were placed in a sonic bath for ˜5 hr, removed, and allowed to equilibrate for 24 hr.

Aliquots were filtered through a 0.22 um PTFE filter, and taken for HPLC to measure the 3-NOP concentration in solution.

The same trend was observed in the adsorbent study. However, 3-NOP is recoverable by washing the activated carbon with acetonitrile or a similar organic solvent with high 3-NOP solubility (ie. ethanol, acetone, ethyl acetate, tetrahydrofuran, chloroform).

TABLE 33
Silica powder release v. activated carbon
release in distilled water and acetonitrile.

| | 24 hr | 24 hr |
|---|---|---|
| | in H2O | in ACN |
| | Integrated | Integrated |
| | Area | Area |
| Si-1 | 8415.7 | 8847 |
| Si-2 | 8507.3 | 8822.2 |
| Average | 8461.5 | 8834.6 |
| AC-1 | 4940.8 | 8103.9 |
| AC-2 | 5577.5 | 8082.8 |
| Average | 5259.2 | 8093.4 |

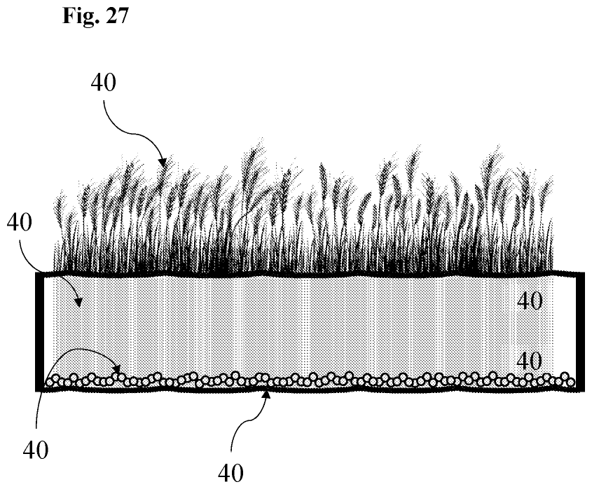

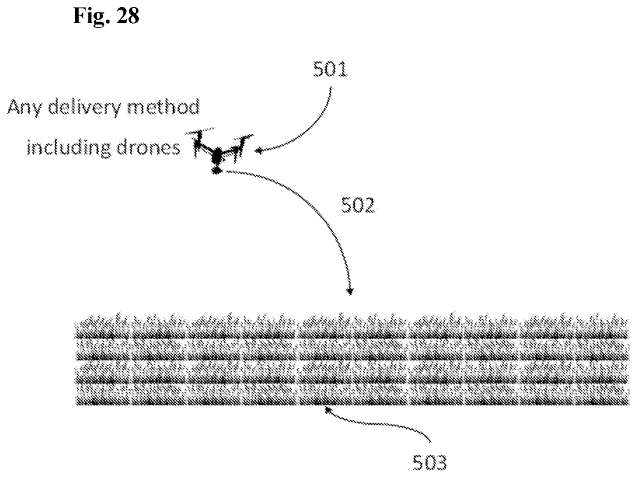

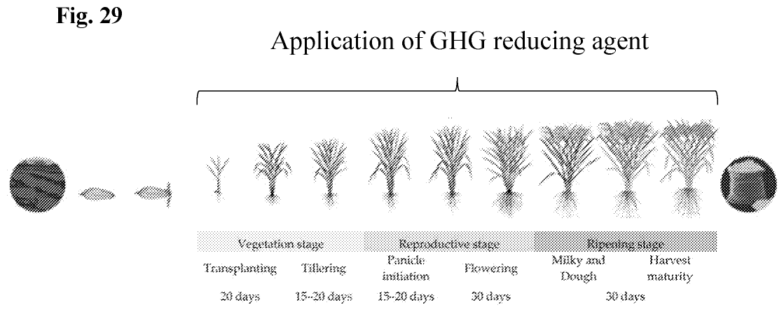

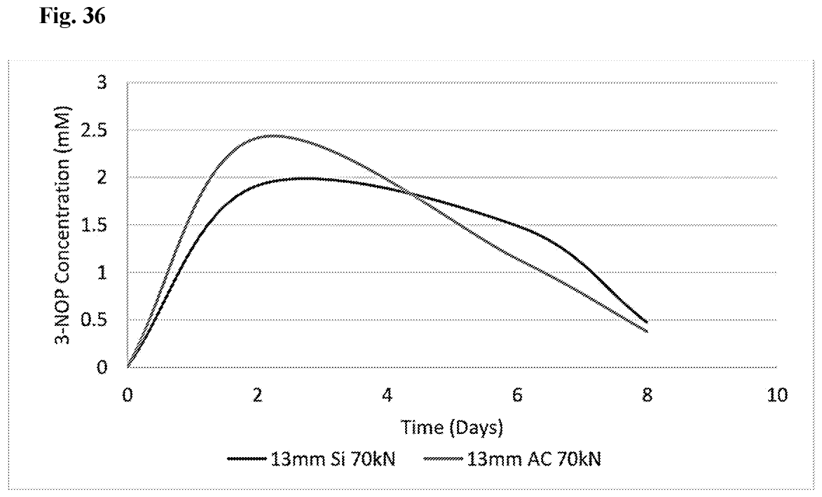

After 24 hr in water, less than 5 wt % 3-NOP remained adsorbed to silica compared to the powder mix placed in acetonitrile. After 24 hr in water, ˜35 wt % 3-NOP remained adsorbed to activated carbon compared to the powder mix placed in acetonitrile. By washing the activated carbon in acetonitrile, the adsorbed 3-NOP can be recovered in solution proving that the remaining 30-50% 3-NOP that is seen in FIG. 27-FIG. 30 remains adsorbed. Further studies need to be conducted to determine the driving forces for 3-NOP desorption from activated carbon under different environmental conditions to utilize adsorptive capacity of activated carbon. This study also confirms that from previous tablet formulations, diffusion is the primary driver for 3-NOP release of unbound 3-NOP. This can be seen by very similar release profiles between silica-based adsorbent tablets and activated carbon-based adsorbent tablets. FIG. 36 compares a silica-based tablet and activated carbon-based tablet over 8 days. Small differences in release profiles could be due to differences in porosity between tablets using silica or activated carbon.

Compounding of 3-NOP

3-NOP was compounded with a variety of matrix materials using a DSM Xplore MC15 conical twin-screw extruder. The extrudate was pelletized and used as is or further compression molded into tablets as a suitable 3-NOP extended-release form.

3-NOP was adsorbed onto the corresponding adsorbent and speed mixed with binder prior to compounding is a DSM Xplore MC15 conical twin-screw extruder at 100C and 50 rpm. The strand was then pelletized, heated, and molded into a 13 mm disc-shaped tablet using a carver press under 40 kN of compaction force. Formula consists of 8.3% 3-NOP, 25.0% arginine, 66.7% CAPA 6800.

TABLE 34
3-NOP release profile of compounded tablets over 24
days. Total concentration of 3-NOP is listed in mM.

| Time | 1 | 3 | 7 | 10 | 14 | 20 | 24 |
|---|---|---|---|---|---|---|---|
| Arg R1 | 8.97 | 16.37 | 22.55 | 23.42 | 24.11 | 23.07 | 24.56 |
| Arg R2 | 12.49 | 15.85 | 20.43 | 21.45 | 21.60 | 19.33 | 19.97 |
| Arg R3 | 9.90 | 17.78 | 24.52 | 24.50 | 24.36 | 22.60 | 22.33 |
| AVG | 10.45 | 16.67 | 22.50 | 23.13 | 23.36 | 21.67 | 22.29 |
| STD | 1.82 | 1.00 | 2.04 | 1.55 | 1.52 | 2.04 | 2.29 |

Formulas consists of 11.1% 3-NOP, 13.9% silica, 75.0% CAPA 6800 denoted “11PCL” and 19.4% 3-NOP, 24.2% silica, 56.5% CAPA 6800 denoted “19PCL.”

TABLE 35
3-NOP release profile of compounded tablets over 24 days.
Total concentration of 3-NOP is listed in mM.

| Time | 1 | 3 | 7 | 13 | 17 |
|---|---|---|---|---|---|
| 11PCL R1 | 9.79 | 18.77 | 24.31 | 29.15 | 27.93 |
| 11PCL R2 | 10.34 | 19.81 | 24.99 | 30.02 | 29.49 |
| 11PCL R3 | | 19.05 | 22.51 | 27.14 | 26.95 |
| AVG | 10.06 | 19.21 | 23.94 | 28.77 | 28.12 |
| STD | 0.39 | 0.54 | 1.28 | 1.48 | 1.28 |
| 19PCL R1 | 23.55 | 40.56 | 49.99 | 55.00 | 3.48 |
| 19PCL R2 | 24.19 | 40.59 | 51.03 | 53.46 | 51.64 |
| 19PCL R3 | 25.05 | 42.76 | 52.21 | 57.93 | 55.77 |
| AVG | 24.26 | 41.30 | 51.08 | 55.47 | 36.96 |
| STD | 0.75 | 1.26 | 1.11 | 2.27 | 29.07 |

Compounded Polyelectrolyte Complexes for Extended Release of 3-NOP

Solid polyelectrolyte complexes can be processible when plasticized with aqueous electrolytes to act as a matrix for 3-NOP molecules. An example of a polyelectrolyte pair for compounding is polydiallyldimethylammonium chloride (PDADMAC), as a cationic polyelectrolyte, and polystyrene sulfonate (PSS), as an anionic polyelectrolyte. At no salt conditions, the complex is brittle and not ideal for processing and the addition of salt, such as sodium chloride, can help with the plasticization and further processing. A trial formulation used for compounding includes mixing of equal charge stoichiometry of PDADMAC and PSS solutions with a concentration of 100 mM, with respect to each polymer, and a total volume of 200 mL (as per preparation method described in ACS Appl. Mater. Interfaces 2015, 7, 895-901). The polyelectrolyte complex can be separated, removed from the supernatant phase, and soaked in salt water with a concentration of 1M for 24 h. The polyelectrolyte complex should be decanted and ready for compounding. A typical formulation used for compounding is summarized in Table 34.

TABLE 36
Compounding formulation with polyelectrolyte
complexes for extended release of
3-NOP. Total materials used 20 g.
3-NOP was mixed with arginine
(as in solid powder) overnight at 30 C.
PEC Compounding Formulation

| | Part per | | Part per |
|---|---|---|---|
| 3-NOP | formulation | | formulation |
| Complex | (wt %) | PEC Complex | (wt %) |
| 3-NOP: | 25 | PDADMAC: | 75 |
| Arginine | | PSS (Equimolar | |
| (ratio 1:3, | | with respect to | |
| wt %) | | the monomer) | |

Table 37 shows release (%) of 3-NOP out of compounded PEC formulated as per Table 27. The compounded PECs were cut into 5-10 mm pellets and soaked in water (10 mL) for the release study.

TABLE 37
Release (%)

| Day 1 | Day 4 | Day 6 | Day 12 |
|---|---|---|---|
| 22.4 | 34.4 | 35 | 35.7 |

Compounded Pellets

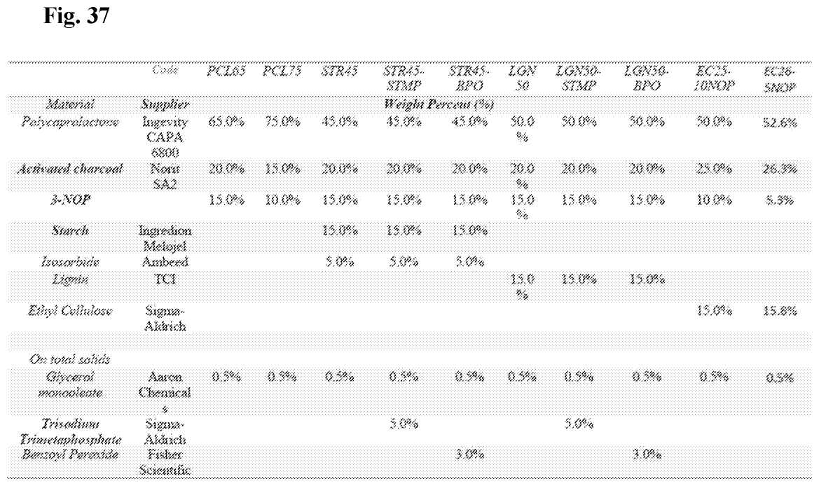

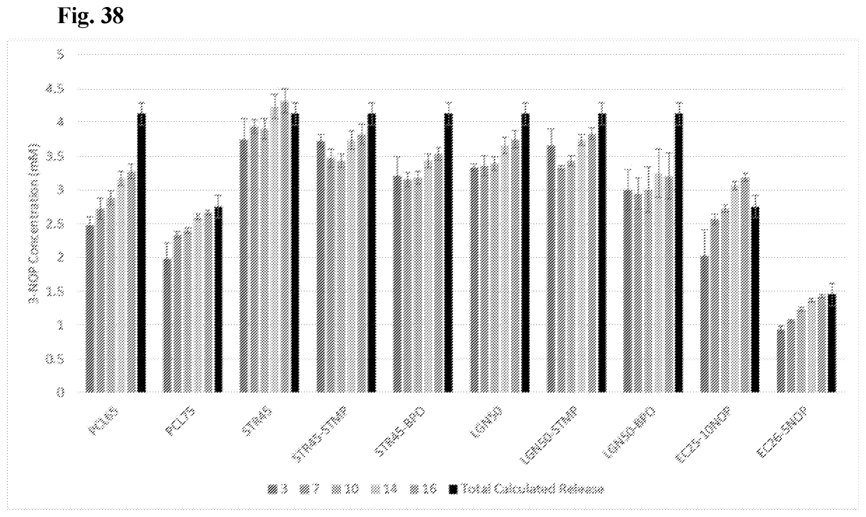

3-NOP was adsorbed onto the corresponding adsorbent and speed mixed with binder prior to compounding is a DSM Xplore MC15 conical twin-screw extruder at temperatures of 110-130C and 100 rpm. The extrudate was then pelletized forming ˜2 mm pellets. Material suppliers are listed in Table 36. Polybutylene succinate grades FZ71, FZ91, FD92 were supplied by Mitsubishi Chemical. Polycaprolactone-based formulations are summarized in the Table in FIG. 37. FIG. 38 shows 3-NOP release in mM for polycaprolactone-based formulations.

TABLE 39
Polybutylene succinate-based formulations

| | | | | |
|---|---|---|---|---|
| Code | 65FZ71 | 65FZ91 | 65FD92a | 65FD92b |

Material

| | | | | |
|---|---|---|---|---|
| Polybutylene succinate | 65.0% | 65.0% | 65.0% | 65.0% |
| Activated charcoal | 20.0% | 20.0% | 20.0% | 20.0% |
| 3-NOP | 15.0% | 15.0% | 15.0% | 15.0% |

On total solids

| | | | | |
|---|---|---|---|---|
| Glycerol monooleate | 0.5% | 0.5% | 0.5% | 0.0% |
| Compounding | 120 C. | 120 C. | 130 C. | 110 C. |
| Temperature | | | | |

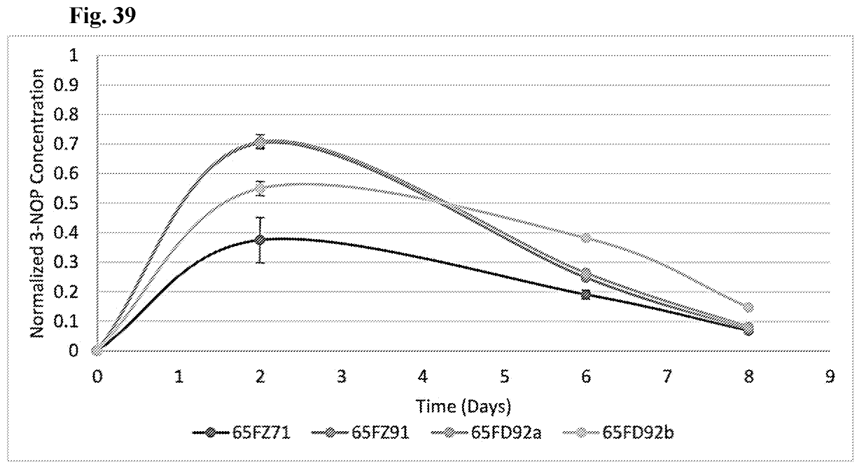

FIG. 39 is chart showing the normalized 3-NOP concentration v. Release time in days of polybutylene succinate-based formulations. Lower 3-NOP loadings (<10%) have a less pronounced burst release over the first 7 days of monitored release. Glycerol monooleate was identified at a potent processing aid at loadings <1%. High levels <25 wt % activated carbon can easily be processed unlike silica which leads to high viscosity and screw torque. Cross-linked systems show slower release in comparison to un-crosslinked systems. High shear conditions of compounding appear to destroy activated carbon structure and adsorption capacity is lost.

Microcapsules for the Encapsulation of 3-NOP

Polyelectrolyte complexes (PECs) are used to form microcapsules and encapsulate 3-NOP. PECs are formed through attractive forces such as ionic interactions, hydrogen bonding, hydrophobic, and pi-interactions between the polyelectrolytes. Polyelectrolytes are polymers with ionic groups bonded to counter ions and can dissociate in a solution to make positively or negatively charged polymers. Herein, PEC microcapsules refer to interconnecting networks of polyelectrolytes formed upon the interaction between oppositely charged polymers.

Examples of polyelectrolytes used in this invention include synthetic polyelectrolytes such as polystyrene sulfonate (PSS), polyacrylic acid (PAA), and polyallylamine hydrochloride (PAH). Further examples include naturally occurring polyelectrolytes such as chitosan and alginate or ionic biopolymers such as proteins, enzymes, and charged polypeptides.

Examples of Polyelectrolytes (charged polymers with positively or negatively charged repeating units) Used:

Synthetic Polyelectrolytes

Polystyrene sulfonate (PSS), molecular weight ˜200,000 Da, Sigma Aldrich

Sodium Lignosulfonate (SLS), molecular weight ˜52000 Da, Sigma Aldrich

Polyethyleneimine (PEI), molecular weight ˜2000 Da, Polysciences

Polyallylamine hydrochloride (PAH), molecular weight ˜17,500 Da, Sigma Aldrich

Commercially Available Peptide Sequences

Poly (L-lysine) (PLK10), degree of polymerization:10, molecular weight: 1600 Da, Alamanda Polymers

Poly (L-lysine) (PLK20), degree of polymerization: 20, molecular weight: 3300 Da, Alamanda Polymers

Poly (L-arginine) (PLR10), degree of polymerization: 10, molecular weight: 1900 Da, Alamanda Polymers

Examples of Crosslinker Used:

Glutaraldehyde, 25% solution in water, Sigma Aldrich

EDC (1-ethyl-3-(3-dimethylaminopropyl) carbodiimide hydrochloride), Thermo Fisher.

PEC Microcapsules Preparation

Equal stoichiometry of oppositely charged polyelectrolytes is sequentially added to water or water-3-NOP solutions followed by vortexing for 10 s, after the addition of each component, to form PEC microcapsules. Some samples are chemically crosslinked (glutaraldehyde is an example of the crosslinker used). The PEC microcapsules may also be formed with non-stoichiometric ratios of polyelectrolytes. While stoichiometric ratios of polyelectrolytes provide almost a neutral microcapsule, microcapsules prepared with non-stoichiometric ratios are positively or negatively charged. All polyelectrolyte solutions are prepared in water and pH adjusted. The concentration of solutions is based on the monomer charge. The stock solution of 3-NOP is prepared in water with a concentration of 100 mM and pH adjusted to 8.

High-performance liquid chromatography (HPLC) is used to determine the concentration of 3-NOP in PEC microcapsules and calculate the release of 3-NOP from the microcapsules. Samples are centrifuged to separate the supernatant phase from the complex phase. The supernatant phase is then removed carefully by using a micropipette and transferred to a 2 mL glass vial for further analysis. The 3-NOP release of some PEC microcapsules prepared at neutral pH conditions and after 96 h of preparation is summarized in Table 38.

TABLE 40
3-NOP release (%) from the PEC microcapsules.
Samples are prepared with a 3-NOP concentration
of 100 μM. Final pH solution ~7. K, L, F, and E refer
to lysine, leucine, phenylalanine, and glutamic acid,
respectively. PSS refers to polystyrene sulfonate.

| | | 3-NOP |
|---|---|---|
| | Release | Concentration in |
| Polyelectrolyte | (%) at 96 h | Microcapsules |
| Pair | timepoint | (μM) |
| (KKLF)3 + (EELF)3 | 63 | 37 |
| (KKLF)3 + PSS | 64 | 36 |
| (kKIF)3 + PSS | 66 | 34 |
| (kKIF)3 + (EELF)3 | 70 | 30 |
| (crosslinked) | | |
| (kKIF)3 + PSS | 72 | 28 |
| (crosslinked) | | |

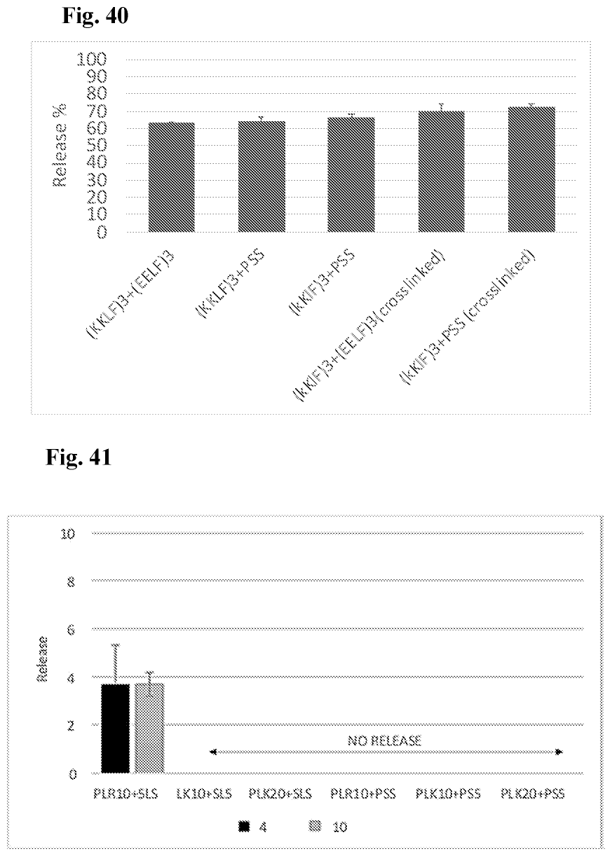

FIG. 40 is a graph showing 3-NOP release (%) from the PEC microcapsules. Samples are prepared with a 3-NOP concentration of 100 μM. Final pH solution ˜7. K, L,F, and E refer to lysine, leucine, phenylalanine, and glutamic acid, respectively. PSS refers to polystyrene sulfonate.

Examples of release profiles of some microcapsules made of positively charged polypeptides with either polystyrene sulfonate (PSS) or sodium lignosulfonate (SLS) are shown in FIG. 41 (3-NOP release (%) from the PEC microcapsules. Samples are prepared with a 3-NOP concentration of 100 μM. Final pH solution ˜7. PLR refers to poly (L-arginine), and PLK refers to poly (L-lysine). SLS and PSS refer to sodium lignosulfonate and polystyrene sulfonate).

The encapsulation efficiency of some PEC microcapsules prepared at neutral pH conditions is summarized in Table 41. Table 42 shows the encapsulation efficiency of samples prepared at pH-8.

TABLE 41
Encapsulation efficiency of the PEC microcapsules.
Samples are prepared with a 3-NOP
concentration of 100 μM. Final pH solution ~7.
Upper and lower cases in (kKIF)3 sequences
represent L and D-chirality, respectively.

| | Total | | |
|---|---|---|---|
| | Concentration | | |
| | (monomer | | 3-NOP |
| | charge | Encapsulation | Concentration in |
| Polyelectrolyte | basis) | Efficiency | Microcapsules |
| Pair | (mM) | (%) | (μM) |
| PLK20 + PRE20 | 5 | 7.4 | 7.4 |
| PLR10 + PRE20 | 5 | 0 | 0 |
| (KKLF)3 + (EELF)3 | 5 | 100 | 100 |
| (kKIF)3 + (EELF)3 | 5 | 22.2 | 22.2 |
| (KKLF)3 + PSS | 5 | 37 | 37 |
| (kKIF)3 + PSS | 5 | 100 | 100 |
| PAH + PSS | 5 | 30.9 | 30.9 |

TABLE 42
Encapsulation efficiency of the PEC microcapsules.
Samples are prepared with a 3-NOP
concentration of 2.5 mM. Final pH solution ~8.
Upper and lower cases in (kKIF)3
sequences represent L and D-chirality
of the amino acid residue, respectively.

| | Total | | |
|---|---|---|---|
| | Concentration | | 3-NOP |
| | (monomer | | Concentration |
| | charge | Encapsulation | in |
| Polyelectrolyte | basis) | Efficiency | Microcapsules |
| Pair | (mM) | (%) | (mM) |
| PLR10 + PRE20 | 5 | 50 | 1.25 |
| PLK20 + PRE20 | 5 | 53.8 | 1.34 |
| (kKIF)3 + PRE20 | 5 | 50.2 | 1.26 |

Physical Crosslinking of 3-NOP with Small Molecules

3-NOP molecules can form hydrogen bonds with small molecules such as amino acids to make a larger size conjugate resulting in enhanced encapsulation efficiency. For example, an equal stoichiometry of 3-NOP and two selective amino acids, arginine and lysine, were reacted in aqueous solution for 24 h, and the solution was used for encapsulation studies. The results of such encapsulation with some PEC microcapsules are shown in Table 43.

TABLE 43
Encapsulation efficiency of the PEC microcapsules.
Samples are prepared with a 3-NOP
concentration of 125 μM. Final pH solution ~7.

| | Encapsulation | | 3-NOP | |
|---|---|---|---|---|
| | Efficiency | | Concentration in | |
| | (%) | | Microcapsules (μM) | |
| Polyelectrolyte | Arginine- | Lysine- | Arginine- | Lysine- |
| Pair | 3-NOP | 3-NOP | 3-NOP | 3-NOP |
| PLR10 + PRE20 | 4.6 | 19.1 | 5.75 | 23.9 |
| PLK20 + PRE20 | 34.3 | 28.4 | 42.9 | 35.5 |
| (KKLF)3 + (EELF)3 | 27.4 | 34.8 | 34.25 | 43.5 |
| PAH + PAA | 30.3 | 47.1 | 37.9 | 58.9 |
| PAH + PSS | 67.4 | 67.6 | 84.3 | 84.5 |

Ranges

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1-202. (canceled)

203. A vaccine comprising at least one ribonucleic acid encoding at least one cell surface protein or a fragment thereof of at least one methanogen.

204. The vaccine of claim 203, wherein the at least one methanogen is selected from Methanobrevibacter ruminantium, Methanobrevibacter gottschalkii, Methanobacterium formicicum, Methanobrevibacter smithii, Methanobrevibacter sp. AbM4, and Methanosarcina mazei.

205. The vaccine of claim 203, wherein the at least one methanogen is Methanobrevibacter ruminantium.

206. The vaccine of claim 203, wherein the at least one methanogen is Methanobrevibacter gottschalkii.

207. The vaccine of claim 203, wherein the vaccine is monovalent.

208. The vaccine of claim 203, wherein the vaccine is multivalent.

209. The vaccine of claim 208, wherein the vaccine comprises at least one ribonucleic acid encoding at least one cell surface protein or a fragment thereof of Methanobrevibacter ruminantium and Methanobrevibacter gottschalkii.

210. The vaccine of claim 203, wherein the at least one cell surface protein or a fragment thereof comprises at least 90% sequence identity to the amino acid sequence set forth in Table 2A, Table 2B, Table 3, Table 19, Table 20, Table 21, or a fragment thereof.

211. The vaccine of claim 203, wherein the at least one ribonucleic acid comprises at least 90% sequence identity to the nucleotide sequence set forth in Table 2A, Table 2B, Table 3, Table 19, Table 20, Table 21, or a fragment thereof.

212. The vaccine of claim 203, wherein the at least one ribonucleic acid is codon-optimized for expression in a ruminant.

213. The vaccine of claim 212, wherein the at least one ribonucleic acid is codon-optimized for expression in Bos taurus.

214. The vaccine of claim 203, wherein the ribonucleic acid comprises the 5′ cap with at least one chemical modification.

215. The vaccine of claim 214, wherein the 5′ cap is selected from:
(a) (Cap 0) m7G(5′)pppN1pN2p;
(b) (Cap 1) m7G(5′)pppN1mpNp;
(c) (Cap 2) m7G(5′)pppN1mpN2mp;
(d) (Cap 4) m7Gpppm(3)(6,6,2′)Apm(2′)Apm(2′)Cpm(2)(3,2′)Up; and
(e) Anti-reverse Cap Analog (ARCA).

216. The vaccine of claim 203, wherein the at least one ribonucleic acid comprises 5′ UTR, 3′ UTR, and/or a polyA tail.

217. The vaccine of claim 216, wherein
(a) the sequence of the 5′ UTR is selected from those listed in Table 4A; and/or
(b) the sequence of the 3′ UTR is selected from those listed in Table 4B.

218. The vaccine of claim 203, wherein the at least one ribonucleic acid comprises a sequence encoding a signal peptide.

219. The vaccine of claim 218, wherein the sequence encodes a signal peptide selected from those listed in Table 5A and Table 5C.

220. The vaccine of claim 203, wherein the at least one ribonucleic acid comprises a sequence encoding a transmembrane domain and/or a cytoplasmic domain.

221. The vaccine of claim 220, wherein the sequence encodes a transmembrane domain and/or a cytoplasmic domain selected from those listed in Table 5B and Table 5C.

222. The vaccine of claim 203, wherein the at least one ribonucleic acid comprises:
(a) a 5′ cap, a 5′ UTR, a sequence encoding at least one cell surface protein or a fragment of at least one methanogen, a 3′ UTR, and a polyA tail;
(b) a 5′ cap, a 5′ UTR, a sequence encoding a signal peptide, a sequence encoding at least one cell surface protein or a fragment thereof of at least one methanogen, a 3′ UTR, and a poly A tail; or
(c) a 5′ cap, a 5′ UTR, a sequence encoding a signal peptide, a sequence encoding at least one cell surface protein or a fragment thereof of at least one methanogen, a sequence encoding a transmembrane domain, optionally further comprising a sequence encoding a cytoplasmic domain, 3′ UTR, and a poly A tail.

223. The vaccine of claim 203, wherein the at least one ribonucleic acid comprises at least one chemical modification in a uracil, optionally wherein the chemical modification is in at least 75% of the uracils in the sequence encoding at least one cell surface protein or a fragment thereof of at least one methanogen.

224. The vaccine of claim 223, wherein the at least one chemical modification is selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, l-methyl-6-thio-guanosine, N2-methyl-6-thio-guanosine, and N2,N2-dimethyl-6-thio-guanosine, and any combination thereof.

225. The vaccine of claim 223, wherein the at least one chemical modification comprises N1-methyl pseudouridine.

226. The vaccine of claim 203, wherein the vaccine is formulated in lipid.

227. The vaccine of claim 226, wherein the vaccine is formulated in a liposome, a lipoplex, or a lipid nanoparticle.

228. The vaccine of claim 227, wherein the vaccine is formulated in a lipid nanoparticle comprising an ionizable lipid, a helper lipid, a PEGylated lipid, a structural lipid, or any combination thereof.

229. The vaccine of claim 227, wherein the nanoparticle has:
(a) a mean diameter of about 50 nm to about 200 nm, optionally about 80 nm to about 100 nm;
(b) a polydispersity index (PDI) of less than 0.4; and/or
(c) a net neutral charge at a neutral pH.

230. A method of inducing an immune response against at least one methanogen in a ruminant, the method comprising administering to the ruminant the vaccine of claim 203.

231. A method of reducing the activity, number, and/or type of methanogens in the gut of a ruminant, the method comprising administering to the ruminant the vaccine of claim 203.

232. A method of reducing the amount of methane produced by a ruminant, the method comprising administering to the ruminant the vaccine of claim 203.
